(19) United States
(12) Patent Application Publication
(10) Pub. No.: US 2025/0099442 A1
(43) Pub. Date: Mar. 27, 2025

(54) COMPOUNDS FOR USE IN TREATING NEUROLOGICAL DISORDERS

(71) Applicant: Prothena Biosciences Limited, Dublin (IE)

(72) Inventor: Stéphane DE LOMBAERT, Chester, CT (US)

(21) Appl. No.: 18/717,618

(22) PCT Filed: Dec. 9, 2022
(86) PCT No.: PCT/US2022/052451

§ 371 Date: Jun. 7, 2024

Related U.S. Application Data
(60) Provisional application No. 63/288,176, filed on Dec. 10, 2021.
(60) Provisional application No. 63/288,193, filed on Dec. 10, 2021.

(51) Int. Cl. A61K 31/437; A61K 31/443; A61K 31/4545; A61K 31/4985; C07D 307/81; C07D 401/12; C07D 417/14; C07D 471/04; C07D 487/04; C07D 491/048; C07D 513/04

(52) U.S. Cl. CPC A61K 31/437; A61K 31/443; A61K 31/4545; A61K 31/4985; C07D 307/81; C07D 401/12; C07D 417/14; C07D 471/04; C07D 487/04; C07D 491/048; C07D 513/04

(57) ABSTRACT

This disclosure provides compounds of formula (I) and pharmaceutically acceptable salts thereof, that inhibit Dual specificity tyrosine-phosphorylation-regulated kinase 1A (DYRK1A). These chemical entities are useful, e.g., for treating a condition, disease or disorder in which increased (e.g., excessive) DYRK1A activation contributes to the pathology and/or symptoms and/or progression of the condition, disease or disorder (e.g., a neurological disorder) in a subject (e.g., a human). This disclosure also provides compositions containing the same as well as methods of using and making the same.

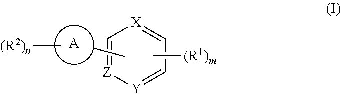

### TECHNICAL FIELD

This disclosure provides compounds and pharmaceutically acceptable salts thereof, that inhibit Dual specificity tyrosine-phosphorylation-regulated kinase 1A (DYRK1A). These compounds are useful, e.g., for treating a condition, disease or disorder in which increased (e.g., excessive) DYRK1A activation contributes to the pathology and/or symptoms and/or progression of the condition, disease or disorder (e.g., a neurological disorder in a subject (e.g., a human). This disclosure also provides compositions containing the same as well as methods of using and making the same.

### BACKGROUND

Dual-specificity tyrosine phosphorylation-regulated kinase 1A (DYRK1A) is a 763 amino acid, 85 kDa serine/threonine/tyrosine kinase located on chromosome 21 (21q22.2). DYRK1A possesses catalytic activity that is regulated by autophosphorylation of a tyrosine residue (Y321) which results in constitutively active serine/threonine kinase activity. See Abbassi, et al., Pharmacology & Therapeutics, 151, 87-98 (2015). Since DYRK1A is constitutively active, its activity is dosage dependent. Thus, both elevated levels and depressed levels of DYRK1A, (relative to wild-type levels) have been shown to lead to neurological impairment. See Duchon and Herault, Front Behav. Neurosci. 10, 104-104 (2016). DYRK1A is also a member of a large family of CMGC kinases, which include cyclin-dependent kinases (CDKs), mitogen-activated protein kinases (MAPKs), glycogen synthase kinases (GSKs), and CDC-like kinases (CLKs).

DYRK1A additionally has been shown to have a role in cell cycle regulation, at least in part by phosphorylating (and thus inhibiting) the nuclear factor of activated T cells (NFAT) family of transcription factors. Additionally, over 20 substrates of DYRK1A have been identified, including cell signaling, chromatin modulation, gene expression, alternative splicing, cytoskeletal, and synaptic function. See Abassi, et al, (2016). DYRK1A dysregulation is implicated in various disease states such as Alzheimer's disease, autism, and Down syndrome. In some cases, novel mutations in DYRK1A have been associated with autism phenotypes. See e.g., Dang, et al., Molecular Psychiatry, 23, 747-758 (2018).

DYRK1A is also known to play an important role in brain development. For example, reduced DYRK1A activity (such has having a single copy of loss of function mutation) during neural development results in intellectual disability phenotypes. Conversely, trisomy 21 in Down syndrome individuals is associated with a triplication of the DYRK1A gene, which results in elevated DYRK1A activity. DYRK1A is located on chromosome 21, specifically within the “Down syndrome critical region” a portion of chromosome 21 that includes genes particularly relevant for developing Down syndrome phenotypes. As a result, individuals with Down syndrome have three copies of DYRK1A, and since DYRK1A is dosage sensitive, the elevated levels of DYRK1A in such individuals markedly affects the localization and function of the DYRK1A protein. The expression of DYRK1A is also elevated in the CNS in individuals with neurodegenerative diseases, such as Parkinson's disease, Pick's disease, and Alzheimer's disease.

Moreover, approximately 50% of individuals with Down syndrome ultimately develop Alzheimer's disease, with symptoms generally beginning between the ages of 40 and 60. DYRK1A phosphorylates amyloid precursor protein (APP) which promotes the production of pathogenic amyloid-β peptide (Aβ). Dyrk1A also phosphorylates tau both directly and indirectly (see Abassi, et al, (2016)). Both amyloid-β and tau pathologies are associated with Down syndrome phenotypes.

Normalization of DYRK1A gene dosage by crossing Ts65Dn mice (DS model) with DYRK1A knockout mice reverses many Alzheimer's-like phenotypes. See Garcia-Cerro et al., 2017. In individuals with Down Syndrome, DYRK1A mRNA levels, protein levels, and kinase activity are increased by ˜50%, reflecting the number of gene copies. See Liu et al., 2008; see also Wegiel et al., 2011.

Because no treatment is available for these neurological disorders, the prognosis for individuals with, for example, Alzheimer's disease is poor. This can be particularly devastating because Alzheimer's disease is responsible for a sharp decline in survival in individuals with Down syndrome that are over 45 years old. Only about 25% of those with Down syndrome live more than 60 years, and most of those have developed Alzheimer's disease.

Across all individuals, dementia remains a significant leading unmet medical need and a costly burden on public health. Currently, 1 in 3 seniors develops dementia, and about 70% of dementia cases are attributed to Alzheimer's disease. Some 11% of Americans over age 65 has AD, which constitutes over 6.2 million in 2021. This figure is projected to exceed 12 million in 2050 (www.Alz.org).

Presently, no therapies have been approved to treat Alzheimer's disease associated with Down syndrome, which represents a significant unmet medical need. Some DYRK1A inhibitors have been tested in vitro or in animal preclinical models to treat Alzheimer's disease or Down syndrome, however, since DYRK1A is a member of the highly conserved CMGC family of kinases, identifying compounds that selectively target DYRK1A has proved challenging. Thus, there remains a need to identify DYRK1A inhibitors to treat Down syndrome, Alzheimer's disease, Alzheimer's disease associated with Down syndrome, and other neurodegenerative and neurological diseases.

### SUMMARY

Some embodiments provide a compound of Formula (I):

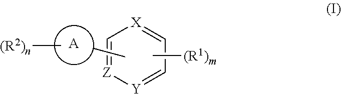

or a pharmaceutically acceptable salt thereof, wherein:
X is CH, N, N—O, or

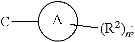

Y is CH, N, or

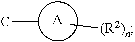

Z is CH, N, or CR¹;
wherein Formula (I) contains one Ring A and not more than two of X, Y, and Z are N or N—O;
each R¹ is independently hydroxyl, cyano, C1-C6 alkyl,

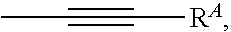

—NHC(═O)R^{A}, C1-C6 alkoxy, —(CH₂)ₚ—NR^{B}R^{C}, C3-C10 cycloalkyloxy optionally substituted with amino, -Q-(CH₂)_{q}—R^{D}, —CO₂R^{B}, —C(═O)NR^{B}R^{C}, —C(═O)-3-6 membered heterocyclyl, or phenoxy;
Ring A is a fused bicyclic 9 membered cycloalkyl, a fused bicyclic 9-10 membered heteroaryl, a 9 membered fused bicyclic heterocyclyl, or a fused tricyclic 13-14 membered heterocyclyl;
each R² is independently halogen, C1-C6 haloalkyl, C1-C6 alkyl optionally substituted with 1-2 substituents independently selected from hydroxyl, C1-C6 alkoxy, and 5-10 membered heteroaryl, —(CH₂)ₜ-Q¹-4-6 membered heterocyclyl optionally substituted with C1-C6 alkyl or —CO₂H, 4-9 membered heterocyclyl optionally substituted with R^{G}, —NHC(═O)R^{E},

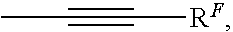

C1-C6 alkoxy, —C(═O)NHR^{H}, phenyl optionally substituted with 1-2 substituents independently selected from hydroxyl and cyano, 5-10 membered heteroaryl optionally substituted with C1-C6 alkyl, —NHSO₂(C1-C6 alkyl), C3-C9 cycloalkyl optionally substituted with hydroxyl or —NR^{B2}R^{C2}, —NR^{B1}R^{C1}, —C(═O)-3-6 membered heterocyclyl optionally substituted with C1-C6 alkyl, or benzyl optionally substituted with C1-C6 alkoxy;
each R^{A} and R^{F} is independently C3-C6 cycloalkyl or 3-6 membered heterocyclyl optionally substituted with 1-2 substituents independently selected from hydroxyl and C1-C6 alkyl;
each R^{B}, R^{B1}, R^{B2}, R^{C}, R^{C1}, and R^{C2} is independently hydrogen or C1-C6 alkyl optionally substituted with hydroxyl;
each R^{D} is independently a 4-6 membered heterocyclyl optionally substituted with C1-C6 alkyl or a phenyl optionally substituted with 1-2 independently selected halogen;
each R^{E} is independently 3-10 membered heterocyclyl optionally substituted with 1-4 independently selected C1-C6 alkyl or a —(CH₂)ᵥphenyl optionally substituted with cyano or halogen;
R^{G} is C1-C6 alkyl, —SO₂(R^{G1}), —C(═O)(C1-C6 alkyl), —C(═O)O—Benzyl, 4-6 membered heterocyclyl optionally substituted with 1-2 substituents independently selected from C1-C6 alkyl, and C3-C6 cycloalkyl optionally substituted with —CO₂H;
R^{G1} is C1-C6 alkyl or phenyl optionally substituted with cyano or halogen;
R^{H} is hydrogen, C1-C6 alkyl, or benzyl optionally substituted with halogen;
Q and Q¹ are independently 0, NH, or a bond;
m, n, p, and t are each independently 0, 1, 2, or 3;
v is 0 or 1; and
q is 1, 2, or 3.

Some embodiments provide a compound of Formula (II):

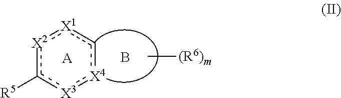

or a pharmaceutically acceptable salt thereof, wherein:
Ring A is aromatic;
Ring B is phenyl, 5-6 membered heteroaryl, or 5-7 membered monocyclic heterocyclyl such that together Ring A and Ring B form a 9-10 membered heteroaryl or a 9-10 membered heterocyclyl ring system;
X¹ is absent, CR¹, N, or NR^{A};
X² is CR², C═O, N, or NR, wherein when X² is C═O, X¹ is NR^{A}, and when X¹ is absent, X² is directly connected to the carbon atom shared by Ring A and Ring B;
X³ is C, CR³ or N;
is C or N;
one of R¹, R², R³, and R⁵ is halogen, cyano, C1-C6 haloalkyl, C1-C6 alkyl optionally substituted with 1-2 substituents independently selected from (i) 5-6 membered heteroaryl optionally substituted with 1-2 independently selected C1-C6 alkyl or 3-6 membered heterocyclyl optionally substituted with C1-C6 alkyl, (ii) 4-6 membered heterocyclyl optionally substituted with benzyl, (iii) cyano, (iv) phenyl optionally substituted with halogen, or (v) —NR^{E}R^{F}; C1-C6 alkoxy, —(CH₂)ₙ-Q-(4-6 membered heterocyclyl optionally substituted with C1-C6 alkyl), —NHC(═O)(CH₂)ₙR^{C}, 4-10 membered heterocyclyl optionally substituted with phenoxy, C1-C6 alkyl, or 5-6 membered heteroaryl; —NR^{E}R^{F}, C3-C6 cycloalkyl, C3-C6 cycloalkyloxy,

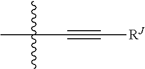

phenoxy optionally substituted with 1-2 substituents independently selected from halogen and C1-C6 haloalkyl; 5-6 membered heteroaryloxy optionally substituted with 1-2 independently selected C1-C6 alkyl, 5-10 membered heteroaryl optionally substituted with C1-C6 alkyl, —SO₂(C1-C6 alkyl), —(CH₂)ₙCO₂R^{D};
and the other of R¹, R², R³, and R⁵ are independently hydrogen, halogen, cyano, C1-C6 alkyl, C1-C6 alkoxy, C1-C6 haloalkyl, C3-C6 cycloalkyloxy, or 4-6 membered heterocyclyl optionally substituted with 5-6 membered heteroaryl; or
R³ and R⁵, together with the carbon atoms to which they are attached form a 6 membered heterocyclyl optionally substituted with C1-C6 alkyl or C3-C6 cycloalkyl; or
R³ and one R⁶ adjacent to Ring A, together with the carbon atoms to which they are attached form a 7 membered heterocyclyl; or
R¹ and one R⁶ adjacent to Ring A, together with the carbon atoms to which they are attached form a 7 membered heterocyclyl; or
R¹ and R², R² and R³, and R³ and R⁵, together with the carbon atoms to which they are attached form a C3-C5 cycloalkyl;
Q and Q¹ are each independently —O— or —C(═O)—;
R^{A} and R^{B} are independently hydrogen, C3-C6 cycloalkyl, or C1-C6 alkyl;
R^{C} is 4-6 membered heterocyclyl optionally substituted with C1-C6 alkyl, or 5-6 membered heteroaryl optionally substituted with C1-C6 alkoxy or C1-C6 alkyl,
R^{D} is hydrogen or C1-C6 alkyl;
R^{E} and R^{F} are independently hydrogen, C1-C6 alkyl, —C(═O)—C1-C6 alkyl, or 4-6 membered heterocyclyl;
R^{G} and R^{H} are independently hydrogen, C1-C6 alkyl, or C3-C6 cycloalkyl;
R^{I} is 5-6 membered heteroaryl optionally substituted with C1-C6 alkyl or halogen; C1-C6 alkyl substituted with 1-2 independently selected amino or phenyl optionally substituted with halogen; or phenyl optionally substituted with halogen;
R^{J} is a 4-6 membered heterocyclyl or a 5-6 membered heteroaryl;
R⁶ is C1-C6 alkyl optionally substituted with (i) 4-10 membered heterocyclyl optionally substituted C1-C6 alkyl or (ii) 9-10 membered heteroaryl; C1-C6 haloalkyl, C3-C6 cycloalkyl optionally substituted with —CO₂R^{D}, —NHC(═O)R^{C}, —NR^{G}R^{H}, 4-10 membered heterocyclyl optionally substituted C1-C6 alkyl, —(CH₂)ₚC(═O)NHR^{I}, 5-10 membered heteroaryl optionally substituted with C1-C6 alkyl, or —(CH₂)ₛ-Q¹-(4-6 membered heterocyclyl optionally substituted with C1-C6 alkyl); or
two geminal R⁶, together with the carbon atom to which they are attached form a C3-C6 spirocycloalkyl;
m is 0, 1, 2, or 3;
n is 0, 1, or 2;
p is 0, 1, 2, 3, or 4; and
s is 0, 1, or 2.

Also provided herein is a pharmaceutical composition comprising a compound of Formula (I) or (II), or a pharmaceutically acceptable salt of any of the foregoing, and a pharmaceutically acceptable carrier.

Provided herein is a method for treating a neurological disorder in a subject in need thereof, the method comprising administering to the subject a therapeutically effective amount of a compound of Formula (I) or (II), or a pharmaceutically acceptable salt of any of the foregoing, or a pharmaceutical composition as provided herein.

Also provided herein is a method for treating a neurological disorder in a subject in need thereof, the method comprising (a) determining that the neurological disorder is associated with a dysregulation of a DYRK1A gene, a DYRK1A protein, or expression or activity or level of any of the same; and (b) administering to the subject a therapeutically effective amount of a compound of Formula (I) or (II), or a pharmaceutically acceptable salt of any of the foregoing, or a pharmaceutical composition as provided herein.

Provided herein is a method of treating a DYRK1A-associated disease or disorder in a subject, the method comprising administering to a subject identified or diagnosed as having a DYRK1A-associated disease or disorder a therapeutically effective amount of a compound of Formula (I) or (II), or a pharmaceutically acceptable salt of any of the foregoing, or a pharmaceutical composition as provided herein.

This disclosure also provides a method of treating a DYRK1A-associated neurological disorder in a subject, the method comprising: determining that the neurological disorder in the subject is a DYRK1A-associated disease or disorder; and administering to the subject a therapeutically effective amount of a compound of Formula (I) or (II), or a pharmaceutically acceptable salt of any of the foregoing, or a pharmaceutical composition as provided herein.

Further provided herein is a method of treating a DYRK1A-associated neurological disorder in a subject, the method comprising administering to a subject identified or diagnosed as having a DYRK1A-associated neurological disorder a therapeutically effective amount of a compound of Formula (I) or (II), or a pharmaceutically acceptable salt of any of the foregoing, or a pharmaceutical composition as provided herein.

This disclosure also provides a method of treating a DYRK1A-associated neurological disorder in a subject, the method comprising: determining that the neurological disorder in the subject is a DYRK1A-associated neurological disorder; and administering to the subject a therapeutically effective amount of a compound of Formula (I) or (II), or a pharmaceutically acceptable salt of any of the foregoing, or a pharmaceutical composition as provided herein.

Provided herein is a method of treating a subject, the method comprising administering a therapeutically effective amount of a compound of Formula (I) or (II), or a pharmaceutically acceptable salt of any of the foregoing, or a pharmaceutical composition as provided herein, to a subject having a clinical record that indicates that the subject has a dysregulation of a DYRK1A gene, a DYRK1A protein, or expression or activity or level of any of the same.

This disclosure also provides a method for inhibiting DYRK1A in a mammalian cell, the method comprising contacting the mammalian cell with a therapeutically effective amount of a compound of Formula (I) or (II), or a pharmaceutically acceptable salt of any of the foregoing.

The details of one or more embodiments of this disclosure are set forth in the accompanying drawings and the description below. Other features and advantages of the present disclosure will be apparent from the description and the claims.

### DETAILED DESCRIPTION

Dual-specificity tyrosine phosphorylation-regulated kinase 1A (DYRK1A) is a member of the dual-specificity tyrosine phosphorylation regulated kinase (DYRK) family, which is also part of the larger CGMC family of kinases. DYRK1A is a 763 amino acid, 85 kDa serine/threonine kinase located on chromosome 21. DYRK1A contains a nuclear targeting signal sequence, a protein kinase domain, a leucine zipper motif, and a highly conservative 13-consecutive-histidine repeat. Alternative splicing DYRK1A generates several transcript variants differing from each other either in either the 5′ untranslated region or in the 3′ coding region resulting in at least five different isoforms.

DYRK1A possesses catalytic activity that is regulated by autophosphorylation of a tyrosine residue (Y321) which results in constitutively active serine/threonine kinase activity. Since DYRK1A is constitutively active, its activity is dosage dependent. Thus, both elevated levels and depressed levels of DYRK1A (relative to wild-type levels) have been shown to lead to neurological impairment.

DYRK1A displays a broad substrate spectrum (e.g., broad range of targets) including splicing factors, synaptic proteins, and transcription factors. It is ubiquitously expressed in all mammalian tissues and cells, although at different levels, with particularly high levels in embryonic and adult brain tissues. The human DYRK1A gene is a candidate gene to treat several Down syndrome characteristics, including intellectual impairment and Alzheimer's disease associated with Down syndrome, due to its localization in the Down syndrome critical region on chromosome 21 and its role in brain function. Notably, Drosophila with deleterious mutations in the ortholog of DYRK1A (“Minibrain”) have a reduced number of neurons in their central nervous system. Likewise, mice heterozygous for a disrupted allele of the Dyrk1a gene exhibit decreased viability, behavioral alterations, and delayed growth. Fotaki, et al., Mol Cell Biol., 22(18): 6636-6647 (2014).

The identification of hundreds of genes deregulated by DYRK1A overexpression and numerous cytosolic, cytoskeletal and nuclear proteins, including transcription factors, phosphorylated by DYRK1A, indicates that DYRK1A overexpression is central for the deregulation of multiple pathways in the developing and aging brain of individuals with Down syndrome. Identifying DYRK1A cell signaling or transduction pathways can lead to a better understanding of how DYRK1A overexpression (or under expression) leads to the various disease states in which it is known to be involved. Specifically, DYRK1A is known to be active in activated PI3K/Akt signaling, a pathway largely involved in neuronal development, growth, and survival. DYRK1A is also known to be active in ASK1/JNK1 activity and inhibitors of DYRK1A may induce neuronal death and apoptosis. DYRK1A is also known to phosphorylate p53 during embryonic brain development, and inhibitors of DYRK1A can prevent neuronal proliferation alteration. DYRK1A also phosphorylates synaptic proteins Amph 1, Dynamin 1, and Synaptojanin, which are involved in the regulation of endocytosis and inhibitors of DYRK1A can retain synaptic plasticity through preventing alteration of the number, size, and morphology of dendritic spines. DYRK1A also phosphorylates inhibit presenilin 1, the catalytic sub-unit of 7-secretase. Ryu, et al., J Neurochem., 115(3): 574-84 (2010).

DYRK1A overexpression leads to structural and functional alterations including intellectual disability and dementia, e.g., Alzheimer's disease. In particular, genes involved in learning disorders, synaptic flexibility changes, memory loss, and abnormal cell cycles, result in neuropathological symptoms similar to dementia associated with Alzheimer's disease. DYRK1A can also affect the proliferation and differentiation of neuronal progenitors, thus influencing neurogenesis and brain growth. It can also affect neurotransmission and dendritic spine formation through its interaction with synaptic proteins and the cytoskeleton.

One potential source of treatment are inhibitors of DYRK1A. Inhibitors that can normalize DYRK1A levels in Down syndrome may improve synaptic plasticity and delay the onset of Alzheimer's disease pathology, including tau hyperphosphorylation. Therefore, inhibiting DYRK1A activity in individuals with Down syndrome might counteract the phenotypic effects of its overexpression and is a potential avenue for the treatment of such developmental defects and prevention and/or mitigation of age-associated neurodegeneration, including Alzheimer's disease associated with Down syndrome. Studies have shown that inhibition of overexpressed DYRK1A resulted in normal DYRK1A levels and been found to improve cognitive and behavioral deficits in transgenic models. See, e.g., Stringer, et al., Mol Genet Genomic Med, 5, 451-465 (2017) and Feki and Hibaoui, Brain Sci, 8, 187 (2018). However, despite promising results there is considerable variation across studies in terms of outcomes. Discrepancies were attributed to differences in model, dose, route of administration, the composition of the inhibitor, and timing of administration.

Epigallocatechin gallate (EGCG) is the primary flavonoid of green tea and has been investigated for its therapeutic effects, which include anti-oxidative, anti-inflammatory, anti-cancer, anti-infective and neuroprotective activity. See, Bhat, et al. Towards the discovery of drug-like epigallocatechin gallate analogs as Hsp90 inhibitors, Bioorg Med Chem Lett, 24, 2263-2266 (2014). EGCG is a non-ATP competitive DYRK1A inhibitor and studies have shown that green tea extract comprising 41% EGCG were able to alleviate cognitive decline seen in transgenic mice over expressing DYRK1A. ECGC has also been shown to improve memory recognition and working memory. However, ECGC is not significantly selective and has numerous off-target effects, thus reducing its potential long-term use.

SM07883 is an orally bioavailable (% F 92% in mice, 109% in monkey), BBB penetrant, DYRK1A inhibitor (IC50 1.6 nM) that also shows potent inhibition for DYRK1B, CLK4, and GSK3β in kinase assays. It was found to protect against tau hyperphosphorylation in mouse models. SM07883 was tested for treatment of Alzheimer's disease in a phase 1 study in Australia (ACTRN12619000327189). However, according to the study description page at www.anzctr.org.au, the date of last data collection was in May 2019 and no results have been published for the trial.

This disclosure provides compounds of Formula (I) or (II) and pharmaceutically acceptable salts of any of the foregoing, that inhibit Dual specificity tyrosine-phosphorylation-regulated kinase 1A (DYRK1A). These chemical entities are useful, e.g., for treating a condition, disease or disorder in which increased (e.g., excessive) DYRK1A activation contributes to the pathology and/or symptoms and/or progression of the condition, disease or disorder (e.g., a neurological disorder in a subject (e.g., a human). This disclosure also provides compositions containing the same as well as methods of using and making the same.

### Definitions

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entireties. In case of conflict, the present specification, including definitions, will control.

The term “compound,” as used herein is meant to include all stereoisomers, geometric isomers, tautomers, and isotopically enriched variants of the structures depicted. Compounds herein identified by name or structure as one particular tautomeric form are intended to include other tautomeric forms unless otherwise specified. For example, if quinazolin-4-ol is encompassed by a claim or embodiment, then quinazolin-4(3H)-one is also covered by the claim or embodiment (see below).

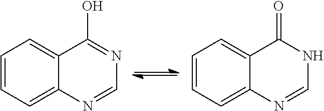 [SMILES: Oc1ncnc2ccccc12 ; O=c1[nH]cnc2ccccc12]

It will be appreciated that certain compounds provided herein may contain one or more centers of asymmetry and may therefore be prepared and isolated in a mixture of isomers such as a racemic mixture, or in an enantiomerically pure form. Unless otherwise indicated, when a disclosed compound is named or depicted by a structure without specifying the stereochemistry (e.g., a “flat” structure) and has one or more chiral centers, it is understood to represent all possible stereoisomers of the compound.

The term “about” when referring to a number or a numerical range means that the number or numerical range referred to is an approximation, for example, within experimental variability and/or statistical experimental error, and thus the number or numerical range may vary up to 10% of the stated number or numerical range.

The term “acceptable” with respect to a formulation, composition or ingredient, as used herein, means having no persistent detrimental effect on the general health of the subject being treated.

The term “inhibit” or “inhibition of” means to reduce by a measurable amount, or to prevent entirely (e.g., 100% inhibition).

The term “therapeutically effective amount,” as used herein, refer to a sufficient amount of a chemical entity being administered which will relieve to some extent one or more of the symptoms of the disease or condition being treated. The result includes reduction and/or alleviation of the signs, symptoms, or causes of a disease, or any other desired alteration of a biological system. For example, a therapeutically effective amount” for therapeutic uses is the amount of the composition comprising a compound as disclosed herein required to provide a clinically significant decrease in disease symptoms. An appropriate “therapeutically effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The term “halogen” refers to fluoro (F), chloro (Cl), bromo (Br), or iodo (I).

The term “oxo” refers to a divalent doubly bonded oxygen atom (i.e., “═O”). As used herein, oxo groups are attached to carbon atoms to form carbonyls.

The term “hydroxyl” refers to an —OH radical.

The term “cyano” refers to a —CN radical.

The term “alkyl” refers to a saturated acyclic hydrocarbon radical that can be straight chain or branched chain, containing the indicated number of carbon atoms. For example, C1-C10 indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein. A “C0” alkyl refers to a bond, e.g., phenyl-(C0 alkyl)-OH corresponds to phenol.

The term “haloalkyl” refers to an alkyl group in which one or more hydrogen atoms is/are replaced with an independently selected halogen.

The term “alkoxy” refers to an —O-alkyl radical (e.g., —OCH₃).

The term “aryl” refers to a 6-20 carbon atom monocyclic, bicyclic, or tricyclic group wherein at least one ring in the system is aromatic. Examples of aryl groups include phenyl, naphthyl, tetrahydronaphthyl, and the like.

The term “cycloalkyl” as used herein refers to cyclic hydrocarbon groups having the indicated number of carbon atoms, e.g., 3 to 20 ring carbons (C3-C20), 3 to 16 ring carbons (C3-C16), 3-10 ring carbons (C3-C10), or 3-6 ring carbons (C3-C6). Cycloalkyl groups are saturated or partially unsatured (but not aromatic). Examples of cycloalkyl groups include, without limitation, cyclopropyl, cyclobutyl, cyclopentyl, cyclopentenyl, cyclohexyl, cyclohexenyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

The term “heteroaryl”, as used herein, means a monocyclic, bicyclic, or tricyclic group having 5 to 20 ring atoms (5-20 membered heteroaryl), such as 5, 6, 9, 10, or 14 ring atoms; wherein at least one ring in the system contains one or more heteroatoms independently selected from the group consisting of N, O, and S and at least one ring in the system is aromatic (but does not have to be a ring which contains a heteroatom, e.g. tetrahydroisoquinolinyl, e.g., tetrahydroquinolinyl). Examples of heteroaryl include thienyl, pyridinyl, furyl, oxazolyl, oxadiazolyl, pyrrolyl, imidazolyl, triazolyl, thiadiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone (e.g.,

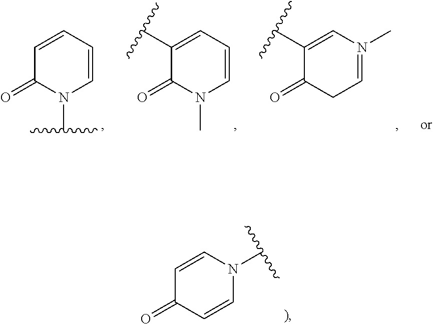

pyrimidone (e.g.,

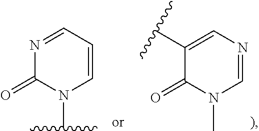

pyridazinone (e.g.,

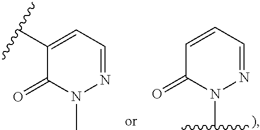

pyrazinone (e.g.,

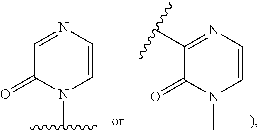

and imidazolone (e.g.,

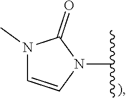 [SMILES: *n1ccn(C)c1=O]

wherein each ring nitrogen adjacent to a carbonyl is tertiary (i.e., the oxo group (i.e., “═O”) herein is a constituent part of the heteroaryl ring).

The term “heterocyclyl” refers to monocyclic, bicyclic, or tricyclic saturated or partially unsaturated ring systems with 3-16 ring atoms (e.g., 5-8 membered monocyclic, 8-12 membered bicyclic, or 11-14 membered tricyclic ring systems) having 1-3 heteroatoms if monocyclic, 1-6 heteroatoms if bicyclic, or 1-9 heteroatoms if tricyclic. The heteroatoms are selected from the group consisting of O, N, or S (e.g., carbon atoms and 1-3, 1-6, or 1-9 heteroatoms of N, O, or S if monocyclic, bicyclic, or tricyclic, respectively), wherein one or more ring atoms may be substituted by 1-3 oxo (forming, e.g., a lactam) and one or more N or S atoms may be substituted by 1-2 oxido (forming, e.g., an N-oxide, an S-oxide, or an S,S-dioxide), valence permitting; and wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, tetrahydropyridyl, dihydropyrazinyl, dihydropyridyl, dihydropyrrolyl, dihydrofuranyl, dihydrothiophenyl, and the like. Heterocyclyl may include multiple fused and bridged rings. Non-limiting examples of fused/bridged heterocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

As used herein, examples of aromatic rings include: benzene, pyridine, pyrimidine, pyrazine, pyridazine, pyridone, pyrrole, pyrazole, oxazole, thioazole, isoxazole, isothiazole, and the like.

The term “saturated” as used in this context means only single bonds present between constituent atoms.

As used herein, when a ring is described as being “partially unsaturated”, it means said ring has one or more additional degrees of unsaturation (in addition to the degree of unsaturation attributed to the ring itself; e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, cycloalkyl, and the like described herein) containing a sufficient number of ring atoms to form bicyclic or higher order ring systems (e.g., tricyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge (e.g.,

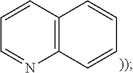

(ii) a single ring atom (spiro-fused ring systems) (e.g.,

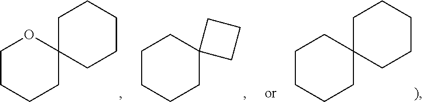

or (iii) a contiguous array of ring atoms (bridged ring systems having all bridge lengths >0) (e.g.,

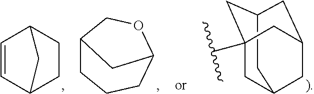

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

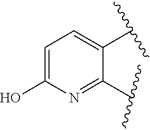 [SMILES: *c1ccc(O)nc1*]

encompasses the tautomeric form containing the moiety:

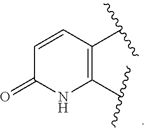 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

Dashed lines in chemical structures, for example,

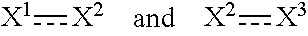

represent single or double bonds. One skilled in the art understands that, in this structure,

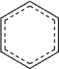

or example, the maximum number of double bonds is three.

### Compounds of Formula (I)

The substituent groups used in this section (e.g., R¹, R², and the like) refer solely to the groups in Formula (I).

Some embodiments provide a compound of Formula (I):

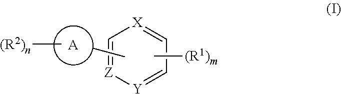

or a pharmaceutically acceptable salt thereof, wherein:
X is CH, N, N—O, or

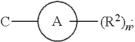

Y is CH, N, or

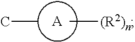

Z is CH, N, or CR¹;
wherein Formula (I) contains one Ring A and not more than two of X, Y, and Z are N or N—O;
each R¹ is independently hydroxyl, cyano, C1-C6 alkyl,

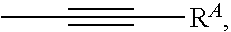

—NHC(═O)R^{A}, C1-C6 alkoxy, —(CH₂)ₚ—NR^{B}R^{C}, C3-C10 cycloalkyloxy optionally substituted with amino, -Q-(CH₂)_{q}—R^{D}, —CO₂R^{B}, —C(═O)NR^{B}R^{C}, —C(═O)-3-6 membered heterocyclyl, or phenoxy;
Ring A is a fused bicyclic 9 membered cycloalkyl, a fused bicyclic 9-10 membered heteroaryl, a 9 membered fused bicyclic heterocyclyl, or a fused tricyclic 13-14 membered heterocyclyl;
each R² is independently halogen, C1-C6 haloalkyl, C1-C6 alkyl optionally substituted with 1-2 substituents independently selected from hydroxyl, C1-C6 alkoxy, and 5-10 membered heteroaryl, —(CH₂)ₜ-Q¹-4-6 membered heterocyclyl optionally substituted with C1-C6 alkyl or —CO₂H, 4-9 membered heterocyclyl optionally substituted with R^{G},

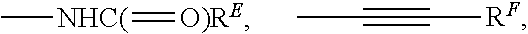

C1-C6 alkoxy, —C(═O)NHR^{H}, phenyl optionally substituted with 1-2 substituents independently selected from hydroxyl and cyano, 5-10 membered heteroaryl optionally substituted with C1-C6 alkyl, —NHSO₂(C1-C6 alkyl), C3-C9 cycloalkyl optionally substituted with hydroxyl or —NR^{B2}R^{C2}, —NR^{B1}R^{C1}, —C(═O)-3-6 membered heterocyclyl optionally substituted with C1-C6 alkyl, or benzyl optionally substituted with C1-C6 alkoxy;
each R^{A} and R^{F} is independently C3-C6 cycloalkyl or 3-6 membered heterocyclyl optionally substituted with 1-2 substituents independently selected from hydroxyl and C1-C6 alkyl;
each R^{B}, R^{B1}, R^{B2}, R^{C}, R^{C1}, and R^{C2} is independently hydrogen or C1-C6 alkyl optionally substituted with hydroxyl;
each R^{D} is independently a 4-6 membered heterocyclyl optionally substituted with C1-C6 alkyl or a phenyl optionally substituted with 1-2 independently selected halogen;
each R^{E} is independently 3-10 membered heterocyclyl optionally substituted with 1-4 independently selected C1-C6 alkyl or a —(CH₂)ᵥphenyl optionally substituted with cyano or halogen;
R^{G} is C1-C6 alkyl, —SO₂(R^{G1}), —C(═O)(C1-C6 alkyl), —C(═O)O—Benzyl, 4-6 membered heterocyclyl optionally substituted with 1-2 substituents independently selected from C1-C6 alkyl, and C3-C6 cycloalkyl optionally substituted with —CO₂H;
R^{G1} is C1-C6 alkyl or phenyl optionally substituted with cyano or halogen;
R^{H} is hydrogen, C1-C6 alkyl, or benzyl optionally substituted with halogen;
Q and Q¹ are independently 0, NH, or a bond;
m, n, p, and t are each independently 0, 1, 2, or 3;
v is 0 or 1; and
q is 1, 2, or 3.

In some embodiments, not more than one of X, Y, and Z are N or N—O.

In some embodiments, none of X, Y, and Z are N or N—O.

In some embodiments, X is CH. In some embodiments, X is N. In some embodiments, X is N—O. In some embodiments, X is

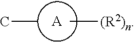

In some embodiments, Y is CH. In some embodiments, Y is N. In some embodiments, Y is

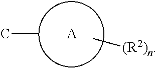

In some embodiments, Z is CH. In some embodiments, Z is N. In some embodiments, Z is CR¹.

In some embodiments,

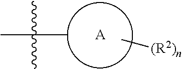

is attached to the position ortho to X and ortho to Z.

In some embodiments,

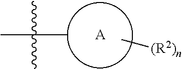

is attached to the position ortho to X and para to Z.

In some embodiments,

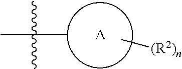

is attached to the position ortho to Y and meta to Z.

In some embodiments, m is 1. In some embodiments, m is 2. In some embodiments, m is 3.

In some embodiments,

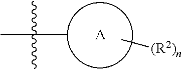

is attached to the position ortho to X and para to Z; Z is N; Y is CH; X is CH; m is 1; and R¹ is attached to the position ortho to X and ortho to Z.

In some embodiments, at least one R¹ is hydroxyl. In some embodiments, at least one R¹ is cyano.

In some embodiments, at least one R¹ is C1-C6 alkyl. In some embodiments, at least one R¹ is C1-C4 alkyl. In some embodiments, at least one R¹ is methyl, ethyl, isopropyl, or isobutyl.

In some embodiments, at least one R¹ is methyl.

In some embodiments, at least one R¹ is

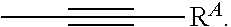

In some embodiments, at least one R¹ is —NHC(═O)R^{A}.

In some embodiments, at least one R^{A} is C3-C6 cycloalkyl. In some embodiments, at least one R^{A} is C3-C4 cycloalkyl. In some embodiments, at least one R^{A} is cyclopropyl. In some embodiments, at least one R^{A} is cyclopentyl.

In some embodiments, at least one R^{A} is 3-6 membered heterocyclyl optionally substituted with 1-2 substituents independently selected from hydroxyl and C1-C6 alkyl. In some embodiments, at least one R^{A} is 3-6 membered heterocyclyl substituted with 1-2 substituents independently selected from hydroxyl and C1-C6 alkyl. In some embodiments, at least one R^{A} is 3-6 membered heterocyclyl substituted with hydroxyl. In some embodiments, at least one R^{A} is 3-6 membered heterocyclyl substituted with C1-C6 alkyl. In some embodiments, at least one R^{A} is 3-6 membered heterocyclyl substituted with methyl or isobutyl. In some embodiments, at least one R^{A} is unsubstituted 3-6 membered heterocyclyl. In some embodiments, the R^{A} 3-6 membered heterocyclyl is piperidinyl or tetrahydropyranyl.

In some embodiments, at least one R¹ is C1-C6 alkoxy. In some embodiments, at least one R¹ is C1-C3 alkoxy. In some embodiments, at least one R¹ is methoxy, ethoxy, or propoxy.

In some embodiments, “at least one” of a specified substituent refers to one or more of that group. In some embodiments, “at least one” of a specified substituent refers to only one of a specified group. For example, at least one R¹ is C1-C6 alkoxy refers to both one R¹ being a C1-C6 alkoxy, as well as more than one R¹ being a C1-C6 alkoxy.

In some embodiments, at least one R¹ is —(CH₂)ₚ—NR^{B}R^{C}.

In some embodiments, p is 0. In some embodiments, p is 1. In some embodiments, p is 2. In some embodiments, p is 3. In some embodiments, p is 0 or 1. In some embodiments, p is 1 or 2.

In some embodiments, R^{B} and R^{C} are hydrogen. In some embodiments, R^{B} is hydrogen and R^{C} is C1-C6 alkyl optionally substituted with hydroxyl. In some embodiments, R^{B} is hydrogen and R^{C} is C1-C6 alkyl substituted with hydroxyl. In some embodiments, R^{B} is hydrogen and R^{C} is unsubstituted C1-C6 alkyl. In some embodiments, R^{B} is C1-C6 alkyl optionally substituted with hydroxyl and R^{C} is hydrogen. In some embodiments, R^{B} is C1-C6 alkyl substituted with hydroxyl and R^{C} is hydrogen. In some embodiments, R^{B} is unsubstituted C1-C6 alkyl and R^{C} is hydrogen.

In some embodiments, R^{B} and R^{C} are each independently selected C1-C6 alkyl optionally substituted with hydroxyl. In some embodiments, R^{B} and R^{C} are each independently selected C1-C6 alkyl substituted with hydroxyl. In some embodiments, R^{B} and R^{C} are each independently selected unsubstituted C1-C6 alkyl. In some embodiments, at least one R^{B} and/or R^{C} C1-C6 alkyl is C1-C4 alkyl. In some embodiments, at least one R^{B} and/or R^{C} C1-C6 alkyl is methyl. In some embodiments, at least one R^{B} and/or R^{C} C1-C6 alkyl is ethyl. In some embodiments, at least one R¹ is C3-C10 cycloalkyloxy optionally substituted with amino.

In some embodiments, at least one R¹ is C3-C10 cycloalkyloxy substituted with amino. In some embodiments, at least one R¹ is C3-C6 cycloalkyloxy substituted with amino. In some embodiments, at least one R¹ is unsubstituted C3-C10 cycloalkyloxy. In some embodiments, the R¹ C3-C10 cycloalkyloxy is cyclobutoxy.

In some embodiments, at least one R¹ is -Q-(CH₂)_{q}—R^{D}. In some embodiments, Q is O. In some embodiments, Q is NH. In some embodiments, Q is bond. In some embodiments, q is 1. In some embodiments, q is 2. In some embodiments, q is 3.

In some embodiments, at least one R^{D} is 4-6 membered heterocyclyl optionally substituted with C1-C6 alkyl. In some embodiments, at least one R^{D} is a phenyl optionally substituted with 1-2 independently selected halogen.

In some embodiments, at least one R¹ is —CO₂R^{B}.

In some embodiments, R^{B} is hydrogen. In some embodiments, R^{B} is C1-C6 alkyl optionally substituted with hydroxyl. In some embodiments, R^{B} is C1-C6 alkyl substituted with hydroxyl. In some embodiments, R^{B} is unsubstituted C1-C6 alkyl. In some embodiments, R^{B} is methyl. In some embodiments, R^{B} is ethyl.

In some embodiments, at least one R¹ is —C(═O)NR^{B}R^{C}.

In some embodiments, R^{B} and R^{C} are hydrogen. In some embodiments, R^{B} is hydrogen and R^{C} is C1-C6 alkyl optionally substituted with hydroxyl. In some embodiments, R^{B} is hydrogen and R^{C} is C1-C6 alkyl substituted with hydroxyl. In some embodiments, R^{B} is hydrogen and R^{C} is unsubstituted C1-C6 alkyl. In some embodiments, R^{B} is C1-C6 alkyl optionally substituted with hydroxyl and R^{C} is hydrogen. In some embodiments, R^{B} is C1-C6 alkyl substituted with hydroxyl and R^{C} is hydrogen. In some embodiments, R^{B} is unsubstituted C1-C6 alkyl and R^{C} is hydrogen. In some embodiments, R^{B} and R^{C} are each independently selected C1-C6 alkyl optionally substituted with hydroxyl. In some embodiments, R^{B} and R^{C} are each independently selected C1-C6 alkyl substituted with hydroxyl. In some embodiments, R^{B} and R^{C} are each independently selected unsubstituted C1-C6 alkyl. In some embodiments, at least one R^{B} and/or R^{C} C1-C6 alkyl is C1-C4 alkyl. In some embodiments, at least one R^{B} and/or R^{C} C1-C6 alkyl is methyl. In some embodiments, at least one R^{B} and/or R^{C} C1-C6 alkyl is ethyl.

In some embodiments, at least one R¹ is —C(═O)-3-6 membered heterocyclyl. In some embodiments, at least one R¹ is —C(═O)-piperazinyl.

In some embodiments, at least one R¹ is phenoxy.

In some embodiments, m is 0.

In some embodiments, Ring A is a fused bicyclic 9 membered cycloalkyl.

In some embodiments, Ring A is 2,3-dihydro-1H-indenyl.

In some embodiments, Ring A is a fused bicyclic 9-10 membered heteroaryl.

In some embodiments, Ring A is pyrazolo[1,5-a]pyridinyl, 3H-imidazo[4,5-b]pyridine, pyrrolo[1,2-a]pyrazine, 7H-pyrrolo[2,3-c]pyridazine, pyrrolo[1,2-c]pyrimidine, 2H-indazolyl, imidazo[1,2-a]pyridine, benzo[d]thiazole, 1H-benzo[d]imidazole, 1H-pyrrolo[2,3-b]pyridine, imidazo[1,2-a]pyridine, 1H-pyrrolo[2,3-c]pyridinyl, pyrazolo[1,5-a]pyrazin-4(5H)-one, isoquinoline, quinoline, quinolin-2(1H)-one, 1,8-naphthyridin-2(1H)-one, 5H-pyrrolo[2,3-b]pyrazinyl, benzo[d]oxazolyl, thiazolo[5,4-b]pyridinyl, benzo[d]isothiazolyl, 2H-pyrazolo[3,4-c]pyridinyl, 1H-indazolyl, pyrazolo[1,5-a]pyrazin-4(5H)-one, furo[2,3-c]pyridinyl, [1,2,4]triazolo[4,3-a]pyridinyl, [1,2,4]triazolo[4,3-a]pyridin-3(2H)-one, benzo[d]isothiazolyl, imidazo[1,5-a]pyridinyl, 4H-pyrido[1,2-a][1,3,5]triazinyl, 1H-pyrrolo[2,3-c]pyridinyl, or 1H-pyrrolo[3,4-c]pyridine-1,3(2H)-dione.

In some embodiments, Ring A is pyrazolo[1,5-a]pyridinyl, 2H-indazolyl, imidazo[1,2-a]pyridine, benzo[d]thiazole, 1H-benzo[d]imidazole, 1H-pyrrolo[2,3-c]pyridinyl, isoquinoline, quinoline, quinolin-2(1H)-one, 1,8-naphthyridin-2(1H)-one, benzo[d]oxazolyl, 2H-pyrazolo[3,4-c]pyridinyl, 1H-indazolyl, 2H-indazolyl, [1,2,4]triazolo[4,3-a]pyridinyl, or 4H-pyrido[1,2-a][1,3,5]triazinyl.

In some embodiments, Ring A is benzo[d]thiazole, pyrrolo[1,2-a]pyrazine, 7H-pyrrolo[2,3-c]pyridazine, thiazolo[5,4-b]pyridinyl, or imidazo[1,5-a]pyridinyl.

In some embodiments, Ring A is 3H-imidazo[4,5-b]pyridine, imidazo[1,2-a]pyridine, or 1H-pyrrolo[2,3-b]pyridine.

In some embodiments, Ring A is 1H-benzo[d]imidazole, 1H-pyrrolo[2,3-b]pyridine, furo[2,3-c]pyridinyl, 1H-indazolyl, pyrazolo[1,5-a]pyrazin-4(5H)-one, or pyrazolo[1,5-a]pyridinyl.

In some embodiments, Ring A is 1H-benzo[d]imidazole, 1H-pyrrolo[2,3-b]pyridine, pyrazolo[1,5-a]pyrazin-4(5H)-one, or 4H-pyrido[1,2-a][1,3,5]triazinyl.

In some embodiments, Ring A is 1H-benzo[d]imidazole.

In some embodiments, Ring A is 1H-pyrrolo[2,3-b]pyridine.

In some embodiments, Ring A is pyrrolo[1,2-c]pyrimidine.

In some embodiments, Ring A is imidazo[1,2-a]pyridine.

In some embodiments, Ring A is a 9 membered fused bicyclic heterocyclyl.

In some embodiments, Ring A is 2,3-dihydrobenzofuranyl, 2-oxo-1,2-dihydro-1,8-naphthyridinyl, 2,3-dihydro-1H-indenyl, 4H-chromen-4-one, 1,3-dihydro-2H-benzo[d]imidazol-2-one, 2-oxo-1,2-dihydro-1,7-naphthyridinyl, 6-oxo-5,6,7,8-tetrahydroimidazo[1,2-a]pyrazinyl, 5,6,7,8-tetrahydroimidazo[1,2-a]pyrazinyl, imidazo[1,5-a]pyridinyl, 4H-pyrido[1,2-a][1,3,5]triazinyl, 4,5,6,7-tetrahydropyrazolo[1,5-a]pyrazinyl, or 1,2-dihydro-3H-pyrrolo[3,4-c]pyridin-3-one.

In some embodiments, Ring A is 5,6,7,8-tetrahydroimidazo[1,2-a]pyrazinyl, 6-oxo-5,6,7,8-tetrahydroimidazo[1,2-a]pyrazinyl, or 4,5,6,7-tetrahydropyrazolo[1,5-a]pyrazinyl.

In some embodiments, Ring A is 1,2-dihydro-3H-pyrrolo[3,4-c]pyridin-3-one.

In some embodiments, Ring A is a fused tricyclic 13-14 membered heterocyclyl.

In some embodiments, Ring A is 1,2,3,4-tetrahydrobenzo[4,5]imidazo[1,2-a]pyrazine.

In some embodiments, Ring A is pyrazolo[1,5-a]quinazolin-5(4H)-one.

In some embodiments, n is 1. In some embodiments, n is 2. In some embodiments, n is 3.

In some embodiments, at least one R² is halogen. In some embodiments, at least one R² is fluoro. In some embodiments, at least one R² is chloro.

In some embodiments, at least one R² is C1-C6 haloalkyl. In some embodiments, at least one R² is difluoromethyl. In some embodiments, at least one R² is trifluoromethyl.

In some embodiments, at least one R² is C1-C6 alkyl optionally substituted with 1-2 substituents independently selected from hydroxyl, C1-C6 alkoxy, and 5-10 membered heteroaryl.

In some embodiments, at least one R² is C1-C6 alkyl substituted with 1-2 substituents independently selected from hydroxyl, C1-C6 alkoxy, and 5-10 membered heteroaryl.

In some embodiments, at least one R² is C1-C6 alkyl substituted with 1-2 substituents independently selected from hydroxyl and indazolyl.

In some embodiments, at least one R² is C1-C6 alkyl substituted with hydroxyl. In some embodiments, at least one R² is C1-C6 alkyl substituted with C1-C6 alkoxy. In some embodiments, at least one R² is C1-C6 alkyl substituted with methoxy.

In some embodiments, at least one R² is C1-C6 alkyl substituted with 5-10 membered heteroaryl. In some embodiments, at least one R² is C1-C6 alkyl substituted with indazolyl.

In some embodiments, the R² C1-C6 alkyl is methyl, ethyl, isopropyl, or isobutyl. In some embodiments, the R² C1-C6 alkyl is methyl.

In some embodiments, at least one R² is —(CH₂)ₜ-Q¹-4-6 membered heterocyclyl optionally substituted with C1-C6 alkyl or —CO₂H. In some embodiments, at least one R² is —(CH₂)ₜ-Q¹-4-6 membered heterocyclyl substituted with C1-C6 alkyl or —CO₂H. In some embodiments, at least one R² is —(CH₂)ₜ-Q¹-4-6 membered heterocyclyl substituted with methyl or —CO₂H. In some embodiments, at least one R² is unsubstituted —(CH₂)ₜ-Q¹-4-6 membered heterocyclyl. In some embodiments, the 4-6 membered heterocyclyl of the R²—(CH₂)ₜ-Q¹-4-6 membered heterocyclyl is piperidinyl.

In some embodiments, Q¹ is 0. In some embodiments, Q¹ is NH. In some embodiments, Q¹ is bond.

In some embodiments, t is 0. In some embodiments, t is 1. In some embodiments, t is 2. In some embodiments, t is 3.

In some embodiments, at least one R² is 4-9 membered heterocyclyl optionally substituted with R^{G}. In some embodiments, at least one R² is 4-9 membered heterocyclyl substituted with R^{G}.

In some embodiments, at least one R² is an unsubstituted 4-9 membered heterocyclyl. In some embodiments, the R² 4-9 membered heterocyclyl is tetrahydropyranyl, piperidinyl, azepanyl, azetidinyl, 2,3-dihydrobenzofuran, or oxetanyl.

In some embodiments, R^{G} is C1-C6 alkyl. In some embodiments, R^{G} is —SO₂(R^{G1}).

In some embodiments, at least one R² is —NHC(═O)R^{E}. In some embodiments, at least one R² is

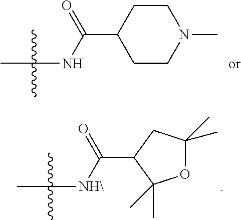

In some embodiments, each R^{E} is independently 3-10 membered heterocyclyl optionally substituted with 1-3 independently selected C1-C6 alkyl or a —(CH₂)ᵥphenyl optionally substituted with cyano or halogen.

In some embodiments, R^{E} is 3-10 membered heterocyclyl optionally substituted with 1-3 independently selected C1-C6 alkyl. In some embodiments, R^{E} is 3-10 membered heterocyclyl substituted with 1-3 independently selected C1-C6 alkyl. In some embodiments, R^{E} is 3-10 membered heterocyclyl substituted with 1-3 methyl. In some embodiments, R^{E} is unsubstituted 3-10 membered heterocyclyl.

In some embodiments, R^{E} is 4-6 membered heterocyclyl optionally substituted with 1-3 independently selected C1-C6 alkyl. In some embodiments, R^{E} is 4-6 membered heterocyclyl substituted with 1-3 independently selected C1-C6 alkyl. In some embodiments, R^{E} is 4-6 membered heterocyclyl substituted with 1-3 methyl. In some embodiments, R^{E} is unsubstituted 4-6 membered heterocyclyl.

In some embodiments, the R^{E} 3-10 membered heterocyclyl is piperidinyl, (1R,5S)-8-azabicyclo[3. 2. 1]octanyl, 3-azabicyclo[3. 1. 0]hexanyl, octahydrocyclopenta[c]pyrrolyl, azetidinyl, or tetrahydropyranyl.

In some embodiments, R^{E} is —(CH₂)ᵥphenyl optionally substituted with cyano or halogen;

In some embodiments, v is 0. In some embodiments, v is 1.

In some embodiments, at least one R² is

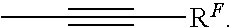

In some embodiments, R^{F} is C3-C6 cycloalkyl. In some embodiments, R^{F} is C3-C4 cycloalkyl. In some embodiments, R^{F} is cyclopropyl.

In some embodiments, R^{F} is 3-6 membered heterocyclyl optionally substituted with 1-2 substituents independently selected from hydroxyl and C1-C6 alkyl. In some embodiments, R^{F} is 3-6 membered heterocyclyl substituted with 1-2 substituents independently selected from hydroxyl and C1-C6 alkyl. In some embodiments, R^{F} is 3-6 membered heterocyclyl substituted with one hydroxyl and one C1-C6 alkyl. In some embodiments, R^{F} is 3-6 membered heterocyclyl substituted with hydroxyl. In some embodiments, R^{F} is 3-6 membered heterocyclyl substituted with C1-C6 alkyl. In some embodiments, R^{F} is 3-6 membered heterocyclyl substituted with methyl, ethyl, isopropyl, or isobutyl. In some embodiments, the R^{F} 3-6 membered heterocyclyl is piperidinyl or tetrahydropyranyl.

In some embodiments, at least one R² is C1-C6 alkoxy. In some embodiments, at least one R² is C1-C3 alkoxy. In some embodiments, at least one R² is methoxy.

In some embodiments, at least one R² is —C(═O)NHR^{H}.

In some embodiments, R^{H} is hydrogen. In some embodiments, R^{H} is C1-C6 alkyl. In some embodiments, R^{H} is C1-C3 alkyl. In some embodiments, R^{H} is methyl.

In some embodiments, R^{H} is benzyl optionally substituted with halogen. In some embodiments, R^{H} is benzyl optionally substituted with fluoro or chloro.

In some embodiments, at least one R² is phenyl optionally substituted with 1-2 substituents independently selected from hydroxyl and cyano. In some embodiments, at least one R² is phenyl substituted with 1-2 substituents independently selected from hydroxyl and cyano. In some embodiments, at least one R² is phenyl substituted with hydroxyl. In some embodiments, at least one R² is phenyl substituted with cyano. In some embodiments, at least one R² is unsubstituted phenyl.

In some embodiments, at least one R² is 5-10 membered heteroaryl optionally substituted with C1-C6 alkyl. In some embodiments, at least one R² is 5-10 membered heteroaryl substituted with C1-C6 alkyl. In some embodiments, at least one R² is 5-10 membered heteroaryl substituted with methyl. In some embodiments, at least one R² is unsubstituted 5-10 membered heteroaryl. In some embodiments, the R² 5-10 membered heteroaryl is quinolinyl, benzimidazolyl, or pyridyl. In some embodiments, the R² 5-10 membered heteroaryl is imidazolyl. In some embodiments, at least one R² is 1-methylimidazol-4-yl.

In some embodiments, at least one R² is 5-6 membered heteroaryl optionally substituted with C1-C6 alkyl. In some embodiments, at least one R² is 5-6 membered heteroaryl substituted with C1-C6 alkyl. In some embodiments, at least one R² is 5-6 membered heteroaryl substituted with methyl. In some embodiments, at least one R² is unsubstituted 5-6 membered heteroaryl. In some embodiments, the R² 5-6 membered heteroaryl is pyridyl.

In some embodiments, at least one R² is —NHSO₂(C1-C6 alkyl). In some embodiments, at least one R² is —NHSO₂methyl.

In some embodiments, at least one R² is C3-C9 cycloalkyl optionally substituted with hydroxyl or —NR^{B2}R^{C2}. In some embodiments, at least one R² is C3-C9 cycloalkyl substituted with hydroxyl or —NR^{B2}R^{C2}. In some embodiments, at least one R² is C3-C9 cycloalkyl substituted with hydroxyl. In some embodiments, at least one R² is C3-C9 cycloalkyl substituted with —NR^{B2}R^{C2}.

In some embodiments, R^{B2} and R^{C2} are hydrogen. In some embodiments, R^{B2} is hydrogen and R^{C2} is C1-C6 alkyl optionally substituted with hydroxyl. In some embodiments, R^{B2} is hydrogen and R^{C2} is C1-C6 alkyl substituted with hydroxyl. In some embodiments, R^{B2} is hydrogen and R^{C2} is unsubstituted C1-C6 alkyl. In some embodiments, R^{B2} is C1-C6 alkyl optionally substituted with hydroxyl and R^{C2} is hydrogen. In some embodiments, R^{B2} is C1-C6 alkyl substituted with hydroxyl and R^{C2} is hydrogen. In some embodiments, R^{B2} is unsubstituted C1-C6 alkyl and R^{C2} is hydrogen. In some embodiments, R^{B2} and R^{C2} are each independently selected C1-C6 alkyl optionally substituted with hydroxyl. In some embodiments, R^{B2} and R^{C2} are each independently selected C1-C6 alkyl substituted with hydroxyl. In some embodiments, R^{B2} and R^{C2} are each independently selected unsubstituted C1-C6 alkyl. In some embodiments, at least one R^{B2} and/or R^{C2} C1-C6 alkyl is C1-C4 alkyl. In some embodiments, at least one R^{B2} and/or R^{C2} C1-C6 alkyl is methyl. In some embodiments, at least one R^{B2} and/or R^{C2} C1-C6 alkyl is ethyl. In some embodiments, the R² C3-C9 cycloalkyl is C3-C6 cycloalkyl. In some embodiments, the R² C3-C9 cycloalkyl is cyclobutyl.

In some embodiments, at least one R² is —NR^{B1}R^{C1}.

In some embodiments, R^{B1} and R^{C1} are hydrogen. In some embodiments, R^{B1} is hydrogen and R^{C1} is C1-C6 alkyl optionally substituted with hydroxyl. In some embodiments, R^{B1} is hydrogen and R^{C1} is C1-C6 alkyl substituted with hydroxyl. In some embodiments, R^{B1} is hydrogen and R^{C1} is unsubstituted C1-C6 alkyl. In some embodiments, R^{B1} is C1-C6 alkyl optionally substituted with hydroxyl and R^{C1} is hydrogen. In some embodiments, R^{B1} is C1-C6 alkyl substituted with hydroxyl and R^{C1} is hydrogen. In some embodiments, R^{B1} is unsubstituted C1-C6 alkyl and R^{C1} is hydrogen. In some embodiments, R^{B1} and R^{C1} are each independently selected C1-C6 alkyl optionally substituted with hydroxyl. In some embodiments, R^{B1} and R^{C1} are each independently selected C1-C6 alkyl substituted with hydroxyl. In some embodiments, R^{B1} and R^{C1} are each independently selected unsubstituted C1-C6 alkyl. In some embodiments, at least one R^{B1} and/or R^{C1} C1-C6 alkyl is C1-C4 alkyl. In some embodiments, at least one R^{B1} and/or R^{C1} C1-C6 alkyl is methyl. In some embodiments, at least one R^{B1} and/or R^{C1} C1-C6 alkyl is ethyl.

In some embodiments, at least one R² is —C(═O)-3-6 membered heterocyclyl optionally substituted with C1-C6 alkyl. In some embodiments, at least one R² is —C(═O)-3-6 membered heterocyclyl substituted with C1-C6 alkyl. In some embodiments, at least one R² is —C(═O)-3-6 membered heterocyclyl substituted with methyl. In some embodiments, at least one R² is unsubstituted —C(═O)-3-6 membered heterocyclyl. In some embodiments, the R²—C(═O)-3-6 membered heterocyclyl is piperidinyl.

In some embodiments, at least one R² is benzyl optionally substituted with C1-C6 alkoxy. In some embodiments, at least one R² is benzyl substituted with C1-C6 alkoxy. In some embodiments, at least one R² is benzyl substituted with methoxy. In some embodiments, at least one R² is unsubstituted benzyl.

In some embodiments, n is 0.

In some embodiments, the compound of Formula (I) is a compound of Formula (I-A):

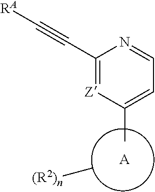

wherein: Z′ is CH or N. In some embodiments, Z′ is CH. In some embodiments, Z′ is N.

In some embodiments, the compound of Formula (I) is a compound of Formula (I-B):

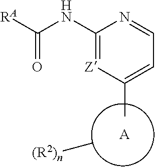

wherein: Z′ is CH or N. In some embodiments, Z′ is CH. In some embodiments, Z′ is N.

In some embodiments, the compound of Formula (I) is a compound of Formula (I-C):

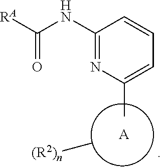

In some embodiments, the compound of Formula (I) is a compound of Formula (I-D):

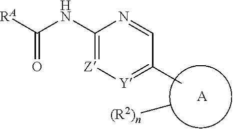

wherein: Y′ is CH or N; and Z′ is CH or N. In some embodiments, Y′ is CH. In some embodiments, Y′ is N. In some embodiments, Z′ is CH. In some embodiments, Z′ is N.

In some embodiments the compound of Formula (I) is a compound of Formula (I-E):

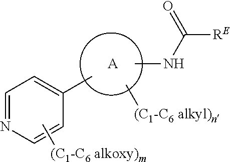

wherein: n′ is 0, 1, or 2. In some embodiments, n′ is 1. In some embodiments, n′ is 2.

In some embodiments, each C1-C6 alkyl attached to Ring A is methyl, ethyl, isopropyl, or isobutyl.

In some embodiments, each C1-C6 alkyl attached to Ring A is methyl.

In some embodiments, n′ is 0.

In some embodiments, m is 0. In some embodiments, m is 1. In some embodiments, m is 2. In some embodiments, m is 3.

In some embodiments, the C1-C6 alkoxy attached to the pyridyl is C1-C3 alkoxy. In some embodiments, the C1-C6 alkoxy attached to the pyridyl is methoxy, ethoxy, or propoxy.

In some embodiments, the compound of Formula (I) is a compound of Formula (I-F):

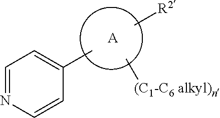

wherein:
R^{2′} is 4-9 membered heterocyclyl optionally substituted with R^{G}, phenyl optionally substituted with 1-2 substituents independently selected from hydroxyl and cyano, 5-10 membered heteroaryl optionally substituted with C1-C6 alkyl, or C3-C9 cycloalkyl optionally substituted with hydroxyl or —NR^{B2}R^{C2}; and
n′ is 0, 1, or 2.

In some embodiments, n′ is 0. In some embodiments, n′ is 1. In some embodiments, n′ is 2.

In some embodiments, each C1-C6 alkyl attached to Ring A is methyl, ethyl, isopropyl, or isobutyl. In some embodiments, each C1-C6 alkyl attached to Ring A is methyl.

In some embodiments, R^{2′} is 4-9 membered heterocyclyl optionally substituted with R^{G}.

In some embodiments, R^{2′} is phenyl optionally substituted with 1-2 substituents independently selected from hydroxyl and cyano. In some embodiments, R^{2′} is phenyl substituted with 1-2 substituents independently selected from hydroxyl and cyano. In some embodiments, R^{2′} is phenyl substituted with hydroxyl. In some embodiments, R^{2′} is phenyl substituted with cyano.

In some embodiments, R^{2′} is unsubstituted phenyl.

In some embodiments, R^{2′} is 5-10 membered heteroaryl optionally substituted with C1-C6 alkyl. In some embodiments, R^{2′} is 5-10 membered heteroaryl substituted with C1-C6 alkyl. In some embodiments, R^{2′} is 5-10 membered heteroaryl substituted with methyl. In some embodiments, R^{2′} is unsubstituted 5-10 membered heteroaryl. In some embodiments, the R^{2′} 5-10 membered heteroaryl is quinolinyl, benzimidazolyl, or pyridyl.

In some embodiments, R^{2′} is C3-C9 cycloalkyl optionally substituted with hydroxyl or —NR^{B2}R^{C2}. In some embodiments, R^{2′} is C3-C9 cycloalkyl substituted with hydroxyl or —N^{B2}R^{C2} In some embodiments, R^{2′} is C3-C9 cycloalkyl substituted with hydroxyl. In some embodiments, R^{2′} is C3-C9 cycloalkyl substituted with —NR^{B2}R^{C2}.

In some embodiments, the compound of Formula (I) is a compound of Formula (I-G):

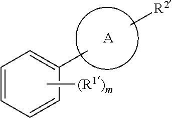

wherein:
each R^{1′} is independently hydroxyl, cyano, —CO₂R^{B}, or —C(═O)NR^{B}R^{C}; and
R^{2′} is 4-9 membered heterocyclyl optionally substituted with R^{G}, phenyl optionally substituted with 1-2 substituents independently selected from hydroxyl and cyano, 5-10 membered heteroaryl optionally substituted with C1-C6 alkyl, or C3-C9 cycloalkyl optionally substituted with hydroxyl or —NR^{B2}R^{C2}.

In some embodiments, m is 0. In some embodiments, m is 1. In some embodiments, m is 2. In some embodiments, m is 3.

In some embodiments, at least one R^{1′} is independently hydroxyl. In some embodiments, at least one R^{1′} is independently cyano. In some embodiments, at least one R^{1′} is independently —CO₂R^{B}.

In some embodiments, at least one R^{1′} is independently —C(═O)NR^{B}R^{C}.

In some embodiments, R^{2′} is 4-9 membered heterocyclyl optionally substituted with R^{G}.

In some embodiments, R^{2′} is phenyl optionally substituted with 1-2 substituents independently selected from hydroxyl and cyano. In some embodiments, R^{2′} is phenyl substituted with 1-2 substituents independently selected from hydroxyl and cyano. In some embodiments, R^{2′} is phenyl substituted with hydroxyl. In some embodiments, R^{2′} is phenyl substituted with cyano. In some embodiments, R^{2′} is unsubstituted phenyl.

In some embodiments, R^{2′} is 5-10 membered heteroaryl optionally substituted with C1-C6 alkyl. In some embodiments, R^{2′} is 5-10 membered heteroaryl substituted with C1-C6 alkyl. In some embodiments, R^{2′} is 5-10 membered heteroaryl substituted with methyl. In some embodiments, R^{2′} is unsubstituted 5-10 membered heteroaryl. In some embodiments, the R^{2′} 5-10 membered heteroaryl is quinolinyl, benzimidazolyl, or pyridyl.

In some embodiments, R^{2′} is C3-C9 cycloalkyl optionally substituted with hydroxyl or —NR^{B2}R^{C2}. In some embodiments, R^{2′} is C3-C9 cycloalkyl substituted with hydroxyl or —NR^{B2}R^{C2}. In some embodiments, R^{2′} is C3-C9 cycloalkyl substituted with hydroxyl. In some embodiments, R^{2′} is C3-C9 cycloalkyl substituted with —NR^{B2}R^{C2}.

In some embodiments, the compound of Formula (I) is a compound of Formula (I-H):

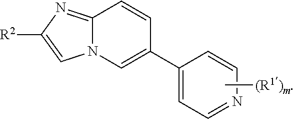

In some embodiments, the compound of Formula (I) is a compound of Formula (I-I):

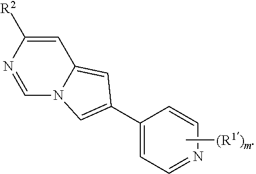

In some embodiments, the compound of Formula (I), or a pharmaceutically acceptable salt thereof, is selected from a compound in Table 1, or a pharmaceutically acceptable salt thereof.

TABLE 1
Selected Compounds of Formula (I)

| Compound | |
|---|---|
| No. | Structure |
| 1 | 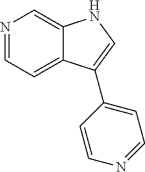 [SMILES: c1cc(-c2c[nH]c3cnccc23)ccn1] |
| 2 | 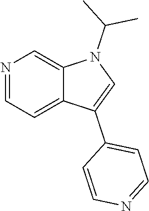 [SMILES: CC(C)n1cc(-c2ccncc2)c2ccncc21] |
| 3 | 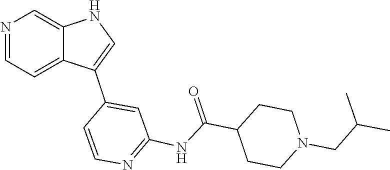 [SMILES: CC(C)CN1CCC(C(=O)Nc2cc(-c3c[nH]c4cnccc34)ccn2)CC1] |
| 4 | 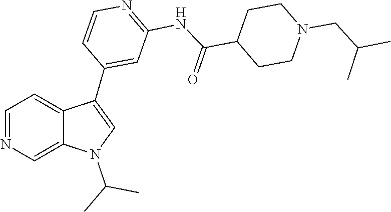 [SMILES: CC(C)CN1CCC(C(=O)Nc2cc(-c3cn(C(C)C)c4cnccc34)ccn2)CC1] |
| 5 | 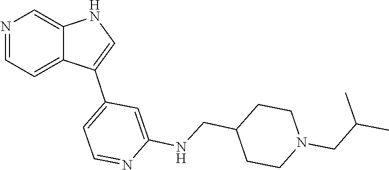 [SMILES: CC(C)CN1CCC(CNc2cc(-c3c[nH]c4cnccc34)ccn2)CC1] |
| 6 | 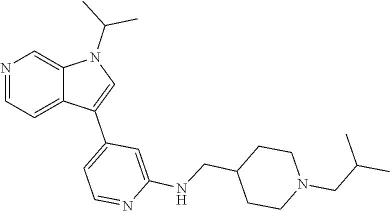 [SMILES: CC(C)CN1CCC(CNc2cc(-c3cn(C(C)C)c4cnccc34)ccn2)CC1] |
| 7 | 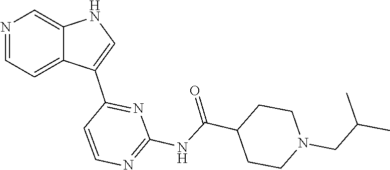 [SMILES: CC(C)CN1CCC(C(=O)Nc2nccc(-c3c[nH]c4cnccc34)n2)CC1] |
| 8 | 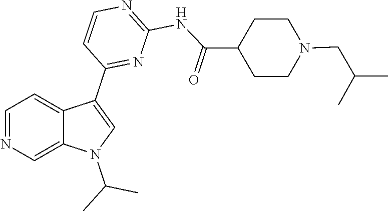 [SMILES: CC(C)CN1CCC(C(=O)Nc2nccc(-c3cn(C(C)C)c4cnccc34)n2)CC1] |
| 9 | 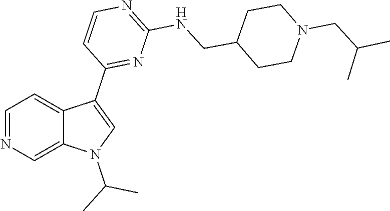 [SMILES: CC(C)CN1CCC(CNc2nccc(-c3cn(C(C)C)c4cnccc34)n2)CC1] |
| 10 | 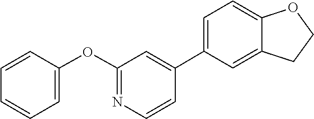 [SMILES: c1ccc(Oc2cc(-c3ccc4c(c3)CCO4)ccn2)cc1] |
| 11 | 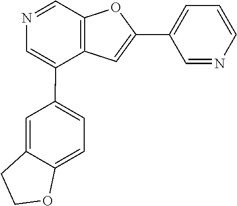 [SMILES: c1cncc(-c2cc3c(-c4ccc5c(c4)CCO5)cncc3o2)c1] |
| 12 | 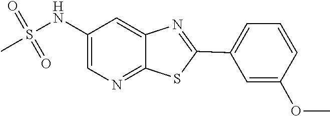 |
| 13 | 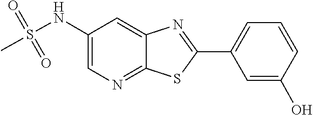 [SMILES: CS(=O)(=O)Nc1cnc2sc(-c3cccc(O)c3)nc2c1] |
| 14 | 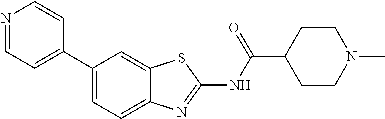 [SMILES: CN1CCC(C(=O)Nc2nc3ccc(-c4ccncc4)cc3s2)CC1] |
| 15 | 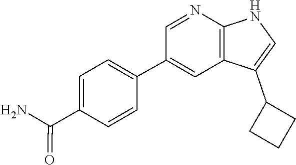 [SMILES: NC(=O)c1ccc(-c2cnc3[nH]cc(C4CCC4)c3c2)cc1] |
| 16 | 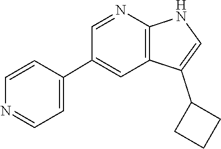 [SMILES: c1cc(-c2cnc3[nH]cc(C4CCC4)c3c2)ccn1] |
| 17 | 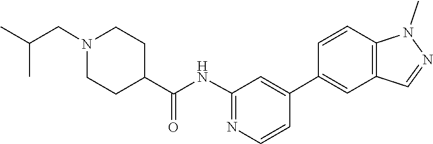 [SMILES: CC(C)CN1CCC(C(=O)Nc2cc(-c3ccc4c(cnn4C)c3)ccn2)CC1] |
| 18 | 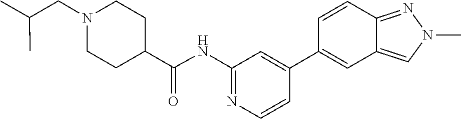 [SMILES: CC(C)CN1CCC(C(=O)Nc2cc(-c3ccc4nn(C)cc4c3)ccn2)CC1] |
| 19 | 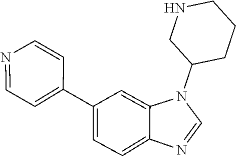 [SMILES: c1cc(-c2ccc3ncn(C4CCCNC4)c3c2)ccn1] |
| 20 | 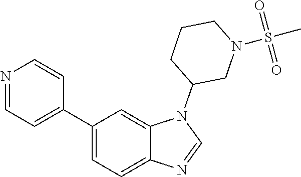 [SMILES: CS(=O)(=O)N1CCCC(n2cnc3ccc(-c4ccncc4)cc32)C1] |
| 21 | 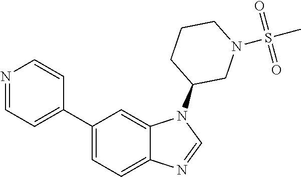 [SMILES: CS(=O)(=O)N1CCC[C@H](n2cnc3ccc(-c4ccncc4)cc32)C1] |
| 22 | 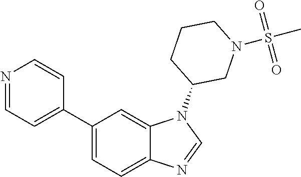 [SMILES: CS(=O)(=O)N1CCC[C@@H](n2cnc3ccc(-c4ccncc4)cc32)C1] |
| 23 | 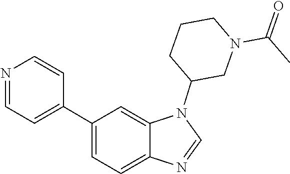 [SMILES: CC(=O)N1CCCC(n2cnc3ccc(-c4ccncc4)cc32)C1] |
| 24 | 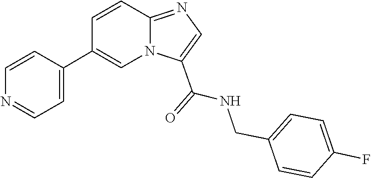 [SMILES: O=C(NCc1ccc(F)cc1)c1cnc2ccc(-c3ccncc3)cn12] |
| 25 | 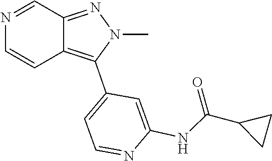 [SMILES: Cn1nc2cnccc2c1-c1ccnc(NC(=O)C2CC2)c1] |
| 26 | 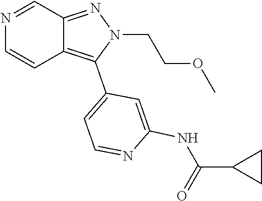 [SMILES: COCCn1nc2cnccc2c1-c1ccnc(NC(=O)C2CC2)c1] |
| 27 | 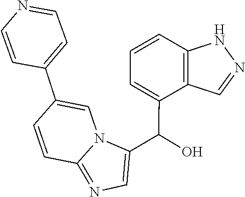 [SMILES: OC(c1cccc2[nH]ncc12)c1cnc2ccc(-c3ccncc3)cn12] |
| 28 | 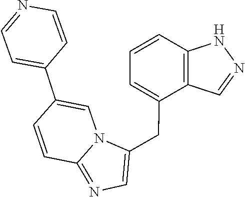 [SMILES: c1cc(Cc2cnc3ccc(-c4ccncc4)cn23)c2cn[nH]c2c1] |
| 29 | 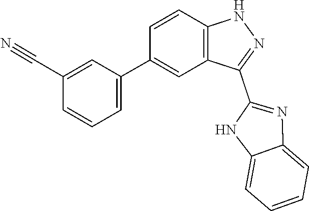 [SMILES: N#Cc1cccc(-c2ccc3[nH]nc(-c4nc5ccccc5[nH]4)c3c2)c1] |
| 30 | 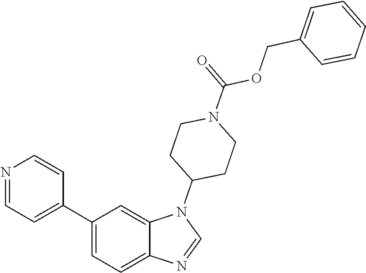 [SMILES: O=C(OCc1ccccc1)N1CCC(n2cnc3ccc(-c4ccncc4)cc32)CC1] |
| 31 | 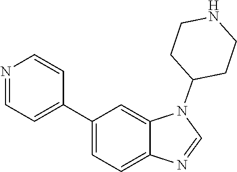 [SMILES: c1cc(-c2ccc3ncn(C4CCNCC4)c3c2)ccn1] |
| 32 | 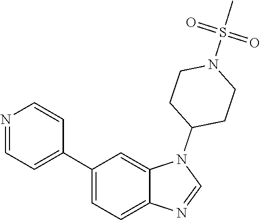 [SMILES: CS(=O)(=O)N1CCC(n2cnc3ccc(-c4ccncc4)cc32)CC1] |
| 33 | 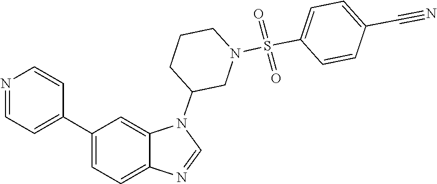 [SMILES: N#Cc1ccc(S(=O)(=O)N2CCCC(n3cnc4ccc(-c5ccncc5)cc43)C2)cc1] |
| 34 | 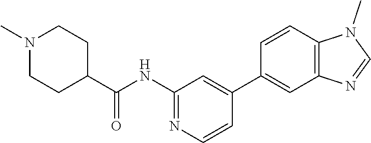 [SMILES: CN1CCC(C(=O)Nc2cc(-c3ccc4c(c3)ncn4C)ccn2)CC1] |
| 35 | 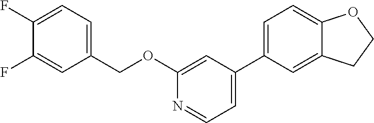 [SMILES: Fc1ccc(COc2cc(-c3ccc4c(c3)CCO4)ccn2)cc1F] |
| 36 | 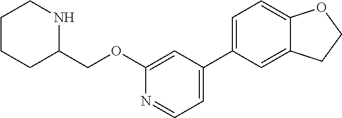 [SMILES: c1cc(-c2ccc3c(c2)CCO3)cc(OCC2CCCCN2)n1] |
| 37 | 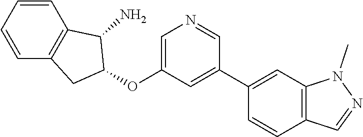 [SMILES: Cn1ncc2ccc(-c3cncc(O[C@@H]4Cc5ccccc5[C@@H]4N)c3)cc21] |
| 38 | 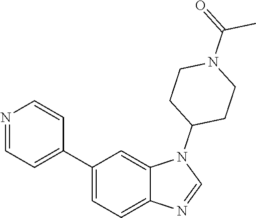 [SMILES: CC(=O)N1CCC(n2cnc3ccc(-c4ccncc4)cc32)CC1] |
| 39 | 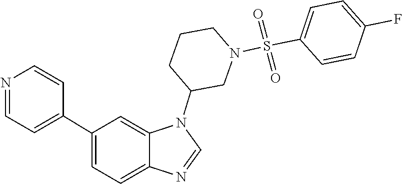 [SMILES: O=S(=O)(c1ccc(F)cc1)N1CCCC(n2cnc3ccc(-c4ccncc4)cc32)C1] |
| 40 | 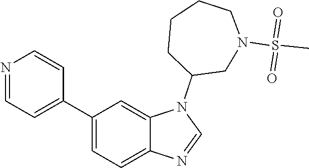 [SMILES: CS(=O)(=O)N1CCCCC(n2cnc3ccc(-c4ccncc4)cc32)C1] |
| 41 | 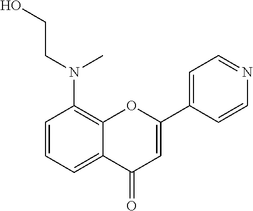 [SMILES: CN(CCO)c1cccc2c(=O)cc(-c3ccncc3)oc12] |
| 42 | 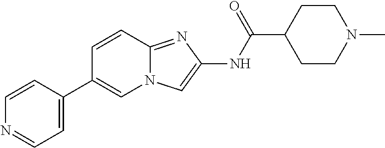 [SMILES: CN1CCC(C(=O)Nc2cn3cc(-c4ccncc4)ccc3n2)CC1] |
| 43 | 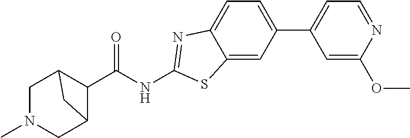 |
| 44 | 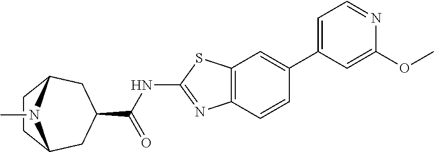 |
| 45 | 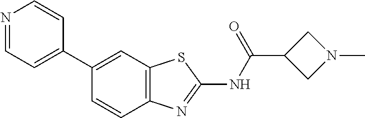 [SMILES: CN1CC(C(=O)Nc2nc3ccc(-c4ccncc4)cc3s2)C1] |
| 46 | 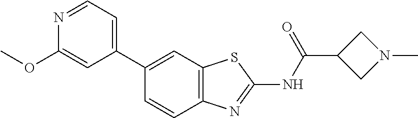 |
| 47 | 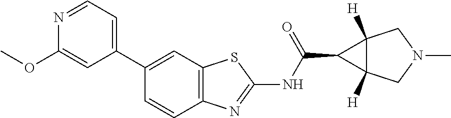 |
| 48 | 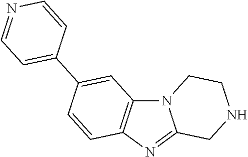 [SMILES: c1cc(-c2ccc3nc4n(c3c2)CCNC4)ccn1] |
| 49 | 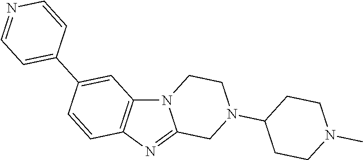 [SMILES: CN1CCC(N2CCn3c(nc4ccc(-c5ccncc5)cc43)C2)CC1] |
| 50 | 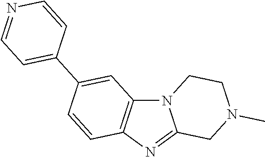 [SMILES: CN1CCn2c(nc3ccc(-c4ccncc4)cc32)C1] |
| 51 | 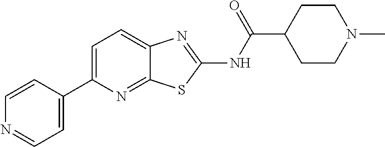 [SMILES: CN1CCC(C(=O)Nc2nc3ccc(-c4ccncc4)nc3s2)CC1] |
| 52 | 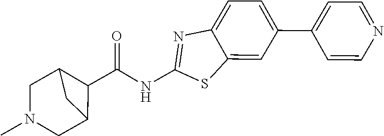 [SMILES: CN1CC2CC(C1)C2C(=O)Nc1nc2ccc(-c3ccncc3)cc2s1] |
| 53 | 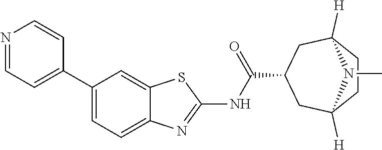 [SMILES: CN1[C@@H]2CC[C@H]1C[C@H](C(=O)Nc1nc3ccc(-c4ccncc4)cc3s1)C2] |
| 54 | 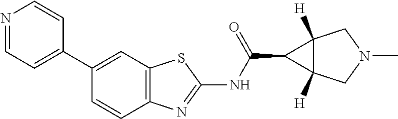 [SMILES: CN1C[C@@H]2[C@H](C1)[C@H]2C(=O)Nc1nc2ccc(-c3ccncc3)cc2s1] |
| 55 | 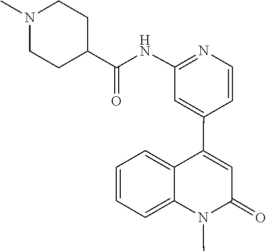 [SMILES: CN1CCC(C(=O)Nc2cc(-c3cc(=O)n(C)c4ccccc34)ccn2)CC1] |
| 56 | 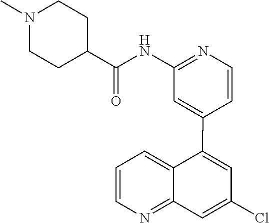 [SMILES: CN1CCC(C(=O)Nc2cc(-c3cc(Cl)cc4ncccc34)ccn2)CC1] |
| 57 | 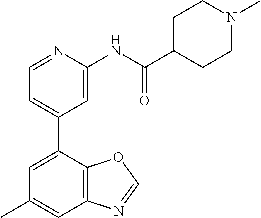 |
| 58 | 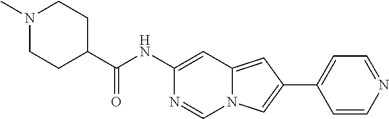 [SMILES: CN1CCC(C(=O)Nc2cc3cc(-c4ccncc4)cn3cn2)CC1] |
| 59 | 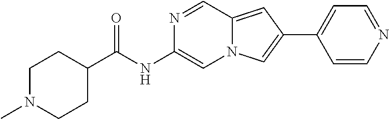 [SMILES: CN1CCC(C(=O)Nc2cn3cc(-c4ccncc4)cc3cn2)CC1] |
| 60 | 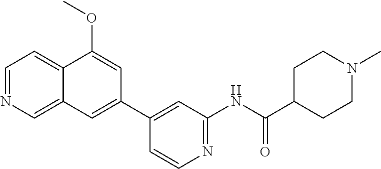 [SMILES: COc1cc(-c2ccnc(NC(=O)C3CCN(C)CC3)c2)cc2cnccc12] |
| 61 | 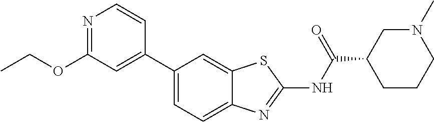 [SMILES: CCOc1cc(-c2ccc3nc(NC(=O)[C@H]4CCCN(C)C4)sc3c2)ccn1] |
| 62 | 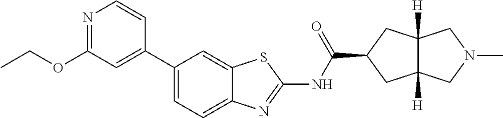 [SMILES: CCOc1cc(-c2ccc3nc(NC(=O)[C@H]4C[C@@H]5CN(C)C[C@@H]5C4)sc3c2)ccn1] |
| 63 | 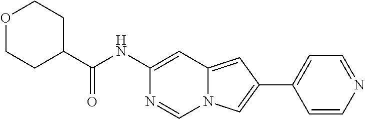 [SMILES: O=C(Nc1cc2cc(-c3ccncc3)cn2cn1)C1CCOCC1] |
| 64 | 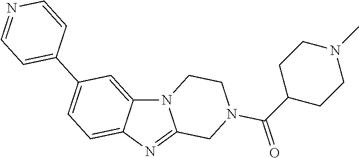 [SMILES: CN1CCC(C(=O)N2CCn3c(nc4ccc(-c5ccncc5)cc43)C2)CC1] |
| 65 | 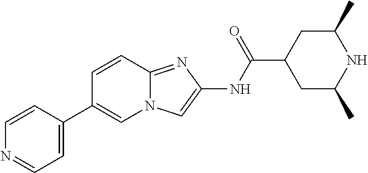 [SMILES: C[C@@H]1CC(C(=O)Nc2cn3cc(-c4ccncc4)ccc3n2)C[C@H](C)N1] |
| 66 | 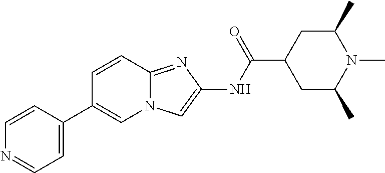 [SMILES: C[C@@H]1CC(C(=O)Nc2cn3cc(-c4ccncc4)ccc3n2)C[C@H](C)N1C] |
| 67 | 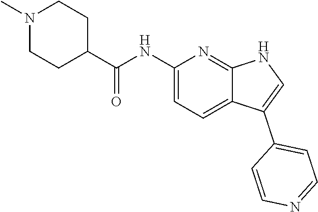 [SMILES: CN1CCC(C(=O)Nc2ccc3c(-c4ccncc4)c[nH]c3n2)CC1] |
| 68 | 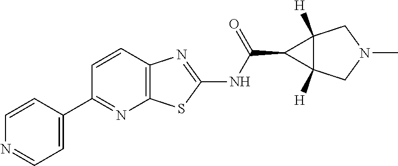 [SMILES: CN1C[C@@H]2[C@H](C1)[C@H]2C(=O)Nc1nc2ccc(-c3ccncc3)nc2s1] |
| 69 | 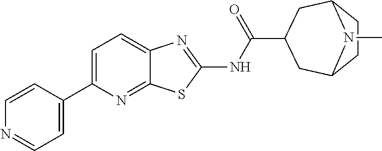 [SMILES: CN1C2CCC1CC(C(=O)Nc1nc3ccc(-c4ccncc4)nc3s1)C2] |
| 70 | 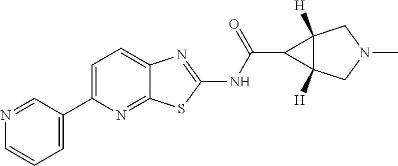 [SMILES: CN1C[C@@H]2C(C(=O)Nc3nc4ccc(-c5cccnc5)nc4s3)[C@@H]2C1] |
| 71 | 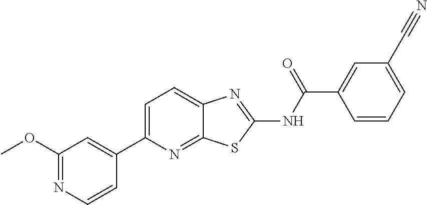 [SMILES: COc1cc(-c2ccc3nc(NC(=O)c4cccc(C#N)c4)sc3n2)ccn1] |
| 72 | 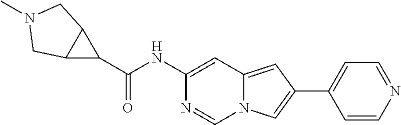 [SMILES: CN1CC2C(C1)C2C(=O)Nc1cc2cc(-c3ccncc3)cn2cn1] |
| 73 | 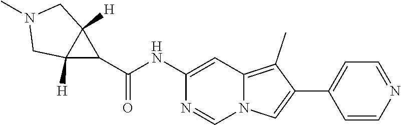 [SMILES: Cc1c(-c2ccncc2)cn2cnc(NC(=O)C3[C@H]4CN(C)C[C@@H]34)cc12] |
| 74 | 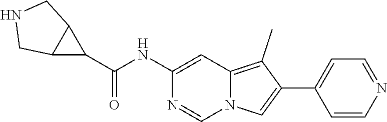 [SMILES: Cc1c(-c2ccncc2)cn2cnc(NC(=O)C3C4CNCC43)cc12] |
| 75 | 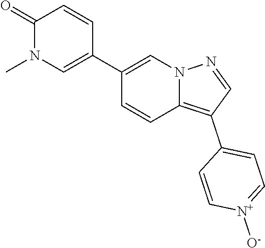 |
| 76 | 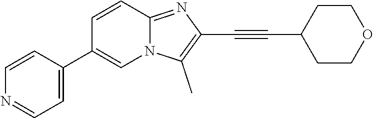 [SMILES: Cc1c(C#CC2CCOCC2)nc2ccc(-c3ccncc3)cn12] |
| 77 | 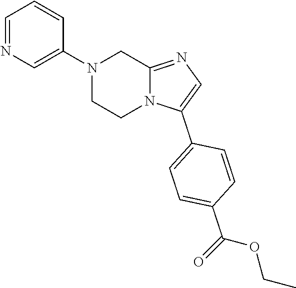 [SMILES: CCOC(=O)c1ccc(-c2cnc3n2CCN(c2cccnc2)C3)cc1] |
| 78 | 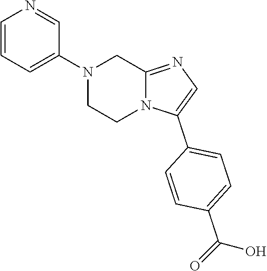 [SMILES: O=C(O)c1ccc(-c2cnc3n2CCN(c2cccnc2)C3)cc1] |
| 79 | 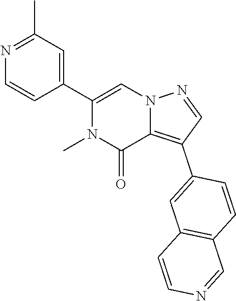 [SMILES: Cc1cc(-c2cn3ncc(-c4ccc5cnccc5c4)c3c(=O)n2C)ccn1] |
| 80 | 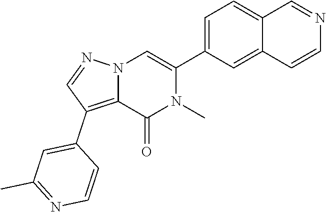 [SMILES: Cc1cc(-c2cnn3cc(-c4ccc5cnccc5c4)n(C)c(=O)c23)ccn1] |
| 81 | 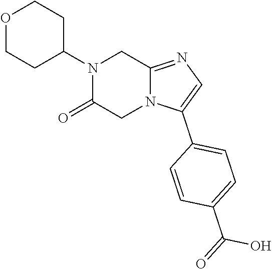 [SMILES: O=C(O)c1ccc(-c2cnc3n2CC(=O)N(C2CCOCC2)C3)cc1] |
| 82 | 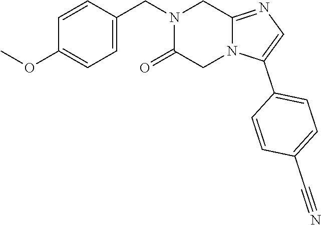 [SMILES: COc1ccc(CN2Cc3ncc(-c4ccc(C#N)cc4)n3CC2=O)cc1] |
| 83 | 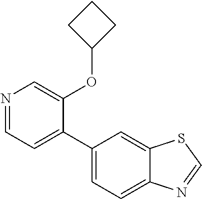 [SMILES: c1cc(-c2ccc3ncsc3c2)c(OC2CCC2)cn1] |
| 84 | 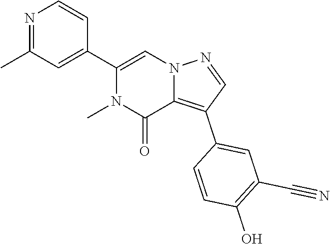 [SMILES: Cc1cc(-c2cn3ncc(-c4ccc(O)c(C#N)c4)c3c(=O)n2C)ccn1] |
| 85 | 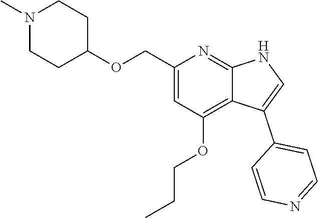 [SMILES: CCCOc1cc(COC2CCN(C)CC2)nc2[nH]cc(-c3ccncc3)c12] |
| 86 | 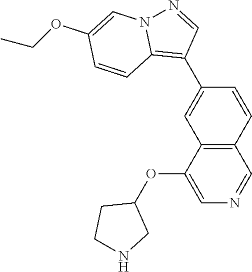 [SMILES: CCOc1ccc2c(-c3ccc4cncc(OC5CCNC5)c4c3)cnn2c1] |
| 87 | 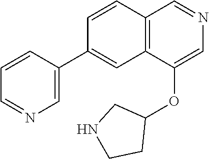 [SMILES: c1cncc(-c2ccc3cncc(OC4CCNC4)c3c2)c1] |
| 88 | 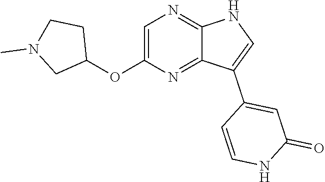 [SMILES: CN1CCC(Oc2cnc3[nH]cc(-c4cc[nH]c(=O)c4)c3n2)C1] |
| 89 | 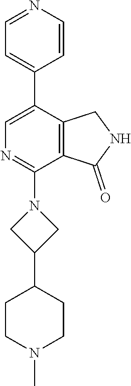 [SMILES: CN1CCC(C2CN(c3ncc(-c4ccncc4)c4c3C(=O)NC4)C2)CC1] |
| 90 | 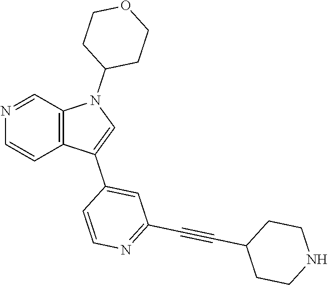 [SMILES: C(#CC1CCNCC1)c1cc(-c2cn(C3CCOCC3)c3cnccc23)ccn1] |
| 91 | 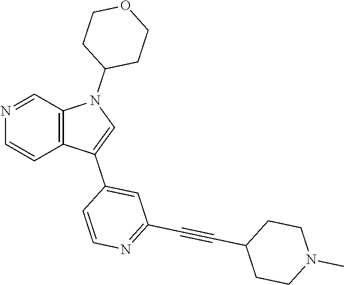 [SMILES: CN1CCC(C#Cc2cc(-c3cn(C4CCOCC4)c4cnccc34)ccn2)CC1] |
| 92 | 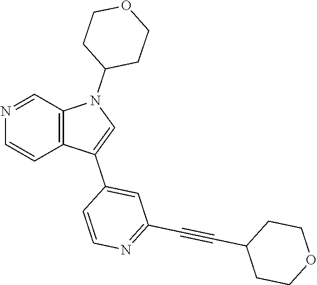 [SMILES: C(#CC1CCOCC1)c1cc(-c2cn(C3CCOCC3)c3cnccc23)ccn1] |
| 93 | 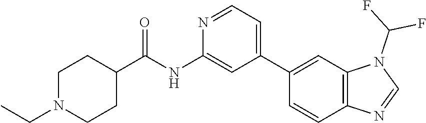 [SMILES: CCN1CCC(C(=O)Nc2cc(-c3ccc4ncn(C(F)F)c4c3)ccn2)CC1] |
| 94 | 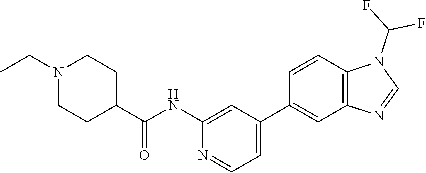 [SMILES: CCN1CCC(C(=O)Nc2cc(-c3ccc4c(c3)ncn4C(F)F)ccn2)CC1] |
| 95 | 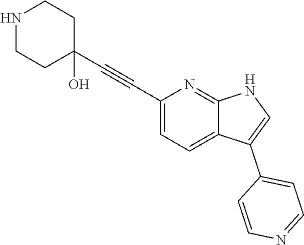 [SMILES: OC1(C#Cc2ccc3c(-c4ccncc4)c[nH]c3n2)CCNCC1] |
| 96 | 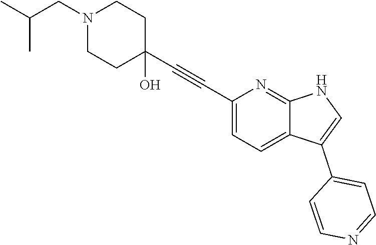 [SMILES: CC(C)CN1CCC(O)(C#Cc2ccc3c(-c4ccncc4)c[nH]c3n2)CC1] |
| 97 | 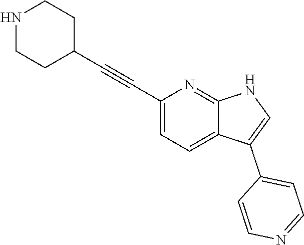 [SMILES: C(#CC1CCNCC1)c1ccc2c(-c3ccncc3)c[nH]c2n1] |
| 98 | 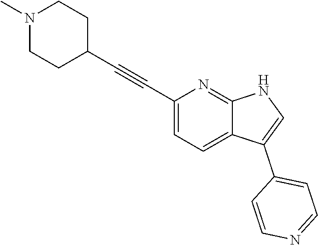 [SMILES: CN1CCC(C#Cc2ccc3c(-c4ccncc4)c[nH]c3n2)CC1] |
| 99 | 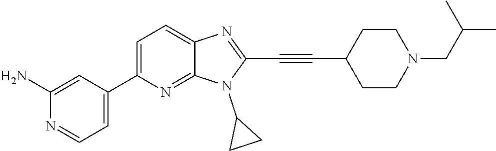 [SMILES: CC(C)CN1CCC(C#Cc2nc3ccc(-c4ccnc(N)c4)nc3n2C2CC2)CC1] |
| 100 | |
| 101 | |
| 102 | |
| 103 | |
| 104 | 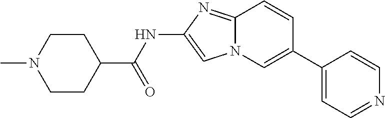 [SMILES: CN1CCC(C(=O)Nc2cn3cc(-c4ccncc4)ccc3n2)CC1] |
| 105 | 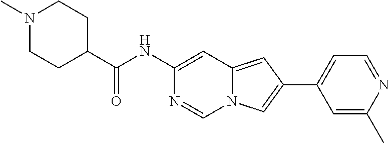 |
| 106 | 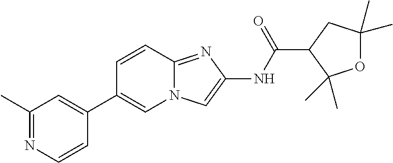 |
| 107 | 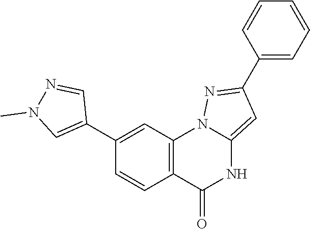 [SMILES: Cn1cc(-c2ccc3c(=O)[nH]c4cc(-c5ccccc5)nn4c3c2)cn1] |

### Compounds of Formula (II)

The substituent groups used in this section (e.g., R¹, R², and the like) refer solely to the groups in Formula (II).

Some embodiments provide a compound of Formula (II):

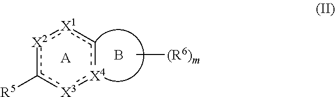

or a pharmaceutically acceptable salt thereof, wherein:
Ring A is aromatic;
Ring B is phenyl, 5-6 membered heteroaryl, or 5-7 membered monocyclic heterocyclyl such that together Ring A and Ring B form a 9-10 membered heteroaryl or a 9-10 membered heterocyclyl ring system;
X¹ is absent, CR¹, N, or NR^{A};
X² is CR², C═O, N, or NR, wherein when X² is C═O, X¹ is NR^{A}, and when X¹ is absent, X² is directly connected to the carbon atom shared by Ring A and Ring B;
X³ is C, CR³ or N;
X⁴ is C or N;
one of R¹, R², R³, and R⁵ is halogen, cyano, C1-C6 haloalkyl, C1-C6 alkyl optionally substituted with 1-2 substituents independently selected from (i) 5-6 membered heteroaryl optionally substituted with 1-2 independently selected C1-C6 alkyl or 3-6 membered heterocyclyl optionally substituted with C1-C6 alkyl, (ii) 4-6 membered heterocyclyl optionally substituted with benzyl, (iii) cyano, (iv) phenyl optionally substituted with halogen, or (v) —NR^{E}R^{F}; C1-C6 alkoxy, —(CH₂)ₙ-Q-(4-6 membered heterocyclyl optionally substituted with C1-C6 alkyl), —NHC(═O)(CH₂)ₙR^{C}, 4-10 membered heterocyclyl optionally substituted with phenoxy, C1-C6 alkyl, or 5-6 membered heteroaryl; —NR^{E}R^{F}, C3-C6 cycloalkyl, C3-C6 cycloalkyloxy,

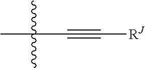

phenoxy optionally substituted with 1-2 substituents independently selected from halogen and C1-C6 haloalkyl; 5-6 membered heteroaryloxy optionally substituted with 1-2 independently selected C1-C6 alkyl, 5-10 membered heteroaryl optionally substituted with C1-C6 alkyl, —SO₂(C1-C6 alkyl), —(CH₂)ₙCO₂R^{D};
and the other of R¹, R², R³, and R⁵ are independently hydrogen, halogen, cyano, C1-C6 alkyl, C1-C6 alkoxy, C1-C6 haloalkyl, C3-C6 cycloalkyloxy, or 4-6 membered heterocyclyl optionally substituted with 5-6 membered heteroaryl; or
R³ and R⁵, together with the carbon atoms to which they are attached form a 6 membered heterocyclyl optionally substituted with C1-C6 alkyl or C3-C6 cycloalkyl; or R³ and one R adjacent to Ring A, together with the carbon atoms to which they are attached form a 7 membered heterocyclyl; or
R¹ and one R⁶ adjacent to Ring A, together with the carbon atoms to which they are attached form a 7 membered heterocyclyl; or
R¹ and R², R² and R³, and R³ and R⁵, together with the carbon atoms to which they are attached form a C3-C5 cycloalkyl;
Q and Q¹ are each independently —O— or —C(═O)—;
   R^{A} and R^{B} are independently hydrogen, C3-C6 cycloalkyl, or C1-C6 alkyl;
R^{C} is 4-6 membered heterocyclyl optionally substituted with C1-C6 alkyl, or 5-6 membered heteroaryl optionally substituted with C1-C6 alkoxy or C1-C6 alkyl,
R^{D} is hydrogen or C1-C6 alkyl;
R^{E} and R^{F} are independently hydrogen, C1-C6 alkyl, —C(═O)—C1-C6 alkyl, or 4-6 membered heterocyclyl;
R^{G} and R^{H} are independently hydrogen, C1-C6 alkyl, or C3-C6 cycloalkyl;
R^{I} is 5-6 membered heteroaryl optionally substituted with C1-C6 alkyl or halogen; C1-C6 alkyl substituted with 1-2 independently selected amino or phenyl optionally substituted with halogen; or phenyl optionally substituted with halogen;
R^{J} is a 4-6 membered heterocyclyl or a 5-6 membered heteroaryl;
R⁶ is C1-C6 alkyl optionally substituted with (i) 4-10 membered heterocyclyl optionally substituted C1-C6 alkyl or (ii) 9-10 membered heteroaryl; C1-C6 haloalkyl, C3-C6 cycloalkyl optionally substituted with —CO₂R^{D}, —NHC(═O)R^{C}, —NR^{G}R^{H}, 4-10 membered heterocyclyl optionally substituted with C1-C6 alkyl, —(CH₂)ₚC(═O)NHR^{I}, 5-10 membered heteroaryl optionally substituted with C1-C6 alkyl, or —(CH₂)ₛ-Q¹-(4-6 membered heterocyclyl optionally substituted with C1-C6 alkyl); or
two geminal R⁶, together with the carbon atom to which they are attached form a C3-C6 spirocycloalkyl;
m is 0, 1, 2, or 3;
n is 0, 1, or 2;
p is 0, 1, 2, 3, or 4; and
s is 0, 1, or 2.

In some embodiments, Ring B is phenyl.

In some embodiments, Ring B is 5-6 membered heteroaryl.

In some embodiments, Ring B is 5-7 membered monocyclic heterocyclyl.

In some embodiments, Ring A and Ring B form a 9-10 membered heteroaryl ring system.

In some embodiments, Ring A and Ring B form a 9-10 membered heterocyclyl ring system.

In some embodiments, X¹ is absent. In some embodiments, X¹ is CR¹. In some embodiments, X¹ is N. In some embodiments, X¹ is NR^{A}.

In some embodiments, X² is CR². In some embodiments, X² is C═O. In some embodiments, X² is N. In some embodiments, X² is NR^{B}.

In some embodiments, X³ is C. In some embodiments, X³ is CR³. In some embodiments, X³ is N. In some embodiments, X⁴ is C. In some embodiments, X⁴ is N.

In some embodiments, R^{A} is hydrogen. In some embodiments, R^{A} is C1-C6 alkyl. In some embodiments, R^{B} is hydrogen. In some embodiments, R^{B} is C1-C6 alkyl. In some embodiments, R^{A} and R^{B} are each hydrogen. In some embodiments, R^{A} and R^{B} are each methyl. In some embodiments, one of R^{A} and R^{B} is hydrogen, and the other of R^{A} and R^{B} is C1-C6 alkyl.

In some embodiments, one of R¹, R², R³, and R⁵ is halogen.

In some embodiments, one of R¹, R², R³, and R⁵ is cyano.

In some embodiments, one of R¹, R², R³, and R⁵ is C1-C6 haloalkyl. In some embodiments, one of R¹, R², R³, and R⁵ is —CF³.

In some embodiments, one of R¹, R², R³, and R⁵ is C1-C6 alkyl optionally substituted with 5-6 membered heteroaryl optionally substituted with 1-2 independently selected C1-C6 alkyl or 3-6 membered heterocyclyl optionally substituted with C1-C6 alkyl.

In some embodiments, one of R¹, R², R³, and R⁵ is C1-C6 alkyl substituted with 5-6 membered heteroaryl optionally substituted with 1-2 independently selected C1-C6 alkyl.

In some embodiments, one of R¹, R², R³, and R⁵ is C1-C6 alkyl substituted with 5-6 membered heteroaryl substituted with 1-2 independently selected C1-C6 alkyl.

In some embodiments, one of R¹, R², R³, and R⁵ is C1-C6 alkyl substituted with 5-6 membered heteroaryl optionally substituted with 3-6 membered heterocyclyl optionally substituted with C1-C6 alkyl.

In some embodiments, one of R¹, R², R³, and R⁵ is C1-C6 alkyl substituted with 5-6 membered heteroaryl substituted with 3-6 membered heterocyclyl optionally substituted with C1-C6 alkyl.

In some embodiments, one of R¹, R², R³, and R⁵ is C1-C6 alkyl substituted with 5-6 membered heteroaryl substituted with 3-6 membered heterocyclyl substituted with C1-C6 alkyl.

In some embodiments, one of R¹, R², R³, and R⁵ is C1-C6 alkyl substituted with an unsubstituted 5-6 membered heteroaryl.

In some embodiments, one of R¹, R², R³, and R⁵ is C1-C6 alkyl optionally substituted with 4-6 membered heterocyclyl optionally substituted with benzyl.

In some embodiments, one of R¹, R², R³, and R⁵ is C1-C6 alkyl substituted with 4-6 membered heterocyclyl optionally substituted with benzyl.

In some embodiments, one of R¹, R², R³, and R⁵ is C1-C6 alkyl substituted with 4-6 membered heterocyclyl substituted with benzyl.

In some embodiments, one of R¹, R², R³, and R⁵ is C1-C6 alkyl substituted with an unsubstituted 4-6 membered heterocyclyl.

In some embodiments, one of R¹, R², R³, and R⁵ is C1-C6 alkyl optionally substituted with cyano.

In some embodiments, one of R¹, R², R³, and R⁵ is C1-C6 alkyl optionally substituted with phenyl optionally substituted with halogen.

In some embodiments, one of R¹, R², R³, and R⁵ is C1-C6 alkyl substituted with phenyl optionally substituted with halogen.

In some embodiments, one of R¹, R², R³, and R⁵ is C1-C6 alkyl substituted with phenyl substituted with halogen.

In some embodiments, one of R¹, R², R³, and R⁵ is C1-C6 alkyl substituted with unsubstituted phenyl.

In some embodiments, one of R¹, R², R³, and R⁵ is C1-C6 alkyl optionally substituted with —NR^{E}R^{F}.

In some embodiments, one of R¹, R², R³, and R⁵ is C1-C6 alkoxy. In some embodiments, one of R¹, R², R³, and R⁵ is —OCH₃.

In some embodiments, one of R¹, R², R³, and R⁵ is —(CH₂)ₙ-Q-(4-6 membered heterocyclyl optionally substituted with C1-C6 alkyl). In some embodiments, one of R¹, R², R³, and R⁵ is —(CH₂)ₙ-Q-(4-6 membered heterocyclyl substituted with C1-C6 alkyl). In some embodiments, one of R¹, R², R³, and R⁵ is —(CH₂)ₙ-Q-(4-6 membered heterocyclyl).

In some embodiments, one of R¹, R², R³, and R⁵ is —NHC(═O)(CH₂)ₙR^{C}.

In some embodiments, one of R¹, R², R³, and R⁵ is 4-10 membered heterocyclyl optionally substituted with phenoxy, C1-C6 alkyl, or 5-6 membered heteroaryl.

In some embodiments, one of R¹, R², R³, and R⁵ is 4-10 membered heterocyclyl substituted with phenoxy, C1-C6 alkyl, or 5-6 membered heteroaryl.

In some embodiments, one of R¹, R², R³, and R⁵ is 4-10 membered heterocyclyl substituted with phenoxy.

In some embodiments, one of R¹, R², R³, and R⁵ is 4-10 membered heterocyclyl substituted with C1-C6 alkyl.

In some embodiments, one of R¹, R², R³, and R⁵ is 4-10 membered heterocyclyl substituted with methyl.

In some embodiments, one of R¹, R², R³, and R⁵ is 4-10 membered heterocyclyl substituted with 5-6 membered heteroaryl.

In some embodiments, one of R¹, R², R³, and R⁵ is —NR^{E}R^{F}.

In some embodiments, one of R¹, R², R³, and R⁵ is C3-C6 cycloalkyl.

In some embodiments, one of R¹, R², R³, and R⁵ is C3-C6 cycloalkyloxy.

In some embodiments, one of R¹, R², R³, and R⁵ is

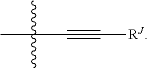

In some embodiments, one of R¹, R², R³, and R⁵ is phenoxy optionally substituted with 1-2 substituents independently selected from halogen and C1-C6 haloalkyl.

In some embodiments, one of R¹, R², R³, and R⁵ is phenoxy substituted with 1-2 substituents independently selected from halogen and C1-C6 haloalkyl.

In some embodiments, one of R¹, R², R³, and R⁵ is phenoxy substituted with 1-2 substituents independently selected halogen.

In some embodiments, one of R¹, R², R³, and R⁵ is phenoxy substituted with 1-2 substituents independently selected C1-C6 haloalkyl.

In some embodiments, one of R¹, R², R³, and R⁵ is unsubstituted phenoxy.

In some embodiments, one of R¹, R², R³, and R⁵ is 5-6 membered heteroaryloxy optionally substituted with 1-2 independently selected C1-C6 alkyl.

In some embodiments, one of R¹, R², R³, and R⁵ is 5-6 membered heteroaryloxy substituted with 1-2 independently selected C1-C6 alkyl.

In some embodiments, one of R¹, R², R³, and R⁵ is 5-6 membered heteroaryloxy substituted with C1-C6 alkyl.

In some embodiments, one of R¹, R², R³, and R⁵ is 5-6 membered heteroaryloxy substituted with two independently selected C1-C6 alkyl.

In some embodiments, one of R¹, R², R³, and R⁵ is 5-6 membered unsubstituted heteroaryloxy.

In some embodiments, one of R¹, R², R³, and R⁵ is 5-10 membered heteroaryl optionally substituted with C1-C6 alkyl. In some embodiments, one of R¹, R², R³, and R⁵ is pyridazinyl (e.g., 4-pyridazinyl) optionally substituted with C1-C6 alkyl. In some embodiments, one of R¹, R², R³, and R⁵ is

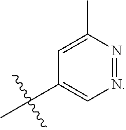 [SMILES: *c1cnnc(C)c1]

In some embodiments, one of R¹, R², R³, and R⁵ is 5-10 membered heteroaryl substituted with C1-C6 alkyl. In some embodiments, one of R¹, R², R³, and R⁵ is 5-10 membered heteroaryl substituted with methyl. In some embodiments, one of R¹, R², R³, and R⁵ is 5-10 membered unsubstituted heteroaryl.

In some embodiments, one of R¹, R², R³, and R⁵ is —SO₂(C1-C6 alkyl). In some embodiments, one of R¹, R², R³, and R⁵ is —SO₂(CH₃).

In some embodiments, one of R¹, R², R³, and R⁵ is —(CH₂)ₙCO₂R^{D}. In some embodiments, R^{D} is hydrogen. In some embodiments, R^{D} is C1-C6 alkyl. In some embodiments, R^{D} is methyl.

In some embodiments, one of R¹, R², R³, and R⁵ is an unsubstituted C1-C6 alkyl.

In some embodiments, one of R¹, R², R³, and R⁵ is methyl.

In some embodiments, the other of R¹, R², R³, and R⁵ are independently hydrogen, halogen, cyano, C1-C6 alkyl, C1-C6 alkoxy, C1-C6 haloalkyl, C3-C6 cycloalkyloxy, or 4-6 membered heterocyclyl optionally substituted with 5-6 membered heteroaryl.

In some embodiments, the other of R¹, R², R³, and R⁵ are independently hydrogen, halogen, cyano, C1-C6 alkyl, C1-C6 alkoxy, or C1-C6 haloalkyl.

In some embodiments, the other of R¹, R², R³, and R⁵ are independently hydrogen, halogen, or cyano.

In some embodiments, the other of R¹, R², R³, and R⁵ are independently hydrogen or halogen.

In some embodiments, the other of R¹, R², R³, and R⁵ are each hydrogen.

In some embodiments, one of R¹, R², R³, and R⁵ is halogen, cyano, C1-C6 alkyl optionally substituted with 1-2 substituents independently selected from 4-6 membered heterocyclyl optionally substituted with benzyl or cyano; C1-C6 alkoxy, —(CH₂)ₙ-Q-(4-6 membered heterocyclyl optionally substituted with C1-C6 alkyl), —NHC(═O)(CH₂)ₙR^{C}, 4-10 membered heterocyclyl optionally substituted with phenoxy or 5-6 membered heteroaryl; —NR^{E}R^{F}, C3-C6 cycloalkyl, C3-C6 cycloalkyloxy,

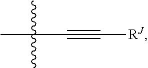

phenoxy optionally substituted with 1-2 substituents independently selected from halogen and C1-C6 haloalkyl; 5-6 membered heteroaryloxy optionally substituted with 1-2 independently selected C1-C6 alkyl; and the other of R¹, R², R³, and R⁵ are independently hydrogen, halogen, cyano, C1-C6 alkyl, C1-C6 alkoxy, C1-C6 haloalkyl, C3-C6 cycloalkyloxy, or 4-6 membered heterocyclyl optionally substituted with 5-6 membered heteroaryl.

In some embodiments, one of R¹, R², R³, and R⁵ is halogen, cyano, C1-C6 alkyl optionally substituted with 1-2 substituents independently selected from 4-6 membered heterocyclyl optionally substituted with benzyl or cyano; C1-C6 alkoxy, —(CH₂)ₙ-Q-(4-6 membered heterocyclyl optionally substituted with C1-C6 alkyl), —NHC(═O)(CH₂)ₙR^{C}, 4-10 membered heterocyclyl optionally substituted with phenoxy or 5-6 membered heteroaryl; —NR^{E}R^{F}, C3-C6 cycloalkyl, C3-C6 cycloalkyloxy,

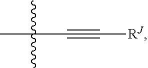

phenoxy optionally substituted with 1-2 substituents independently selected from halogen and C1-C6 haloalkyl; 5-6 membered heteroaryloxy optionally substituted with 1-2 independently selected C1-C6 alkyl; and the other of R¹, R², R³, and R⁵ are independently hydrogen, halogen, C1-C6 alkyl, C1-C6 alkoxy, C1-C6 haloalkyl, or 4-6 membered heterocyclyl.

In some embodiments, one of R¹, R², R³, and R⁵ is halogen, cyano, C1-C6 alkyl optionally substituted with 1-2 substituents independently selected from 4-6 membered heterocyclyl optionally substituted with benzyl or cyano; C1-C6 alkoxy, —(CH₂)ₙ-Q-(4-6 membered heterocyclyl optionally substituted with C1-C6 alkyl), —NHC(═O)(CH₂)ₙR^{C}, 4-10 membered heterocyclyl optionally substituted with phenoxy or 5-6 membered heteroaryl; —NR^{E}R^{F}, C3-C6 cycloalkyl, C3-C6 cycloalkyloxy,

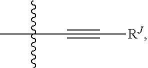

phenoxy optionally substituted with 1-2 substituents independently selected from halogen and C1-C6 haloalkyl; 5-6 membered heteroaryloxy optionally substituted with 1-2 independently selected C1-C6 alkyl; and the other of R¹, R², R³, and R⁵ are hydrogen.

In some embodiments, R³ and R₅, together with the carbon atoms to which they are attached form a 6 membered heterocyclyl optionally substituted with C1-C6 alkyl or C3-C6 cycloalkyl. In some embodiments, R³ and R₅, together with the carbon atoms to which they are attached form a 6 membered heterocyclyl substituted with C1-C6 alkyl. In some embodiments, R³ and R₅, together with the carbon atoms to which they are attached form a 6 membered heterocyclyl substituted with C3-C6 cycloalkyl. In some embodiments, R³ and R₅, together with the carbon atoms to which they are attached form a 6 membered heterocyclyl.

In some embodiments, R³ and one R⁶ adjacent to Ring A, together with the carbon atoms to which they are attached form a 7 membered heterocyclyl.

In some embodiments, R¹ and one R⁶ adjacent to Ring A, together with the carbon atoms to which they are attached form a 7 membered heterocyclyl.

In some embodiments, R¹ and R² together with the carbon atoms to which they are attached form a C3-C5 cycloalkyl.

In some embodiments, R² and R³ together with the carbon atoms to which they are attached form a C3-C5 cycloalkyl.

In some embodiments, R³ and R₅, together with the carbon atoms to which they are attached form a C3-C5 cycloalkyl.

In some embodiments, Q is —O—. In some embodiments, Q is —C(═O)—. In some embodiments, Q¹ is —O—. In some embodiments, Q¹ is —C(═O)—.

In some embodiments, R^{C} is 4-6 membered heterocyclyl optionally substituted with C1-C6 alkyl. In some embodiments, R^{C} is 4-6 membered heterocyclyl substituted with C1-C6 alkyl. In some embodiments, R^{C} is an unsubstituted 4-6 membered heterocyclyl.

In some embodiments, R^{C} is 5-6 membered heteroaryl optionally substituted with C1-C6 alkoxy. In some embodiments, R^{C} is 5-6 membered heteroaryl substituted with C1-C6 alkoxy. In some embodiments, R^{C} is 5-6 membered heteroaryl optionally substituted with C1-C6 alkyl. In some embodiments, R^{C} is 5-6 membered heteroaryl substituted with C1-C6 alkyl. In some embodiments, R^{C} is an unsubstituted 5-6 membered heteroaryl.

In some embodiments, R^{E} is hydrogen. In some embodiments, R^{E} is C1-C6 alkyl. In some embodiments, R^{E} is —C(═O)—C1-C6 alkyl. In some embodiments, R^{E} is 4-6 membered heterocyclyl. In some embodiments, R^{F} is hydrogen. In some embodiments, R^{F} is C1-C6 alkyl. In some embodiments, R^{F} is —C(═O)—C1-C6 alkyl. In some embodiments, R^{F} is 4-6 membered heterocyclyl. In some embodiments, R^{E} and R^{F} are each hydrogen. In some embodiments, R^{E} and R^{F} are each independently C1-C6 alkyl. In some embodiments, R^{E} and R^{F} are each methyl. In some embodiments, one of R^{E} and R^{F} is hydrogen, and the other of R^{E} and R^{F} is C1-C6 alkyl. In some embodiments, one of R^{E} and R^{F} is hydrogen, and the other of R^{E} and R^{F} is —C(═O)—C1-C6 alkyl. In some embodiments, one of R^{E} and R^{F} is hydrogen, and the other of R^{E} and R^{F} is 4-6 membered heterocyclyl.

In some embodiments, R^{J} is 4-6 membered heterocyclyl. In some embodiments, R^{J} is 5-6 membered heteroaryl.

In some embodiments, R⁶ is C1-C6 alkyl optionally substituted with (i) 4-10 membered heterocyclyl optionally substituted C1-C6 alkyl or (ii) 9-10 membered heteroaryl; C1-C6 haloalkyl, C3-C6 cycloalkyl optionally substituted with —CO₂R^{D}, —NHC(═O)R^{C}, —NR^{G}R^{H}, 4-10 membered heterocyclyl optionally substituted C1-C6 alkyl, —(CH₂)ₚC(═O)NHR^{I}, 5-10 membered heteroaryl optionally substituted with C1-C6 alkyl, or —(CH₂)ₛ-Q¹-(4-6 membered heterocyclyl optionally substituted with C1-C6 alkyl).

In some embodiments, R⁶ is C1-C6 alkyl optionally substituted with (i) 4-10 membered heterocyclyl optionally substituted C1-C6 alkyl or (ii) 9-10 membered heteroaryl.

In some embodiments, R⁶ is C1-C6 alkyl substituted with 4-10 membered heterocyclyl optionally substituted C1-C6 alkyl.

In some embodiments, R⁶ is C1-C6 alkyl substituted with 9-10 membered heteroaryl.

In some embodiments, R⁶ is C1-C6 alkyl.

In some embodiments, R⁶ is C1-C6 haloalkyl.

In some embodiments, R⁶ is C3-C6 cycloalkyl substituted with —CO₂R^{D}. In some embodiments, R⁶ is C3-C6 cycloalkyl.

In some embodiments, R⁶ is —NHC(═O)R^{C}.

In some embodiments, R⁶ is NR^{G}R^{H}.

In some embodiments, R⁶ is 4-10 membered heterocyclyl substituted C1-C6 alkyl. In some embodiments, R⁶ is 4-10 membered heterocyclyl (i.e., an unsubstituted 4-10 membered heterocyclyl). In some embodiments, R⁶ is imidazol-2-onyl. In some embodiments, R⁶ is

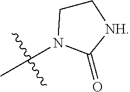 [SMILES: *N1CCNC1=O]

In some embodiments, R⁶ is —(CH₂)ₚC(═O)NHR^{I}.

In some embodiments, R⁶ is 5-10 membered heteroaryl substituted with C1-C6 alkyl. In some embodiments, R⁶ is an unsubstituted 5-10 membered heteroaryl.

In some embodiments, two geminal R⁶, together with the carbon atom to which they are attached form a C3-C6 spirocycloalkyl.

In some embodiments, R^{G} is hydrogen. In some embodiments, R^{G} is C1-C6 alkyl. In some embodiments, R^{G} is C3-C6 cycloalkyl.

In some embodiments, R^{H} is hydrogen. In some embodiments, R^{H} is C1-C6 alkyl. In some embodiments, R^{H} is C3-C6 cycloalkyl.

In some embodiments, R^{I} is 5-6 membered heteroaryl optionally substituted with C1-C6 alkyl or halogen.

In some embodiments, R^{I} is 5-6 membered heteroaryl substituted with C1-C6 alkyl. In some embodiments, R^{I} is 5-6 membered heteroaryl substituted with halogen. In some embodiments, R^{I} is 5-6 membered heteroaryl (i.e., an unsubstituted 5-6 membered heteroaryl).

In some embodiments, R^{I} is phenyl.

In some embodiments, R^{I} is phenyl substituted with halogen.

In some embodiments, m is 0. In some embodiments, m is 1. In some embodiments, m is 2. In some embodiments, m is 3.

In some embodiments, n is 0. In some embodiments, n is 1. In some embodiments, n is 2.

In some embodiments, p is 1. In some embodiments, p is 2. In some embodiments, p is 3. In some embodiments, p is 4.

In some embodiments, s is 0. In some embodiments, s is 1. In some embodiments, s is 2.

In some embodiments, Ring A and Ring B form a ring system which is:

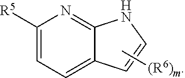

In some embodiments, Ring A and Ring B form a ring system which is:

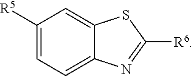

In some embodiments, Ring A and Ring B form a ring system which is:

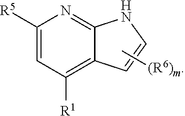

In some embodiments, Ring A and Ring B form a ring system which is:

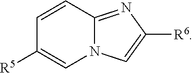

In some embodiments, Ring A and Ring B form a ring system which is:

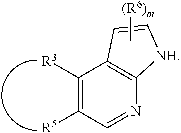

In some embodiments, Ring A and Ring B form a ring system which is:

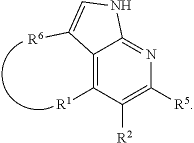

In some embodiments, Ring A and Ring B form a ring system which is:

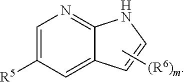

In some embodiments, Ring A and Ring B form a ring system which is:

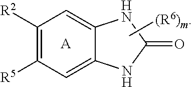

In some embodiments, Ring A and Ring B form a ring system which is:

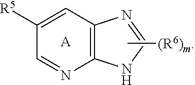

In some embodiments, Ring A and Ring B form a ring system which is:

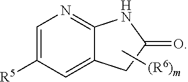

In some embodiments, Ring A and Ring B form a ring system which is:

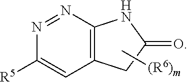

In some embodiments, Ring A and Ring B form a ring system which is:

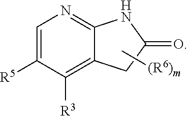

In some embodiments, Ring A and Ring B form a ring system which is:

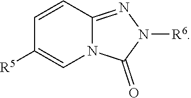

In some embodiments, Ring A and Ring B form a ring system which is:

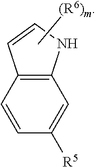

In some embodiments, Ring A and Ring B form a ring system which is:

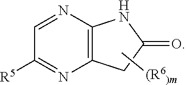

In some embodiments, Ring A and Ring B form a ring system which is:

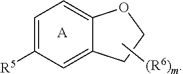

In some embodiments, Ring A and Ring B form a ring system which is:

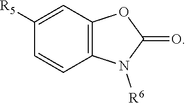

In some embodiments, Ring A and Ring B form a ring system which is:

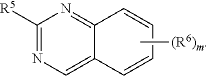

In some embodiments, Ring A and Ring B form a ring system which is:

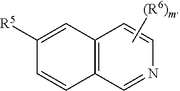

In some embodiments, Ring A and Ring B form a ring system which is:

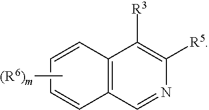

In some embodiments, Ring A and Ring B form a ring system which is:

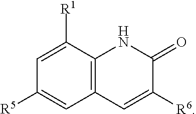

In some embodiments, Ring A and Ring B form a ring system which is:

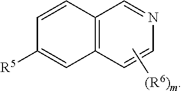

In some embodiments, Ring A and Ring B form a ring system which is:

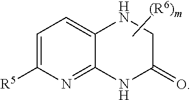

In some embodiments, Ring A and Ring B form a ring system which is:

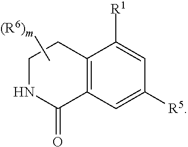

In some embodiments, Ring A and Ring B form a ring system which is:

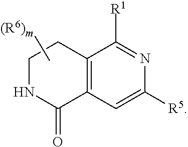

In some embodiments, Ring A and Ring B form a ring system which is:

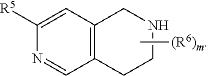

In some embodiments, Ring A and Ring B form a ring system which is:

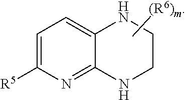

In some embodiments, the compound of Formula (II), or a pharmaceutically acceptable salt thereof, is selected from a compound in Table 2, or a pharmaceutically acceptable salt thereof.

TABLE 2
Selected Compounds of Formula (II)

| Compound | |
|---|---|
| No. | Structure |
| 1 | 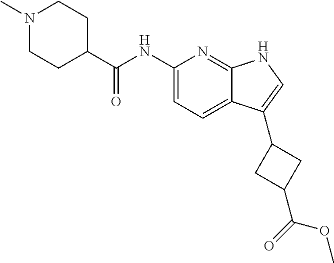 |
| 2 | 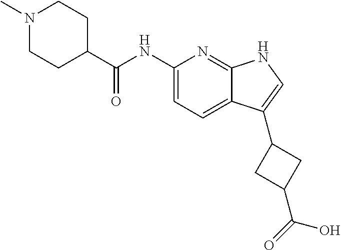 [SMILES: CN1CCC(C(=O)Nc2ccc3c(C4CC(C(=O)O)C4)c[nH]c3n2)CC1] |
| 3 | 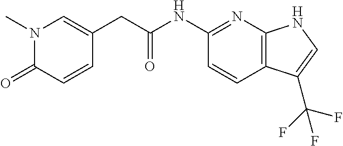 [SMILES: Cn1cc(CC(=O)Nc2ccc3c(C(F)(F)F)c[nH]c3n2)ccc1=O] |
| 4 | 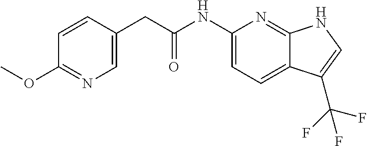 [SMILES: COc1ccc(CC(=O)Nc2ccc3c(C(F)(F)F)c[nH]c3n2)cn1] |
| 5 | 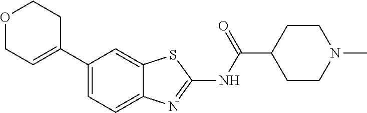 [SMILES: CN1CCC(C(=O)Nc2nc3ccc(C4=CCOCC4)cc3s2)CC1] |
| 6 | 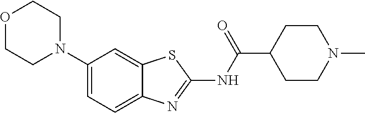 [SMILES: CN1CCC(C(=O)Nc2nc3ccc(N4CCOCC4)cc3s2)CC1] |
| 7 | 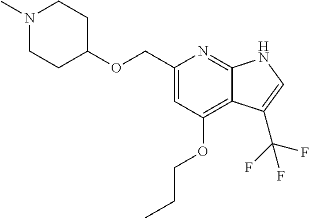 [SMILES: CCCOc1cc(COC2CCN(C)CC2)nc2[nH]cc(C(F)(F)F)c12] |
| 8 | 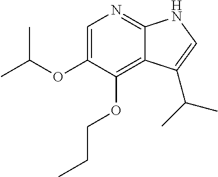 [SMILES: CCCOc1c(OC(C)C)cnc2[nH]cc(C(C)C)c12] |
| 9 | 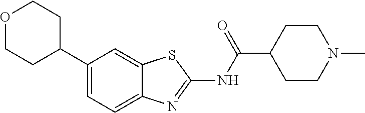 [SMILES: CN1CCC(C(=O)Nc2nc3ccc(C4CCOCC4)cc3s2)CC1] |
| 10 | 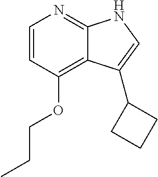 [SMILES: CCCOc1ccnc2[nH]cc(C3CCC3)c12] |
| 11 | 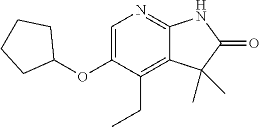 [SMILES: CCc1c(OC2CCCC2)cnc2c1C(C)(C)C(=O)N2] |
| 12 | 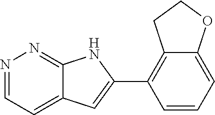 [SMILES: c1cc2c(c(-c3cc4ccnnc4[nH]3)c1)CCO2] |
| 13 | 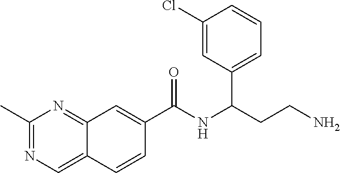 [SMILES: Cc1ncc2ccc(C(=O)NC(CCN)c3cccc(Cl)c3)cc2n1] |
| 14 | 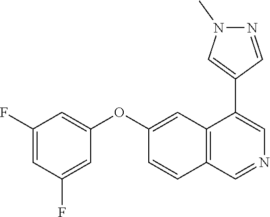 [SMILES: Cn1cc(-c2cncc3ccc(Oc4cc(F)cc(F)c4)cc23)cn1] |
| 15 | 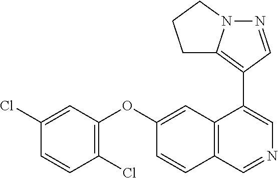 [SMILES: Clc1ccc(Cl)c(Oc2ccc3cncc(-c4cnn5c4CCC5)c3c2)c1] |
| 16 | 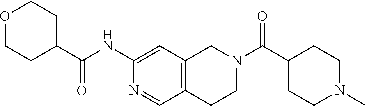 [SMILES: CN1CCC(C(=O)N2CCc3cnc(NC(=O)C4CCOCC4)cc3C2)CC1] |
| 17 | 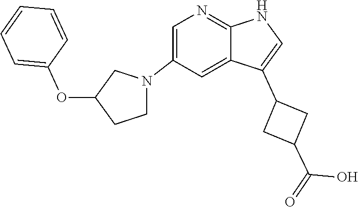 [SMILES: O=C(O)C1CC(c2c[nH]c3ncc(N4CCC(Oc5ccccc5)C4)cc23)C1] |
| 18 | 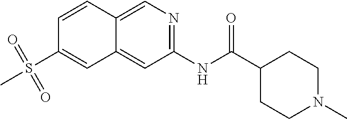 |
| 19 | 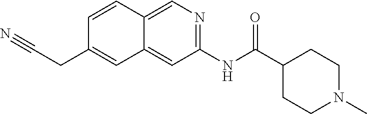 [SMILES: CN1CCC(C(=O)Nc2cc3cc(CC#N)ccc3cn2)CC1] |
| 20 | 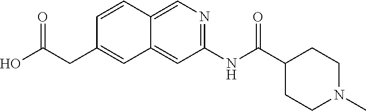 [SMILES: CN1CCC(C(=O)Nc2cc3cc(CC(=O)O)ccc3cn2)CC1] |
| 21 | 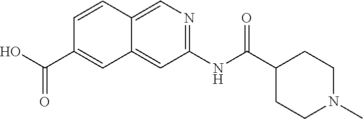 [SMILES: CN1CCC(C(=O)Nc2cc3cc(C(=O)O)ccc3cn2)CC1] |
| 22 | 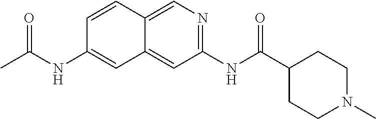 [SMILES: CC(=O)Nc1ccc2cnc(NC(=O)C3CCN(C)CC3)cc2c1] |
| 23 | 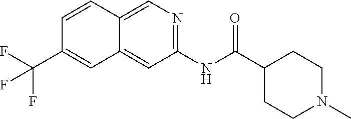 [SMILES: CN1CCC(C(=O)Nc2cc3cc(C(F)(F)F)ccc3cn2)CC1] |
| 24 | 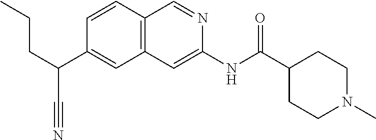 [SMILES: CCCC(C#N)c1ccc2cnc(NC(=O)C3CCN(C)CC3)cc2c1] |
| 25 | 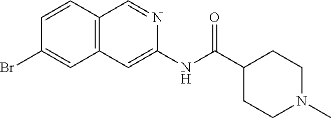 [SMILES: CN1CCC(C(=O)Nc2cc3cc(Br)ccc3cn2)CC1] |
| 26 | 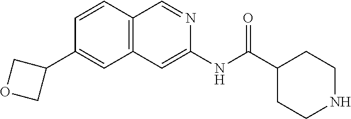 [SMILES: O=C(Nc1cc2cc(C3COC3)ccc2cn1)C1CCNCC1] |
| 27 | 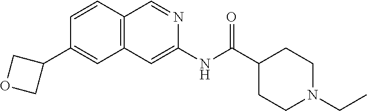 [SMILES: CCN1CCC(C(=O)Nc2cc3cc(C4COC4)ccc3cn2)CC1] |
| 28 | 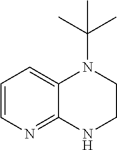 |
| 29 | 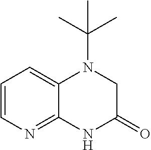 |
| 30 | 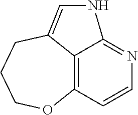 [SMILES: c1cc2c3c(c[nH]c3n1)CCCO2] |
| 31 | 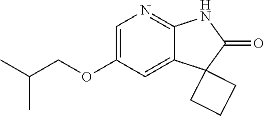 [SMILES: CC(C)COc1cnc2c(c1)C1(CCC1)C(=O)N2] |
| 32 | 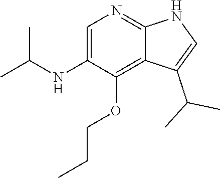 [SMILES: CCCOc1c(NC(C)C)cnc2[nH]cc(C(C)C)c12] |
| 33 | 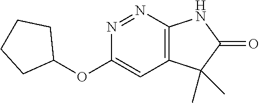 [SMILES: CC1(C)C(=O)Nc2nnc(OC3CCCC3)cc21] |
| 34 | 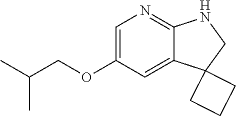 [SMILES: CC(C)COc1cnc2c(c1)C1(CCC1)CN2] |
| 35 | 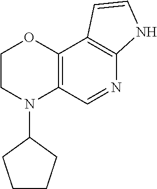 [SMILES: c1cc2c3c(cnc2[nH]1)N(C1CCCC1)CCO3] |
| 36 | 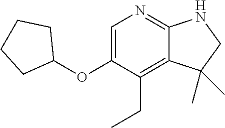 [SMILES: CCc1c(OC2CCCC2)cnc2c1C(C)(C)CN2] |
| 37 | 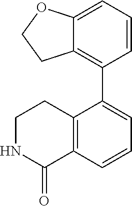 [SMILES: O=C1NCCc2c1cccc2-c1cccc2c1CCO2] |
| 38 | 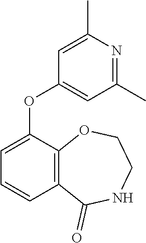 [SMILES: Cc1cc(Oc2cccc3c2OCCNC3=O)cc(C)n1] |
| 39 | 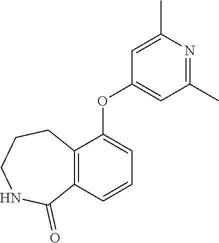 [SMILES: Cc1cc(Oc2cccc3c2CCCNC3=O)cc(C)n1] |
| 40 | 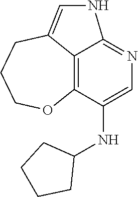 [SMILES: c1nc2[nH]cc3c2c(c1NC1CCCC1)OCCC3] |
| 41 | 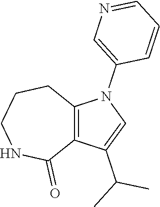 [SMILES: CC(C)c1cn(-c2cccnc2)c2c1C(=O)NCCC2] |
| 42 | 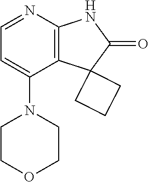 [SMILES: O=C1Nc2nccc(N3CCOCC3)c2C12CCC2] |
| 43 | 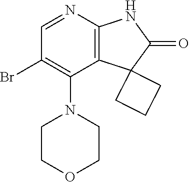 [SMILES: O=C1Nc2ncc(Br)c(N3CCOCC3)c2C12CCC2] |
| 44 | 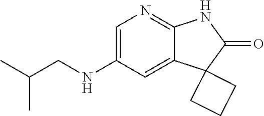 [SMILES: CC(C)CNc1cnc2c(c1)C1(CCC1)C(=O)N2] |
| 45 | 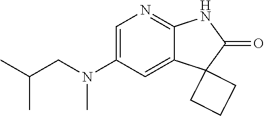 [SMILES: CC(C)CN(C)c1cnc2c(c1)C1(CCC1)C(=O)N2] |
| 46 | 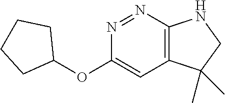 [SMILES: CC1(C)CNc2nnc(OC3CCCC3)cc21] |
| 47 | 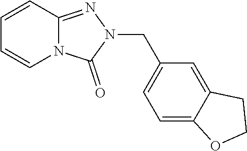 [SMILES: O=c1n(Cc2ccc3c(c2)CCO3)nc2ccccn12] |
| 48 | 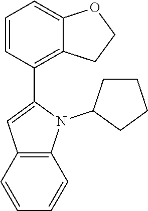 [SMILES: c1cc2c(c(-c3cc4ccccc4n3C3CCCC3)c1)CCO2] |
| 49 | 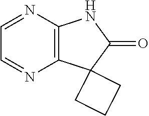 [SMILES: O=C1Nc2nccnc2C12CCC2] |
| 50 | 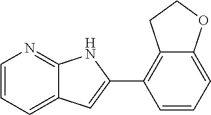 [SMILES: c1cc2c(c(-c3cc4cccnc4[nH]3)c1)CCO2] |
| 51 | 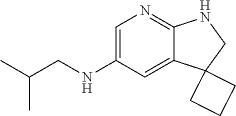 [SMILES: CC(C)CNc1cnc2c(c1)C1(CCC1)CN2] |
| 52 | 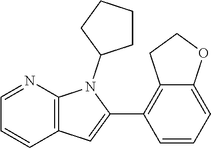 [SMILES: c1cc2c(c(-c3cc4cccnc4n3C3CCCC3)c1)CCO2] |
| 53 | 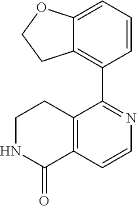 [SMILES: O=C1NCCc2c1ccnc2-c1cccc2c1CCO2] |
| 54 | 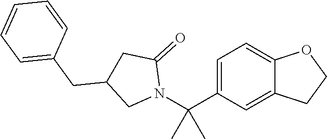 [SMILES: CC(C)(c1ccc2c(c1)CCO2)N1CC(Cc2ccccc2)CC1=O] |
| 55 | 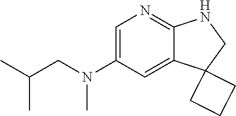 [SMILES: CC(C)CN(C)c1cnc2c(c1)C1(CCC1)CN2] |
| 56 | 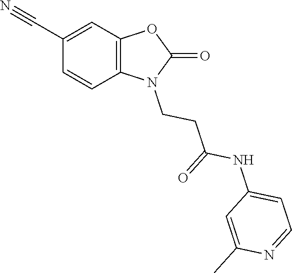 [SMILES: Cc1cc(NC(=O)CCn2c(=O)oc3cc(C#N)ccc32)ccn1] |
| 57 | 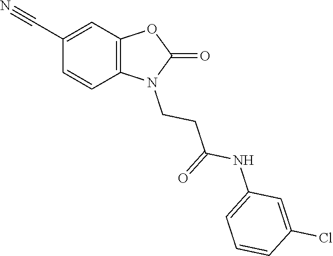 [SMILES: N#Cc1ccc2c(c1)oc(=O)n2CCC(=O)Nc1cccc(Cl)c1] |
| 58 | 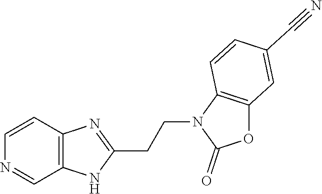 [SMILES: N#Cc1ccc2c(c1)oc(=O)n2CCc1nc2ccncc2[nH]1] |
| 59 | 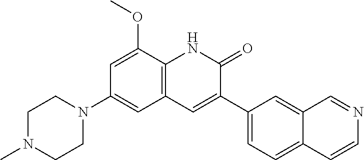 [SMILES: COc1cc(N2CCN(C)CC2)cc2cc(-c3ccc4ccncc4c3)c(=O)[nH]c12] |
| 60 | 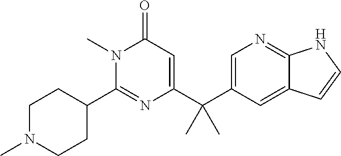 [SMILES: CN1CCC(c2nc(C(C)(C)c3cnc4[nH]ccc4c3)cc(=O)n2C)CC1] |
| 61 | 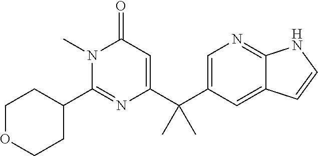 [SMILES: Cn1c(C2CCOCC2)nc(C(C)(C)c2cnc3[nH]ccc3c2)cc1=O] |
| 62 | 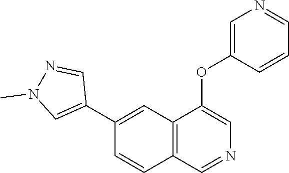 [SMILES: Cn1cc(-c2ccc3cncc(Oc4cccnc4)c3c2)cn1] |
| 63 | 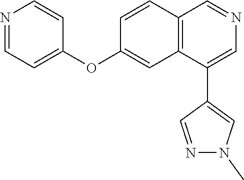 [SMILES: Cn1cc(-c2cncc3ccc(Oc4ccncc4)cc23)cn1] |
| 64 | 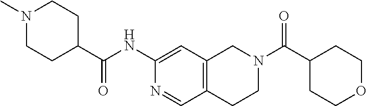 [SMILES: CN1CCC(C(=O)Nc2cc3c(cn2)CCN(C(=O)C2CCOCC2)C3)CC1] |
| 65 | 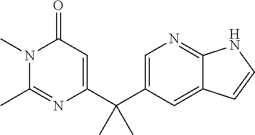 [SMILES: Cc1nc(C(C)(C)c2cnc3[nH]ccc3c2)cc(=O)n1C] |
| 66 | 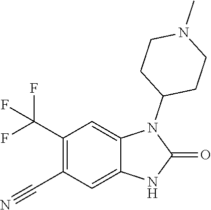 [SMILES: CN1CCC(n2c(=O)[nH]c3cc(C#N)c(C(F)(F)F)cc32)CC1] |
| 67 | 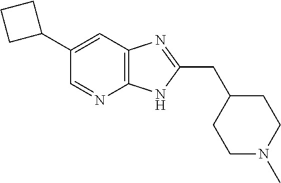 [SMILES: CN1CCC(Cc2nc3cc(C4CCC4)cnc3[nH]2)CC1] |
| 68 | 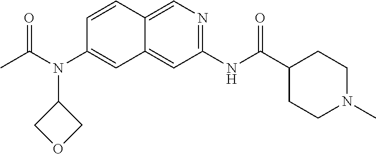 [SMILES: CC(=O)N(c1ccc2cnc(NC(=O)C3CCN(C)CC3)cc2c1)C1COC1] |
| 69 | 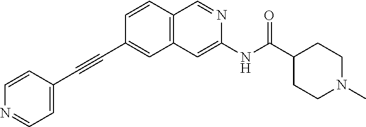 [SMILES: CN1CCC(C(=O)Nc2cc3cc(C#Cc4ccncc4)ccc3cn2)CC1] |
| 70 | 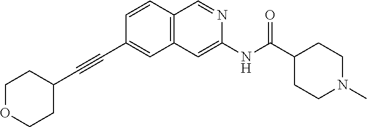 [SMILES: CN1CCC(C(=O)Nc2cc3cc(C#CC4CCOCC4)ccc3cn2)CC1] |
| 71 | 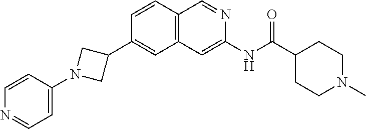 [SMILES: CN1CCC(C(=O)Nc2cc3cc(C4CN(c5ccncc5)C4)ccc3cn2)CC1] |
| 72 | 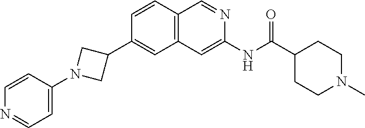 [SMILES: CN1CCC(C(=O)Nc2cc3cc(C4CN(c5ccncc5)C4)ccc3cn2)CC1] |
| 73 | 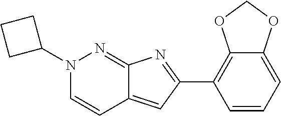 [SMILES: c1cc2c(c(-c3cc4ccn(C5CCC5)nc-4n3)c1)OCO2] |
| 74 | 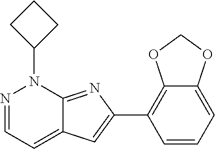 [SMILES: c1cc2c(c(-c3cc4ccnn(C5CCC5)c-4n3)c1)OCO2] |
| 75 | 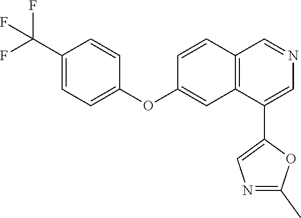 [SMILES: Cc1ncc(-c2cncc3ccc(Oc4ccc(C(F)(F)F)cc4)cc23)o1] |
| 76 | 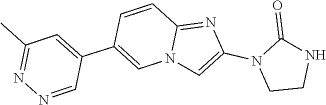 [SMILES: Cc1cc(-c2ccc3nc(N4CCNC4=O)cn3c2)cnn1] |

### Pharmaceutical Compositions and Administration

### General

In some embodiments, the compounds described herein (e.g., compounds of Formula (I) or (II) and pharmaceutically acceptable salts of any of the foregoing), are administered as a pharmaceutical composition that includes the chemical compound and one or more pharmaceutically acceptable excipients. In some embodiments, the compound or a pharmaceutically acceptable salt thereof is a compound of Formula (I), or a pharmaceutically acceptable salt thereof. In some embodiments, the compound or a pharmaceutically acceptable salt thereof is a compound of Formula (II), or a pharmaceutically acceptable salt thereof.

In some embodiments, the compounds can be administered in combination with one or more conventional pharmaceutical excipients as described herein. Dosage forms or compositions containing a chemical entity as described herein in the range of 0.005% to 100% with the balance made up from one or more pharmaceutically acceptable excipients may be prepared. The contemplated compositions may contain 0.001%-100% of a compound (or pharmaceutically acceptable salt thereof) provided herein, for example, from 0.1-95%, 75-85%, or 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

### Routes of Administration and Composition Components

In some embodiments, the compounds described herein or a pharmaceutical composition thereof can be administered to subject in need thereof by any accepted route of administration. Acceptable routes of administration include, but are not limited to, buccal, epidural, intracerebral, intradural, intramedullary, intrameningeal, intramuscular, intraspinal, intravascular, intravenous, nasal, oral, parenteral, peridural, respiratory (inhalation), subcutaneous, sublingual, submucosal, topical, transdermal, and transmucosal. In certain embodiments, a preferred route of administration is parenteral. In certain embodiments, a preferred route of administration is oral.

Compositions can be formulated for parenteral administration, e.g., formulated for injection via the intravenous, intramuscular, or sub-cutaneous routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the compounds is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In some embodiments, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds (i.e., excipients) include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

### Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 1 mg/kg; from about 0.1 mg/kg to about 0.5 mg/kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### Methods of Treatment

### Indications

This disclosure provides compounds of Formula (I) or (II), and pharmaceutically acceptable salts of any of the foregoing, that inhibit Dual specificity tyrosine-phosphorylation-regulated kinase 1A (DYRK1A). These compounds are useful for treating neurological disorders, e.g., DYRK1A-associated neurological disorders. In some embodiments, the compound or a pharmaceutically acceptable salt thereof is a compound of Formula (I), or a pharmaceutically acceptable salt thereof. In some embodiments, the compound or a pharmaceutically acceptable salt thereof is a compound of Formula (II), or a pharmaceutically acceptable salt thereof.

“Neurological disorder” refers to any disease or disorder of the nervous system and/or visual system. “Neurological disease” or “neurological disorder” are used interchangeably herein, and include diseases or disorders that involve the central nervous system (CNS; e.g., brain, brainstem and cerebellum), the peripheral nervous system (PNS; including cranial nerves), and the autonomic nervous system (parts of which are located in both the CNS and PNS), including both structural and/or functional diseases and disorders (e.g., neurological syndrome).

Examples of neurological disorders include, but are not limited to, headache, stupor and coma, dementia, seizure, sleep disorders, trauma, infections, neoplasms, neuroopthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological disorder. The following is a list of several neurological disorders, symptoms, signs and syndromes that can be treated using compositions and methods according to the present invention: acquired epileptiform aphasia; acute disseminated encephalomyelitis; adrenoleukodystrophy; agenesis of the corpus callosum; agnosia: Aicardi syndrome; Alexander disease; Alpers' disease; alternating hemiplegia; vascular dementia; amyotrophic lateral sclerosis; anencephaly; Angelman syndrome; angiomatosis; anoxia; aphasia; apraxia; arachnoid cysts; arachnoiditis; Anronl-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm: Bloch Sulzberger syndrome; brachial plexus injury; brain abscess; brain injury; Brown-Sequard syndrome; Cana van disease; carpal tunnel syndrome; causalgia; central pain syndrome; central pontine myelinolysis: cephalic disorder; cerebral aneurysm; cerebral arteriosclerosis: cerebral atrophy; cerebral gigantism; cerebral palsy; Charcot-Marie-Tooth disease; Chiari malformation; chorea; chronic inflammatory demyelinating polyneuropathy; chronic pain; chronic regional pain syndrome; Coffin Lowry syndrome; coma, including persistent vegetative state; congenital facial diplegia; corticobasal degeneration: cranial arteritis; craniosynostosis; Creutzfeldt-Jakob disease; cumulative trauma disorders; Cushing's syndrome; cytomegalic inclusion body disease; cytomegalovirus infection; dancing eyes-dancing feet syndrome; DandyWalker syndrome; Dawson disease; De Moisier's syndrome; Dejerine-Klumke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; fronto-temporal dementia; Gaucher's disease; Gerstmann's syndrome; giant cell arteritis; giant cell inclusion disease; globoid cell leukodystrophy; Guillain-Barre syndrome; HTLV-1-associated myelopathy; Hallervorden-Spatz disease; head injury; headache; hemifacial spasm; hereditary spastic paraplegia: heredopathia atactic a polyneuritiformis; herpes zoster oticus; herpes zoster; Hirayama syndrome; HIV associated dementia and neuropathy (also neurological manifestations of AIDS); holoprosencephaly; Huntington's disease and other polyglutamine repeat diseases; hydranencephaly: hydrocephalus; hypercortisolism; hypoxia; immune-mediated encephalomyelitis; inclusion body myositis; incontinentia pigmenti; infantile phytanic acid storage disease; infantile spasms; inflammatory myopathy; intracranial cyst; intracranial hypertension; Joubert syndrome; Kearns-Sayre syndrome; Kennedy disease Kinsboume syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gustaut syndrome; Lesch-Nyhan syndrome; leukodystrophy; Lewy body dementia; Lissencephaly; locked-in syndrome; Lou Gehrig's disease (i.e., motor neuron disease or amyotrophic lateral sclerosis); lumbar disc disease; Lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neuron disease; Moyamoya disease; mucopolysaccharidoses; milti-infarct dementia; multifocal motor neuropathy; multiple sclerosis and other demyelinating disorders; multiple system atrophy with postural hypotension; p muscular dystrophy; myasthenia gravis; myelmociastic diffuse sclerosis; myoclonic encephalopathy of infants; myoclonus; myopathy; myotonia congenital; narcolepsy: neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Neurodegenerative disease or disorder (Parkinson's disease, Huntington's disease, Alzheimer's disease, amyotrophic lateral sclerosis (ALS), dementia, multiple sclerosis and other diseases and disorders associated with neuronal cell death); paramyotonia congenital; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; porencephaly; post-polio syndrome; postherpetic neuralgia; postinfectious encephalomyelitis; postural hypotension; Prader-Willi syndrome; primary lateral sclerosis; prion diseases; progressive hemifacial atrophy; progressive multifocalleukoencephalopathy; progressive sclerosing poliodystrophy; progressive supranuclear palsy; Ramsay-Hunt syndrome (types I and I1); Rasmussen's encephalitis: reflex sympathetic dystrophy syndrome; Refsum disease; repetitive motion disorders, repetitive stress injuries; restless legs syndrome; retrovirus-associated myelopathy; Rett syndrome; Reye's syndrome; Saint Vitus dance; Sandhoff disease; Schilder's disease; schizencephaly; septo-optic dysplasia: shaken baby syndrome: shingles: Shy-Drager syndrome; Sjogren's syndrome; Soto's syndrome; spasticity; spina bifida; spinal cord injury; spinal muscular atrophy; Stiff-Person syndrome; stroke; Sturge-Weber syndrome; subacute sclerosing panencephalitis; subcortical arteriosclerotic encephalopathy; Sydenham chorea; syncope; syringomyelia; tardive dyskinesia; Tay-Sachs disease; temporal arteritis; tethered spinal cord syndrome; Thomsen disease; thoracic outlet syndrome; Tic Douloureux; Todd's paralysis; Tourette syndrome; transient ischemic attack; transmissible spongiform encephalopathies; transverse myelitis; traumatic brain injury; tremor, trigeminal neuralgia; tropical spastic paraparesis: tuberous sclerosis; vascular dementia (multi-infarct dementia); vasculitis including temporal arteritis; Von Hippel-Liodau disease; Wallenberg's syndrome; Werdnig-Hoffman disease; West syndrome; Williams syndrome; Wildoris disease; and Zellweger syndrome.

In some embodiments, the neurological disease or neurological disorder is Alzheimer's disease, Down syndrome, Alzheimer's disease associated with Down syndrome, Parkinson's disease, ALS, dementia, Huntington's disease, multiple sclerosis, proximal lateral sclerosis, stroke, stroke, or mild cognitive impairment.

In some embodiments, the dementia may be Alzheimer's dementia, cerebrovascular dementia, dementia due to head injury, multi-infarct dementia, mixed or alcoholic dementia of Alzheimer's disease and multi-infarct dementia.

The ability of test compounds to act as inhibitors of DYRK1A may be demonstrated by assays known in the art. The activity of the compounds and compositions provided herein as DYRK1A inhibitors can be assayed in vitro, in vivo, or in a cell line. In vitro assays include assays that determine inhibition of the kinase. Alternate in vitro assays quantitate the ability of the inhibitor to bind to the protein kinase and can be measured either by radio labelling the compound prior to binding, isolating the compound/kinase complex and determining the amount of radio label bound, or by running a competition experiment where new compounds are incubated with the kinase bound to known radio ligands.

Potency of a DYRK1A inhibitor as provided herein can be determined by EC₅₀ or IC₅₀ values. A compound with a lower EC₅₀ or IC₅₀ value, as determined under substantially similar conditions, is a more potent inhibitor relative to a compound with a higher EC₅₀ or IC₅₀ value. In some embodiments, the substantially similar conditions comprise determining a DYRK1A-dependent phosphorylation level, in vitro or in vivo (e.g., in neural cells, such as neurons, astrocytes, oligodendrocytes, microglia, ependymal cells, Schwann cells, and satellite cells, expressing a wild type DYRK1A, a mutant DYRK1A, or a fragment of any thereof).

Potency of a DYRK1A inhibitor as provided herein can also be determined by IC₅₀ value. A compound with a lower IC₅₀ value, as determined under substantially similar conditions, is a more potent inhibitor relative to a compound with a higher IC₅₀ value. In some embodiments, the substantially similar conditions comprise determining a DYRK1A-dependent phosphorylation level, in vitro or in vivo (e.g., in neural cells, such as neurons, astrocytes, oligodendrocytes, microglia, ependymal cells, Schwann cells, and satellite cells, expressing a wild type DYRK1A, a mutant DYRK1A, or a fragment of any thereof).

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject,” “individual,” or “patient,” are used interchangeably, refers to any animal, including mammals such as mice, rats, other rodents, rabbits, dogs, cats, swine, cattle, sheep, horses, primates, and humans. In some embodiments, the subject is a human. In some embodiments, the subject has experienced and/or exhibited at least one symptom of the disease or disorder to be treated and/or prevented.

In some embodiments, the subject has been identified or diagnosed as having a neurological disorder with a dysregulation of a DYRK1A gene, a DYRK1A protein, or expression or activity, or level of any of the same (a DYRK1A-associated neurological disorder) (e.g., as determined using a regulatory agency-approved, e.g., FDA-approved, assay or kit). The subject can be a subject that is positive for a dysregulation of a DYRK1A gene, a DYRK1A protein, or expression or activity, or level of any of the same (e.g., identified as positive using a regulatory agency-approved, e.g., FDA-approved, assay or kit). In some embodiments, the subject is suspected of having a DYRK1A-associated neurological disorder. In some embodiments, the subject has a clinical record indicating that the subject has a neurological disorder that has a dysregulation of a DYRK1A gene, a DYRK1A protein, or expression or activity, or level of any of the same (and optionally the clinical record indicates that the subject should be treated with any of the compositions provided herein).

In certain embodiments, compounds of Formula (I) or (II), or pharmaceutically acceptable salts of any of the foregoing, are useful for preventing neurological disorders as defined herein (for example, Alzheimer's disease). The term “preventing” as used herein means to delay the onset, recurrence or spread, in whole or in part, of the disease or condition as described herein, or a symptom thereof.

The term “DYRK1A-associated neurological disorder” as used herein refers disorders associated with or having a dysregulation of a DYRK1A gene, a DYRK1A protein, or the expression or activity or level of any (e.g., one or more) of the same (e.g., any of the types of dysregulation of a DYRK1A gene, or a DYRK1A protein, or the expression or activity or level of any of the same described herein). Non-limiting examples of a DYRK1A-associated disease or disorder include, for example, Down Syndrome, Alzheimer's disease, and Alzheimer's disease associated with Down Syndrome.

The phrase “dysregulation of a DYRK1A gene, a DYRK1A protein, or the expression or activity or level of any of the same” refers to a gene duplication (or multiplication) that results in an increased level of DYRK1A in a cell, or a mutation in a regulatory sequence (e.g., a promoter and/or enhancer) that results in an increased level of DYRK1A in a cell), or increased expression (e.g., increased levels) of a wild type DYRK1A in a mammalian cell due to aberrant cell signaling and/or dysregulated autocrine/paracrine signaling (e.g., as compared to a control cell lacking the aberrant signaling).

Some embodiments provide a method for treating a neurological disorder in a subject in need thereof, the method comprises administering to the subject a therapeutically effective amount of a compound of Formula (I) or (II), or a pharmaceutically acceptable salt of any of the foregoing, or a pharmaceutical composition comprising Formula (I) or (II), or a pharmaceutically acceptable salt of any of the foregoing.

In some embodiments, the method for treating a neurological disorder in a subject in need thereof, comprises (a) determining that the neurological disorder is associated with a dysregulation of a DYRK1A gene, a DYRK1A protein, or expression or activity or level of any of the same; and (b) administering to the subject a therapeutically effective amount of a compound of Formula (I) or (II), or a pharmaceutically acceptable salt of any of the foregoing, or a pharmaceutical composition comprising Formula (I) or (II), or a pharmaceutically acceptable salt of any of the foregoing.

Some embodiments provide a method of treating a DYRK1A-associated neurological disorder in a subject, the method comprises administering to a subject identified or diagnosed as having a DYRK1A-associated neurological disorder a therapeutically effective amount of a compound of Formula (I) or (II), or a pharmaceutically acceptable salt of any of the foregoing, or a pharmaceutical composition comprising Formula (I) or (II), or a pharmaceutically acceptable salt of any of the foregoing.

In some embodiments, the method of treating a DYRK1A-associated neurological disorder in a subject, comprises:
(a) determining that the neurological disorder in the subject is a DYRK1A-associated neurological disorder; and
(b) administering to the subject a therapeutically effective amount of a compound of Formula (I) or (II), or a pharmaceutically acceptable salt of any of the foregoing, or a pharmaceutical composition comprising Formula (I) or (II), or a pharmaceutically acceptable salt of any of the foregoing.

Some embodiments provide a method of treating a subject, the method comprises administering a therapeutically effective amount of a compound of Formula (I) or (II), or a pharmaceutically acceptable salt of any of the foregoing, or a pharmaceutical composition comprising Formula (I) or (II), or a pharmaceutically acceptable salt of any of the foregoing, to a subject having a clinical record that indicates that the subject has a dysregulation of a DYRK1A gene, DYRK1A protein, or expression or activity or level of any of the same.

In some embodiments, the method comprises the step of determining that the neurological disorder in the subject is a DYRK1A-associated neurological disorder and includes performing an assay to detect dysregulation in a DYRK1A gene, a DYRK1A protein, or expression or activity or level of any of the same in a sample from the subject.

Some embodiments provide a method for treating a neurological disorder in a subject in need thereof, the method comprising (a) determining that the neurological disorder is associated with Down Syndrome; and (b) administering to the subject a therapeutically effective amount of a compound of Formula (I) or (II), or a pharmaceutically acceptable salt of any of the foregoing, or a pharmaceutical composition comprising Formula (I) or (II), or a pharmaceutically acceptable salt of any of the foregoing.

In some embodiments, the step of determining that the neurological disorder in the subject is associated with Down Syndrome includes performing an assay on a sample from the subject.

In some embodiments, the method further comprises obtaining a sample from the subject. In some embodiments, the sample is a blood sample. In some embodiments, the sample is a sample of cerebrospinal fluid (CSF).

In some embodiments, the assay is selected from the group consisting of sequencing, immunohistochemistry, enzyme-linked immunosorbent assay, and fluorescence in situ hybridization (FISH).

In some embodiments, the FISH is break apart FISH analysis. In some embodiments, the sequencing is pyrosequencing or next generation sequencing.

In some embodiments, the DYRK1A-associated neurological disorder is selected from the group consisting of Down Syndrome, Alzheimer's disease, and Alzheimer's disease associated with Down syndrome. In some embodiments, the DYRK1A-associated neurological disorder is Alzheimer's disease associated with Down syndrome.

In some embodiments, the method further comprises administering to the subject an additional therapy or therapeutic agent as described herein.

Some embodiments provide a method for modulating DYRK1A in a mammalian cell, the method comprises contacting the mammalian cell with a therapeutically effective amount of a compound of a Formula (I) or (II), or a pharmaceutically acceptable salt of any of the foregoing.

In some embodiments, the contacting occurs in vivo. In some embodiments, the contacting occurs in vitro. In some embodiments, the mammalian cell is a mammalian neural cell. In some embodiments, the mammalian neural cell is a mammalian DYRK1A-associated neural cell. In some embodiments, the cell has a dysregulation of a DYRK1A gene, a DYRK1A protein, or expression or activity or level of any of the same. In some embodiments, the cell has a chromosomal abnormality associated with Down Syndrome.

Exemplary Sequence of Human Dual specificity tyrosine-phosphorylation-regulated kinase 1A (UniProtKB entry Q13627) (SEQ ID NO: 1)

MHTGGETSAC KPSSVRLAPS FSFHAAGLQM AGQMPHSHQY
SDRRQPNISD QQVSALSYSD QIQQPLTNQV MPDIVMLQRR
MPQTFRDPAT APLRKLSVDL IKTYKHINEV YYAKKKRRHQ
QGQGDDSSHK KERKVYNDGY DDDNYDYIVK NGEKWMDRYE
IDSLIGKGSE GQVVKAYDRV EQEWVAIKII KNKKAFINQA
QIEVRLLELM NKHDTEMKYY IVHLKRHEMF RNHLCLVFEM
LSYNLYDLLR NTNFRGVSLN LTRKFAQQMC TALLFLATPE
LSIIHCDLKP ENILLCNPKR SAIKIVDEGS SCQLGQRIYQ
YIQSRFYRSP EVLLGMPYDL AIDMWSLGCI LVEMHTGEPL
FSGANEVDQM NKIVEVLGIP PAHILDQAPK ARKFFEKLPD
GTWNIKKTKD GKREYKPPGT RKLHNILGVE TGGPGGRRAG
ESGHTVADYL KFKDLILRML DYDPKTRIQP YYALQHSFFK
KTADEGTNTS NSVSTSPAME QSQSSGTTSS TSSSSGGSSG
TSNSGRARSD PTHQHRHSGG HETAAVQAMD CETHSPQVRQ
QFPAPLGWSG TEAPTQVTVE THPVQETTFH VAPQQNALHH
HHGNSSHHHH HHHHHHHHHG QQALGNRTRP RVYNSPINSS
STQDSMEVGH SHHSMTSLSS STTSSSTSSS STGNQGNQAY
QNRPVAANTI DFGQNGAMDV NLTVYSNPRQ ETGIAGHPTY
QFSANTGPAH YMTEGHLTMR QGADREESPM TGVCVQQSPV
ASS

In some embodiments, compounds of Formula (I) or (II), or pharmaceutically acceptable salt of any of the foregoing, are useful for treating a neurological disorder that has been identified as being associated with dysregulation of DYRK1A. Accordingly, provided herein are methods for treating a subject diagnosed with (or identified as having) a neurological disorder that include administering to the subject a therapeutically effective amount of a compound of Formula (I) or (II), or a pharmaceutically acceptable salt of any of the foregoing.

Also provided herein are methods for treating a subject identified or diagnosed as having a DYRK1A-associated neurological disorder that include administering to the subject a therapeutically effective amount of a compound of Formula (I) or (II), or a pharmaceutically acceptable salt of any of the foregoing, or a pharmaceutical composition thereof. In some embodiments, the subject that has been identified or diagnosed as having a DYRK1A-associated neurological disorder through the use of a regulatory agency-approved, e.g., FDA-approved test or assay for identifying dysregulation of a DYRK1A gene, a DYRK1A protein, or expression or activity or level of any of the same, in a subject or a biological sample (e.g., blood and/or CSF) from the subject or by performing any of the non-limiting examples of assays described herein. In some embodiments, the test or assay is provided as a kit. In some embodiments, the neurological disorder is a DYRK1A-associated neurological disorder.

The term “regulatory agency” refers to a country's agency for the approval of the medical use of pharmaceutical agents with the country. For example, a non-limiting example of a regulatory agency is the U.S. Food and Drug Administration (FDA).

Also provided is a method for inhibiting DYRK1A activity in a cell, comprising contacting the cell with a compound of Formula (I) or (II), or a pharmaceutically acceptable salt of any of the foregoing. In some embodiments, the contacting is in vitro. In some embodiments, the contacting is in vivo. In some embodiments, the contacting is in vivo, wherein the method comprises administering a therapeutically effective amount of a compound of Formula (I) or (II), or a pharmaceutically acceptable salt of any of the foregoing, to a subject having a cell having aberrant DYRK1A activity. In some embodiments, the cell is a neural cell. In some embodiments, the neural cell is a DYRK1A-associated neural cell.

As used herein, the term “contacting” refers to the bringing together of indicated moieties in an in vitro system or an in vivo system. For example, “contacting” a DYRK1A protein with a compound provided herein includes the administration of a compound provided herein to an individual or subject, such as a human, having a DYRK1A protein, as well as, for example, introducing a compound provided herein into a sample containing a cellular or purified preparation containing the DYRK1A protein.

The phrase “therapeutically effective amount” means an amount of compound that, when administered to a subject in need of such treatment, is sufficient to (I) or (II) treat a DYRK1A protein-associated disease or disorder, (ii) attenuate, ameliorate, or eliminate one or more symptoms of the particular disease, condition, or disorder, or (iii) delay the onset of one or more symptoms of the particular disease, condition, or disorder described herein. The amount of a compound of Formula (I) or (II), or a pharmaceutically acceptable salt of any of the foregoing, that will correspond to such an amount will vary depending upon factors such as the particular compound, disease condition and its severity, the identity (e.g., weight) of the subject in need of treatment, but can nevertheless be routinely determined by one skilled in the art.

When employed as pharmaceuticals, the compounds of Formula (I) or (II), including pharmaceutically acceptable salts thereof, can be administered in the form of pharmaceutical compositions as described herein.

### Combinations

In some embodiments, of any of the methods described herein, the compound of Formula (I) or (II), or a pharmaceutically acceptable salt of any of the foregoing, is administered in combination with a therapeutically effective amount of at least one additional therapeutic agent selected from one or more additional therapies or therapeutic agents.

In some embodiments, the methods described herein further comprise administering one or more additional therapies selected from typical antipsychotics, atypical antipsychotics, antidepressants, electroconvulsive therapy, transcranial magnetic stimulation, benzodiazepines, mood stabilizers, cholinesterase inhibitors, memantine, NSAIDs, analgesics, anxiolytics, gabapentin and pregabalin.

In some embodiments, the methods described herein further comprise providing cognitive behavior therapy to the subject.

In some embodiments, the one or more additional therapies is a standard of care treatment for neuropathic pain. In some embodiments, the one or more additional therapies is a standard of care treatment for Alzheimer's disease. In some embodiments, the one or more additional therapies is a standard of care treatment for Alzheimer's disease associated with Down Syndrome.

In some embodiments, the one or more additional therapies is a typical antipsychotic. Representative typical antipsychotics include, but are not limited to chlorpromazine, chlorprothixene, levomepromazine, mesoridazine, periciazine, promazine, loxapine, molindone, perphenazine, thiothixene, droperidol, flupentixol, fluphenazine, haloperidol, pimozide, prochlorperazine, thioproperazine, trifluoperazine, and zuclopenthixol.

In some embodiments, the one or more additional therapies is an atypical antipsychotic. Representative atypical antipsychotics include, but are not limited to aripiprazole, risperidone, olanzapine, quetiapine, asenapine, paliperidone, ziprasidone, or lurasidone.

In some embodiments, the one or more additional therapies is an antidepressant. In some embodiments, the antidepressant is an atypical antidepressant, a selective serotonin reuptake inhibitor, a selective serotonin and norepinephrine reuptake inhibitor, a monoamine oxidase inhibitor, a selective norepinephrine reuptake inhibitor, or a tricyclic antidepressant.

In some embodiments, the compound of Formula (I) or (II), or a pharmaceutically acceptable salt of any of the foregoing, and the one or more additional therapies are administered as separate dosages sequentially in any order. In some embodiments, the compound of Formula (I) or (II), or a pharmaceutically acceptable salt of any of the foregoing, and the one or more additional therapies are administered as a single dosage form.

In some embodiments, the antidepressant is an atypical antidepressant. Representative atypical antidepressants include, but are not limited to mirtazapine, mianserin, bupropion, trazodone, nefazodone, tianeptine, opipramol, agomelatine, vilazodone, and vortioxetine.

In some embodiments, the antidepressant is a selective serotonin reuptake inhibitor. Representative selective serotonin reuptake inhibitors include, but are not limited to citalopram, escitalopram, fluoxetine, fluvoxamine, paroxetine, and sertraline.

In some embodiments, the antidepressant is a selective serotonin and norepinephrine reuptake inhibitor. Representative selective serotonin and norepinephrine reuptake inhibitors include, but are not limited to atomoxetine, desvenlafaxine, duloxetine, levomilnacipran, milnacipran, sibutramine, tramadol, and venlafaxine.

In some embodiments, the antidepressant is a monoamine oxidase inhibitor. Representative monoamine oxidase inhibitors include, but are not limited to moclobemide, rasagiline, selegiline, or safinamide.

In some embodiments, the antidepressant is a selective norepinephrine reuptake inhibitor. Representative selective norepinephrine reuptake inhibitors include, but are not limited to reboxetine.

In some embodiments, the antidepressant is a tricyclic antidepressant. Representative tricyclic antidepressants include, but are not limited to amineptine, amitriptyline, amoxapine, butriptyline, clomipramine, desipramine, dibenzepin, dosulepin, doxepin, imipramine, iprindole, lofepramine, maprotiline, norclomipramine, northiaden, nortriptyline, pipramol, protriptyline, tianeptine, and trimipramine.

In some embodiments, the one or more additional therapies is a benzodiazepine. Representative benzodiazepines include, but are not limited to alprazolam, bromazepam, chlordiazepoxide, clonazepam, clorazepate, diazepam, flurazepam, lorazepam, oxazepam, temazepam, or triazolam.

In some embodiments, the one or more additional therapies is a mood stabilizer. Representative mood stabilizers include, but are not limited to lithium, valproic acid, lamotrigine, or carbamazepine. In some embodiments, the one or more additional therapies is electroconvulsive therapy or transcranial magnetic stimulation.

In some embodiments, the one or more additional therapies is sertraline. In some embodiments, the one or more additional therapies is venlafaxine.

In some embodiments, the one or more additional therapies is a cholinesterase inhibitor. Representative cholinesterase inhibitors include, but are not limited to donepezil, galantamine, and rivastigmine.

In some embodiments, the one or more additional therapies is memantine.

In some embodiments, the one or more additional therapies is an NSAID. Representative NSAIDs include, but are not limited to clonixin, licofelone, salicylates (such as aspirin and diflunisal), propionic acid derivative (such as ibuprofen, dexibuprofen, naproxen, fenoprofen, ketoprofen, dexketoprofen, flurbiprofen, and oxaprozin), acetic acid derivatives (such as indomethacin, tolmetin, sulindac, etodolac, ketorolac, diclofenac, aceclofenac, and bromfenac), and COX-2 inhibitors (such as celecoxib).

In some embodiments, the one or more additional therapies is an analgesic. Representative analgesics include, but are not limited to nefopam, flupiritine, ziconotide, acetaminophen, and opioids (such as morphine, oxycodone, methadone, codeine, fentanyl, hydrocodone, and tramadol).

In some embodiments, the one or more additional therapies is an anxiolytic. Representative anxiolytics include, but are not limited to alnespirone, adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, and zolazepam.

In some embodiments, the one or more additional therapies is gabapentin or pregabalin.

In some embodiments, the one or more additional therapies is one additional therapy. In some embodiments, the one or more additional therapies is two, three, or four additional therapies. Some embodiments provide a method of treating a neurological disorder, comprising administering a compound of Formula (I) or (II), or a pharmaceutically acceptable salt of any of the foregoing, and one or more additional therapies selected from typical antipsychotics, atypical antipsychotics, antidepressants, electroconvulsive therapy, transcranial magnetic stimulation, benzodiazepines, mood stabilizers, cholinesterase inhibitors, memantine, NSAIDs, analgesics, anxiolytics, gabapentin and pregabalin, to a subject in need thereof.

In some embodiments, the subject was being administered the one or more additional therapies prior to initiation of treatment with a compound of Formula (I) or (II), or a pharmaceutically acceptable salt of any of the foregoing. In some embodiments, the subject was being administered the one or more additional therapies prior to initiation of treatment with a compound of Formula (I) or (II), or a pharmaceutically acceptable salt of any of the foregoing, but after treatment with a compound of Formula (I) or (II), or a pharmaceutically acceptable salt of any of the foregoing, for a period of time, the subject is no longer administered the one or more additional therapies. In some embodiments, of this paragraph, the period of time is about 1 month to about 1 year, for example, about 1 month to about 5 months, about 3 months to about 8 months, about 7 months to about 1 year, about 1 month, about 2 months, about 3 months, about 4 months, about 5 months, about 6 months, about 7 months, about 8 months, about 9 months, about 10 months, about 11 months, about 12 months, or any value in between. In some embodiments, the amount of the one or more additional therapies is decreased during the period of time, to zero at the end of the period of time.

In some embodiments, the subject has previously been administered one or more additional therapies selected from typical antipsychotics, atypical antipsychotics, antidepressants, electroconvulsive therapy, transcranial magnetic stimulation, benzodiazepines, mood stabilizers, cholinesterase inhibitors, memantine, NSAIDs, analgesics, anxiolytics, gabapentin, and pregabalin; wherein the subject was not responsive to the previous one or more therapies.

In some embodiments, the subject has previously been administered a standard of care treatment for neuropathic pain and the subject was not responsive to the previous therapy. In some embodiments, the subject has previously been administered a standard of care treatment for Alzheimer's disease and the subject was not responsive to the previous therapy. In some embodiments, the subject has previously been administered a standard of care treatment for Alzheimer's disease associated with Down Syndrome and the subject was not responsive to the previous therapy.

In some embodiments, the subject has previously been administered one or more additional therapies selected from typical antipsychotics, atypical antipsychotics, antidepressants, electroconvulsive therapy, transcranial magnetic stimulation, benzodiazepines, mood stabilizers, cholinesterase inhibitors, memantine, NSAIDs, analgesics, anxiolytics, gabapentin, and pregabalin, and was not responsive to the previous therapy.

In some embodiments, the subject has previously been administered one or more typical antipsychotics such as chlorpromazine, chlorprothixene, levomepromazine, mesoridazine, periciazine, promazine, loxapine, molindone, perphenazine, thiothixene, droperidol, flupentixol, fluphenazine, haloperidol, pimozide, prochlorperazine, thioproperazine, trifluoperazine, and zuclopenthixol, and was not responsive to the previous therapy.

In some embodiments, the subject has previously been administered one or more atypical antipsychotics, such as aripiprazole, risperidone, olanzapine, quetiapine, asenapine, paliperidone, ziprasidone, or lurasidone, and was not responsive to the previous therapy.

In some embodiments, the subject has previously been administered one or more antidepressants and was not responsive to the previous therapy. In some embodiments, the antidepressant is an atypical antidepressant, a selective serotonin reuptake inhibitor, a selective serotonin and norepinephrine reuptake inhibitor, a monoamine oxidase inhibitor, a selective norepinephrine reuptake inhibitor, or a tricyclic antidepressant, and was not responsive to the previous therapy.

In some embodiments, the subject has previously been administered one or more atypical antidepressants, such as mirtazapine, mianserin, bupropion, trazodone, nefazodone, tianeptine, opipramol, agomelatine, vilazodone, and vortioxetine, and was not responsive to the previous therapy.

In some embodiments, the subject has previously been administered one or more selective serotonin reuptake inhibitors, such as citalopram, escitalopram, fluoxetine, fluvoxamine, paroxetine, and sertraline, and was not responsive to the previous therapy.

In some embodiments, the subject has previously been administered one or more selective serotonin and norepinephrine reuptake inhibitors, such as atomoxetine, desvenlafaxine, duloxetine, levomilnacipran, milnacipran, sibutramine, tramadol, and venlafaxine, and was not responsive to the previous therapy.

In some embodiments, the subject has previously been administered one or more monoamine oxidase inhibitors, such as moclobemide, rasagiline, selegiline, or safinamide, and was not responsive to the previous therapy.

In some embodiments, the subject has previously been administered one or more selective norepinephrine reuptake inhibitors, such as reboxetine, and was not responsive to the previous therapy.

In some embodiments, the subject has previously been administered one or more tricyclic antidepressants, such as amineptine, amitriptyline, amoxapine, butriptyline, clomipramine, desipramine, dibenzepin, dosulepin, doxepin, imipramine, iprindole, lofepramine, maprotiline, norclomipramine, northiaden, nortriptyline, pipramol, protriptyline, tianeptine, and trimipramine, and was not responsive to the previous therapy.

In some embodiments, the subject has previously been administered one or more benzodiazepines, such as alprazolam, bromazepam, chlordiazepoxide, clonazepam, clorazepate, diazepam, flurazepam, lorazepam, oxazepam, temazepam, or triazolam, and was not responsive to the previous therapy.

In some embodiments, the subject has previously been administered one or more mood stabilizers, such as lithium, valproic acid, lamotrigine, or carbamazepine, and was not responsive to the previous therapy.

In some embodiments, the one or more additional therapies is electroconvulsive therapy or transcranial magnetic stimulation, and was not responsive to the previous therapy.

In some embodiments, the subject has previously been administered sertraline, and was not responsive to the previous therapy. In some embodiments, the subject has previously been administered venlafaxine, and was not responsive to the previous therapy.

In some embodiments, the subject has previously been administered one or more cholinesterase inhibitors such as donepezil, galantamine, or rivastigmine, and was not responsive to the previous therapy.

In some embodiments, the subject has previously been administered memantine, and was not responsive to the previous therapy.

In some embodiments, the subject has previously been administered one or more NSAIDs such as clonixin, licofelone, aspirin, diflunisal, ibuprofen, dexibuprofen, naproxen, fenoprofen, ketoprofen, dexketoprofen, flurbiprofen, oxaprozin, indomethacin, tolmetin, sulindac, etodolac, ketorolac, diclofenac, aceclofenac, bromfenac), or celecoxib, and was not responsive to the previous therapy.

In some embodiments, the subject has previously been administered one or more analgesics such as nefopam, flupiritine, ziconotide, acetaminophen, morphine, oxycodone, methadone, codeine, fentanyl, hydrocodone, or tramadol, and was not responsive to the previous therapy.

In some embodiments, the subject has previously been administered one or more anxiolytics, such as alnespirone, adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, or zolazepam, and was not responsive to the previous therapy.

In some embodiments, the subject has previously been administered gabapentin or pregabalin, and was not responsive to the previous therapy.

In some embodiments, the one or more additional therapies previously administered to the subject is 1-3 additional therapies. In some embodiments, the one or more additional therapies previously administered to the subject is one additional therapy. In some embodiments, the one or more additional therapies previously administered to the subject is two additional therapies. In some embodiments, the one or more additional therapies previously administered to the subject is three additional therapies.

Subjects that were “non-responsive” to a previous therapy includes subjects where the previous therapy lacked sufficient clinical efficacy, subjects that experienced an unacceptable number and/or severity of side effects due to the previous therapy (sufficient to require discontinuation of treatment), and subjects that experienced both of the foregoing. Side effects include, but are not limited to weight gain, flattened affect, tardive dyskinesia, drowsiness, nausea, vomiting, constipation, dry mouth, restlessness, dizziness, loss of sexual desire, erectile dysfunction, insomnia, and blurred vision.

### EMBODIMENTS

### Exemplary Embodiments of Compounds of Formula (I)

Embodiment 1: A compound of Formula (I):

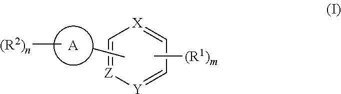

or a pharmaceutically acceptable salt thereof, wherein:
X is CH, N, N—O, or

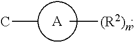

Y is CH, N, or

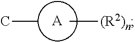

Z is CH, N, or CR¹;
wherein Formula (I) contains one Ring A and not more than two of X, Y, and Z are N or N—O;
each R¹ is independently hydroxyl, cyano, C1-C6 alkyl,

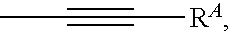

—NHC(═O)R^{A}, C1-C6 alkoxy, —(CH₂)ₚ—NR^{B}R^{C}, C3-C10 cycloalkyloxy optionally substituted with amino, -Q-(CH₂)_{q}—R^{D}, —CO₂R^{B}, —C(═O)NR^{B}R^{C}, —C(═O)-3-6 membered heterocyclyl, or phenoxy;
Ring A is a fused bicyclic 9 membered cycloalkyl, a fused bicyclic 9-10 membered heteroaryl, a 9 membered fused bicyclic heterocyclyl, or a fused tricyclic 13-14 membered heterocyclyl;
each R² is independently halogen, C1-C6 haloalkyl, C1-C6 alkyl optionally substituted with 1-2 substituents independently selected from hydroxyl, C1-C6 alkoxy, and 5-10 membered heteroaryl, —(CH₂)ₜ-Q¹-4-6 membered heterocyclyl optionally substituted with C1-C6 alkyl or —CO₂H, 4-9 membered heterocyclyl optionally substituted with R^{G}, —NHC(═O)R^{E},

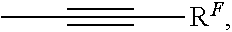

C1-C6 alkoxy, —C(═O)NHR^{H}, phenyl optionally substituted with 1-2 substituents independently selected from hydroxyl and cyano, 5-10 membered heteroaryl optionally substituted with C1-C6 alkyl, —NHSO₂(C1-C6 alkyl), C3-C9 cycloalkyl optionally substituted with hydroxyl or —NR^{B2}R^{C2}, —NR^{B1}R^{C1}, —C(═O)-3-6 membered heterocyclyl optionally substituted with C1-C6 alkyl, or benzyl optionally substituted with C1-C6 alkoxy;
each R^{A} and R^{F} is independently C3-C6 cycloalkyl or 3-6 membered heterocyclyl optionally substituted with 1-2 substituents independently selected from hydroxyl and C1-C6 alkyl;
each R^{B}, R^{B1}, R^{B2}, R^{C}, R^{C1}, and R^{C2} is independently hydrogen or C1-C6 alkyl optionally substituted with hydroxyl;
each R^{D} is independently a 4-6 membered heterocyclyl optionally substituted with C1-C6 alkyl or a phenyl optionally substituted with 1-2 independently selected halogen;
each R^{E} is independently 3-10 membered heterocyclyl optionally substituted with 1-3 independently selected C1-C6 alkyl or a —(CH₂)ᵥphenyl optionally substituted with cyano or halogen;
R^{G} is C1-C6 alkyl, —SO₂(R^{G1}), —C(═O)(C1-C6 alkyl), —C(═O)O—Benzyl, 4-6 membered heterocyclyl optionally substituted with 1-2 substituents independently selected from C1-C6 alkyl, and C3-C6 cycloalkyl optionally substituted with —CO₂H;
R^{G1} is C1-C6 alkyl or phenyl optionally substituted with cyano or halogen;
R^{H} is hydrogen, C1-C6 alkyl, or benzyl optionally substituted with halogen;
Q and Q¹ are independently 0, NH, or a bond;
m, n, p, and t are each independently 0, 1, 2, or 3;
v is 0 or 1; and
q is 1, 2, or 3.

Embodiment 2: The compound of embodiment 1, wherein not more than one of X, Y, and Z are N or N—O.

Embodiment 3: The compound of embodiment 1, wherein none of X, Y, and Z are N or N—O.

Embodiment 4: The compound of any one of embodiments 1-3, wherein X is CH.

Embodiment 5: The compound of any one of embodiments 1-3, wherein X is N.

Embodiment 6: The compound of any one of embodiments 1-3, wherein X is N—O.

Embodiment 7: The compound of any one of embodiments 1-3, wherein X is

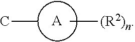

Embodiment 8: The compound of any one of embodiments 1-7, wherein Y is CH.

Embodiment 9: The compound of any one of embodiments 1-7, wherein Y is N.

Embodiment 10: The compound of any one of embodiments 1-6, wherein Y is

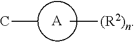

Embodiment 11: The compound of any one of embodiments 1-10, wherein Z is CH.

Embodiment 12: The compound of any one of embodiments 1-10, wherein Z is N.

Embodiment 13: The compound of any one of embodiments 1-10, wherein Z is CR¹.

Embodiment 14: The compound of any one of embodiments 1-6, 8-9, and 11-13, wherein

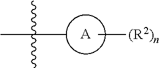

is attached to the position ortho to X and ortho to Z.

Embodiment 15: The compound of any one of embodiments 1-6, 8-9, and 11-13, wherein

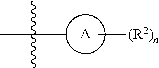

is attached to the position ortho to X and para to Z.

Embodiment 16: The compound of any one of embodiments 1-6, 8-9, and 11-13, wherein

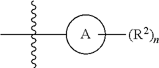

is attached to the position ortho to Y and meta to Z.

Embodiment 17: The compound of any one of embodiments 1-16, wherein m is 1.

Embodiment 18: The compound of any one of embodiments 1-16, wherein m is 2.

Embodiment 19: The compound of any one of embodiments 1-13, wherein m is 3.

Embodiment 20: The compound of any one of embodiments 1-19, wherein at least one R¹ is hydroxyl.

Embodiment 21: The compound of any one of embodiments 1-19, wherein at least one R¹ is cyano.

Embodiment 22: The compound of any one of embodiments 1-19, wherein at least one R¹ is C1-C6 alkyl.

Embodiment 23: The compound of any one of embodiments 1-19, wherein at least one R¹ is C1-C4 alkyl.

Embodiment 24: The compound of any one of embodiments 1-19, wherein at least one R¹ is methyl, ethyl, isopropyl, or isobutyl.

Embodiment 25: The compound of any one of embodiments 1-19, wherein at least one R¹ is

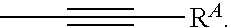

Embodiment 26: The compound of any one of embodiments 1-19, wherein at least one R¹ is —NHC(═O)R^{A}.

Embodiment 27: The compound of any one of embodiments 1-19 and 26, wherein at least one R^{A} is C3-C6 cycloalkyl.

Embodiment 28: The compound of any one of embodiments 1-19 and 26, wherein at least one R^{A} is C3-C4 cycloalkyl.

Embodiment 29: The compound of any one of embodiments 1-19 and 26, wherein at least one R^{A} is cyclopropyl.

Embodiment 30: The compound of any one of embodiments 1-19 and 26, wherein at least one R^{A} is cyclopentyl.

Embodiment 31: The compound of any one of embodiments 1-19 and 26, wherein at least one R^{A} is 3-6 membered heterocyclyl optionally substituted with 1-2 substituents independently selected from hydroxyl and C1-C6 alkyl.

Embodiment 32: The compound of any one of embodiments 1-19 and 26, wherein at least one R^{A} is 3-6 membered heterocyclyl substituted with 1-2 substituents independently selected from hydroxyl and C1-C6 alkyl.

Embodiment 33: The compound of any one of embodiments 1-19 and 26, wherein at least one R^{A} is 3-6 membered heterocyclyl substituted with hydroxyl.

Embodiment 34: The compound of any one of embodiments 1-19 and 26, wherein at least one R^{A} is 3-6 membered heterocyclyl substituted with C1-C6 alkyl.

Embodiment 35: The compound of any one of embodiments 1-19 and 26, wherein at least one R^{A} is 3-6 membered heterocyclyl substituted with methyl or isobutyl.

Embodiment 36: The compound of any one of embodiments 1-19 and 26, wherein at least one R^{A} is unsubstituted 3-6 membered heterocyclyl.

Embodiment 37: The compound of any one of embodiments 31-36, wherein the R^{A} 3-6 membered heterocyclyl is piperidinyl or tetrahydropyranyl.

Embodiment 38: The compound of any one of embodiments 1-19, wherein at least one R¹ is C1-C6 alkoxy.

Embodiment 39: The compound of any one of embodiments 1-19, wherein at least one R¹ is C1-C3 alkoxy.

Embodiment 40: The compound of any one of embodiments 1-19, wherein at least one R¹ is methoxy, ethoxy, or propoxy.

Embodiment 41: The compound of any one of embodiments 1-19, wherein at least one R¹ is —(CH₂)ₚ—NR^{B}R^{C}.

Embodiment 42: The compound of any one of embodiments 1-19 and 41, wherein p is 0.

Embodiment 43: The compound of any one of embodiments 1-19 and 41, wherein p is 1.

Embodiment 44: The compound of any one of embodiments 1-19 and 41, wherein p is 2.

Embodiment 45; The compound of any one of embodiments 1-19 and 41, wherein p is 3.

Embodiment 46: The compound of any one of embodiments 1-19 and 41-45, wherein R^{B} and R^{C} are hydrogen.

Embodiment 47: The compound of any one of embodiments 1-19 and 41-45, wherein R^{B} is hydrogen and R^{C} is C1-C6 alkyl optionally substituted with hydroxyl.

Embodiment 48: The compound of any one of embodiments 1-19 and 41-45, wherein R^{B} is hydrogen and R^{C} is C1-C6 alkyl substituted with hydroxyl.

Embodiment 49: The compound of any one of embodiments 1-19 and 41-45, wherein R^{B} is hydrogen and R^{C} is unsubstituted C1-C6 alkyl.

Embodiment 50: The compound of any one of embodiments 1-19 and 41-45, wherein R^{B} is C1-C6 alkyl optionally substituted with hydroxyl and R^{C} is hydrogen.

Embodiment 51: The compound of any one of embodiments 1-19 and 41-45, wherein R^{B} is C1-C6 alkyl substituted with hydroxyl and R^{C} is hydrogen.

Embodiment 52: The compound of any one of embodiments 1-19 and 41-45, wherein R^{B} is unsubstituted C1-C6 alkyl and R^{C} is hydrogen.

Embodiment 53: The compound of any one of embodiments 1-19 and 41-45, wherein R^{B} and R^{C} are each independently selected C1-C6 alkyl optionally substituted with hydroxyl.

Embodiment 54: The compound of any one of embodiments 1-19 and 41-45, wherein R^{B} and R^{C} are each independently selected C1-C6 alkyl substituted with hydroxyl.

Embodiment 55: The compound of any one of embodiments 1-19 and 41-45, wherein R^{B} and R^{C} are each independently selected unsubstituted C1-C6 alkyl.

Embodiment 56: The compound of any one of embodiments 47-55, wherein at least one R^{B} and/or R^{C} C1-C6 alkyl is C1-C4 alkyl.

Embodiment 57: The compound of any one of embodiments 47-55, wherein at least one R^{B} and/or R^{C} C1-C6 alkyl is methyl.

Embodiment 58: The compound of any one of embodiments 47-55, wherein at least one R^{B} and/or R^{C} C1-C6 alkyl is ethyl.

Embodiment 59: The compound of any one of embodiments 1-19, wherein at least one R¹ is C3-C10 cycloalkyloxy optionally substituted with amino.

Embodiment 60: The compound of any one of embodiments 1-19, wherein at least one R¹ is C3-C10 cycloalkyloxy substituted with amino.

Embodiment 61: The compound of any one of embodiments 1-19, wherein at least one R¹ is unsubstituted C3-C10 cycloalkyloxy.

Embodiment 62: The compound of any one of embodiments 1-19, wherein the R¹ C3-C10 cycloalkyloxy is cyclobutoxy.

Embodiment 63: The compound of any one of embodiments 1-19, wherein at least one R¹ is -Q-(CH₂)_{q}—R^{D}.

Embodiment 64: The compound of any one of embodiments 1-19 and 63, wherein Q is O.

Embodiment 65: The compound of any one of embodiments 1-19 and 63, wherein Q is NH.

Embodiment 66: The compound of any one of embodiments 1-19 and 63, wherein Q is bond.

Embodiment 67: The compound of any one of embodiments 1-19 and 63-66, wherein q is 1.

Embodiment 68: The compound of any one of embodiments 1-19 and 63-66, wherein q is 2.

Embodiment 69: The compound of any one of embodiments 1-19 and 63-66, wherein q is 3.

Embodiment 70: The compound of any one of embodiments 1-19 and 63-69, wherein at least one R^{D} is 4-6 membered heterocyclyl optionally substituted with C1-C6 alkyl.

Embodiment 71: The compound of any one of embodiments 1-19 and 63-69, wherein at least one R^{D} is a phenyl optionally substituted with 1-2 independently selected halogen.

Embodiment 72: The compound of any one of embodiments 1-19, wherein at least one R¹ is —CO₂R^{B}.

Embodiment 73: The compound of any one of embodiments 1-19 and 72, wherein R^{B} is hydrogen.

Embodiment 74: The compound of any one of embodiments 1-19 and 72, wherein R^{B} is C1-C6 alkyl optionally substituted with hydroxyl.

Embodiment 75: The compound of any one of embodiments 1-19 and 72, wherein R^{B} is C1-C6 alkyl substituted with hydroxyl.

Embodiment 76: The compound of any one of embodiments 1-19 and 72, wherein R^{B} is unsubstituted C1-C6 alkyl.

Embodiment 77: The compound of any one of embodiments 1-19 and 72, wherein R^{B} is methyl.

Embodiment 78: The compound of any one of embodiments 1-19 and 72, wherein R^{B} is ethyl.

Embodiment 79: The compound of any one of embodiments 1-19, wherein at least one R¹ is —C(═O)NR^{B}R^{C}.

Embodiment 80: The compound of any one of embodiments 1-19 and 79, wherein R^{B} and R^{C} are hydrogen.

Embodiment 81: The compound of any one of embodiments 1-19 and 79, wherein R^{B} is hydrogen and R^{C} is C1-C6 alkyl optionally substituted with hydroxyl.

Embodiment 82: The compound of any one of embodiments 1-19 and 79, wherein R^{B} is hydrogen and R^{C} is C1-C6 alkyl substituted with hydroxyl.

Embodiment 83: The compound of any one of embodiments 1-19 and 79, wherein R^{B} is hydrogen and R^{C} is unsubstituted C1-C6 alkyl.

Embodiment 84: The compound of any one of embodiments 1-19 and 79, wherein R^{B} is C1-C6 alkyl optionally substituted with hydroxyl and R^{C} is hydrogen.

Embodiment 85: The compound of any one of embodiments 1-19 and 79, wherein R^{B} is C1-C6 alkyl substituted with hydroxyl and R^{C} is hydrogen.

Embodiment 86: The compound of any one of embodiments 1-19 and 79, wherein R^{B} is unsubstituted C1-C6 alkyl and R^{C} is hydrogen.

Embodiment 87: The compound of any one of embodiments 1-19 and 79, wherein R^{B} and R^{C} are each independently selected C1-C6 alkyl optionally substituted with hydroxyl.

Embodiment 88: The compound of any one of embodiments 1-19 and 79, wherein R^{B} and R^{C} are each independently selected C1-C6 alkyl substituted with hydroxyl.

Embodiment 89: The compound of any one of embodiments 1-19 and 79, wherein R^{B} and R^{C} are each independently selected unsubstituted C1-C6 alkyl.

Embodiment 90: The compound of any one of embodiments 81-89, wherein at least one R^{B} and/or R^{C} C1-C6 alkyl is C1-C4 alkyl.

Embodiment 91: The compound of any one of embodiments 81-89, wherein at least one R^{B} and/or R^{C} C1-C6 alkyl is methyl.

Embodiment 92: The compound of any one of embodiments 81-89, wherein at least one R^{B} and/or R^{C} C1-C6 alkyl is ethyl.

Embodiment 93: The compound of any one of embodiments 1-19, wherein at least one R¹ is —C(═O)-3-6 membered heterocyclyl.

Embodiment 94: The compound of any one of embodiments 1-19, wherein at least one R¹ is —C(═O)-piperazinyl.

Embodiment 95: The compound of any one of embodiments 1-19, wherein at least one R¹ is phenoxy.

Embodiment 96: The compound of any one of embodiments 1-16, wherein m is 0.

The compound of any one of embodiments 1-96, wherein Ring A is a fused bicyclic 9 membered cycloalkyl.

Embodiment 98: The compound of any one of embodiments 1-97, wherein Ring A is 2,3-dihydro-1H-indenyl.

Embodiment 99: The compound of any one of embodiments 1-96, wherein Ring A is a fused bicyclic 9-10 membered heteroaryl.

Embodiment 100: The compound of any one of embodiments 1-96 and 99, wherein Ring A is pyrazolo[1,5-a]pyridinyl, 3H-imidazo[4,5-b]pyridine, pyrrolo[1,2-a]pyrazine, 7H-pyrrolo[2,3-c]pyridazine, 2H-indazolyl, imidazo[1,2-a]pyridine, benzo[d]thiazole, 1H-benzo[d]imidazole, 1H-pyrrolo[2,3-b]pyridine, imidazo[1,2-a]pyridine, 1H-pyrrolo[2,3-c]pyridinyl, pyrazolo[1,5-a]pyrazin-4(5H)-one, isoquinoline, quinoline, quinolin-2(1H)-one, 1,8-naphthyridin-2(1H)-one, 5H-pyrrolo[2,3-b]pyrazinyl, benzo[d]oxazolyl, thiazolo[5,4-b]pyridinyl, benzo[d]isothiazolyl, 2H-pyrazolo[3,4-c]pyridinyl, 1H-indazolyl, pyrazolo[1,5-a]pyrazin-4(5H)-one, furo[2,3-c]pyridinyl, [1,2,4]triazolo[4,3-a]pyridinyl, [1,2,4]triazolo[4,3-a]pyridin-3(2H)-one, benzo[d]isothiazolyl, imidazo[1,5-a]pyridinyl, 4H-pyrido[1,2-a][1,3,5]triazinyl, 1H-pyrrolo[2,3-c]pyridinyl, or 1H-pyrrolo[3,4-c]pyridine-1,3(2H)-dione.

Embodiment 101: The compound of any one of embodiments 1-96 and 99, wherein Ring A is pyrazolo[1,5-a]pyridinyl, 2H-indazolyl, imidazo[1,2-a]pyridine, benzo[d]thiazole, 1H-benzo[d]imidazole, 1H-pyrrolo[2,3-c]pyridinyl, isoquinoline, quinoline, quinolin-2(1H)-one, 1,8-naphthyridin-2(1H)-one, benzo[d]oxazolyl, 2H-pyrazolo[3,4-c]pyridinyl, 1H-indazolyl, 2H-indazolyl, [1,2,4]triazolo[4,3-a]pyridinyl, or 4H-pyrido[1,2-a][1,3,5]triazinyl.

Embodiment 102: The compound of any one of embodiments 1-96 and 99, wherein Ring A is benzo[d]thiazole, pyrrolo[1,2-a]pyrazine, 7H-pyrrolo[2,3-c]pyridazine, thiazolo[5,4-b]pyridinyl, or imidazo[1,5-a]pyridinyl.

Embodiment 103: The compound of any one of embodiments 1-96 and 99, wherein Ring A is 3H-imidazo[4,5-b]pyridine, imidazo[1,2-a]pyridine, or 1H-pyrrolo[2,3-b]pyridine.

Embodiment 104: The compound of any one of embodiments 1-96 and 99, wherein Ring A is 1H-benzo[d]imidazole, 1H-pyrrolo[2,3-b]pyridine, furo[2,3-c]pyridinyl, 1H-indazolyl, pyrazolo[1,5-a]pyrazin-4(5H)-one, or pyrazolo[1,5-a]pyridinyl.

Embodiment 105: The compound of any one of embodiments 1-96 and 99, wherein Ring A is 1H-benzo[d]imidazole, 1H-pyrrolo[2,3-b]pyridine, pyrazolo[1,5-a]pyrazin-4(5H)-one, or 4H-pyrido[1,2-a][1,3,5]triazinyl.

Embodiment 106: The compound of any one of embodiments 1-96 and 99, wherein Ring A is 1H-benzo[d]imidazole.

Embodiment 107: The compound of any one of embodiments 1-96 and 99, wherein Ring A is 1H-pyrrolo[2,3-b]pyridine.

Embodiment 108: The compound of any one of embodiments 1-96, wherein Ring A is a 9 membered fused bicyclic heterocyclyl.

Embodiment 109: The compound of any one of embodiments 1-96 and 108, wherein Ring A is 2,3-dihydrobenzofuranyl, 2-oxo-1,2-dihydro-1,8-naphthyridinyl, 2,3-dihydro-1H-indenyl, 4H-chromen-4-one, 1,3-dihydro-2H-benzo[d]imidazol-2-one, 2-oxo-1,2-dihydro-1,7-naphthyridinyl, 6-oxo-5,6,7,8-tetrahydroimidazo[1,2-a]pyrazinyl, 5,6,7,8-tetrahydroimidazo[1,2-a]pyrazinyl, imidazo[1,5-a]pyridinyl, 4H-pyrido[1,2-a][1,3,5]triazinyl, 4,5,6,7-tetrahydropyrazolo[1,5-a]pyrazinyl, or 1,2-dihydro-3H-pyrrolo[3,4-c]pyridin-3-one.

Embodiment 110: The compound of any one of embodiments 1-96 and 108, wherein Ring A is 5,6,7,8-tetrahydroimidazo[1,2-a]pyrazinyl, 6-oxo-5,6,7,8-tetrahydroimidazo[1,2-a]pyrazinyl, or 4,5,6,7-tetrahydropyrazolo[1,5-a]pyrazinyl.

Embodiment 111: The compound of any one of embodiments 1-96 and 108, wherein Ring A is 1,2-dihydro-3H-pyrrolo[3,4-c]pyridin-3-one.

Embodiment 112: The compound of any one of embodiments 1-96, wherein Ring A is a fused tricyclic 13-14 membered heterocyclyl.

Embodiment 113: The compound of any one of embodiments 1-96 and 112, wherein Ring A is 1,2,3,4-tetrahydrobenzo[4,5]imidazo[1,2-a]pyrazine.

Embodiment 114: The compound of any one of embodiments 1-113, wherein n is 1.

Embodiment 115: The compound of any one of embodiments 1-113, wherein n is 2.

Embodiment 116: The compound of any one of embodiments 1-113, wherein n is 3.

Embodiment 117: The compound of any one of embodiments 1-116, wherein at least one R² is halogen.

Embodiment 118: The compound of any one of embodiments 1-116, wherein at least one R² is fluoro.

Embodiment 119: The compound of any one of embodiments 1-116, wherein at least one R² is chloro.

Embodiment 120: The compound of any one of embodiments 1-116, wherein at least one R² is C1-C6 haloalkyl.

Embodiment 121: The compound of any one of embodiments 1-116, wherein at least one R² is difluoromethyl.

Embodiment 122: The compound of any one of embodiments 1-116, wherein at least one R² is trifluoromethyl.

Embodiment 123: The compound of any one of embodiments 1-116, wherein at least one R² is C1-C6 alkyl optionally substituted with 1-2 substituents independently selected from hydroxyl, C1-C6 alkoxy, and 5-10 membered heteroaryl.

Embodiment 124: The compound of any one of embodiments 1-116, wherein at least one R² is C1-C6 alkyl substituted with 1-2 substituents independently selected from hydroxyl, C1-C6 alkoxy, and 5-10 membered heteroaryl.

Embodiment 125: The compound of any one of embodiments 1-116, wherein at least one R² is C1-C6 alkyl substituted with 1-2 substituents independently selected from hydroxyl and indazolyl.

Embodiment 126: The compound of any one of embodiments 1-116, wherein at least one R² is C1-C6 alkyl substituted with hydroxyl.

Embodiment 127: The compound of any one of embodiments 1-116, wherein at least one R² is C1-C6 alkyl substituted with C1-C6 alkoxy.

Embodiment 128: The compound of any one of embodiments 1-116, wherein at least one R² is C1-C6 alkyl substituted with methoxy.

Embodiment 129: The compound of any one of embodiments 1-116, wherein at least one R² is C1-C6 alkyl substituted with 5-10 membered heteroaryl.

Embodiment 130: The compound of any one of embodiments 1-116, wherein at least one R² is C1-C6 alkyl substituted with indazolyl.

Embodiment 131: The compound of any one of embodiments 1-116, wherein the R² C1-C6 alkyl is methyl, ethyl, isopropyl, or isobutyl.

Embodiment 132: The compound of any one of embodiments 1-116, wherein the R² C1-C6 alkyl is methyl.

Embodiment 133: The compound of any one of embodiments 1-116, wherein at least one R² is —(CH₂)ₜ-Q¹-4-6 membered heterocyclyl optionally substituted with C1-C6 alkyl or —CO₂H.

Embodiment 134: The compound of any one of embodiments 1-116, wherein at least one R² is —(CH₂)ₜ-Q¹-4-6 membered heterocyclyl substituted with C1-C6 alkyl or —CO₂H.

Embodiment 135: The compound of any one of embodiments 1-116, wherein at least one R² is —(CH₂)ₜ-Q¹-4-6 membered heterocyclyl substituted with methyl or —CO₂H.

Embodiment 136: The compound of any one of embodiments 1-116, wherein at least one R² is unsubstituted —(CH₂)ₜ-Q¹-4-6 membered heterocyclyl.

Embodiment 137: The compound of any one of embodiments 1-116 and 133-136, wherein the 4-6 membered heterocyclyl of the R²—(CH₂)ₜ-Q¹-4-6 membered heterocyclyl is piperidinyl.

Embodiment 138: The compound of any one of embodiments 1-116 and 133-137, wherein Q¹ is O.

Embodiment 139: The compound of any one of embodiments 1-116 and 133-137, wherein Q¹ is NH.

Embodiment 140: The compound of any one of embodiments 1-116 and 133-137, wherein Q¹ is bond.

Embodiment 141: The compound of any one of embodiments 1-116 and 133-140, wherein t is 0.

Embodiment 142: The compound of any one of embodiments 1-116 and 133-140, wherein t is 1.

Embodiment 143: The compound of any one of embodiments 1-116 and 133-140, wherein t is 2.

Embodiment 144: The compound of any one of embodiments 1-116 and 133-140, wherein t is 3.

Embodiment 145: The compound of any one of embodiments 1-116, wherein at least one R² is 4-9 membered heterocyclyl optionally substituted with R^{G}.

Embodiment 146: The compound of any one of embodiments 1-116, wherein at least one R² is 4-9 membered heterocyclyl substituted with R^{G}.

Embodiment 147: The compound of any one of embodiments 1-116 and 146, wherein R^{G} is C1-C6 alkyl.

Embodiment 148: The compound of any one of embodiments 1-116 and 146, wherein R^{G} is —SO₂(R^{G1}).

Embodiment 149: The compound of any one of embodiments 1-116, 146, and 148 wherein R^{G1} is C1-C6 alkyl.

Embodiment 150: The compound of any one of embodiments 1-116, 146, and 148 wherein R^{G1} is methyl.

Embodiment 151: The compound of any one of embodiments 1-116, 146, and 148 wherein R^{G1} is phenyl optionally substituted with cyano or halogen.

Embodiment 152: The compound of any one of embodiments 1-116, 146, and 148 wherein R^{G1} is phenyl substituted with cyano or halogen.

Embodiment 153: The compound of any one of embodiments 1-116, 146, and 148 wherein R^{G1} is phenyl substituted with cyano.

Embodiment 154: The compound of any one of embodiments 1-116, 146, and 148 wherein R^{G1} is phenyl substituted with halogen.

Embodiment 155: The compound of any one of embodiments 1-116, 146, and 148 wherein R^{G1} is phenyl substituted with fluoro.

Embodiment 156: The compound of any one of embodiments 1-116, 146, and 148 wherein R^{G1} is unsubstituted phenyl.

Embodiment 157: The compound of any one of embodiments 1-116 and 146 wherein R^{G} is —C(═O)(C1-C6 alkyl).

Embodiment 158: The compound of any one of embodiments 1-116 and 146 wherein R^{G} is —C(═O)methyl.

Embodiment 159: The compound of any one of embodiments 1-116 and 146 wherein R^{G} is —C(═O)O—Benzyl.

Embodiment 160: The compound of any one of embodiments 1-116 and 146 wherein R^{G} is 4-6 membered heterocyclyl optionally substituted with 1-2 independently selected C1-C6 alkyl.

Embodiment 161: The compound of any one of embodiments 1-116 and 146 wherein R^{G} is 4-6 membered heterocyclyl substituted with 1-2 independently selected C1-C6 alkyl.

Embodiment 162: The compound of any one of embodiments 1-116 and 146 wherein R^{G} is 4-6 membered heterocyclyl substituted with 1-2 methyl.

Embodiment 163: The compound of any one of embodiments 1-116 and 146 wherein R^{G} is unsubstituted 4-6 membered heterocyclyl.

Embodiment 164: The compound of any one of embodiments 1-116 and 146 wherein the R^{G} 4-6 membered heterocyclyl is piperidinyl.

Embodiment 165: The compound of any one of embodiments 1-116 and 146 wherein R^{G} is C3-C6 cycloalkyl optionally substituted with —CO₂H.

Embodiment 166: The compound of any one of embodiments 1-116 and 146 wherein R^{G} is C3-C6 cycloalkyl substituted with —CO₂H.

Embodiment 167: The compound of any one of embodiments 1-116 and 146 wherein R^{G} is C3-C4 cycloalkyl substituted with —CO₂H.

Embodiment 168: The compound of any one of embodiments 1-116 and 146 wherein R^{G} is cyclobutyl optionally substituted with —CO₂H.

Embodiment 169: The compound of any one of embodiments 1-116 wherein at least one R² is unsubstituted 4-9 membered heterocyclyl.

Embodiment 170: The compound of any one of embodiments 1-116 and 169, wherein the R² 4-9 membered heterocyclyl is tetrahydropyranyl, piperidinyl, azepanyl, azetidinyl, 2,3-dihydrobenzofuran, or oxetanyl.

Embodiment 171: The compound of any one of embodiments 1-116, wherein at least one R² is —NHC(═O)R^{E}.

Embodiment 172: The compound of any one of embodiments 1-116 and 171, wherein R^{E} is 3-10 membered heterocyclyl optionally substituted with 1-3 independently selected C1-C6 alkyl.

Embodiment 173: The compound of any one of embodiments 1-116 and 171, wherein R^{E} is 3-10 membered heterocyclyl substituted with 1-3 independently selected C1-C6 alkyl.

Embodiment 174: The compound of any one of embodiments 1-116 and 171, wherein R^{E} is 3-10 membered heterocyclyl substituted with 1-3 methyl.

Embodiment 175: The compound of any one of embodiments 1-116 and 171, wherein R^{E} is unsubstituted 3-10 membered heterocyclyl.

Embodiment 176: The compound of any one of embodiments 172-175, wherein the R^{E} 3-10 membered heterocyclyl is piperidinyl, (1R,5S)-8-azabicyclo[3.2.1]octanyl, 3-azabicyclo[3.1.0]hexanyl, octahydrocyclopenta[c]pyrrolyl, azetidinyl, or tetrahydropyranyl.

Embodiment 177: The compound of any one of embodiments 1-116, wherein R^{E} is —(CH₂)ᵥphenyl optionally substituted with cyano or halogen;

Embodiment 178: The compound of any one of embodiments 1-116 and 177, wherein v is 0.

Embodiment 179: The compound of any one of embodiments 1-116 and 177, wherein v is 1.

Embodiment 180: The compound of any one of embodiments 1-116, wherein at least one R² is

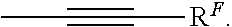

Embodiment 181: The compound of any one of embodiments 1-116 and 180, wherein R^{F} is C3-C6 cycloalkyl.

Embodiment 182: The compound of any one of embodiments 1-116 and 180, wherein R^{F} is C3-C4 cycloalkyl.

Embodiment 183: The compound of any one of embodiments 1-116 and 180, wherein R^{F} is cyclopropyl.

Embodiment 184: The compound of any one of embodiments 1-116 and 180, wherein R^{F} is 3-6 membered heterocyclyl optionally substituted with 1-2 substituents independently selected from hydroxyl and C1-C6 alkyl.

Embodiment 185: The compound of any one of embodiments 1-116 and 180, wherein R^{F} is 3-6 membered heterocyclyl substituted with 1-2 substituents independently selected from hydroxyl and C1-C6 alkyl.

Embodiment 186: The compound of any one of embodiments 1-116 and 180, wherein R^{F} is 3-6 membered heterocyclyl substituted with one hydroxyl and one C1-C6 alkyl.

Embodiment 187: The compound of any one of embodiments 1-116 and 180, wherein R^{F} is 3-6 membered heterocyclyl substituted with hydroxyl.

Embodiment 188: The compound of any one of embodiments 1-116 and 180, wherein R^{F} is 3-6 membered heterocyclyl substituted with C1-C6 alkyl.

Embodiment 189: The compound of any one of embodiments 1-116 and 180, wherein R^{F} is 3-6 membered heterocyclyl substituted with methyl, ethyl, isopropyl, or isobutyl.

Embodiment 190: The compound of any one of embodiments 184-189, wherein the R^{F} 3-6 membered heterocyclyl is piperidinyl or tetrahydropyranyl.

Embodiment 191: The compound of any one of embodiments 1-116, wherein at least one R² is C1-C6 alkoxy.

Embodiment 192: The compound of any one of embodiments 1-116, wherein at least one R² is C1-C3 alkoxy.

Embodiment 193: The compound of any one of embodiments 1-116, wherein at least one R² is methoxy.

Embodiment 194: The compound of any one of embodiments 1-116, wherein at least one R² is —C(═O)NHR^{H}.

Embodiment 195: The compound of any one of embodiments 1-116 and 194, wherein R^{H} is hydrogen.

Embodiment 196: The compound of any one of embodiments 1-116 and 194, wherein R^{H} is C1-C6 alkyl.

Embodiment 197: The compound of any one of embodiments 1-116 and 194, wherein R^{H} is C1-C3 alkyl.

Embodiment 198: The compound of any one of embodiments 1-116 and 194, wherein R^{H} is methyl.

Embodiment 199: The compound of any one of embodiments 1-116 and 194, wherein R^{H} is benzyl optionally substituted with halogen.

Embodiment 200: The compound of any one of embodiments 1-116 and 194, wherein R^{H} is benzyl optionally substituted with fluoro or chloro.

Embodiment 201: The compound of any one of embodiments 1-116, wherein at least one R² is phenyl optionally substituted with 1-2 substituents independently selected from hydroxyl and cyano.

Embodiment 202: The compound of any one of embodiments 1-116, wherein at least one R² is phenyl substituted with 1-2 substituents independently selected from hydroxyl and cyano.

Embodiment 203: The compound of any one of embodiments 1-116, wherein at least one R² is phenyl substituted with hydroxyl.

Embodiment 204: The compound of any one of embodiments 1-116, wherein at least one R² is phenyl substituted with cyano.

Embodiment 205: The compound of any one of embodiments 1-116, wherein at least one R² is unsubstituted phenyl.

Embodiment 206: The compound of any one of embodiments 1-116, wherein at least one R² is 5-10 membered heteroaryl optionally substituted with C1-C6 alkyl.

Embodiment 207: The compound of any one of embodiments 1-116, wherein at least one R² is 5-10 membered heteroaryl substituted with C1-C6 alkyl.

Embodiment 208: The compound of any one of embodiments 1-116, wherein at least one R² is 5-10 membered heteroaryl substituted with methyl.

Embodiment 209: The compound of any one of embodiments 1-116, wherein at least one R² is unsubstituted 5-10 membered heteroaryl.

Embodiment 210: The compound of any one of embodiments 1-116, wherein the R² 5-10 membered heteroaryl is quinolinyl, benzimidazolyl, or pyridyl.

Embodiment 211: The compound of any one of embodiments 1-116, wherein at least one R² is —NHSO₂(C1-C6 alkyl).

Embodiment 212: The compound of any one of embodiments 1-116, wherein at least one R² is —NHSO₂methyl.

Embodiment 213: The compound of any one of embodiments 1-116, wherein at least one R² is C3-C9 cycloalkyl optionally substituted with hydroxyl or —NR^{B2}R^{C2}.

Embodiment 214: The compound of any one of embodiments 1-116, wherein at least one R² is C3-C9 cycloalkyl substituted with hydroxyl or —NR^{B2}R^{C2}.

Embodiment 215: The compound of any one of embodiments 1-116, wherein at least one R² is C3-C9 cycloalkyl substituted with hydroxyl.

Embodiment 216: The compound of any one of embodiments 1-116, wherein at least one R² is C3-C9 cycloalkyl substituted with —NR^{B2}R^{C2}.

Embodiment 217: The compound of any one of embodiments 1-116 and 216, wherein R^{B2} and R^{C2} are hydrogen.

Embodiment 218: The compound of any one of embodiments 1-116 and 216, wherein R^{B2} is hydrogen and R^{C2} is C1-C6 alkyl optionally substituted with hydroxyl.

Embodiment 219: The compound of any one of embodiments 1-116 and 216, wherein R^{B2} is hydrogen and R^{C2} is C1-C6 alkyl substituted with hydroxyl.

Embodiment 220: The compound of any one of embodiments 1-116 and 216, wherein R^{B2} is hydrogen and R^{C2} is unsubstituted C1-C6 alkyl.

Embodiment 221: The compound of any one of embodiments 1-116 and 216, wherein R^{B2} is C1-C6 alkyl optionally substituted with hydroxyl and R^{C2} is hydrogen.

Embodiment 222: The compound of any one of embodiments 1-116 and 216, wherein R^{B2} is C1-C6 alkyl substituted with hydroxyl and R^{C2} is hydrogen.

Embodiment 223: The compound of any one of embodiments 1-116 and 216, wherein R^{B2} is unsubstituted C1-C6 alkyl and R^{C2} is hydrogen.

Embodiment 224: The compound of any one of embodiments 1-116 and 216, wherein R^{B2} and R^{C2} are each independently selected C1-C6 alkyl optionally substituted with hydroxyl.

Embodiment 225: The compound of any one of embodiments 1-116 and 216, wherein R^{B2} and R^{C2} are each independently selected C1-C6 alkyl substituted with hydroxyl.

Embodiment 226: The compound of any one of embodiments 1-116 and 216, wherein R^{B2} and R^{C2} are each independently selected unsubstituted C1-C6 alkyl.

Embodiment 227: The compound of any one of embodiments 218-226, wherein at least one R^{B2} and/or R^{C2} C1-C6 alkyl is C1-C4 alkyl.

Embodiment 228: The compound of any one of embodiments 218-226, wherein at least one R^{B2} and/or R^{C2} C1-C6 alkyl is methyl.

Embodiment 229: The compound of any one of embodiments 218-226, wherein at least one R^{B2} and/or R^{C2} C1-C6 alkyl is ethyl.

Embodiment 230: The compound of any one of embodiments 1-116, wherein the R² C3-C9 cycloalkyl is C3-C6 cycloalkyl.

Embodiment 231: The compound of any one of embodiments 1-116, wherein the R² C3-C9 cycloalkyl is cyclobutyl.

Embodiment 232: The compound of any one of embodiments 1-116, wherein at least one R² is —NR^{B1}R^{C1}.

Embodiment 233: The compound of any one of embodiments 1-116 and 232, wherein R^{B1} and R^{C1} are hydrogen.

Embodiment 234: The compound of any one of embodiments 1-116 and 232, wherein R^{B1} is hydrogen and R^{C1} is C1-C6 alkyl optionally substituted with hydroxyl.

Embodiment 235: The compound of any one of embodiments 1-116 and 232, wherein R^{B1} is hydrogen and R^{C1} is C1-C6 alkyl substituted with hydroxyl.

Embodiment 236: The compound of any one of embodiments 1-116 and 232, wherein R^{B1} is hydrogen and R^{C1} is unsubstituted C1-C6 alkyl.

Embodiment 237: The compound of any one of embodiments 1-116 and 232, wherein R^{B1} is C1-C6 alkyl optionally substituted with hydroxyl and R^{C1} is hydrogen.

Embodiment 238: The compound of any one of embodiments 1-116 and 232, wherein R^{B1} is C1-C6 alkyl substituted with hydroxyl and R^{C1} is hydrogen.

Embodiment 239: The compound of any one of embodiments 1-116 and 232, wherein R^{B1} is unsubstituted C1-C6 alkyl and R^{C1} is hydrogen.

Embodiment 240: The compound of any one of embodiments 1-116 and 232, wherein R^{B1} and R^{C1} are each independently selected C1-C6 alkyl optionally substituted with hydroxyl.

Embodiment 241: The compound of any one of embodiments 1-116 and 232, wherein R^{B1} and R^{C1} are each independently selected C1-C6 alkyl substituted with hydroxyl.

Embodiment 242: The compound of any one of embodiments 1-116 and 232, wherein R^{B1} and R^{C1} are each independently selected unsubstituted C1-C6 alkyl.

Embodiment 243: The compound of any one of embodiments 234-242, wherein at least one R^{B1} and/or R^{C1} C1-C6 alkyl is C1-C4 alkyl.

Embodiment 244: The compound of any one of embodiments 234-242, wherein at least one R^{B1} and/or R^{C1} C1-C6 alkyl is methyl.

Embodiment 245: The compound of any one of embodiments 234-242, wherein at least one R^{B1} and/or R^{C1} C1-C6 alkyl is ethyl.

Embodiment 246: The compound of any one of embodiments 1-116, wherein at least one R² is —C(═O)-3-6 membered heterocyclyl optionally substituted with C1-C6 alkyl.

Embodiment 247: The compound of any one of embodiments 1-116, wherein at least one R² is —C(═O)-3-6 membered heterocyclyl substituted with C1-C6 alkyl.

Embodiment 248: The compound of any one of embodiments 1-116, wherein at least one R² is —C(═O)-3-6 membered heterocyclyl substituted with methyl.

Embodiment 249: The compound of any one of embodiments 1-116, wherein at least one R² is unsubstituted —C(═O)-3-6 membered heterocyclyl.

Embodiment 250: The compound of any one of embodiments 1-116 and 246-249, wherein the R²—C(═O)-3-6 membered heterocyclyl is piperidinyl.

Embodiment 251: The compound of any one of embodiments 1-116, wherein at least one R² is benzyl optionally substituted with C1-C6 alkoxy.

Embodiment 252: The compound of any one of embodiments 1-116, wherein at least one R² is benzyl substituted with C1-C6 alkoxy.

Embodiment 253: The compound of any one of embodiments 1-116, wherein at least one R² is benzyl substituted with methoxy.

Embodiment 254: The compound of any one of embodiments 1-116, wherein at least one R² is unsubstituted benzyl.

Embodiment 255: The compound of any one of embodiments 1-113, wherein n is 0.

Embodiment 256: The compound of embodiment 1, wherein the compound of Formula (I) is a compound of Formula (I-A):

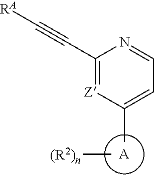

wherein:
   Z′ is CH or N.

Embodiment 257: The compound of embodiment 256, wherein Z′ is CH.

Embodiment 258: The compound of embodiment 256, wherein Z′ is N.

Embodiment 259: The compound of any one of embodiments 256-258, wherein R^{A} is as defined in any one of embodiments 27-37.

Embodiment 260: The compound of any one of embodiments 256-259, wherein Ring A is as defined in any one of embodiments 97-113.

Embodiment 261: The compound of any one of embodiments 256-258, wherein n and R² are as defined in any one of embodiments 114-255.

Embodiment 262: The compound of embodiment 1, wherein the compound of Formula (I) is a compound of Formula (I-B):

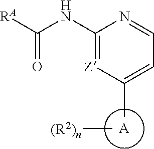

wherein:
Z′ is CH or N.

Embodiment 263: The compound of embodiment 262, wherein Z′ is CH.

Embodiment 264: The compound of embodiment 262, wherein Z′ is N.

Embodiment 265: The compound of any one of embodiments 262-264, wherein R^{A} is as defined in any one of embodiments 27-37.

Embodiment 266: The compound of any one of embodiments 262-265, wherein Ring A is as defined in any one of embodiments 97-113.

Embodiment 267: The compound of any one of embodiments 262-266, wherein n and R² are as defined in any one of embodiments 114-255.

Embodiment 268: The compound of embodiment 1, wherein the compound of Formula (I) is a compound of Formula (I-C):

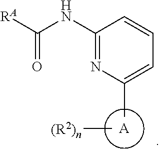

Embodiment 269: The compound of embodiment 268, wherein R^{A} is as defined in any one of embodiments 27-37.

Embodiment 270: The compound of any one of embodiments 268-269, wherein Ring A is as defined in any one of embodiments 97-113.

Embodiment 271: The compound of any one of embodiments 268-270, wherein n and R² are as defined in any one of embodiments 114-255.

Embodiment 272: The compound of embodiment 1, wherein the compound of Formula (I) is a compound of Formula (I-D):

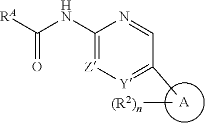

wherein:
Y′ is CH or N; and
Z′ is CH or N.

Embodiment 273: The compound of embodiment 272, wherein Y′ is CH.

Embodiment 274: The compound of embodiment 272, wherein Y′ is N.

Embodiment 275: The compound of any one of embodiments 272-274, wherein Z′ is CH.

Embodiment 276: Embodiment 276: The compound of any one of embodiments 272-274, wherein Z′ is N.

Embodiment 277: The compound of any one of embodiments 272-276, wherein R^{A} is as defined in any one of embodiments 27-37.

Embodiment 278: The compound of any one of embodiments 272-277, wherein Ring A is as defined in any one of embodiments 97-113.

Embodiment 279: The compound of any one of embodiments 272-278, wherein n and R² are as defined in any one of embodiments 114-255.

Embodiment 280: The compound of embodiment 1, wherein the compound of Formula (I) is a compound of Formula (I-E):

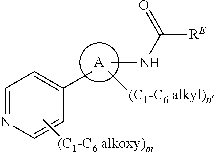

wherein:
n′ is 0, 1, or 2.

Embodiment 281: The compound of embodiment 280, wherein n′ is 1.

Embodiment 282: The compound of embodiment 280, wherein n′ is 2.

Embodiment 283: The compound of any one of embodiments 280-282, wherein each C1-C6 alkyl attached to Ring A is methyl, ethyl, isopropyl, or isobutyl.

Embodiment 284: The compound of any one of embodiments 280-282, wherein each C1-C6 alkyl attached to Ring A is methyl.

Embodiment 285: The compound of embodiment 280, wherein n′ is 0.

Embodiment 286: The compound of any one of embodiments 280-285, wherein m is 1.

Embodiment 287: The compound of any one of embodiments 280-285, wherein m is 2.

Embodiment 288: The compound of any one of embodiments 280-285, wherein m is 3.

Embodiment 289: The compound of any one of embodiments 280-288, wherein the C1-C6 alkoxy attached to the pyridyl is C1-C3 alkoxy.

Embodiment 290: The compound of any one of embodiments 280-288, wherein the C1-C6 alkoxy attached to the pyridyl is methoxy, ethoxy, or propoxy.

Embodiment 291: The compound of any one of embodiments 280-285, wherein m is 0.

Embodiment 292: The compound of any one of embodiments 280-291, wherein Ring A is as defined in any one of embodiments 97-113.

Embodiment 293: The compound of any one of embodiments 280-292, wherein R^{E} is as defined in any one of embodiments 172-179.

Embodiment 294: The compound of embodiment 1, wherein the compound of Formula (I) is a compound of Formula (I-F):

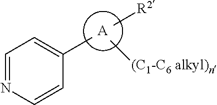

wherein:
R^{2′} is 4-9 membered heterocyclyl optionally substituted with R^{G}, phenyl optionally substituted with 1-2 substituents independently selected from hydroxyl and cyano, 5-10 membered heteroaryl optionally substituted with C1-C6 alkyl, or C3-C9 cycloalkyl optionally substituted with hydroxyl or —NR^{B2}R^{C2}; and
n′ is 0, 1, or 2.

Embodiment 295: The compound of embodiment 294, wherein n′ is 1.

Embodiment 296: The compound of embodiment 294, wherein n′ is 2.

Embodiment 297: The compound of any one of embodiments 294-296, wherein each C1-C6 alkyl attached to Ring A is methyl, ethyl, isopropyl, or isobutyl.

Embodiment 298: The compound of any one of embodiments 294-296, wherein each C1-C6 alkyl attached to Ring A is methyl.

Embodiment 299: The compound of embodiment 294, wherein n′ is 0.

Embodiment 300: The compound of any one of embodiments 294-299, wherein Ring A is as defined in any one of embodiments 97-113.

Embodiment 301: The compound of any one of embodiments 318-329, wherein R^{2′} is 4-9 membered heterocyclyl optionally substituted with R^{G}.

Embodiment 302: The compound of any one of embodiments 294-301, wherein R^{G} is as defined in any one of embodiments 147-168.

Embodiment 303: The compound of any one of embodiments 318-329, wherein R^{2′} is phenyl optionally substituted with 1-2 substituents independently selected from hydroxyl and cyano.

Embodiment 304: The compound of any one of embodiments 318-329, wherein R^{2′} is phenyl substituted with 1-2 substituents independently selected from hydroxyl and cyano.

Embodiment 305: The compound of any one of embodiments 318-329, wherein R^{2′} is phenyl substituted with hydroxyl.

Embodiment 306: The compound of any one of embodiments 318-329, wherein R^{2′} is phenyl substituted with cyano.

Embodiment 307: The compound of any one of embodiments 318-329, wherein R^{2′} is unsubstituted phenyl.

Embodiment 308: The compound of any one of embodiments 318-329, wherein R^{2′} is 5-10 membered heteroaryl optionally substituted with C1-C6 alkyl.

Embodiment 309: The compound of any one of embodiments 318-329, wherein R^{2′} is 5-10 membered heteroaryl substituted with C1-C6 alkyl.

Embodiment 310: The compound of any one of embodiments 318-329, wherein R^{2′} is 5-10 membered heteroaryl substituted with methyl.

Embodiment 311: The compound of any one of embodiments 318-329, wherein R^{2′} is unsubstituted 5-10 membered heteroaryl.

Embodiment 312: The compound of any one of embodiments 318-329 and 308-311, wherein the R^{2′} 5-10 membered heteroaryl is quinolinyl, benzimidazolyl, or pyridyl.

Embodiment 313: The compound of any one of embodiments 318-329, wherein R^{2′} is C3-C9 cycloalkyl optionally substituted with hydroxyl or —NR^{B2}R^{C2}.

Embodiment 314: The compound of any one of embodiments 318-329, wherein R^{2′} is C3-C9 cycloalkyl substituted with hydroxyl or —NR^{B2}R^{C2}.

Embodiment 315: The compound of any one of embodiments 318-329, wherein R^{2′} is C3-C9 cycloalkyl substituted with hydroxyl.

Embodiment 316: The compound of any one of embodiments 318-329, wherein R^{2′} is C3-C9 cycloalkyl substituted with —NR^{B2}R^{C2}.

Embodiment 317: The compound of any one of embodiments 318-329 and 313-316, wherein R^{B2} and R^{C2} are as defined in any one of embodiments 217-229.

Embodiment 318: The compound of embodiment 1, wherein the compound of Formula (I) is a compound of Formula (I-G):

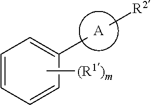

wherein:
each R^{1′} is independently hydroxyl, cyano, —CO₂R^{B}, or —C(═O)NR^{B}R^{C}; and
R^{2′} is 4-9 membered heterocyclyl optionally substituted with R^{G}, phenyl optionally substituted with 1-2 substituents independently selected from hydroxyl and cyano, 5-10 membered heteroaryl optionally substituted with C1-C6 alkyl, or C3-C9 cycloalkyl optionally substituted with hydroxyl or —NR^{B2}R^{C2}.

Embodiment 319: The compound of embodiment 318, wherein m is 1.

Embodiment 320: The compound of embodiment 318, wherein m is 2.

Embodiment 321: The compound of embodiment 318, wherein m is 3.

Embodiment 322: The compound of any one of embodiments 318-321, wherein at least one R^{1′} is independently hydroxyl.

Embodiment 323: The compound of any one of embodiments 318-321, wherein at least one R^{1′} is independently cyano.

Embodiment 324: The compound of any one of embodiments 318-321, wherein at least one R^{1′} is independently —CO₂R^{B}.

Embodiment 325: The compound of any one of embodiments 318-321 and 324, wherein R^{B} is as defined in any one of embodiments 73-78.

Embodiment 326: The compound of any one of embodiments 318-321, wherein at least one R^{1′} is independently —C(═O)NR^{B}R^{C}.

Embodiment 327: The compound of any one of embodiments 318-321 and 326, wherein R^{B} and R^{C} are as defined in any one of embodiments 80-92.

Embodiment 328: The compound of embodiment 318, wherein m is 0.

Embodiment 329: The compound of any one of embodiments 318-328, wherein Ring A is as defined in any one of embodiments 97-113.

Embodiment 330: The compound of any one of embodiments 318-329, wherein R^{2′} is 4-9 membered heterocyclyl optionally substituted with R^{G}.

Embodiment 331: The compound of any one of embodiments 318-330, wherein R^{G} is as defined in any one of embodiments 147-168.

Embodiment 332: The compound of any one of embodiments 318-329, wherein R^{2′} is phenyl optionally substituted with 1-2 substituents independently selected from hydroxyl and cyano.

Embodiment 333: The compound of any one of embodiments 318-329, wherein R^{2′} is phenyl substituted with 1-2 substituents independently selected from hydroxyl and cyano.

Embodiment 334: The compound of any one of embodiments 318-329, wherein R^{2′} is phenyl substituted with hydroxyl.

Embodiment 335: The compound of any one of embodiments 318-329, wherein R^{2′} is phenyl substituted with cyano.

Embodiment 336: The compound of any one of embodiments 318-329, wherein R^{2′} is unsubstituted phenyl.

Embodiment 337: The compound of any one of embodiments 318-329, wherein R^{2′} is 5-10 membered heteroaryl optionally substituted with C1-C6 alkyl.

Embodiment 338: The compound of any one of embodiments 318-329, wherein R^{2′} is 5-10 membered heteroaryl substituted with C1-C6 alkyl.

Embodiment 339: The compound of any one of embodiments 318-329, wherein R^{2′} is 5-10 membered heteroaryl substituted with methyl.

Embodiment 340: The compound of any one of embodiments 318-329, wherein R^{2′} is unsubstituted 5-10 membered heteroaryl.

Embodiment 341: The compound of any one of embodiments 318-329 and 337-340, wherein the R^{2′} 5-10 membered heteroaryl is quinolinyl, benzimidazolyl, or pyridyl.

Embodiment 342: The compound of any one of embodiments 318-329, wherein R^{2′} is C3-C9 cycloalkyl optionally substituted with hydroxyl or —NR^{B2}R^{C2}.

Embodiment 343: The compound of any one of embodiments 318-329, wherein R^{2′} is C3-C9 cycloalkyl substituted with hydroxyl or —NR^{B2}R^{C2}.

Embodiment 344: The compound of any one of embodiments 318-329, wherein R^{2′} is C3-C9 cycloalkyl substituted with hydroxyl.

Embodiment 345: The compound of any one of embodiments 318-329, wherein R^{2′} is C3-C9 cycloalkyl substituted with —NR^{B2}R^{C2}.

Embodiment 346: The compound of any one of embodiments 318-329, 342-343, and 345, wherein R^{B2} and R^{C2} are as defined in any one of embodiments 217-229.

### Exemplary Embodiments of Compounds of Formula (II)

Embodiment 1: A compound of Formula (II):

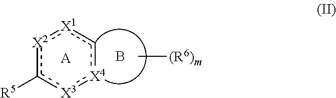

or a pharmaceutically acceptable salt thereof, wherein:
Ring A is aromatic;
Ring B is phenyl, 5-6 membered heteroaryl, or 5-7 membered monocyclic heterocyclyl such that together Ring A and Ring B form a 9-10 membered heteroaryl or a 9-10 membered heterocyclyl ring system;
X¹ is absent, CR¹, N, or NR^{A};
X² is CR², C═O, N, or NR, wherein when X² is C═O, X¹ is NR^{A}, and when X¹ is absent, X² is directly connected to the carbon atom shared by Ring A and Ring B;
X³ is C, CR³ or N;
is C or N;
one of R¹, R², R³, and R⁵ is halogen, cyano, C1-C6 haloalkyl, C1-C6 alkyl optionally substituted with 1-2 substituents independently selected from (i) 5-6 membered heteroaryl optionally substituted with 1-2 independently selected C1-C6 alkyl or 3-6 membered heterocyclyl optionally substituted with C1-C6 alkyl, (ii) 4-6 membered heterocyclyl optionally substituted with benzyl, (iii) cyano, (iv) phenyl optionally substituted with halogen, or (v) —NR^{E}R^{F}; C1-C6 alkoxy, —(CH₂)ₙ-Q-(4-6 membered heterocyclyl optionally substituted with C1-C6 alkyl), —NHC(═O)(CH₂)ₙR^{C}, 4-10 membered heterocyclyl optionally substituted with phenoxy, C1-C6 alkyl, or 5-6 membered heteroaryl; —NR^{E}R^{F}, C3-C6 cycloalkyl, C3-C6 cycloalkyloxy,

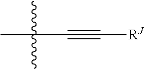

phenoxy optionally substituted with 1-2 substituents independently selected from halogen and C1-C6 haloalkyl; 5-6 membered heteroaryloxy optionally substituted with 1-2 independently selected C1-C6 alkyl, 5-10 membered heteroaryl optionally substituted with C1-C6 alkyl, —SO₂(C1-C6 alkyl), —(CH₂)ₙCO₂R^{D};
and the other of R¹, R², R³, and R⁵ are independently hydrogen, halogen, cyano, C1-C6 alkyl, C1-C6 alkoxy, C1-C6 haloalkyl, C3-C6 cycloalkyloxy, or 4-6 membered heterocyclyl optionally substituted with 5-6 membered heteroaryl; or
R³ and R⁵, together with the carbon atoms to which they are attached form a 6 membered heterocyclyl optionally substituted with C1-C6 alkyl or C3-C6 cycloalkyl; or
R³ and one R⁶ adjacent to Ring A, together with the carbon atoms to which they are attached form a 7 membered heterocyclyl; or
R¹ and one R⁶ adjacent to Ring A, together with the carbon atoms to which they are attached form a 7 membered heterocyclyl; or
R¹ and R², R² and R³, and R³ and R⁵, together with the carbon atoms to which they are attached form a C3-C5 cycloalkyl;
Q and Q¹ are each independently —O— or —C(═O)—;
R^{A} and R^{B} are independently hydrogen, C3-C6 cycloalkyl, or C1-C6 alkyl;
R^{C} is 4-6 membered heterocyclyl optionally substituted with C1-C6 alkyl, or 5-6 membered heteroaryl optionally substituted with C1-C6 alkoxy or C1-C6 alkyl,
R^{D} is hydrogen or C1-C6 alkyl;
R^{E} and R^{F} are independently hydrogen, C1-C6 alkyl, —C(═O)—C1-C6 alkyl, or 4-6 membered heterocyclyl;
R^{G} and R^{H} are independently hydrogen, C1-C6 alkyl, or C3-C6 cycloalkyl;
R^{I} is 5-6 membered heteroaryl optionally substituted with C1-C6 alkyl or halogen; C1-C6 alkyl substituted with 1-2 independently selected amino or phenyl optionally substituted with halogen; or phenyl optionally substituted with halogen;
R^{J} is a 4-6 membered heterocyclyl or a 5-6 membered heteroaryl;
R⁶ is C1-C6 alkyl optionally substituted with (i) 4-10 membered heterocyclyl optionally substituted C1-C6 alkyl or (ii) 9-10 membered heteroaryl; C1-C6 haloalkyl, C3-C6 cycloalkyl optionally substituted with —CO₂R^{D}, —NHC(═O)R^{C}, —NR^{G}R^{H}, 4-10 membered heterocyclyl optionally substituted C1-C6 alkyl, —(CH₂)ₚC(═O)NHR^{I}, 5-10 membered heteroaryl optionally substituted with C1-C6 alkyl, or —(CH₂)ₛ-Q¹-(4-6 membered heterocyclyl optionally substituted with C1-C6 alkyl); or
two geminal R⁶, together with the carbon atom to which they are attached form a C3-C6 spirocycloalkyl;
m is 0, 1, 2, or 3;
n is 0, 1, or 2;
p is 0, 1, 2, 3, or 4; and
s is 0, 1, or 2.

Embodiment 2: The compound of embodiment 1, wherein Ring B is phenyl.

Embodiment 3: The compound of embodiment 1, wherein Ring B is 5-6 membered heteroaryl.

Embodiment 4: The compound of embodiment 1, wherein Ring B is 5-7 membered monocyclic heterocyclyl.

Embodiment 5: The compound of embodiment 1, wherein Ring A and Ring B form a 9-10 membered heteroaryl ring system.

Embodiment 6: The compound of embodiment 1, wherein Ring A and Ring B form a 9-10 membered heterocyclyl ring system.

Embodiment 7: The compound of any one of embodiments 1-6, wherein X¹ is absent.

Embodiment 8: The compound of any one of embodiments 1-6, wherein X¹ is CR¹.

Embodiment 9: The compound of any one of embodiments 1-6, wherein X¹ is N.

Embodiment 10: The compound of any one of embodiments 1-6, wherein X¹ is NR^{A}.

Embodiment 11: The compound of any one of embodiments 1-6, wherein X² is CR².

Embodiment 12: The compound of any one of embodiments 1-6, wherein X² is C═O.

Embodiment 13: The compound of any one of embodiments 1-6, wherein X² is N.

Embodiment 14: The compound of any one of embodiments 1-6, wherein X² is NR^{B}.

Embodiment 15: The compound of any one of embodiments 1-6, wherein X³ is C.

Embodiment 16: The compound of any one of embodiments 1-6, wherein X³ is CR³.

Embodiment 17: The compound of any one of embodiments 1-6, wherein X³ is N.

Embodiment 18: The compound of any one of embodiments 1-6, wherein X⁴ is C.

Embodiment 19: The compound of any one of embodiments 1-6, wherein X⁴ is N.

Embodiment 20: The compound of any one of embodiments 1-6 and 8-19, wherein R^{A} is hydrogen.

Embodiment 21: The compound of any one of embodiments 1-6 and 8-19, wherein R^{A} is C1-C6 alkyl.

Embodiment 22: The compound of any one of embodiments 1-6 and 8-21, wherein R^{B} is hydrogen.

Embodiment 23: The compound of any one of embodiments 1-6 and 8-21, wherein R^{B} is C1-C6 alkyl.

Embodiment 24: The compound of any one of embodiments 1-6, 8-20 and 22, wherein 25 R^{A} and R^{B} are each hydrogen.

Embodiment 25: The compound of any one of embodiments 1-6, 8-19, 21 and 23 wherein R^{A} and R^{B} are each independently C1-C6 alkyl.

Embodiment 26: The compound of any one of embodiments 1-6 and 25, wherein R^{A} and R^{B} are each methyl.

Embodiment 27: The compound of any one of embodiments 1-6 and 8-26, wherein one of R^{A} and R^{B} is hydrogen, and the other of R^{A} and R^{B} is C1-C6 alkyl.

Embodiment 28: The compound of any one of embodiments 1-27, wherein one of R¹, R², R³, and R⁵ is halogen.

Embodiment 29: The compound of any one of embodiments 1-27, wherein one of R¹, R², R³, and R⁵ is cyano.

Embodiment 30: The compound of any one of embodiments 1-27, wherein one of R¹, R², R³, and R⁵ is C1-C6 haloalkyl.

Embodiment 31: The compound of any one of embodiments 1-27, wherein one of R¹, R², R³, and R⁵ is —CF³.

Embodiment 32: The compound of any one of embodiments 1-27, wherein one of R¹, R², R³, and R⁵ is C1-C6 alkyl optionally substituted with 5-6 membered heteroaryl optionally substituted with 1-2 independently selected C1-C6 alkyl or 3-6 membered heterocyclyl optionally substituted with C1-C6 alkyl.

Embodiment 33: The compound of any one of embodiments 1-27 and 32, wherein one of R¹, R², R³, and R⁵ is C1-C6 alkyl substituted with 5-6 membered heteroaryl optionally substituted with 1-2 independently selected C1-C6 alkyl.

Embodiment 34: The compound of any one of embodiments 1-27 and 32, wherein one of R¹, R², R³, and R⁵ is C1-C6 alkyl substituted with 5-6 membered heteroaryl substituted with 1-2 independently selected C1-C6 alkyl.

Embodiment 35: The compound of any one of embodiments 1-27 and 32, wherein one of R¹, R², R³, and R⁵ is C1-C6 alkyl substituted with 5-6 membered heteroaryl optionally substituted with 3-6 membered heterocyclyl optionally substituted with C1-C6 alkyl.

Embodiment 36: The compound of any one of embodiments 1-27 and 32, wherein one of R¹, R², R³, and R⁵ is C1-C6 alkyl substituted with 5-6 membered heteroaryl substituted with 3-6 membered heterocyclyl optionally substituted with C1-C6 alkyl.

Embodiment 37: The compound of any one of embodiments 1-27 and 32, wherein one of R¹, R², R³, and R⁵ is C1-C6 alkyl substituted with 5-6 membered heteroaryl substituted with 3-6 membered heterocyclyl substituted with C1-C6 alkyl.

Embodiment 38: The compound of any one of embodiments 1-27 and 32, wherein one of R¹, R², R³, and R⁵ is C1-C6 alkyl substituted with an unsubstituted 5-6 membered heteroaryl.

Embodiment 39: The compound of any one of embodiments 1-27, wherein one of R¹, R², R³, and R⁵ is C1-C6 alkyl optionally substituted with 4-6 membered heterocyclyl optionally substituted with benzyl.

Embodiment 40: The compound of any one of embodiments 1-27 and 39, wherein one of R¹, R², R³, and R⁵ is C1-C6 alkyl substituted with 4-6 membered heterocyclyl optionally substituted with benzyl.

Embodiment 41: The compound of any one of embodiments 1-27 and 39, wherein one of R¹, R², R³, and R⁵ is C1-C6 alkyl substituted with 4-6 membered heterocyclyl substituted with benzyl.

Embodiment 42: The compound of any one of embodiments 1-27 and 39, wherein one of R¹, R², R³, and R⁵ is C1-C6 alkyl substituted with an unsubstituted 4-6 membered heterocyclyl.

Embodiment 43: The compound of any one of embodiments 1-27, wherein one of R¹, R², R³, and R⁵ is C1-C6 alkyl optionally substituted with cyano.

Embodiment 44: The compound of any one of embodiments 1-27, wherein one of R¹, R², R³, and R⁵ is C1-C6 alkyl optionally substituted with phenyl optionally substituted with halogen.

Embodiment 45: The compound of any one of embodiments 1-27, wherein one of R¹, R², R³, and R⁵ is C1-C6 alkyl substituted with phenyl optionally substituted with halogen.

Embodiment 46: The compound of any one of embodiments 1-27, wherein one of R¹, R², R³, and R⁵ is C1-C6 alkyl substituted with phenyl substituted with halogen.

Embodiment 47: The compound of any one of embodiments 1-27, wherein one of R¹, R², R³, and R⁵ is C1-C6 alkyl substituted with unsubstituted phenyl.

Embodiment 48: The compound of any one of embodiments 1-27, wherein one of R¹, R², R³, and R⁵ is C1-C6 alkyl optionally substituted with —NR^{E}R^{F}.

Embodiment 49: The compound of any one of embodiments 1-27 wherein the substituted C1-C6 alkyl of one of R¹, R², R³, and R⁵ is a substituted C1-C2 alkyl.

Embodiment 50: The compound of any one of embodiments 1-27, wherein one of R¹, R², R³, and R⁵ is C1-C6 alkoxy.

Embodiment 51: The compound of any one of embodiments 1-27 and 50, wherein one of R¹, R², R³, and R⁵ is —OCH₃.

Embodiment 52: The compound of any one of embodiments 1-27, wherein one of R¹, R², R³, and R⁵ is —(CH₂)ₙ-Q-(4-6 membered heterocyclyl optionally substituted with C1-C6 alkyl).

Embodiment 53: The compound of any one of embodiments 1-27, wherein one of R¹, R², R³, and R⁵ is —NHC(═O)(CH₂)ₙR^{C}.

Embodiment 54: The compound of any one of embodiments 1-27, wherein one of R¹, R², R³, and R⁵ is 4-10 membered heterocyclyl optionally substituted with phenoxy, C1-C6 alkyl, or 5-6 membered heteroaryl.

Embodiment 55: The compound of any one of embodiments 1-27, wherein one of R¹, R², R³, and R⁵ is 4-10 membered heterocyclyl substituted with phenoxy, C1-C6 alkyl, or 5-6 membered heteroaryl.

Embodiment 56: The compound of any one of embodiments 1-27 and 54-55, wherein one of R¹, R², R³, and R⁵ is 4-10 membered heterocyclyl substituted with phenoxy.

Embodiment 57: The compound of any one of embodiments 1-27 and 54-55, wherein one of R¹, R², R³, and R⁵ is 4-10 membered heterocyclyl substituted with C1-C6 alkyl.

Embodiment 58: The compound of any one of embodiments 1-27, 54-55 and 57, wherein one of R¹, R², R³, and R⁵ is 4-10 membered heterocyclyl substituted with methyl.

Embodiment 59: The compound of any one of embodiments 1-27 and 54-55, wherein one of R¹, R², R³, and R⁵ is 4-10 membered heterocyclyl substituted with 5-6 membered heteroaryl.

Embodiment 60: The compound of any one of embodiments 1-27, wherein one of R¹, R², R³, and R⁵ is —NR^{E}R^{F}.

Embodiment 61: The compound of any one of embodiments 1-27, wherein one of R¹, R², R³, and R⁵ is C3-C6 cycloalkyl.

Embodiment 62: The compound of any one of embodiments 1-27, wherein one of R¹, R², R³, and R⁵ is C3-C6 cycloalkyloxy.

Embodiment 63: The compound of any one of embodiments 1-27, wherein one of R¹, R², R³, and R⁵ is

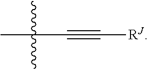

Embodiment 64: The compound of any one of embodiments 1-27, wherein one of R¹, R², R³, and R⁵ is phenoxy optionally substituted with 1-2 substituents independently selected from halogen and C1-C6 haloalkyl.

Embodiment 65: The compound of any one of embodiments 1-27, wherein one of R¹, R², R³, and R⁵ is phenoxy substituted with 1-2 substituents independently selected from halogen and C1-C6 haloalkyl.

Embodiment 66: The compound of any one of embodiments 1-27, wherein one of R¹, R², R³, and R⁵ is phenoxy substituted with 1-2 substituents independently selected halogen.

Embodiment 67: The compound of any one of embodiments 1-27, wherein one of R¹, R², R³, and R⁵ is phenoxy substituted with 1-2 substituents independently selected C1-C6 haloalkyl.

Embodiment 68: The compound of any one of embodiments 1-27, wherein one of R¹, R², R³, and R⁵ is unsubstituted phenoxy.

Embodiment 69: The compound of any one of embodiments 1-27, wherein one of R¹, R², R³, and R⁵ is 5-6 membered heteroaryloxy optionally substituted with 1-2 independently selected C1-C6 alkyl.

Embodiment 70: The compound of any one of embodiments 1-27, wherein one of R¹, R², R³, and R⁵ is 5-6 membered heteroaryloxy substituted with 1-2 independently selected C1-C6 alkyl.

Embodiment 71: The compound of any one of embodiments 1-27 and 69-70, wherein one of R¹, R², R³, and R⁵ is 5-6 membered heteroaryloxy substituted with C1-C6 alkyl.

Embodiment 72: The compound of any one of embodiments 1-27 and 69-71, wherein one of R¹, R², R³, and R⁵ is 5-6 membered heteroaryloxy substituted with two independently selected C1-C6 alkyl.

Embodiment 73: The compound of any one of embodiments 1-27, wherein one of R¹, R², R³, and R⁵ is 5-6 membered unsubstituted heteroaryloxy.

Embodiment 74: The compound of any one of embodiments 1-27, wherein one of R¹, R², R³, and R⁵ is 5-10 membered heteroaryl optionally substituted with C1-C6 alkyl.

Embodiment 75: The compound of any one of embodiments 1-27, wherein one of R¹, R², R³, and R⁵ is 5-10 membered heteroaryl substituted with C1-C6 alkyl.

Embodiment 76: The compound of any one of embodiments 1-27 and 75, wherein one of R¹, R², R³, and R⁵ is 5-10 membered heteroaryl substituted with methyl.

Embodiment 77: The compound of any one of embodiments 1-27, wherein one of R¹, R², R³, and R⁵ is 5-10 membered unsubstituted heteroaryl.

Embodiment 78: The compound of any one of embodiments 1-27, wherein one of R¹, R², R³, and R⁵ is —SO₂(C1-C6 alkyl).

Embodiment 79: The compound of any one of embodiments 1-27 and 78, wherein one of R¹, R², R³, and R⁵ is —SO₂(CH₃).

Embodiment 80: The compound of any one of embodiments 1-27, wherein one of R¹, R², R³, and R⁵ is —(CH₂)ₙCO₂R^{D}.

Embodiment 81: The compound of any one of embodiments 1-27 and 80, wherein R^{D} is hydrogen.

Embodiment 82: The compound of any one of embodiments 1-27 and 80, wherein R^{D} is C1-C6 alkyl.

Embodiment 83: The compound of any one of embodiments 1-27, 80 and 82, wherein R^{D} is methyl.

Embodiment 84: The compound of any one of embodiments 1-27, wherein one of R¹, R², R³, and R⁵ is an unsubstituted C1-C6 alkyl.

Embodiment 85: The compound of any one of embodiments 1-27 and 84, wherein one of R¹, R², R³, and R⁵ is methyl.

Embodiment 86: The compound of any one of embodiments 1-27, wherein the other of R¹, R², R³, and R⁵ are independently hydrogen, halogen, cyano, C1-C6 alkyl, C1-C6 alkoxy, C1-C6 haloalkyl, C3-C6 cycloalkyloxy, or 4-6 membered heterocyclyl optionally substituted with 5-6 membered heteroaryl.

Embodiment 87: The compound of any one of embodiments 1-27 and 86, wherein the other of R¹, R², R³, and R⁵ are independently hydrogen, halogen, cyano, C1-C6 alkyl, C1-C6 alkoxy, or C1-C6 haloalkyl.

Embodiment 88: The compound of any one of embodiments 1-27 and 86-87, wherein the other of R¹, R², R³, and R⁵ are independently hydrogen, halogen, or cyano.

Embodiment 89: The compound of any one of embodiments 1-27 and 86-88, wherein the other of R¹, R², R³, and R⁵ are independently hydrogen or halogen.

Embodiment 90: The compound of any one of embodiments 1-27 and 89, wherein the other of R¹, R², R³, and R⁵ are each hydrogen.

Embodiment 91: The compound of any one of embodiments 1-27, wherein one of R¹, R², R³, and R⁵ is halogen, cyano, C1-C6 alkyl optionally substituted with 1-2 substituents independently selected from 4-6 membered heterocyclyl optionally substituted with benzyl or cyano; C1-C6 alkoxy, —(CH₂)ₙ-Q-(4-6 membered heterocyclyl optionally substituted with C1-C6 alkyl), —NHC(═O)(CH₂)ₙR^{C}, 4-10 membered heterocyclyl optionally substituted with phenoxy or 5-6 membered heteroaryl; —NR^{E}R^{F}, C3-C6 cycloalkyl, C3-C6 cycloalkyloxy,

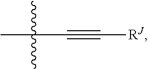

phenoxy optionally substituted with 1-2 substituents independently selected from halogen and C1-C6 haloalkyl; 5-6 membered heteroaryloxy optionally substituted with 1-2 independently selected C1-C6 alkyl;
and the other of R¹, R², R³, and R⁵ are independently hydrogen, halogen, cyano, C1-C6 alkyl, C1-C6 alkoxy, C1-C6 haloalkyl, C3-C6 cycloalkyloxy, or 4-6 membered heterocyclyl optionally substituted with 5-6 membered heteroaryl.

Embodiment 92: The compound of any one of embodiments 1-27 and 91, wherein one of R¹, R², R³, and R⁵ is halogen, cyano, C1-C6 alkyl optionally substituted with 1-2 substituents independently selected from 4-6 membered heterocyclyl optionally substituted with benzyl or cyano; C1-C6 alkoxy, —(CH₂)ₙ-Q-(4-6 membered heterocyclyl optionally substituted with C1-C6 alkyl), —NHC(═O)(CH₂)ₙR^{C}, 4-10 membered heterocyclyl optionally substituted with phenoxy or 5-6 membered heteroaryl; —NR^{E}R^{F}, C3-C6 cycloalkyl, C3-C6 cycloalkyloxy,

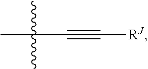

phenoxy optionally substituted with 1-2 substituents independently selected from halogen and C1-C6 haloalkyl; 5-6 membered heteroaryloxy optionally substituted with 1-2 independently selected C1-C6 alkyl;
and the other of R¹, R², R³, and R⁵ are independently hydrogen, halogen, C1-C6 alkyl, C1-C6 alkoxy, C1-C6 haloalkyl, or 4-6 membered heterocyclyl.

Embodiment 93: The compound of any one of embodiments 1-27 and 92, wherein one of R¹, R², R³, and R⁵ is halogen, cyano, C1-C6 alkyl optionally substituted with 1-2 substituents independently selected from 4-6 membered heterocyclyl optionally substituted with benzyl or cyano; C1-C6 alkoxy, —(CH₂)ₙ-Q-(4-6 membered heterocyclyl optionally substituted with C1-C6 alkyl), —NHC(═O)(CH₂)ₙR^{C}, 4-10 membered heterocyclyl optionally substituted with phenoxy or 5-6 membered heteroaryl; —NR^{E}R^{F}, C3-C6 cycloalkyl, C3-C6 cycloalkyloxy,

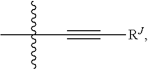

phenoxy optionally substituted with 1-2 substituents independently selected from halogen and C1-C6 haloalkyl; 5-6 membered heteroaryloxy optionally substituted with 1-2 independently selected C1-C6 alkyl;
and the other of R¹, R², R³, and R⁵ are hydrogen.

Embodiment 94: The compound of any one of embodiments 1-27, wherein R³ and R⁵, together with the carbon atoms to which they are attached form a 6 membered heterocyclyl optionally substituted with C1-C6 alkyl or C3-C6 cycloalkyl.

Embodiment 95: The compound of any one of embodiments 1-27 and 94, wherein R³ and R⁵, together with the carbon atoms to which they are attached form a 6 membered heterocyclyl substituted with C1-C6 alkyl.

Embodiment 96: The compound of any one of embodiments 1-27 and 94, wherein R³ and R⁵, together with the carbon atoms to which they are attached form a 6 membered heterocyclyl substituted with C3-C6 cycloalkyl.

Embodiment 97: The compound of any one of embodiments 1-27 and 94, wherein R³ and R⁵, together with the carbon atoms to which they are attached form a 6 membered heterocyclyl.

Embodiment 98: The compound of any one of embodiments 1-27, wherein R³ and one R⁶ adjacent to Ring A, together with the carbon atoms to which they are attached form a 7 membered heterocyclyl.

Embodiment 99: The compound of any one of embodiments 1-27, wherein R³ and one R⁶ adjacent to Ring A, together with the carbon atoms to which they are attached form a 7 membered heterocyclyl.

Embodiment 100: The compound of any one of embodiments 1-27, wherein R¹ and R² together with the carbon atoms to which they are attached form a C3-C5 cycloalkyl.

Embodiment 101: The compound of any one of embodiments 1-27, wherein R² and R³ together with the carbon atoms to which they are attached form a C3-C5 cycloalkyl.

Embodiment 102: The compound of any one of embodiments 1-27, wherein R³ and R⁵, together with the carbon atoms to which they are attached form a C3-C5 cycloalkyl.

Embodiment 103: The compound of any one of embodiments 1-31, wherein Q is —O—.

Embodiment 104: The compound of any one of embodiments 1-31, wherein Q is —C(═O)—.

Embodiment 105: The compound of any one of embodiments 1-31, wherein Q¹ is —O—.

Embodiment 106: The compound of any one of embodiments 1-31, wherein Q¹ is —C(═O)—.

Embodiment 107: The compound of any one of embodiments 1-31 and 39-42, wherein R^{C} is 4-6 membered heterocyclyl optionally substituted with C1-C6 alkyl.

Embodiment 108: The compound of any one of embodiments 1-31 and 39-42, wherein R^{C} is 4-6 membered heterocyclyl substituted with C1-C6 alkyl.

Embodiment 109: The compound of any one of embodiments 1-31 and 39-42, wherein R^{C} is an unsubstituted 4-6 membered heterocyclyl.

Embodiment 110: The compound of any one of embodiments 1-31 and 39-42, wherein R^{C} is 5-6 membered heteroaryl optionally substituted with C1-C6 alkoxy.

Embodiment 111: The compound of any one of embodiments 1-31 and 39-42, wherein R^{C} is 5-6 membered heteroaryl substituted with C1-C6 alkoxy.

Embodiment 112: The compound of any one of embodiments 1-31 and 39-42, wherein R^{C} is 5-6 membered heteroaryl optionally substituted with C1-C6 alkyl.

Embodiment 113: The compound of any one of embodiments 1-31 and 39-42, wherein R^{C} is 5-6 membered heteroaryl substituted with C1-C6 alkyl.

Embodiment 114: The compound of any one of embodiments 1-31 and 39-42, wherein R^{C} is an unsubstituted 5-6 membered heteroaryl.

Embodiment 115: The compound of any one of embodiments 1-31 and 39-49, wherein R^{E} is hydrogen.

Embodiment 116: The compound of any one of embodiments 1-31 and 39-49, wherein R^{E} is C1-C6 alkyl.

Embodiment 117: The compound of any one of embodiments 1-31 and 39-49, wherein R^{E} is —C(═O)—C1-C6 alkyl.

Embodiment 118: The compound of any one of embodiments 1-31 and 39-49, wherein R^{E} is 4-6 membered heterocyclyl.

Embodiment 119: The compound of any one of embodiments 1-31 and 39-53, wherein R^{F} is hydrogen.

Embodiment 120: The compound of any one of embodiments 1-31 and 39-53, wherein R^{F} is C1-C6 alkyl.

Embodiment 121: The compound of any one of embodiments 1-31 and 39-53, wherein R^{F} is —C(═O)—C1-C6 alkyl.

Embodiment 122: The compound of any one of embodiments 1-31 and 39-53, wherein R^{F} is 4-6 membered heterocyclyl.

Embodiment 123: The compound of any one of embodiments 1-31 and 39-49, wherein R^{E} and R^{F} are each hydrogen.

Embodiment 124: The compound of any one of embodiments 1-31 and 39-49, wherein R^{E} and R^{F} are each independently C1-C6 alkyl.

Embodiment 125: The compound of any one of embodiments 1-31 and 39-49, wherein R^{E} and R^{F} are each methyl.

Embodiment 126: The compound of any one of embodiments 1-31 and 39-49, wherein one of R^{E} and R^{F} is hydrogen, and the other of R^{E} and R^{F} is C1-C6 alkyl.

Embodiment 127: The compound of any one of embodiments 1-31 and 39-49, wherein one of R^{E} and R^{F} is hydrogen, and the other of R^{E} and R^{F} is —C(═O)—C1-C6 alkyl.

Embodiment 128: The compound of any one of embodiments 1-31 and 39-49, wherein one of R^{E} and R^{F} is hydrogen, and the other of R^{E} and R^{F} is 4-6 membered heterocyclyl.

Embodiment 129: The compound of any one of embodiments 1-31 and 63, wherein R^{J} is 4-6 membered heterocyclyl.

Embodiment 130: The compound of any one of embodiments 1-31 and 63, wherein R^{J} is 5-6 membered heteroaryl.

Embodiment 131: The compound of any one of embodiments 1-70, wherein R⁶ is C1-C6 alkyl optionally substituted with (i) 4-10 membered heterocyclyl optionally substituted C1-C6 alkyl or (ii) 9-10 membered heteroaryl; C1-C6 haloalkyl, C3-C6 cycloalkyl optionally substituted with —CO₂R^{D}, —NHC(═O)R^{C}, —NR^{G}R^{H}, 4-10 membered heterocyclyl optionally substituted C1-C6 alkyl, —(CH₂)ₚC(═O)NHR, 5-10 membered heteroaryl optionally substituted with C1-C6 alkyl, or —(CH₂)ₛ-Q¹-(4-6 membered heterocyclyl optionally substituted with C1-C6 alkyl).

Embodiment 132: The compound of any one of embodiments 1-70 and 131, wherein R⁶ is C1-C6 alkyl optionally substituted with (i) 4-10 membered heterocyclyl optionally substituted C1-C6 alkyl or (ii) 9-10 membered heteroaryl.

Embodiment 133: The compound of any one of embodiments 1-70 and 131-132, wherein R⁶ is C1-C6 alkyl substituted with 4-10 membered heterocyclyl optionally substituted C1-C6 alkyl.

Embodiment 134: The compound of any one of embodiments 1-70 and 131-132, wherein R⁶ is C1-C6 alkyl substituted with 9-10 membered heteroaryl.

Embodiment 135; The compound of any one of embodiments 1-70 and 131, wherein R⁶ is C1-C6 alkyl.

Embodiment 136: The compound of any one of embodiments 1-70 and 131, wherein R⁶ is C1-C6 haloalkyl.

Embodiment 137: The compound of any one of embodiments 1-70 and 131, wherein R⁶ is C3-C6 cycloalkyl substituted with —CO₂R^{D}.

Embodiment 138: The compound of any one of embodiments 1-70 and 131, wherein R⁶ is C3-C6 cycloalkyl.

Embodiment 139: The compound of any one of embodiments 1-70 and 131, wherein R⁶ is —NHC(═O)R^{C}.

Embodiment 140: The compound of any one of embodiments 1-70 and 131, wherein R is NR^{G}R^{H}.

Embodiment 141: The compound of any one of embodiments 1-70 and 131-133, wherein R⁶ is 4-10 membered heterocyclyl substituted C1-C6 alkyl.

Embodiment 142: The compound of any one of embodiments 1-70 and 131-132, wherein R⁶ is 4-10 membered heterocyclyl.

Embodiment 143: The compound of any one of embodiments 1-70 and 131, wherein R is —(CH₂)ₚC(═O)NHR.

Embodiment 144: The compound of any one of embodiments 1-70 and 131, wherein R is 5-10 membered heteroaryl substituted with C1-C6 alkyl.

Embodiment 145: The compound of any one of embodiments 1-70, wherein and 131 R⁶ is 5-10 membered heteroaryl.

Embodiment 146: The compound of any one of embodiments 1-70, and 131 wherein R⁶ is —(CH₂)ₛ-Q¹-(4-6 membered heterocyclyl optionally substituted with C1-C6 alkyl).

Embodiment 147: The compound of any one of embodiments 1-70 and 131, wherein two geminal R⁶, together with the carbon atom to which they are attached form a C3-C6 spirocycloalkyl.

Embodiment 148: The compound of any one of embodiments 1-57, wherein R^{G} is hydrogen.

Embodiment 149: The compound of any one of embodiments 1-57, wherein R^{G} is C1-C6 alkyl.

Embodiment 150: The compound of any one of embodiments 1-57, wherein R^{G} is C3-C6 cycloalkyl.

Embodiment 151: The compound of any one of embodiments 1-60, wherein R^{H} is hydrogen.

Embodiment 152: The compound of any one of embodiments 1-60, wherein R^{H} is C1-C6 alkyl.

Embodiment 153: The compound of any one of embodiments 1-60, wherein R^{H} is C3-C6 cycloalkyl.

Embodiment 154: The compound of any one of embodiments 1-63, wherein R^{I} is 5-6 membered heteroaryl optionally substituted with C1-C6 alkyl or halogen.

Embodiment 155: The compound of any one of embodiments 1-63, wherein R^{I} is 5-6 membered heteroaryl substituted with C1-C6 alkyl.

Embodiment 156: The compound of any one of embodiments 1-63, wherein R^{I} is 5-6 membered heteroaryl substituted with halogen.

Embodiment 157: The compound of any one of embodiments 1-63, wherein R^{I} is 5-6 membered heteroaryl.

Embodiment 158: The compound of any one of embodiments 1-63, wherein R^{I} is phenyl.

Embodiment 159: The compound of any one of embodiments 1-63, wherein R^{I} is phenyl substituted with halogen.

Embodiment 160: The compound of any one of embodiments 1-159, wherein m is 0.

Embodiment 161: The compound of any one of embodiments 1-159, wherein m is 1.

Embodiment 162: The compound of any one of embodiments 1-159, wherein m is 2.

Embodiment 163: The compound of any one of embodiments 1-159, wherein m is 3.

Embodiment 164: The compound of any one of embodiments 1-163, wherein n is 0.

Embodiment 165: The compound of any one of embodiments 1-163, wherein n is 1.

Embodiment 166: The compound of any one of embodiments 1-163, wherein n is 2.

Embodiment 167: The compound of any one of embodiments 1-166, wherein p is 1.

Embodiment 168: The compound of any one of embodiments 1-166, wherein p is 2.

Embodiment 169: The compound of any one of embodiments 1-166, wherein p is 3.

Embodiment 170: The compound of any one of embodiments 1-166, wherein p is 4.

Embodiment 171: The compound of any one of embodiments 1-170, wherein s is 0.

Embodiment 172: The compound of any one of embodiments 1-170, wherein s is 1.

Embodiment 173: The compound of any one of embodiments 1-170, wherein s is 2.

Embodiment 174: The compound of embodiment 1, wherein Ring A and Ring B form a ring system which is:

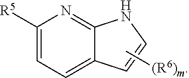

Embodiment 175: The compound of embodiment 1, wherein Ring A and Ring B form a ring system which is:

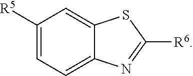

Embodiment 176: The compound of embodiment 1, wherein Ring A and Ring B form a ring system which is:

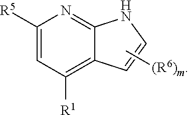

Embodiment 177: The compound of embodiment 1, wherein Ring A and Ring B form a ring system which is:

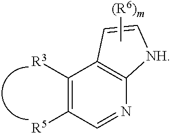

Embodiment 178: The compound of embodiment 1, wherein Ring A and Ring B form a ring system which is:

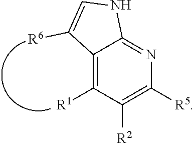

Embodiment 179: The compound of embodiment 1, wherein Ring A and Ring B form a ring system which is:

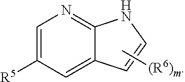

Embodiment 180: The compound of embodiment 1, wherein Ring A and Ring B form a ring system which is:

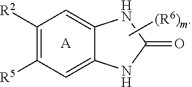

Embodiment 181: The compound of embodiment 1, wherein Ring A and Ring B form a ring system which is:

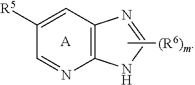

Embodiment 182: The compound of embodiment 1, wherein Ring A and Ring B form a ring system which is:

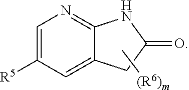

Embodiment 183: The compound of embodiment 1, wherein Ring A and Ring B form a ring system which is:

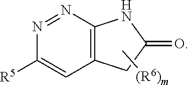

Embodiment 184: The compound of embodiment 1, wherein Ring A and Ring B form a ring system which is:

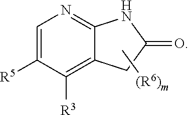

Embodiment 185: The compound of embodiment 1, wherein Ring A and Ring B form a ring system which is:

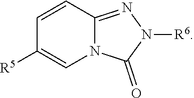

Embodiment 186: The compound of embodiment 1, wherein Ring A and Ring B form a ring system which is:

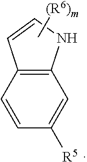

Embodiment 187: The compound of embodiment 1, wherein Ring A and Ring B form a ring system which is:

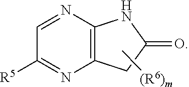

Embodiment 188: The compound of embodiment 1, wherein Ring A and Ring B form a ring system which is:

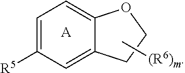

Embodiment 189: The compound of embodiment 1, wherein Ring A and Ring B form a ring system which is:

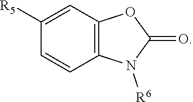

Embodiment 190: The compound of embodiment 1, wherein Ring A and Ring B form a ring system which is:

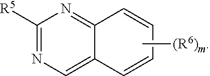

Embodiment 191: The compound of embodiment 1, wherein Ring A and Ring B form a ring system which is:

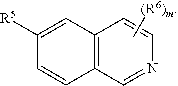

Embodiment 192: The compound of embodiment 1, wherein Ring A and Ring B form a ring system which is:

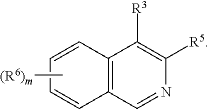

Embodiment 193: The compound of embodiment 1, wherein Ring A and Ring B form a ring system which is:

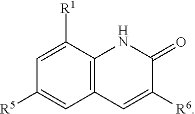

Embodiment 194: The compound of embodiment 1, wherein Ring A and Ring B form a ring system which is:

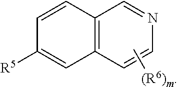

Embodiment 195: The compound of embodiment 1, wherein Ring A and Ring B form a ring system which is:

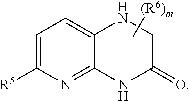

Embodiment 196: The compound of embodiment 1, wherein Ring A and Ring B form a ring system which is:

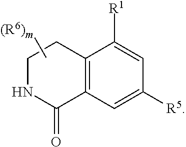

Embodiment 197: The compound of embodiment 1, wherein Ring A and Ring B form a ring system which is:

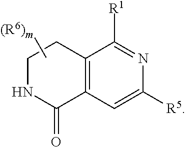

Embodiment 198: The compound of embodiment 1, wherein Ring A and Ring B form a ring system which is:

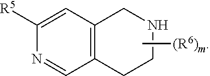

Embodiment 199: The compound of embodiment 1, wherein Ring A and Ring B form a ring system which is:

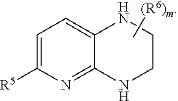

Further exemplary embodiments of compounds of Formulae (I) and (II):

Embodiment 1: A compound of any one of the Embodiments of Formulae (I) and (II), wherein the compound is selected from a compound in Table 1, Table 2, or Table 3, or a pharmaceutically acceptable salt of any of the foregoing.

Embodiment 2: A pharmaceutical composition comprising a compound of Embodiment 1 or any one of the embodiments of compounds of Formulae (I) and (II), or a pharmaceutically acceptable salt thereof, and pharmaceutically acceptable diluent or carrier.

Embodiment 3: A method for treating a neurological disorder in a subject in need thereof, the method comprising administering to the subject a therapeutically effective amount of a compound of Embodiment 1 or any one of the embodiments of compounds of Formulae (I) and (II), or a pharmaceutically acceptable salt thereof, or a pharmaceutical composition according to Embodiment 2.

Embodiment 4: The method of Embodiment 3, wherein the neurological disorder is selected from the group consisting of Down Syndrome, Alzheimer's disease, and Alzheimer's disease associated with Down Syndrome.

Embodiment 5: The method of Embodiment 3 or 4, wherein the neurological disorder is selected Alzheimer's disease associated with Down syndrome.

Embodiment 6: A method of treating a subject, the method comprising administering a therapeutically effective amount of a compound of Embodiment 1 or any one of the embodiments of compounds of Formulae (I) and (II), or a pharmaceutically acceptable salt thereof, or a pharmaceutical composition according to Embodiment 2, to a subject having a clinical record that indicates that the subject has a dysregulation of a DYRK1A gene, DYRK1A protein, or expression or activity or level of any of the same.

Embodiment 7: A method of treating a DYRK1A-associated neurological disorder in a subject, the method comprising administering to a subject identified or diagnosed as having a DYRK1A-associated neurological disorder a therapeutically effective amount of a compound of Embodiment 1 or any one of the embodiments of compounds of Formulae (I) and (II), or a pharmaceutically acceptable salt thereof, or a pharmaceutical composition according to Embodiment 2.

Embodiment 8: A method of treating a DYRK1A-associated neurological disorder in a subject, the method comprising:
(a) determining that the neurological disorder in the subject is a DYRK1A-associated neurological disorder; and
(b) administering to the subject a therapeutically effective amount of a compound of Embodiment 1 or any one of the embodiments of compounds of Formulae (I) and (II), or a pharmaceutically acceptable salt thereof, or a pharmaceutical composition according to Embodiment 2.

Embodiment 9: A method for treating a neurological disorder in a subject in need thereof, the method comprising (a) determining that the neurological disorder is associated with a dysregulation of a DYRK1A gene, a DYRK1A protein, or expression or activity or level of any of the same; and (b) administering to the subject a therapeutically effective amount of Embodiment 1 or a compound of any one of the embodiments of compounds of Formulae (I) and (II), or a pharmaceutically acceptable salt thereof, or a pharmaceutical composition according to Embodiment 2.

Embodiment 10: The method of Embodiment 5853 or 9, wherein the step of determining that the neurological disorder in the subject is a DYRK1A-associated neurological disorder includes performing an assay to detect dysregulation in a DYRK1A gene, a DYRK1A protein, or expression or activity or level of any of the same in a sample from the subject.

Embodiment 11: A method for treating a neurological disorder in a subject in need thereof, the method comprising (a) determining that the neurological disorder is associated with Down Syndrome; and (b) administering to the subject a therapeutically effective amount of Embodiment 1 or a compound of any one of the embodiments of compounds of Formulae (I) and (II), or a pharmaceutically acceptable salt thereof, or a pharmaceutical composition according to Embodiment 2.

Embodiment 12: The method of Embodiment 11, wherein the step of determining that the neurological disorder in the subject is associated with Down Syndrome includes performing an assay on a sample from the subject.

Embodiment 13: The method of any one of Embodiments 9-11, wherein the assay is selected from the group consisting of sequencing, immunohistochemistry, enzyme-linked immunosorbent assay, and fluorescence in situ hybridization (FISH).

Embodiment 14: The method of any one of embodiments 7, 8, or 555, wherein the DYRK1A-associated neurological disorder is selected from the group consisting of Down Syndrome, Alzheimer's disease, and Alzheimer's disease associated with Down Syndrome.

Embodiment 15: The method of any one of embodiments 7, 8, 555, or 559, wherein the DYRK1A-associated neurological disorder is Alzheimer's disease associated with Down syndrome.

Embodiment 16: A method for modulating DYRK1A in a mammalian cell, the method comprising contacting the mammalian cell with a therapeutically effective amount of Embodiment 1 or a compound of any one of the embodiments of compounds of Formulae (I) and (II), or a pharmaceutically acceptable salt thereof.

Embodiment 17: The method of embodiment 16, wherein the contacting occurs in vivo.

Embodiment 18: The method of embodiment 16, wherein the contacting occurs in vitro.

Embodiment 19: The method of any one of embodiments 16-18, wherein the mammalian cell is a mammalian neural cell.

Embodiment 20: The method of embodiment 19, wherein the mammalian neural cell is a mammalian DYRK1A-associated neural cell.

Embodiment 21: The method of any one of embodiments 16-20, wherein the cell has a dysregulation of a DYRK1A gene, a DYRK1A protein, or expression or activity or level of any of the same.

Embodiment 22: The method of any one of embodiments 16-20, wherein the cell has a chromosomal abnormality associated with Down Syndrome.

### EXAMPLES

### Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. The synthesis of the compounds disclosed herein can be achieved by generally following the schemes provided herein, with modification for specific desired substituents.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

### Examples

### General

¹H NMR spectra were recorded on Bruker Avance 400 MHz.

Unless otherwise indicated, LCMS was taken on a quadruple Mass Spectrometer on Shimadzu LCMS 2010 (Column: Shim-pack XR-ODS (3.0×30 mm, 2.2 m)) operating in ESI (+) ionization mode. Flow Rate: 0.8 mL/min, Acquire Time: 2 min or 3 min, Wavelength: UV220, Oven Temp.: 50° C.

Prep-HPLC was performed at conditions:

Method A: Column: Fuji C18 (300×25), YMC 250×20; Wavelength: 220 nm; Mobile phase: A CH₃CN (0.05% FA as an additive); B water (0.05% FA as an additive); Flow rate: 25 mL/min; Injection Volume: 2 mL; Run time: 20 min; Equilibration: 3 min.

Method B: Column: Fuji C18 (300×25), YMC 250×20; Wavelength: 220 nm; Mobile phase: A CH₃CN (0.05% NH₃·H₂O as an additive); B water (0.05% NH₃·H₂O as an additive); Flow rate: 25 mL/min; Injection Volume: 2 mL; Run time: 20 min; Equilibration: 3 min.

Method C: Column: Phenomenex luna C18 (100×25), YMC (250×20); Wavelength: 220 nm; Mobile phase: A CH₃CN; B water (0.225% FA); Flow rate: 25 mL/min; Injection Volume: 2 mL; Run time: 10 min; Equilibration: 3 min.

Method D: Phenomenex luna C18 (100×25), YMC (250×20); Wavelength: 220 nm; Mobile phase: A CH₃CN; B water (0.04% HCl); Flow rate: 25 mL/min; Injection Volume: 2 mL; Run time: 10 min; Equilibration: 3 min.

### Abbreviations

The following abbreviations have the indicated meanings:
AcCl=acetyl chloride
ACN=Acetonitrile
BINAP=(±)-2,2′-Bis(diphenylphosphino)-1,1′-binaphthalene
Bis-Pin=Bis(pinacolato)diboron
BnBr=(bromomethyl)benzene
Boc=t-butyloxy carbonyl
Boc₂O=di-tert-butyl dicarbonate
BocNH₂=tert-butyl carbamate
BrettPhos Pd G3=methanesulfonato(2-dicyclohexylphosphino-3,6-dimethoxy-2′,4′,6′-tri-i-propyl-1,1′-biphenyl)(2′-amino-1,1′-biphenyl-2-yl)palladium(II)
BrettPhos=2-dicyclohexylphosphino-2′,4′,6′-triisopropyl-3,6-dimethoxybiphenyl
CbzCl=benzyl carbonochloridate
Cbz=carbobenzyloxy
CDI=1,1′-Carbonyldiimidazole
CuCN=Copper(I) cyanide
CuI=Copper (I) iodide
CPME=Cyclopentyl methyl ether
CyJohnphos=(2-Biphenyl)dicyclohexylphosphine
DAST=diethylaminosulfur trifluoride
DBU=1,8-Diazabicyclo(5.4.0)undec-7-ene
DCE=1,2-dichloroethane
DCM=dichloromethane
DIEA=N, N-diisopropylethylamine
DIPEA=N, N-diisopropylethylamine
DIAD=diisopropyl azodicarboxylate
DEAD=diethyl azodicarboxylate
DMF=N, N-dimethylformamide
DMAP=N, N-dimethylpyridin-4-amine
DMSO=dimethyl sulfoxide
Dioxane=1,4-dioxane
DPPA=Diphenylphosphoryl azide
Dtbpy=4,4′-di-tert-butyl-2,2′dipyridine
EDCI=1-(3-dimethylaminopropyl)-3-ethylcarbodiimide hydrochloride
EtI=Ethyl iodide
EtOAc=ethyl acetate
FA=formic acid
Grubb's II catalyst=Benzylidene[1,3-bis(2,4,6-trimethylphenyl)-2-imidazolidinylidene]dichloro(tricyclohexylphosphine)ruthenium
HATU=N, N, N, N-tetramethyl-o-(7-azabenzotriazol-1-yl)uronium hexafluorophosphate
HBPin=4,4,5,5-Tetramethyl-1,3,2-dioxaborolane
HF/Py=hydrogen fluoride-pyridine
HOAc=acetic acid
HOBt=1-hydroxybenzotriazole hydrate
HPLC=high performance liquid chromatography
[Ir(OMe)(COD)]₂=Bis(1,5-cyclooctadiene)di-μ-methoxydiiridium(I)
KOAc=potassium acetate
LAH/THF=lithium aluminium hydride, 1M solution in THF
LiHMDS=lithium bis(trimethylsilyl)amide
LC-MS=liquid chromatography-mass spectrometry
M-CPBA=3-Chloroperoxybenzoic acid
Me=methyl
MeOH=methanol
MeCN=acetonitrile
Me₂SO₄=Dimethyl sulfate
NaBH₃CN=Sodium cyanoborohydride
NaOAc=Sodium acetate
NBS=N-bromosuccinimide
n-BuLi=Butyl lithium
NIS=N-iodosuccinimide
NMI=1-methylimidazole
NMP=N-methylpyrrolidone
NMR=nuclear magnetic resonance
PCy₃=tricyclohexyl phosphine
Pd/C=Palladium (0) on activated carbon
Pd₂(dba)₃-CHCl₃=tris(dibenzylideneacetone)dipalladium(0)-chloroform adduct
Pd(dppf)Cl₂—CH₂Cl₂=1,1′-bis(diphenylphosphino)ferrocene-palladium(II)dichloride dichloromethane complex
Pd(dtbpf)Cl₂=[1,1′-Bis(di-tert-butylphosphino)ferrocene]dichloropalladium(II)
Pd(dppf)Cl₂=[1,1′-bis(diphenylphosphino)ferrocene]dichloropalladium(II)
Pd(OAc)₂=Palladium(II) acetate
Pd(PCy₃)₂Cl₂=Dichlorobis(tricyclohexylphosphine)palladium(ii)
Pd(PPh₃)₂Cl₂=bis(triphenylphosphine)palladium chloride
Pd(PPh₃)₄=Tetrakis-(triphenylphosphine)-palladium
PE=petroleum ether
PPA=Polyphosphoric acid
PPTS=Pyridinium p-toluenesulfonate
PtO₂=Platinum(IV) Oxide
Pre-HPLC=preparative high performance liquid chromatography
RT=room temperature
Ruphos-Pd-G2=Chloro(2-dicyclohexylphosphino-2′,6′-diisopropoxy-1,1′-biphenyl)[2-(2′-amino-1,1′-biphenyl)]palladium(II)
Ruphos=2-Dicyclohexylphosphino-2′,6′-diisopropoxybiphenyl
Sat.=saturated
SEMCl=2-(trimethylsilyl)ethoxymethyl chloride
Sodium phosphate buffer=sodium phosphate
Speedvac=Savant SC250EXP SpeedVac Concentrator
Sulfolane=1λ⁶-Thiolane-1,1-dione
Trt=Trityl protecting group
T₃P=propanephosphonic acid cyclic anhydride
TABF=tetrabutylammonium fluoride trihydrate
TBAB=Tetrabutylammonium bromide
TBSCl=tert-butyldimethylsilyl chloride
TCFH=chloro-N,N,N,N-tetramethylformamidinium hexafluorophosphate
t-BuOK=potassium tert-butoxide
TCFH=N, N, N, N-tetramethylchloroformamidinium hexafluorophosphate
TEA=trimethylamine
TEMPO=2,2,6,6-tetramethylpiperidinooxy
TFA=trifluoroacetic acid
TFAA=trifluoroacetic anhydride
THF=tetrahydrofuran
TLC=thin layer chromatography
TMAD=Tetramethylazodicarboxamide
TMSCHNH₂=Trimethylsilyldiazomethane
TsCl=4-Toluenesulfonyl chloride
t-BuOK=potassium tert-butoxide
t-BuONO=tert-Butyl nitrite
t-Bu₃PHBF₄=Tri-tert-butylphosphonium tetrafluoroborate
UV=ultraviolet
XantPhos Pd G2=chloro[(4,5-bis(diphenylphosphino)-9,9-dimethylxanthene)-2-(2-amino-1,1-biphenyl)]palladium(II)
XantPhos=4,5-bis(diphenylphosphino)-9,9-dimethylxanthene
Xphos=2-dicyclohexylphosphino-2′,4′,6′-triisopropylbiphenyl
Xphos Pd G3=methanesulfonato(2-dicyclohexylphosphino-2′,4′,6′-tri-i-propyl-1,1′-biphenyl) (2′-amino-1,1′-biphenyl-2-yl)palladium(II) dichloromethane adduct
×3=three times
×2=two times

### Intermediates of Formula (I)

### Intermediate 1

### 3-iodo-1-tosyl-1H-pyrrolo[2,3-c]pyridine

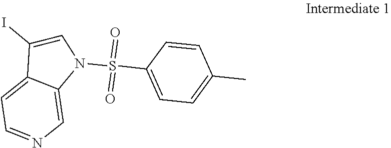 [SMILES: Cc1ccc(S(=O)(=O)n2cc(I)c3ccncc32)cc1]

To a solution of 3-iodo-1H-pyrrolo[2,3-c]pyridine (2.20 g, 9.02 mmol) in DCM (22 mL) was added DIEA (3.50 g, 27.1 mmol), TsCl (1.27 g, 18.0 mmol) and DMAP (110 mg, 0.902 mmol) at 25° C. The mixture was stirred at 25° C. for 0.5 hour. The reaction mixture was concentrated and the residue was purified by silica gel column (PE/EtOAc=10/1 to 3/1) to give 3-iodo-1-tosyl-1H-pyrrolo[2,3-c]pyridine (3.50 g, yield: 97%) as a white solid.

¹H NMR (400 MHz, CDCl₃) δ 2.30 (3H, s), 7.16-7.28 (3H, m), 7.69-7.80 (3H, m), 8.42 (1H, d, J=5.2 Hz), 9.19 (1H, d, J=0.8 Hz).

### Intermediate 2

### 4-1-tosyl-1H-pyrrolo[2,3-c]pyridin-3-yl)pyridin-2-amine

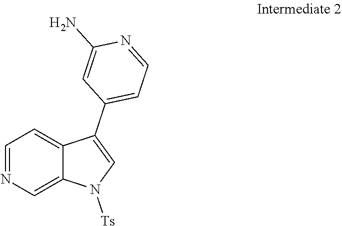 [SMILES: Cc1ccc(S(=O)(=O)n2cc(-c3ccnc(N)c3)c3ccncc32)cc1]

Step 1. Synthesis of tert-butyl (4-(1-tosyl-1H-pyrrolo[2,3-c]pyridin-3-yl)pyridin-2-yl)carbamate A mixture of Intermediate 1 (900 mg, 2.26 mmol), tert-butyl (4-(4,4,5,5-tetramethyl-1,3,2-dioxaborolan-2-yl)pyridin-2-yl)carbamate (796 mg, 2.49 mmol), K₂CO₃ (625 mg, 4.52 mmol) and Pd(dppf)Cl₂ (165 mg, 0.226 mmol) in dioxane (20 mL) and H₂O (5 mL) was degassed and purged with N₂ for 3 times. Then the reaction mixture was stirred at 80° C. for 3 hours under N₂ atmosphere. The reaction mixture was diluted with water (20 mL) and extracted with EtOAc (25 mL×3). The combined organic layer was washed with brine (20 mL), dried over anhydrous Na₂SO₄, filtered and concentrated. The residue was purified by silica gel column (PE/EtOAc=1/1 to 0/1) to give tert-butyl (4-(1-tosyl-1H-pyrrolo[2,3-c]pyridin-3-yl)pyridin-2-yl)carbamate (1.10 g, yield: 86%) as a yellow solid.

¹H NMR (400 MHz, DMSO-d₆) δ 1.49 (9H, s), 2.32 (3H, s), 7.40-7.50 (3H, m), 7.88 (1H, d, J=5.2 Hz), 8.09 (2H, d, J=8.4 Hz), 8.15 (1H, s), 8.33 (1H, d, J=5.6 Hz), 8.52 (1H, d, J=5.6 Hz), 8.61 (1H, s), 9.31 (1H, s), 9.94 (1H, s).

### Step 2. Synthesis of 4-(1-tosyl-1H-pyrrolo[2,3-c]pyridin-3-yl)pyridin-2-amine

To a solution of tert-butyl (4-(1-tosyl-1H-pyrrolo[2,3-c]pyridin-3-yl)pyridin-2-yl)carbamate (500 mg, 1.08 mmol) in DCM (5 mL) was added TFA (5 mL). The mixture was stirred at 25° C. for 2 hours. The reaction mixture was concentrated and the residue was basified with saturated aqueous NaHCO₃ to pH=8, then extracted with EtOAc (50 mL×3). The combined organic layer was washed with brine (20 mL), dried over anhydrous Na₂SO₄, filtered and concentrated to give 4-(1-tosyl-1H-pyrrolo[2,3-c]pyridin-3-yl)pyridin-2-amine (400 mg, crude) as a yellow solid.

### Intermediate 3

### 3-iodo-1-isopropyl-1H-pyrrolo[2,3-c]pyridine

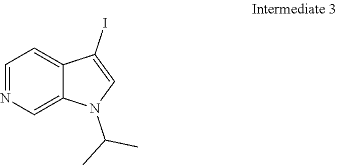

To a solution of 3-iodo-1H-pyrrolo[2,3-c]pyridine (2.00 g, 8.20 mmol) in DMF (20 mL) was added NaH (393 mg, 9.83 mmol, 60% dispersion in mineral oil) at 0° C. The mixture was stirred at 0° C. for 0.5 hour. Then 2-iodopropane (1.53 g, 9.02 mmol) was added at 0° C. The reaction mixture was stirred at 25° C. for 12 hours. The reaction mixture was quenched with H₂O (20 mL) and extracted with EtOAc (50 mL×3). The combined organic layer was washed with brine (20 mL), dried over anhydrous Na₂SO₄, filtered and concentrated. The residue was purified by silica gel column (PE/EtOAc=5/1 to 1/1) to give compound to give 3-iodo-1-isopropyl-1H-pyrrolo[2,3-c]pyridine (1.30 g, yield: 55%) as a yellow solid.

### Intermediate 4

### 4-(1-isopropyl-1H-pyrrolo[2,3-c]pyridin-3-yl)pyridin-2-amine

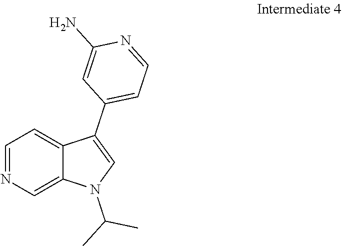 [SMILES: CC(C)n1cc(-c2ccnc(N)c2)c2ccncc21]

### Step 1. Synthesis of tert-butyl (4-(1-isopropyl-1H-pyrrolo[2,3-c]pyridin-3-yl)pyridin-2-yl)carbamate

A mixture of Intermediate 3 (700 mg, 2.45 mmol), tert-butyl (4-(4,4,5,5-tetramethyl-1,3,2-dioxaborolan-2-yl)pyridin-2-yl)carbamate (862 mg, 2.69 mmol), K₂CO₃ (676 mg, 4.89 mmol) and Pd(dppf)Cl₂ (179 mg, 0.245 mmol) in dioxane (10 mL) and H₂O (2.5 mL) was degassed and purged with N₂ for 3 times. Then the reaction mixture was stirred at 90° C. for 3 hours under N₂ atmosphere. The reaction mixture was diluted with water (20 mL) and extracted with EtOAc (50 mL×3). The combined organic layer was washed with brine (20 mL), dried over anhydrous Na₂SO₄, filtered and concentrated. The residue was purified by silica gel column (PE/EtOAc=1/1 to 0/1) to give tert-butyl (4-(1-isopropyl-1H-pyrrolo[2,3-c]pyridin-3-yl)pyridin-2-yl)carbamate (600 mg, yield: 70%) as a brown solid.

¹H NMR (400 MHz, CD₃OD) δ 1.60 (9H, s), 1.67 (6H, d, J=6.8 Hz), 4.95-5.05 (1H, m), 7.43 (1H, dd, J=5.2, 1.2 Hz), 8.05 (1H, d, J=6.0 Hz), 8.22-8.30 (4H, m), 8.94 (1H, s).

### Step 2. Synthesis of 4-(1-isopropyl-1H-pyrrolo[2,3-c]pyridin-3-yl)pyridin-2-amine

To a solution of tert-butyl (4-(1-isopropyl-1H-pyrrolo[2,3-c]pyridin-3-yl)pyridin-2-yl)carbamate (600 mg, 1.70 mmol) in DCM (0.5 mL) was added TFA (0.5 mL). The mixture was stirred at 25° C. for 0.5 hour. The reaction mixture was basified with saturated aqueous Na₂CO₃ to pH=10 and extracted with EtOAc (50 mL×3). The combined organic layer was washed with brine (20 mL), dried over anhydrous Na₂SO₄, filtered and concentrated to give 4-(1-isopropyl-1H-pyrrolo[2,3-c]pyridin-3-yl)pyridin-2-amine (400 mg, yield: 93%) as a yellow solid.

### Intermediate 5

### 3-(4,4,5,5-tetramethyl-1,3,2-dioxaborolan-2-yl)-1-tosyl-1H-pyrrolo[2,3-c]pyridine

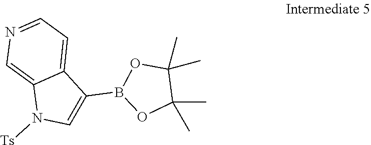 [SMILES: Cc1ccc(S(=O)(=O)n2cc(B3OC(C)(C)C(C)(C)O3)c3ccncc32)cc1]

A mixture of Intermediate 1 (7.50 g, 18.8 mmol), Bis-Pin (23.9 g, 94.2 mmol), Pd(dppf)Cl₂ (1.38 g, 1.88 mmol) and KOAc (5.55 g, 56.5 mmol) in anhydrous DMF (150 mL) was degassed and purged with N₂ for 3 times. Then the resulting reaction mixture was stirred at 100° C. for 20 hours under N₂ atmosphere. The reaction mixture was concentrated and the residue was diluted with water (100 mL) and extracted with EtOAc (100 mL×3). The combined organic layer was washed with brine (50 mL), dried over anhydrous Na₂SO₄, filtered and concentrated. The residue was purified by silica gel column (PE/EtOAc=3/1 to 1/1) to give 3-(4,4,5,5-tetramethyl-1,3,2-dioxaborolan-2-yl)-1-tosyl-1H-pyrrolo[2,3-c]pyridine (4.00 g, yield: 53%) as a brown solid.

### Intermediate 6

### tert-butyl 4-(((4-(4,4,5,5-tetramethyl-1,3,2-dioxaborolan-2-yl)pyridin-2-yl)amino)methyl)piperidine-1-carboxylate

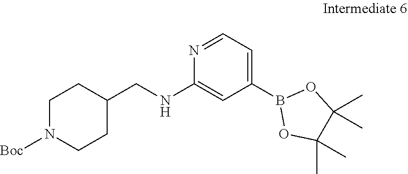 [SMILES: CC(C)(C)OC(=O)N1CCC(CNc2cc(B3OC(C)(C)C(C)(C)O3)ccn2)CC1]

### Step 1. Synthesis of tert-butyl 4-(((4-bromopyridin-2-yl)amino)methyl)piperidine-1-carboxylate

A mixture of 4-bromo-2-fluoropyridine (500 mg, 2.84 mmol), tert-butyl 4-(aminomethyl)piperidine-1-carboxylate (913 mg, 4.26 mmol) and Cs₂CO₃ (2.78 g, 8.52 mmol) in DMSO (5 mL) was stirred at 100° C. for 2 hours. The reaction mixture was diluted with water (30 mL) and extracted with EtOAc (50 mL×3). The combined organic layer was washed with brine (15 mL×3), dried over anhydrous Na₂SO₄, filtered and concentrated. The residue was purified by silica gel column (PE/EtOAc=5/1 to 1/1) to give of tert-butyl 4-(((4-bromopyridin-2-yl)amino)methyl)piperidine-1-carboxylate (700 mg, yield: 67%) as colorless oil.

¹H NMR (400 MHz, CDCl₃) δ 1.08-1.22 (2H, m), 1.46 (9H, s), 1.70-1.80 (3H, m), 2.65-2.75 (2H, m), 3.15-3.20 (2H, m), 4.05-4.15 (2H, m), 4.70 (1H, t, J=5.6 Hz), 6.54 (1H. s), 6.71 (1H, dd, J=5.2, 1.6 Hz), 7.88 (1H, d, J=5.6 Hz).

### Step 2. Synthesis of tert-butyl 4-(((4-(4,4,5,5-tetramethyl-1,3,2-dioxaborolan-2-yl)pyridin-2-yl)amino)methyl)piperidine-1-carboxylate

A mixture of tert-butyl 4-(((4-bromopyridin-2-yl)amino)methyl)piperidine-1-carboxylate (1.00 g, 2.70 mmol), Bis-Pin (892 mg, 3.51 mmol), Pd(dppf)Cl₂ (198 mg, 0.270 mmol) and KOAc (795 mg, 8.10 mmol) in anhydrous dioxane (10 mL) was degassed and purged with N₂ for 3 times. Then the resulting reaction mixture was stirred at 100° C. for 12 hours under N₂ atmosphere. The reaction mixture was concentrated and the residue was diluted with water (20 mL) and extracted with EtOAc (25 mL×3). The combined organic layer was washed with brine (25 mL), dried over anhydrous Na₂SO₄, filtered and concentrated to give tert-butyl 4-(((4-(4,4,5,5-tetramethyl-1,3,2-dioxaborolan-2-yl)pyridin-2-yl)amino)methyl)piperidine-1-carboxylate (1.10 g, crude) as a black gum.

### Intermediate 7

### 4-(1-tosyl-1H-pyrrolo[2,3-c]pyridin-3-yl)pyrimidin-2-amine

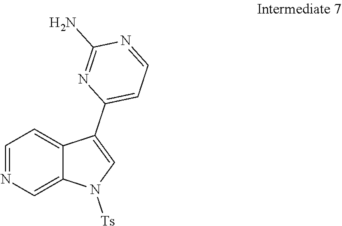 [SMILES: Cc1ccc(S(=O)(=O)n2cc(-c3ccnc(N)n3)c3ccncc32)cc1]

A mixture of Intermediate 5 (922 mg, 2.32 mmol), 4-chloropyrimidin-2-amine (500 mg, 3.86 mmol), Pd(dppf)Cl₂ (282 mg, 0.386 mmol) and K₂CO₃ (800 mg, 5.79 mmol) in dioxane (10 mL) and H₂O (2 mL) was degassed and purged with N₂ for 3 times. Then the reaction mixture was stirred at 100° C. for 3 hours under N₂ atmosphere. The reaction mixture was diluted with water (20 mL) and extracted with EtOAc (25 mL×3). The combined organic layer was washed with brine (20 mL), dried over anhydrous Na₂SO₄, filtered and concentrated. The residue was purified by silica gel column (PE/EtOAc=1/1 to 0/1) to give 4-(1-tosyl-1H-pyrrolo[2,3-c]pyridin-3-yl)pyrimidin-2-amine (500 mg, yield: 35%) as a brown solid.

¹H NMR (400 MHz, CDCl₃) δ 2.39 (3H, s) 5.10 (2H, brs), 7.00 (1H, d, J=5.6 Hz), 7.25-7.30 (2H, m), 7.89 (2H, d, J=8.0 Hz), 8.22-8.32 (2H, m), 8.38 (1H, d, J=5.2 Hz), 8.53 (1H, d, J=5.6 Hz), 9.37 (1H, s).

### Intermediate 8

### 1-isopropyl-3-(4,4,5,5-tetramethyl-1,3,2-dioxaborolan-2-yl)-1H-pyrrolo[2,3-c]pyridine

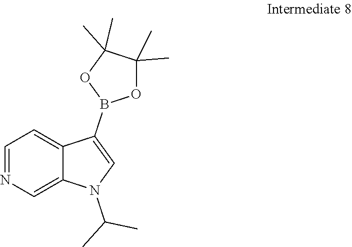 [SMILES: CC(C)n1cc(B2OC(C)(C)C(C)(C)O2)c2ccncc21]

A mixture of Intermediate 3 (4.00 g, 14.0 mmol), Bis-Pin (24.9 g, 97.9 mmol), Pd(dppf)Cl₂ (1.02 g, 1.40 mmol) and KOAc (4.12 g, 41.9 mmol) in anhydrous DMF (50 mL) was degassed and purged with N₂ for 3 times. Then the resulting reaction mixture was stirred at 100° C. for 20 hours under N₂ atmosphere. The reaction mixture was concentrated and the residue was diluted with water (100 mL) and extracted with EtOAc (100 mL×3). The combined organic layer was washed with brine (50 mL×3), dried over anhydrous Na₂SO₄, filtered and concentrated. The residue was purified by silica gel column (DCM/MeOH=1/0 to 20/1) to give 1-isopropyl-3-(4,4,5,5-tetramethyl-1,3,2-dioxaborolan-2-yl)-1H-pyrrolo[2,3-c]pyridine (2.00 g, yield: 50%) as a black gum.

### Intermediate 9

### 3-(2-chloropyrimidin-4-yl)-1-isopropyl-1H-pyrrolo[2,3-c]pyridine

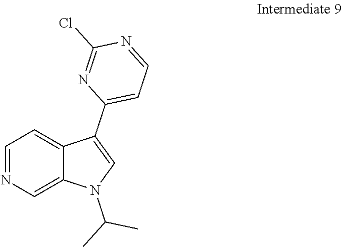 [SMILES: CC(C)n1cc(-c2ccnc(Cl)n2)c2ccncc21]

A mixture of Intermediate 8 (1.92 g, 6.71 mmol), 2, 4-dichloropyrimidine (2.00 g, 13.4 mmol), Pd(dppf)Cl₂ (491 mg, 0.671 mmol) and K₂CO₃ (2.78 g, 20.1 mmol) in dioxane (40 mL) and H₂O (10 mL) was degassed and purged with N₂ for 3 times. Then the resulting reaction mixture was stirred at 90° C. for 6 hours under N₂ atmosphere. The reaction mixture was diluted with water (50 mL) and extracted with EtOAc (70 mL×3). The combined organic layer was washed with brine (50 mL), dried over anhydrous Na₂SO₄, filtered and concentrated. The residue was purified by silica gel column (DCM/MeOH=I/O to 20/1) to give 3-(2-chloropyrimidin-4-yl)-1-isopropyl-1H-pyrrolo[2,3-c]pyridine (500 mg, yield: 14%) as a brown gum.

### Intermediate 10

### 4-(1-isopropyl-1H-pyrrolo[2,3-c]pyridin-3-yl)pyrimidin-2-amine

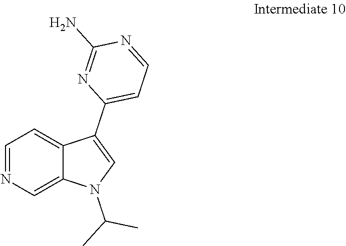 [SMILES: CC(C)n1cc(-c2ccnc(N)n2)c2ccncc21]

A solution of Intermediate 9 (400 mg, 1.47 mmol) in 28% aqueous NH₃·H₂O (10 mL) and EtOH (5 mL) was stirred in a sealed tube at 100° C. for 24 hours. The reaction mixture was concentrated and the residue was purified by silica gel column (DCM/MeOH=1/0 to 20/1) to give -(1-isopropyl-1H-pyrrolo[2,3-c]pyridin-3-yl)pyrimidin-2-amine (250 mg, yield: 67%) as a brown gum.

### Intermediate 11

### 5-bromo-3-cyclobutyl-1H-pyrrolo[2,3-b]pyridine

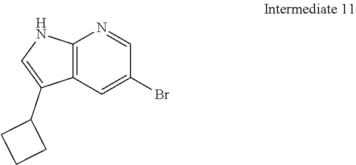 [SMILES: Brc1cnc2[nH]cc(C3CCC3)c2c1]

### Step 1. Synthesis of 5-bromo-3-iodo-J-tosyl-1H-pyrrolo[2,3-b]pyridine

To a solution of 5-bromo-3-iodo-1H-pyrrolo[2,3-b]pyridine (2.10 g, 6.50 mmol), DIPEA (2.52 g, 19.5 mmol), DMAP (16 mg, 0.13 mmol) in DCM (30 mL) was added TsCl (2.48 g, 13.0 mmol). The mixture was stirred at 20° C. for 3 hours. The reaction mixture was poured into water (40 mL) and extracted with EtOAc (40 mL×3). The combined organic layer was washed with brine (50 mL), dried over anhydrous Na₂SO₄, filtered and concentrated. The residue was purified by flash silica gel chromatography (Eluent of 0˜80% Ethyl acetate/Petroleum ether gradient) to give 5-bromo-3-iodo-1-tosyl-1H-pyrrolo[2,3-b]pyridine (2.90 g, yield: 93%) as a yellow solid.

¹H NMR (400 MHz, CDCl₃) δ 2.40 (3H, s), 7.31 (2H, d, J=8.0 Hz), 7.82 (1H, d, J=2.4 Hz), 7.87 (1H, s), 8.06 (2H, d, J=8.4 Hz), 8.46 (1H, d, J=2.0 Hz).

### Step 2. Synthesis of 1-(5-bromo-1-tosyl-1H-pyrrolo[2,3-b]pyridin-3-yl)cyclobutan-1-ol

To a solution of 5-bromo-3-iodo-1-tosyl-1H-pyrrolo[2,3-b]pyridine (2.40 g, 5.03 mmol) in THF (25 mL) was added i-PrMgCl (2.3 mL, 4.60 mmol, 2M in THF) dropwise over 0.5 hour at 0° C. The mixture was stirred at 0° C. for another 1 hour. Cyclobutanone (1.76 g, 25.2 mmol) was added to the above mixture dropwise at 0° C. and the resulting mixture was stirred at 0° C. for 1 hour. The reaction mixture was quenched with saturated aqueous NH₄Cl (40 mL), and extracted with EtOAc (40 mL×3). The combined organic layer was washed with brine (50 mL), dried over anhydrous Na₂SO₄, filtered and concentrated. The residue was purified by flash silica gel chromatography (Eluent of 0˜25% Ethyl acetate/Petroleum ether gradient) to give 1-(5-bromo-1-tosyl-1H-pyrrolo[2,3-b]pyridin-3-yl)cyclobutan-1-ol (1.40 g, yield: 66%) as yellow oil.

¹H NMR (400 MHz, CDCl₃) δ 1.89-2.03 (2H, m), 2.38-2.45 (5H, m), 2.47-2.56 (2H, m), 7.27-7.33 (2H, m), 7.69 (1H, s), 8.03-8.10 (2H, m), 8.17 (1H, d, J=2.0 Hz), 8.45 (1H, d, J=2.4 Hz).

### Step 3. Synthesis of 5-bromo-3-cyclobutyl-1-tosyl-1H-pyrrolo[2,3-b]pyridine

To a solution of 1-(5-bromo-1-tosyl-1H-pyrrolo[2,3-b]pyridin-3-yl)cyclobutan-1-ol (700 mg, 1.66 mmol) and Et₃SiH (2 mL) in DCM (2 mL) was added TFA (2 mL), then stirred at 20° C. for 16 hours. The reaction mixture was poured into water (20 mL), basified with 1N aqueous NaOH to pH=9 and extracted with DCM (25 mL×3). The combined organic layer was washed with brine (30 mL), dried over anhydrous Na₂SO₄, filtered and concentrated. The residue was purified by flash silica gel chromatography (Eluent of 0˜30% Ethyl acetate/Petroleum ether gradient) to give 5-bromo-3-cyclobutyl-1-tosyl-1H-pyrrolo[2,3-b]pyridine (500 mg, yield: 74%) as a white solid.

¹H NMR (400 MHz, CDCl₃) δ 1.89-1.99 (1H, m), 2.05-2.21 (3H, m), 2.34-2.42 (5H, m), 3.49-3.60 (1H, m), 7.23-7.26 (2H, m), 7.47 (1H, d, J=1.2 Hz), 7.85 (1H, d, J=2.4 Hz), 7.99-8.04 (2H, m), 8.40 (1H, d, J=2.0 Hz).

### Step 4. Synthesis of 5-bromo-3-cyclobutyl-1H-pyrrolo[2,3-b]pyridine

To a solution of 5-bromo-3-cyclobutyl-1-tosyl-1H-pyrrolo[2,3-b]pyridine (800 mg, 1.97 mmol) in MeOH (20 mL) was added 2N aqueous NaOH (5 mL), the mixture was stirred at 100° C. for 2 hours. After cooled to room temperature, the reaction mixture was poured into water (50 mL), and extracted with EtOAc (40 mL×3), the combined organic layer was washed with brine (60 mL), dried over anhydrous Na₂SO₄, filtered and concentrated. The residue was purified by flash silica gel chromatography (Eluent of 0˜18% Ethyl acetate/Petroleum ether gradient) to give 5-bromo-3-cyclobutyl-1H-pyrrolo[2,3-b]pyridine (400 mg, yield: 81%) as a white solid.

### Intermediate 12

### tert-butyl 3-(6-(pyridin-4-yl)-1H-benzo[d]imidazol-1-yl)piperidine-1-carboxylate

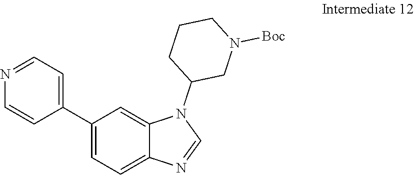 [SMILES: CC(C)(C)OC(=O)N1CCCC(n2cnc3ccc(-c4ccncc4)cc32)C1]

### Step 1. Synthesis of tert-butyl 3-((5-bromo-2-nitrophenyl)amino)piperidine-1-carboxylate

A mixture of 4-bromo-2-fluoro-1-nitrobenzene 0.0 g, 45.5 mmol), tert-butyl 3-aminopiperidine-1-carboxylate oxalic acid salt (13.2 g, 45.5 mmol,), Na₂CO₃ (21.7 g, 205 mmol) in DMF (150 mL) was stirred at 80° C. for 2 hours under N₂ atmosphere. The reaction mixture was diluted with water (500 mL) and extracted with EtOAc (250 mL×3). The combined organic layer was washed with brine (200 mL×3), dried over anhydrous Na₂SO₄, filtered and concentrated to give tert-butyl 3-((5-bromo-2-nitrophenyl)amino)piperidine-1-carboxylate (15.0 g, yield: 82%) as a yellow solid.

¹H NMR (400 MHz, CDCl₃) δ 1.48 (9H, s) 1.60-1.80 (4H, m), 3.10-3.30 (2H, m), 3.50-3.75 (2H, m), 3.90-3.95 (1H, m), 6.80 (1H, d, J=9.2 Hz), 7.10 (1H, s), 8.07 (1H, d, J=9.2 Hz), 8.16 (1H, brs).

### Step 2. Synthesis of tert-butyl 3-((2-amino-5-bromophenyl)amino)piperidine-1-carboxylate

A mixture of tert-butyl 3-((5-bromo-2-nitrophenyl)amino)piperidine-1-carboxylate (1.90 g, 4.75 mmol), Fe powder (1.06 g, 19.0 mmol) and NH₄Cl (1.02 g, 19.0 mmol) in EtOH (18 mL) and H₂O (18 mL) was stirred at 75° C. for 1 hour. The reaction was filtered and the solid was washed with hot EtOH (10 mL×3). The filtrate was concentrated and the residue was purified by flash silica gel chromatography (ISCO®; 40 g SepaFlash® Silica Flash Column, Eluent of 0˜55% Ethyl acetate/Petroleum ether gradient @50 mL/min) to give tert-butyl 3-((2-amino-5-bromophenyl)amino)piperidine-1-carboxylate (1.50 g, 3.77 mmol, 79.31% yield, 93% purity) was obtained as a gray solid.

¹H NMR (400 MHz, CDCl₃) δ 1.39 (9H, s) 1.50-1.60 (2H, m), 1.62-1.95 (2H, m), 2.87-3.13 (2H, m), 3.15-3.20 (2H, m), 3.23-3.47 (2H, m), 3.50-3.85 (2H, m), 6.52 (1H, d, J=8.0 Hz), 6.65-6.75 (2H, m).

### Step 3. Synthesis of tert-butyl 3-(6-bromo-1H-benzo[d]imidazol-1-yl)piperidine-1-carboxylate

A mixture of tert-butyl 3-((2-amino-5-bromophenyl)amino)piperidine-1-carboxylate (200 mg, 0.540 mmol) and trimethoxymethane (4.84 g, 45.6 mmol) was stirred at 90° C. for 16 hours under N₂ atmosphere. The reaction mixture was concentrated and the residue was purified by flash silica gel chromatography (ISCO®; 10 g SepaFlash® Silica Flash Column, Eluent of 0˜30% Ethyl acetate/Petroleum ether gradient @20 mL/min) to give tert-butyl 3-(6-bromo-1H-benzo[d]imidazol-1-yl)piperidine-1-carboxylate (200 mg, yield: 97%) as a gray solid.

### Step 4. Synthesis of tert-butyl 3-(6-(pyridin-4-yl)-1H-benzo[d]imidazol-1-yl)piperidine-1-carboxylate

A mixture of tert-butyl 3-(6-bromo-1H-benzo[d]imidazol-1-yl)piperidine-1-carboxylate (700 mg, 1.84 mmol), 4-pyridylboronic acid (339 mg, 2.76 mmol), Pd(dppf)Cl₂ (269 mg, 0.368 mmol) and Na₂CO₃ (585 mg, 5.52 mmol) in dioxane (4 mL) and water (1 mL) was degassed and purged with N₂ for 3 times. Then the mixture was stirred at 90° C. for 16 hours under N₂ atmosphere. The reaction mixture was concentrated and the residue was purified by flash silica gel chromatography (ISCO®; 12 g SepaFlash® Silica Flash Column, Eluent of 0˜50% Ethyl acetate/Petroleum ether gradient @25 mL/min) to give tert-butyl 3-(6-(pyridin-4-yl)-1H-benzo[d]imidazol-1-yl)piperidine-1-carboxylate (200 mg, yield: 29%) as a yellow solid.

### Intermediate 13

### 6-(pyridin-4-yl)imidazo[1,2-a]pyridine-3-carbaldehyde

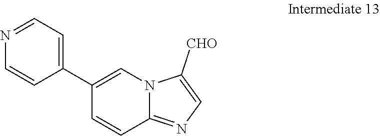 [SMILES: O=Cc1cnc2ccc(-c3ccncc3)cn12]

### Step 1. Synthesis of [3,4′-bipyridin]-6-amine

A mixture of 5-bromopyridin-2-amine (3.73 g, 30.3 mmol), pyridin-4-ylboronic acid (5.00 g, 28.9 mmol), Pd(dppf)Cl₂ (1.06 g, 1.44 mmol), K₂CO₃ (7.99 g, 57.8 mmol) in dioxane (40 mL) and H₂O (10 mL) was degassed and purged with N₂ for 3 times, then the mixture was stirred at 105° C. for 12 hours under N₂ atmosphere. The reaction mixture was diluted with water (100 mL) and extracted with EtOAc (100 mL×3). The combined organic layer was washed with brine (100 mL), dried over anhydrous Na₂SO₄, filtered and concentrated to give [3,4′-bipyridin]-6-amine (1.50 g, yield: 27%) as a brown solid.

¹H NMR (400 MHz, CDCl₃) δ 4.53-4.78 (2H, brs), 6.61 (1H, d, J=8.4 Hz), 7.38-7.46 (2H, m), 7.73 (1H, dd, J=8.4, 2.4 Hz), 8.42 (1H, d, J=2.0 Hz), 8.58-8.67 (2H, m).

### Step 2. Synthesis of 6-(pyridin-4-yl)imidazo[1,2-a]pyridine-3-carbaldehyde

A mixture of [3,4′-bipyridin]-6-amine (1.48 g, 8.66 mmol) and 2-bromomalonaldehyde (1.57 g, 10.4 mmol) in CH₃CN (30 mL) was stirred at 85° C. for 2 hours under N₂ atmosphere. The reaction mixture was diluted with water (50 mL) and extracted with EtOAc (50 mL×3). The combined organic layer was washed with brine (50 mL), dried over anhydrous Na₂SO₄, filtered and concentrated. The residue was purified by Combi Flash (0% to 8% MeOH in DCM) to give 6-(pyridin-4-yl)imidazo[1,2-a]pyridine-3-carbaldehyde (800 mg, yield: 37%) as an off-white solid.

¹H NMR (400 MHz, CDCl₃) δ 7.55-7.65 (2H, m), 7.85 (1H, d, J=9.2 Hz), 7.94 (1H, d, J=9.6 Hz), 8.40 (1H, s), 8.75-8.85 (2H, m), 9.85 (1H, d, J=1.2 Hz), 10.06 (1H, s).

### Intermediate 14

### 4-bromo-1-(tetrahydro-2H-pyran-2-yl)-1H-indazole

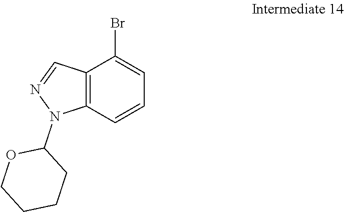 [SMILES: Brc1cccc2c1cnn2C1CCCCO1]

A mixture of 4-bromo-1H-indazole (2.00 g, 10.1 mmol), DHP (Int14b; 1.02 g, 12.1 mmol) and PPTS (25 mg, 0.102 mmol) in anhydrous DCM (5 mL) was stirred at 25° C. for 14 hours under N₂ atmosphere. The reaction mixture was diluted with water (50 mL) and extracted with DCM (50 mL×2). The combined organic layer was washed with brine (50 mL), dried over anhydrous Na₂SO₄, filtered and concentrated. The residue was purified by Combi Flash (0% to 15% EtOAc in PE) to give 4-bromo-1-(tetrahydro-2H-pyran-2-yl)-1H-indazole (1.90 g, yield: 63%) as yellow oil.

### Intermediate 15

### benzyl 4-(6-bromo-1H-benzo[d]imidazol-1-yl)piperidine-1-carboxylate

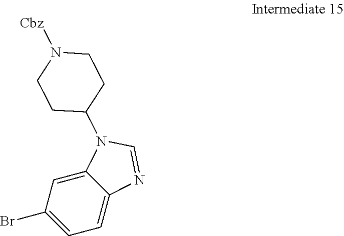 [SMILES: O=C(OCc1ccccc1)N1CCC(n2cnc3ccc(Br)cc32)CC1]

### Step 1. Synthesis of benzyl 4-((5-bromo-2-nitrophenyl)amino)piperidine-1-carboxylate

A mixture of 4-bromo-2-fluoro-1-nitrobenzene (2.00 g, 9.09 mmol), benzyl 4-aminopiperidine-1-carboxylate (2.13 g, 9.09 mmol), Na₂CO₃ (4.82 g, 45.5 mmol) in DMF (15 mL) was stirred at 80° C. for 1 hour under N₂ atmosphere. The reaction mixture was diluted with water (100 mL) and extracted with EtOAc (50 mL×3). The combined organic layer was washed with brine (100 mL×3), dried over anhydrous Na₂SO₄, filtered and concentrated to give benzyl 4-((5-bromo-2-nitrophenyl)amino)piperidine-1-carboxylate (3.90 g, yield: 99%) as a yellow solid.

¹H NMR (400 MHz, DMSO-d₆) δ 1.45-1.55 (2H, m), 1.90-2.00 (2H, m), 3.00-3.10 (2H, m), 3.25-3.28 (1H, m), 3.90-4.05 (3H, m), 5.09 (2H, s), 6.86 (1H, dd, J=8.8, 2.0 Hz), 7.34-7.40 (4H, m), 7.43 (1H, s), 7.95 (1H, d, J=8.4 Hz), 8.00 (1H, d, J=9.2 Hz).

### Step 2. Synthesis of benzyl 4-((2-amino-5-bromophenyl)amino)piperidine-1-carboxylate

To a solution of benzyl 4-((5-bromo-2-nitrophenyl)amino)piperidine-1-carboxylate (4.00 g, 9.21 mmol), Fe powder (2.06 g, 36.8 mmol) and NH₄Cl (1.97 g, 36.8 mmol) in EtOH (20 mL) and H₂O (20 mL) was stirred at 75° C. for 1 hour. The reaction was filtered and the solid was washed with hot EtOH (20 mL×3). The filtrate was concentrated and the residue was diluted with EtOAc (100 mL) and washed with water (60 mL×3), dried over anhydrous Na₂SO₄, filtered and concentrated to give benzyl 4-((2-amino-5-bromophenyl)amino)piperidine-1-carboxylate (3 g, yield: 81%) as a black brown gum, which was directly used for the next step without further purification.

### Step 3. Synthesis of benzyl 4-(6-bromo-1H-benzo[d]imidazol-1-yl)piperidine-1-carboxylate

A mixture of benzyl 4-((2-amino-5-bromophenyl)amino)piperidine-1-carboxylate (1.00 g, 2.47 mmol) and PPTS (62 mg, 0.25 mmol) in trimethoxymethane (9.68 g, 91.2 mmol) was stirred at 90° C. for 16 hours under N₂ atmosphere. The reaction mixture was concentrated and the residue was purified by flash silica gel chromatography (ISCO®; 12 g SepaFlash® Silica Flash Column, Eluent of 0˜10% Ethyl acetate/Petroleum ether gradient @20 mL/min) to give benzyl 4-(6-bromo-1H-benzo[d]imidazol-1-yl)piperidine-1-carboxylate (880 mg, yield: 86%) as a brown gum.

¹H NMR (400 MHz, CDCl₃) δ 1.95-2.09 (2H, m), 2.15-2.25 (2H, m), 3.00-3.10 (2H, m), 4.30-4.35 (1H, m), 4.45-4.50 (2H, m), 5.21 (2H, s), 7.33-7.47 (6H, m), 7.59 (1H, d, J=1.6 Hz), 7.70 (1H, d, J=8.4 Hz), 7.95 (1H, s).

### Intermediate 16

### 6-(2-methoxypyridin-4-yl)benzo[d]thiazol-2-amine

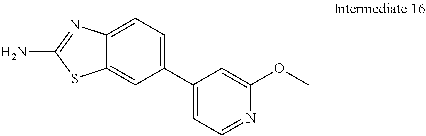 [SMILES: COc1cc(-c2ccc3nc(N)sc3c2)ccn1]

A mixture of 6-bromobenzo[d]thiazol-2-amine (2.00 g, 8.73 mmol), (2-methoxypyridin-4-yl)boronic acid (2.67 g, 17.5 mmol), K₂CO₃ (2.41 g, 17.5 mmol), (t-Bu₃P)₂Pd (569 mg, 0.873 mmol) in dioxane (40 mL) and H₂O (10 mL) was degassed and purged with N₂ for 3 times. The reaction mixture was stirred at 120° C. for 1 hour under N₂ atmosphere. The reaction mixture was filtered and the filtrate was concentrated. The residue was purified by flash silica gel chromatography (ISCO®; 20 g SepaFlash® Silica Flash Column, Eluent of 0˜100% Ethyl acetate/Petroleum ether gradient @40 mL/min) to give 6-(2-methoxypyridin-4-yl)benzo[d]thiazol-2-amine (1.90 g, yield: 85%) as a yellow solid.

### Intermediate 17

### 6-(pyridin-4-yl)benzo[d]thiazol-2-amine

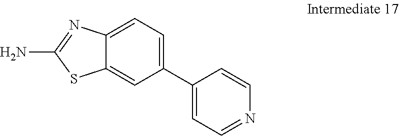 [SMILES: Nc1nc2ccc(-c3ccncc3)cc2s1]

A mixture of 6-bromobenzo[d]thiazol-2-amine (2.00 g, 8.73 mmol), pyridin-4-ylboronic acid (2.15 g, 17.5 mmol), (t-Bu₃P)₂Pd (569 mg, 0.873 mmol) and K₂CO₃ (2.41 g, 17.5 mmol) in dioxane (40 mL) and H₂O (10 mL) was degassed and purged with N₂ for 3 times, then the mixture was stirred at 90° C. for 16 hours under N₂ atmosphere. The reaction mixture was filtered and the filtrate was concentrated. The residue was purified by flash silica gel chromatography (ISCO®; 25 g SepaFlash® Silica Flash Column, Eluent of 0˜100% Ethyl acetate/Petroleum ether gradient @30 mL/min) to give 6-(pyridin-4-yl)benzo[d]thiazol-2-amine (600 mg, yield: 30%) as a yellow solid.

¹H NMR (400 MHz, DMSO-d₆) δ 7.43 (1H, d, J=8.4 Hz), 7.63-7.74 (5H, m), 8.19 (1H, d, J=1.6 Hz), 8.55-8.60 (2H, m).

### Intermediate 18

### tert-butyl (6-bromopyrrolo[1,2-c]pyrimidin-3-yl)carbamate

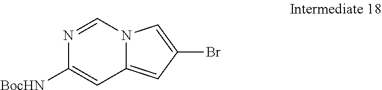 [SMILES: CC(C)(C)OC(=O)Nc1cc2cc(Br)cn2cn1]

### Step 1. Synthesis of ethyl 6-bromopyrrolo[1,2-c]pyrimidine-3-carboxylate

To a solution of 4-bromo-1H-pyrrole-2-carbaldehyde (4.00 g, 23.0 mmol) and DBU (3.50 g, 23.0 mmol) in DCM (40 mL) was added ethyl 2-isocyanoacetate (2.60 g, 23.0 mmol) at 25° C. The mixture was stirred at 60° C. for 4 hours. The reaction mixture was diluted with 10% aqueous HOAc (80 mL) and extracted with DCM (100 mL×3). The combined organic layers were washed with H₂O (100 mL×3), dried over anhydrous Na₂SO₄, filtered and concentrated. The residue was purified by Combi Flash (0% to 60% EtOAc in PE) to give ethyl 6-bromopyrrolo[1,2-c]pyrimidine-3-carboxylate (3.30 g, yield: 53%) as a yellow solid.

### Step 2. Synthesis of 6-bromopyrrolo[1,2-c]pyrimidine-3-carboxylic Acid

To a solution of ethyl 6-bromopyrrolo[1,2-c]pyrimidine-3-carboxylate (3.30 g, 12.3 mmol) in THF (16 mL) was added a solution of LiOH·H₂O (1.17 g, 49.1 mmol) in H₂O (4 mL). The mixture was stirred at 25° C. for 2 hours. The reaction mixture was acidified with 1N aqueous HCl to pH=6-7. The solid was filtered and dried to give 6-bromopyrrolo[1,2-c]pyrimidine-3-carboxylic acid (2.38 g, yield: 80%) as a yellow solid.

### Step 3. Synthesis of tert-butyl (6-bromopyrrolo[1,2-c]pyrimidin-3-yl)carbamate

To a solution of 6-bromopyrrolo[1,2-c]pyrimidine-3-carboxylic acid (1.00 g, 4.15 mmol), DPPA (3.43 g, 12.5 mmol) and TEA (1.26 g, 12.5 mmol) in THF (20 mL) was added t-BuOH (60 mL) and Boc₂O (3.62 g, 16.6 mmol). The mixture was stirred at 90° C. for 16 hours. The reaction mixture was concentrated and the residue was diluted with H₂O (50 mL) and extracted with EtOAc (100 mL×3). The combined organic layers were washed with brine (100 mL), dried over anhydrous Na₂SO₄, filtered and concentrated. The residue was purified by Combi Flash (0% to 15% EtOAc in PE) twice to give tert-butyl (6-bromopyrrolo[1,2-c]pyrimidin-3-yl)carbamate (550 mg, yield: 42%) as a light yellow solid.

¹H NMR (400 MHz, DMSO-d₆) δ 1.48 (9H, s), 6.39 (1H, s), 7.57 (1H, s), 7.66 (1H, d, J=0.9 Hz), 8.91 (1H, s), 9.61 (1H, s).

### Intermediate 19

### 6-(pyridin-4-yl)pyrrolo[1,2-c]pyrimidin-3-amine

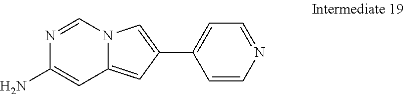 [SMILES: Nc1cc2cc(-c3ccncc3)cn2cn1]

### Step 1. Synthesis of tert-butyl (6-(pyridin-4-yl)pyrrolo[1,2-c]pyrimidin-3-yl)carbamate

A mixture of Intermediate 18 (550 mg, 1.76 mmol), pyridin-4-ylboronic acid (325 mg, 2.64 mmol), Pd(dppf)Cl₂ (129 mg, 0.176 mmol) and Na₂CO₃ (374 mg, 3.52 mmol) in 1,4-dioxane (10 mL) and H₂O (1 mL) was degassed and purged with N₂ for 3 times, and then the mixture was stirred at 90° C. for 16 hours under N₂ atmosphere. The reaction mixture was quenched by addition H₂O (25 mL) and extracted with EtOAc (50 mL×2). The combined organic layers were washed with brine (50 mL), dried over anhydrous Na₂SO₄, filtered and concentrated. The residue was purified by Combi Flash (0% to 100% EA in PE) to give tert-butyl (6-(pyridin-4-yl)pyrrolo[1,2-c]pyrimidin-3-yl)carbamate (350 mg, yield: 64%) as a yellow solid.

### Step 2. Synthesis of 6-(pyridin-4-yl)pyrrolo[1,2-c]pyrimidin-3-amine

A solution of tert-butyl (6-(pyridin-4-yl)pyrrolo[1,2-c]pyrimidin-3-yl)carbamate (350 mg, 1.13 mmol) in DCM (4 mL) and TFA (4 mL) was stirred at 20° C. for 4 hours. To the reaction mixture was added saturated aqueous NaHCO₃ to pH=8 and extracted with DCM (50 mL×2). The combined organic layers were washed with brine (50 mL), dried over anhydrous Na₂SO₄, filtered and concentrated to give 6-(pyridin-4-yl)pyrrolo[1,2-c]pyrimidin-3-amine (300 mg, yield: 82%) as a yellow solid.

### Intermediate 20

### 6-(2-ethoxypyridin-4-yl)benzo[d]thiazol-2-amine

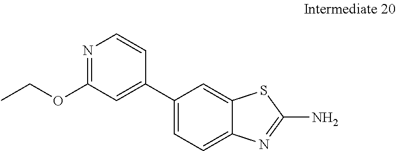 [SMILES: CCOc1cc(-c2ccc3nc(N)sc3c2)ccn1]

A mixture of 6-bromobenzo[d]thiazol-2-amine (700 mg, 2.81 mmol), 2-ethoxy-4-(4,4,5,5-tetramethyl-1,3,2-dioxaborolan-2-yl)pyridine (773 mg, 3.37 mmol), Pd(dppf)Cl₂ (206 mg, 0.281 mmol), Na₂CO₃ (893 mg, 8.43 mmol) in dioxane (8 mL) and H₂O (2 mL) was degassed and purged with N₂ for 3 times, and then the mixture was stirred at 95° C. for 2 hours under N₂ atmosphere. The reaction mixture was filtered and the filtrate was concentrated. The residue was purified by flash silica gel chromatography (ISCO®; 12 g SepaFlash® Silica Flash Column, Eluent of 0˜50% Ethyl acetate/Petroleum ether gradient @40 mL/min) to give compound 3 (230 mg, yield: 30%) as a yellow solid.

¹H NMR (400 MHz, DMSO-d₆) δ 1.34 (3H, t, J=7.2 Hz), 4.34 (2H, q, J=7.2 Hz), 7.07 (1H, s), 7.30 (1H, dd, J=7.2, 1.6 Hz), 7.40 (1H, d, J=8.4 Hz), 7.63-7.72 (3H, m), 8.12-8.22 (2H, m).

### Intermediate 21

### cis-(2.4.6)-1-(tert-butoxycarbonyl)-2,6-dimethylpiperidine-4-carboxylic Acid

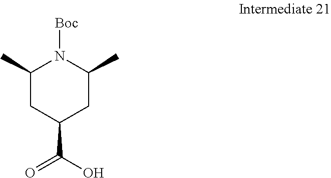

### Step 1. Synthesis of cis-(2,4,6) methyl-2,6-dimethylpiperidine-4-carboxylate

A solution of methyl 2,6-dimethylisonicotinate (2.00 g, 12.1 mmol) in 4M HCl/MeOH (5 mL) was stirred at 25° C. for 0.5 hour. The mixture was concentrated and dissolved in MeOH (30 mL). Then PtO₂ (500 mg, 2.20 mmol) was added to the reaction mixture under N₂ atmosphere. The reaction mixture was degassed and purged with H₂ for 3 times. The mixture was hydrogenated (50 psi) at 25° C. for 5 hours. The reaction mixture was filtered and the filtrate was concentrated to give cis-(2,4,6) methyl-2,6-dimethylpiperidine-4-carboxylate (2.50 g, yield: 99%, HCl salt) as a white solid.

¹H NMR (400 MHz, DMSO-d₆) δ 1.22 (d, J=6.4 Hz, 6H), 1.40-1.50 (2H, m), 1.95-2.00 (2H, m), 2.60-2.80 (1H, m), 3.05-3.20 (2H, m), 3.58 (3H, s), 8.51 (1H, brs), 9.21 (1H, brs).

### Step 2. Synthesis of cis-(2,4,6)-1-(tert-butyl) 4-methyl-2,6-dimethylpiperidine-1,4-dicarboxylate

To a solution of cis-(2,4,6) methyl-2,6-dimethylpiperidine-4-carboxylate (2.00 g, 9.63 mmol, HCl salt) in dioxane (20 mL) was added Na₂CO₃ (3.06 g, 28.9 mmol) at 25° C. The mixture was stirred at 25° C. for 0.5 hour. Then Boc₂O (3.15 g, 14.4 mmol) was added to the reaction mixture and the reaction mixture was stirred at 25° C. for another 36 hours. The reaction mixture was acidified with 1 N aqueous HCl to pH=5 and extracted with EtOAc (50 mL×3). The combined organic layer was washed with brine (30 mL), dried over anhydrous Na₂SO₄, filtered and concentrated to give cis-(2,4,6)-1-(tert-butyl) 4-methyl-2,6-dimethylpiperidine-1,4-dicarboxylate (3.00 g, crude) as a colorless oil.

### Step 3. Synthesis of cis-(2,4,6)-1-(tert-butoxycarbonyl)-2,6-dimethylpiperidine-4-carboxylic Acid

A solution of cis-(2,4,6)-1-(tert-butyl) 4-methyl-2,6-dimethylpiperidine-1,4-dicarboxylate (3.00 g, crude) and LiOH·H₂O (2.65 g, 111 mmol) in MeOH (5 mL), THF (5 mL) and H₂O (5 mL) was stirred at 50° C. for 1 hour. The reaction mixture was diluted with water (15 mL) and washed with DCM (30 mL). The aqueous layer were acidified with 1 N aqueous HCl to pH=3 and extracted with EtOAc (50 mL×3). The combined organic layer was dried over anhydrous Na₂SO₄, filtered and concentrated to give cis-(2,4,6)-1-(tert-butoxycarbonyl)-2,6-dimethylpiperidine-4-carboxylic acid (700 mg, yield: 25%) as a colorless gum.

¹H NMR (400 MHz, CD₃OD) δ 1.28 (6H, d, J=6.8 Hz), 1.49 (9H, s), 1.62-1.68 (2H, m), 2.18-2.30 (2H, m), 2.40-2.50 (1H, m), 4.18-4.29 (2H, m).

### Intermediate 22

### 6-(pyridin-4-ylimidazo[1,2-a]pyridin-2-amine

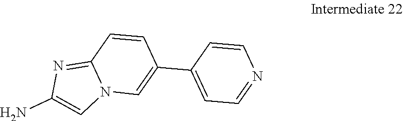 [SMILES: Nc1cn2cc(-c3ccncc3)ccc2n1]

A mixture of 6-bromoimidazo[1,2-a]pyridin-2-amine (500 mg, 2.36 mmol), pyridine-4-boronic acid (870 mg, 7.07 mmol), Pd(dppf)Cl₂ (173 mg, 0.236 mmol), Na₂CO₃ (750 mg, 7.07 mmol) in dioxane (20 mL) and H₂O (6 mL) was degassed and purged with N₂ for 3 times. Then the mixture was stirred at 100° C. for 12 hours under N₂ atmosphere. The reaction mixture was diluted with H₂O (30 mL) and extracted with EtOAc (50 mL×3). The combined organic layer was washed with brine (30 mL), dried over anhydrous Na₂SO₄, filtered and concentrated. The residue was purified by silica gel column (DCM/MeOH=20/1 to 10/1) to give 6-(pyridin-4-yl)imidazo[1,2-a]pyridin-2-amine (300 mg, yield: 61%) as a brown solid.

¹H NMR (400 MHz, DMSO-d₆) δ 5.20 (2H, brs), 7.06 (1H, s), 7.29 (1H, d, J=9.2 Hz), 7.47 (1H, dd, J=8.8, 1.6 Hz), 7.66-7.74 (2H, m), 8.55-8.65 (2H, m), 8.90 (1H, d, J=1.2 Hz).

### Intermediate 23

### 5-methyl-6-(pyridin-4-yl)pyrrolo[1,2-c]pyrimidin-3-amine

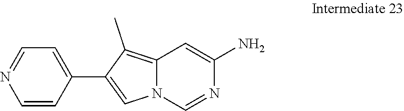 [SMILES: Cc1c(-c2ccncc2)cn2cnc(N)cc12]

### Step 1. Synthesis of 3-methyl-1H-pyrrole-2-carbaldehyde

To a solution of ethyl 3-methyl-1H-pyrrole-2-carboxylate (8.30 g, 54.2 mmol) in anhydrous THF (100 mL) was added LiAlH₄ (6.17 g, 163 mmol) portion-wise at 0° C. After the addition, the reaction mixture was stirred at 0° C. for 2 hours under N₂ atmosphere. The reaction mixture was quenched by addition water (20 mL), 15% aqueous NaOH (20 mL) and water (60 mL) at 0° C., then filtered. The filtrate was concentrated and the residue (6.00 g) was dissolved in DCM (180 mL), then MnO₂ (46.9 g, 540 mmol) was added and the mixture was stirred at 25° C. for 12 hours. The reaction mixture was filtered and the filtrate was concentrated. The residue was purified by flash silica gel column (PE/EtOAc=1/1) to afford 3-methyl-1H-pyrrole-2-carbaldehyde (1.89 g, yield: 32%) as a brown solid. ¹H NMR (400 MHz, DMSO-d₆) δ 2.25 (3H, s), 6.04 (1H, d, J=2.0 Hz), 7.03 (1H, d, J=2.0 Hz), 9.50 (1H, s).

### Step 2. Synthesis of 4-bromo-3-methyl-1H-pyrrole-2-carbaldehyde

To a solution of 3-methyl-1H-pyrrole-2-carbaldehyde (1.85 g, 17.0 mmol) in THF (20 mL) was stirred at 0° C. for 0.5 hour and then NBS (2.72 g, 15.3 mmol) was added. The mixture was stirred at 0° C. for another 0.5 hour. The reaction mixture was concentrated and the residue was diluted with saturated aqueous NaHCO₃ (50 mL), then extracted with EtOAc (50 mL×4). The combined organic layer was dried over anhydrous Na₂SO₄, filtered and concentrated. The residue was purified by flash silica gel column (PE/EtOAc=3/1) to afford 4-bromo-3-methyl-1H-pyrrole-2-carbaldehyde as a yellow solid (2.34 g, yield: 74%). ¹H NMR (400 MHz, DMSO-d₆) δ 2.24 (3H, s), 7.31 (1H, d, J=4.0 Hz), 9.59 (1H, s), 12.22 (1H, brs,).

### Step 3. Synthesis of ethyl 6-bromo-5-methylpyrrolo[1,2-c]pyrimidine-3-carboxylate

To a solution of 4-bromo-3-methyl-1H-pyrrole-2-carbaldehyde (600 mg, 2.11 mmol) and ethyl 2-isocyanoacetate (238 mg, 2.11 mmol) in DCM (6 mL) was added DBU (321 mg, 2.11 mmol). The mixture was stirred at 60° C. for 6 hours. The reaction mixture was quenched with 10% aqueous HOAc (10 mL) and extracted with DCM (15 mL×3). The combined organic layer was washed with water (10 mL×2), dried over anhydrous Na₂SO₄, filtered and concentrated. The residue was purified by flash silica gel column (PE/EtOAc=3/1) to afford ethyl 6-bromo-5-methylpyrrolo[1,2-c]pyrimidine-3-carboxylate (350 mg, yield: 49%) as a yellow solid. ¹H NMR (400 MHz, DMSO-d₆) δ 1.33 (3H, t, J=7.2 Hz,), 2.25 (3H, s), 4.31 (2H, q, J=7.2 Hz), 8.05 (1H, s), 8.19 (1H, s), 9.02 (1H, s).

### Step 4. Synthesis of 6-bromo-5-methylpyrrolo[1,2-c]pyrimidine-3-carboxylic Acid

To a solution 6-bromo-5-methylpyrrolo[1,2-c]pyrimidine-3-carboxylate (1.90 g, 6.71 mmol) in THF (15 mL) and H₂O (5 ml) was added LiOH·H₂O (964 mg, 23.0 mmol). The mixture was stirred at 25° C. for 4 hours. The mixture was acidified with 1N aqueous HCl to pH=5, then filtered and dried to give 6-bromo-5-methylpyrrolo[1,2-c]pyrimidine-3-carboxylic acid (1.50 g, yield: 87%) as a white solid. ¹H NMR (400 MHz, DMSO-d₆) δ 2.23 (3H, s), 7.85-7.95 (2H, m), 8.98 (1H, s).

### Step 5. Synthesis of tert-butyl (6-bromo-5-methylpyrrolo[1,2-c]pyrimidin-3-yl)carbamate

A mixture of 6-bromo-5-methylpyrrolo[1,2-c]pyrimidine-3-carboxylic acid (100 mg, 0.390 mmol), DPPA (286 mg, 1.04 mmol) and Et₃N (119 mg, 1.18 mmol) in THF (4 mL) was stirred at 25° C. for 0.5 hour, then Boc₂O (342 mg, 1.57 mmol) and t-BuOH (12 mL) were added. The mixture was stirred at 90° C. for 16 hours under N₂ atmosphere to give a black solution. The reaction mixture was concentrated and the residue was diluted with water (15 mL) and extracted with EtOAc (15 mL×3). The combined organic layer was washed with brine (10 mL×2), dried over anhydrous Na₂SO₄, filtered and concentrated. The residue was purified by flash silica gel column (PE/EtOAc=5/1) afford tert-butyl (6-bromo-5-methylpyrrolo[1,2-c]pyrimidin-3-yl)carbamate (54 mg, yield: 42%) as light yellow solid. ¹H NMR (400 MHz, DMSO-d₆) δ 1.48 (9H, s), 2.13 (3H, s), 7.50-7.52 (1H, m), 7.66 (1H, s), 8.84 (1H, d, J=1.6 Hz), 9.54 (1H, brs).

### Step 6. Synthesis of tert-butyl (5-methyl-6-(pyridin-4-yl)pyrrolo[1,2-c]pyrimidin-3-yl)carbamate

A mixture of tert-butyl (6-bromo-5-methylpyrrolo[1,2-c]pyrimidin-3-yl)carbamate (300 mg, 0.911 mmol), 4-pyridylboronic acid (170 mg, 1.38 mmol), XPhos-Pd-G₃ (78 mg, 0.092 mmol), K₂CO₃ (254 mg, 1.84 mmol) in dioxane (12 mL) and H₂O (1.5 mL) was degassed and purged with N₂ for 3 times, and then the mixture was stirred at 100° C. for 16 hours under N₂ atmosphere. The reaction mixture was quenched with water (20 mL), then extracted with EtOAc (20 mL×3). The combined organic layer was dried over anhydrous Na₂SO₄, filtered and concentrated. The residue was purified by flash silica gel column (PE/EtOAc=1/2) to afford tert-butyl (5-methyl-6-(pyridin-4-yl)pyrrolo[1,2-c]pyrimidin-3-yl)carbamate (230 mg, yield: 77%) as a yellow solid. ¹H NMR (400 MHz, DMSO-d₆) δ 1.49 (9H, s), 2.33 (3H, s), 7.54-7.57 (2H, m), 7.58 (1H, d, J=3.2 Hz), 7.85 (1H, s), 8.62 (2H, d, J=6.0 Hz), 8.94 (1H, s), 9.53 (1H, brs).

### Step 7. Synthesis of 5-methyl-6-(pyridin-4-yl)pyrrolo[1,2-c]pyrimidin-3-amine

To a solution of tert-butyl (5-methyl-6-(pyridin-4-yl)pyrrolo[1,2-c]pyrimidin-3-yl)carbamate (230 mg, 0.710 mmol) in DCM (6 mL) was added TFA (3 mL). The mixture was stirred at 25° C. for 2 hours. The reaction mixture was concentrated and the residue was diluted with water (15 mL), then basified with saturated aqueous NaHCO₃ to pH=8 and extracted with DCM/MeOH (50 mL×2, 10/1). The combined organic layer was dried over anhydrous Na₂SO₄, filtered and concentrated to give 5-methyl-6-(pyridin-4-yl)pyrrolo[1,2-c]pyrimidin-3-amine (90 mg, yield: 57%) as a yellow solid. ¹H NMR (400 MHz, DMSO-d₆) δ 2.23 (3H, s), 5.43 (2H, brs), 6.06 (1H, s), 7.51-7.55 (2H, m), 7.63 (1H, s), 8.56-8.60 (2H, m), 8.76-8.79 (1H, m).

### Intermediate 24

### tert-butyl 3-((6-bromoisoquinolin-4-yl)oxy)pyrrolidine-1-carboxylate

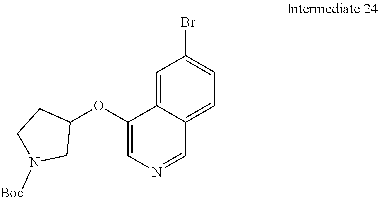 [SMILES: CC(C)(C)OC(=O)N1CCC(Oc2cncc3ccc(Br)cc23)C1]

To a solution of 6-bromoisoquinolin-4-ol (310 mg, 1.38 mmol), tert-butyl 3-hydroxypyrrolidine-1-carboxylate (389 mg, 2.08 mmol) and tributylphosphane (560 mg, 2.77 mmol) in toluene (8 mL) was added TMAD (476 mg, 2.77 mmol) in toluene (6 mL) under N₂ atmosphere, the mixture was stirred at 110° C. for 20 hours under N₂ atmosphere. The reaction mixture was turned into yellow solution. The reaction mixture was concentrated and the residue was purified by flash silica gel chromatography (ISCO®; 12 g SepaFlash® Silica Flash Column, Eluent of ˜40% Ethyl acetate/Petroleum ether gradient @35 mL/min) to give tert-butyl 3-((6-bromoisoquinolin-4-yl)oxy)pyrrolidine-1-carboxylate (530 mg, yield: 96%) as yellow gum.

### Intermediate 25

### 3-(2-chloropyridin-4-yl)-1-(tetrahydro-2H-pyran-4-yl)-1H-pyrrolo[2,3-c]pyridine

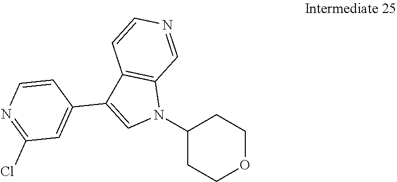 [SMILES: Clc1cc(-c2cn(C3CCOCC3)c3cnccc23)ccn1]

### Step 1. Synthesis of 3-bromo-1-(tetrahydro-2H-pyran-4-yl)-1H-pyrrolo[2,3-c]pyridine

A mixture of 3-bromo-1H-pyrrolo[2,3-c]pyridine (1.00 g, 5.08 mmol), tetrahydropyran-4-ol (1.56 g, 15.2 mmol), DIAD (2.05 g, 10.2 mmol) and PPh₃ (2.66 g, 10.2 mmol) in THF (80 mL) was degassed and purged with N₂ for 3 times, then the mixture was stirred at 60° C. for 16 hours under N₂ atmosphere. The reaction mixture was concentrated and the residue was diluted with H₂O (20 mL), then extracted with DCM (40 mL×3). The combined organic layer was dried over anhydrous Na₂SO₄, filtered and concentrated. The residue was purified by flash silica gel column (MeOH/DCM=1/10) to afford 3-bromo-1-(tetrahydro-2H-pyran-4-yl)-1H-pyrrolo[2,3-c]pyridine (534 mg, yield: 37%) as a light yellow solid. ¹H NMR (400 MHz, DMSO-d₆) δ 1.90-2.10 (4H, m), 3.52-3.62 (3H, m), 3.97-4.04 (2H, m), 7.41 (1H, d, J=5.2 Hz), 8.06 (1H, s), 8.24 (1H, d, J=5.2 Hz), 9.07 (1H, s).

### Step 2. Synthesis of 3-(2-chloropyridin-4-yl)-1-(tetrahydro-2H-pyran-4-yl)-1H-pyrrolo[2,3-c]pyridine

A mixture 3-bromo-1-(tetrahydro-2H-pyran-4-yl)-1H-pyrrolo[2,3-c]pyridine (530 mg, 1.89 mmol), (2-chloro-4-pyridyl)boronic acid (356 mg, 2.26 mmol), Pd(dppf)Cl₂ (138 mg, 0.189 mmol) and Na₂CO₃ (599 mg, 5.66 mmol) in dioxane (15 mL) and H₂O (2 mL) was degassed and purged with N₂ for 3 times, then the mixture was stirred at 90° C. for 16 hours under N₂ atmosphere. The reaction mixture was concentrated and the residue was diluted with H₂O (30 mL) and extracted with DCM (20 mL×3). The combined organic layer was dried over anhydrous Na₂SO₄, filtered and concentrated. The residue was purified by flash silica gel column (MeOH/DCM=1/20) to afford 3-(2-chloropyridin-4-yl)-1-(tetrahydro-2H-pyran-4-yl)-1H-pyrrolo[2,3-c]pyridine (410 mg, yield: 49%) as a red gum.

### Intermediate 26b and 26c

### 6-bromo-1-(difluoromethyl)-1H-benzo[d]imidazole and 5-bromo-1-(difluoromethyl)-1H-benzo[d]imidazole

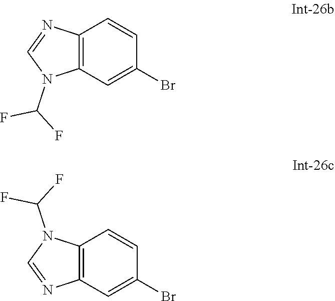

A solution of 6-bromo-1H-benzo[d]imidazole (2.00 g, 10.2 mmol) and KF (1.18 g, 20.3 mmol) in CH₃CN (60 mL) was degassed and purged with N₂ for 3 times, then diethyl (bromodifluoromethyl)phosphonate (2.71 g, 10.2 mmol) was added to the reaction mixture and the reaction mixture was stirred at 20° C. for 16 hours under N₂ atmosphere. The reaction mixture was filtered and the filtrate was concentrated. The residue was purified by flash silica gel chromatography (ISCO®; 12 g SepaFlash® Silica Flash Column, Eluent of 1% MeOH/DCM gradient @38 mL/min) to give a mixture of compound Int-26b and Int-26c (2.10 g, yield: 42%, purity: 50%) as a yellow solid.

### Intermediate 27

### tert-butyl 4-((4-bromopyridin-2-yl)carbamoyl)piperidine-1-carboxylate

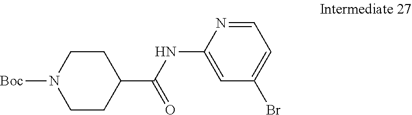 [SMILES: CC(C)(C)OC(=O)N1CCC(C(=O)Nc2cc(Br)ccn2)CC1]

To a solution of 1-(tert-butoxycarbonyl)piperidine-4-carboxylic acid (5.00 g, 21.8 mmol) and 4-bromopyridin-2-amine (4.15 g, 24.0 mmol) in pyridine (40 mL) was added Et₃N (6.62 g, 65.4 mmol) and T₃P (27.8 g, 43.6 mmol, 50% purity in EtOAc) at 25° C. The mixture was stirred at 25° C. for 3 hours. The reaction mixture was quenched with H₂O (50 mL) and extracted with EtOAc (50 mL×3). The combined organic layer was washed with brine (50 mL), dried over anhydrous Na₂SO₄, filtered and concentrated. The residue was purified by silica gel column (PE/EtOAc=3/1 to 1/1) to give tert-butyl 4-((4-bromopyridin-2-yl)carbamoyl)piperidine-1-carboxylate (7.00 g, yield: 84%) as a yellow solid. ¹H NMR (400 MHz, CDCl₃) δ 1.47 (9H, s), 1.63-1.80 (2H, m), 1.82-1.95 (2H, m), 2.35-2.45 (1H, m), 2.75-2.85 (2H, m), 4.10-4.20 (2H, m), 7.21 (1H, dd, J=5.2, 1.6 Hz), 8.07 (1H, d, J=5.2 Hz), 8.19 (1H, s), 8.49 (1H, d, J=1.6 Hz).

### Intermediate 28

### 3-bromo-2-methyl-2H-pyrazolo[3,4-c]pyridine

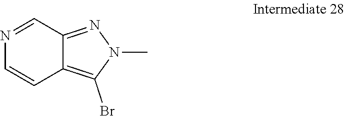

### Step 1: Synthesis of 2-methyl-2H-pyrazolo[3,4-c]pyridine

To a solution of 2H-pyrazolo[3,4-c]pyridine (1.50 g, 12.6 mmol) in anhydrous DMF (30 mL) was added NaH (1.02 g, 25.5 mmol, 60% dispersion in mineral oil) in portions at 0° C. and the mixture was stirred at 0° C. for 30 minutes. MeI (1.79 g, 12.6 mmol) was added to the reaction mixture and the mixture was stirred at 0° C. for another 1 hour. The reaction mixture was quenched with water (30 mL) at 0° C. and extracted with EtOAc (20 mL×3). The combined organic layer was washed with brine (50 mL), dried over anhydrous Na₂SO₄, filtered and concentrated. The residue was purified by flash silica gel chromatography (ISCO®; 12 g SepaFlash® Silica Flash Column, Eluent of 0˜10% MeOH/DCM gradient @30 mL/min) to give 2-methyl-2H-pyrazolo[3,4-c]pyridine (300 mg, yield: 18%) as a yellow solid. ¹H NMR (400 MHz, DMSO-d₆) δ 4.26 (3H, s), 7.65 (1H, dd, J=4.4, 1.6 Hz), 8.06 (1H, d, J=5.6 Hz), 8.48 (1H, s), 9.13 (1H, s).

### Step 2. Synthesis of 3-bromo-2-methyl-2H-pyrazolo[3,4-c]pyridine

To a solution of 2-methyl-2H-pyrazolo[3,4-c]pyridine (240 mg, 1.80 mmol) in DMF (5 mL) was added NBS (321 mg, 1.80 mmol) and the mixture was stirred at 80° C. for 2 hours. The reaction mixture was concentrated and the residue was dissolved in EtOAc (20 mL) and washed with 4% aqueous LiCl (10 mL×3), brine (10 mL), dried over anhydrous Na₂SO₄, filtered and concentrated. The residue was purified by flash silica gel chromatography (ISCO®; 12 g SepaFlash® Silica Flash Column, Eluent of 0˜10% MeOH/DCM gradient @30 mL/min) to give 3-bromo-2-methyl-2H-pyrazolo[3,4-c]pyridine (300 mg, yield: 60%) as a red solid.

¹H NMR (400 MHz, DMSO-d₆) δ 4.24 (3H, s), 7.52 (1H, dd, J=6.0, 1.2 Hz), 8.16 (1H, d, J=6.0 Hz), 9.20 (1H, s).

### Intermediate 29

### tert-butyl 7-bromo-3,4-dihydrobenzo[4,5]imidazo[1,2-a]pyrazine-2(1H)-carboxylate

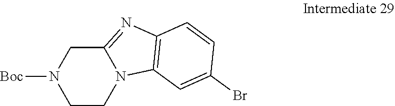 [SMILES: CC(C)(C)OC(=O)N1CCn2c(nc3ccc(Br)cc32)C1]

### Step 1. Synthesis of 2-((5-bromo-2-nitrophenyl)amino)ethan-1-ol

A solution of 4-bromo-2-fluoro-1-nitrobenzene (11.0 g, 50.0 mmol), 2-aminoethan-1-ol (3.36 g, 55.0 mmol) and K₂CO₃ (20.7 g, 150 mmol) in DMSO (100 mL) was stirred at 100° C. for 2 hours. The reaction mixture was diluted with EtOAc (500 mL), washed with H₂O (500 mL×2), brine (500 mL), dried over anhydrous Na₂SO₄, filtered and concentrated to give 2-((5-bromo-2-nitrophenyl)amino)ethan-1-ol (12.5 g, crude) as a yellow solid, which was used into the next step without further purification.

### Step 2. Synthesis of 2-((2-amino-5-bromophenyl)amino)ethan-1-ol

To a solution of 2-((5-bromo-2-nitrophenyl)amino)ethan-1-ol (12.5 g, crude) and NH₄Cl (15.4 g, 287 mmol) in EtOH (250 mL) and H₂O (60 mL) was added Fe powder (10.7 g, 192 mmol) at 20° C., the mixture was stirred at 80° C. for 2 hours. The reaction mixture was filtered through a pad of celite and the solid was washed with EtOH (100 mL×2). The filtrate was concentrated and the residue was purified by flash silica gel chromatography (ISCO®; 40 g SepaFlash® Silica Flash Column, Eluent of 50˜100% Ethyl acetate/Petroleum ether gradient @60 mL/min) to give 2-((2-amino-5-bromophenyl)amino)ethan-1-ol (7.30 g, yield: 63% for two steps) as a gray solid. ¹H NMR (400 MHz, DMSO-d₆) δ 3.05-3.08 (2H, m), 3.55-3.61 (2H, m), 4.61-4.69 (3H, m), 4.71 (1H, t, J=5.6 Hz), 6.44-6.49 (2H, m), 6.50-6.54 (1H, m).

### Step 3. Synthesis of tert-butyl (2-((4-bromo-2-((2-hydroxyethyl)amino)phenyl)amino)-2-oxoethyl)carbamate

To a solution of 2-((2-amino-5-bromophenyl)amino)ethan-1-ol (9.00 g, 39.0 mmol) and (tert-butoxycarbonyl)glycine (6.82 g, 39.0 mmol) in pyridine (250 mL) was added EDCI (9.71 g, 50.6 mmol) at 0° C., the mixture was stirred at 0° C. for 2 hours and stirred at 20° C. for 10 hours. The reaction mixture was concentrated and the residue was purified by flash silica gel chromatography (ISCO®; 40 g SepaFlash® Silica Flash Column, Eluent of 70˜100% Ethyl acetate/Petroleum ether gradient @70 mL/min) to give tert-butyl (2-((4-bromo-2-((2-hydroxyethyl)amino)phenyl)amino)-2-oxoethyl)carbamate (14.4 g, yield: 95%) as a yellow solid.

### Step 4. Synthesis of 2-(2-((((λ¹-methyl)(λ¹-oxidaneyl)boraneyl)amino)methyl)-6-bromo-1H-benzo[d]imidazol-1-yl)ethan-1-ol

A solution of tert-butyl (2-((4-bromo-2-((2-hydroxyethyl)amino)phenyl)amino)-2-oxoethyl)carbamate (14.4 g, 37.1 mmol) in HOAc (300 mL) was stirred at 100° C. for 1 hour. The reaction mixture was concentrated and the residue was basified with saturated aqueous NaHCO₃ to pH=8 and extracted with EtOAc (400 mL×3). The combined organic layer was concentrated and the residue was purified by flash silica gel chromatography (ISCO®; 40 g SepaFlash® Silica Flash Column, Eluent of 4˜10% MeOH/DCM gradient @60 mL/min) to give 2-(2-((((λ¹-methyl)(λ¹-oxidaneyl)boraneyl)amino)methyl)-6-bromo-1H-benzo[d]imidazol-1-yl)ethan-1-ol (11.7 g, yield: 82%) as a yellow solid.

### Step 5. Synthesis of 2-(2-((((λ¹-methyl)(λ¹-oxidaneyl)boraneyl)amino)methyl)-6-bromo-1H-benzo[d]imidazol-1-yl)ethyl Methanesulfonate

To a solution of compound 7 (6.70 g, 18.1 mmol) and TEA (9.16 g, 90.5 mmol) in DCM (300 mL) was added MsCl (6.83 g, 59.6 mmol) at 0° C., the mixture was stirred at 0° C. for 2 hours. The reaction mixture was quenched with saturated aqueous NaHCO₃ (150 mL) and separated. The aqueous phase was extracted with DCM (200 mL×3), the combined organic layer was dried over anhydrous Na₂SO₄, filtered and concentrated to give 2-(2-((((λ¹-methyl)(λ¹-oxidaneyl)boraneyl)amino)methyl)-6-bromo-1H-benzo[d]imidazol-1-yl)ethyl methanesulfonate (8.90 g, crude) as a yellow solid, which was used into the next step without further purification.

### Step 6. Synthesis of 2-(2-(aminomethyl)-6-bromo-1H-benzo[d]imidazol-1-yl)ethyl Methanesulfonate

A solution of 2-(2-((((λ¹-methyl)(λ¹-oxidaneyl)boraneyl)amino)methyl)-6-bromo-1H-benzo[d]imidazol-1-yl)ethyl methanesulfonate (8.90 g, crude) in TFA (70 mL) was stirred at 0° C. for 0.5 hour and stirred at 20° C. for 2 hours. The reaction mixture was concentrated to give 2-(2-(aminomethyl)-6-bromo-1H-benzo[d]imidazol-1-yl)ethyl methanesulfonate (14.0 g, crude) as brown gum, which was used into the next step without further purification.

### Step 7. Synthesis of tert-butyl 7-bromo-3,4-dihydrobenzo[4,5]imidazo[1,2-a]pyrazine-2(1H)-carboxylate

To a solution of 2-(2-(aminomethyl)-6-bromo-1H-benzo[d]imidazol-1-yl)ethyl methanesulfonate (14.0 g, crude) in i-PrOH (80 mL) and H₂O (320 mL) was added K₂CO₃ (28.5 g, 206 mmol), the mixture was stirred at 105° C. for 1 hour. Then Boc₂O (10.0 g, 45.8 mmol) was added to the mixture at 0° C. and the mixture was stirred at 0° C. for another 0.5 hour. The reaction mixture was extracted with EtOAc (300 mL×2), the combined organic layer was washed with brine (400 mL), dried over anhydrous Na₂SO₄, filtered and concentrated. The residue was purified by flash silica gel chromatography (ISCO®; 40 g SepaFlash® Silica Flash Column, Eluent of 30˜40% Ethyl acetate/Petroleum ether gradient @60 mL/min) to give tert-butyl 7-bromo-3,4-dihydrobenzo[4,5]imidazo[1,2-a]pyrazine-2(1H)-carboxylate (3.70 g, yield: 57% for three steps) as a light yellow solid.

¹H NMR (400 MHz, CD₃OD) δ 1.51 (9H, s), 4.00 (2H, t, J=5.2 Hz), 4.18 (2H, t, J=5.6 Hz), 4.86 (2H, s), 7.39 (1H, dd, J=8.4, 1.6 Hz), 7.51 (1H, d, J=8.8 Hz), 7.72 (1H, d, J=1.6 Hz).

### Intermediate 30

### tert-butyl (7-bromopyrrolo[1,2-a]pyrazin-3-yl)carbamate

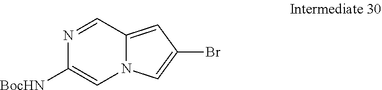 [SMILES: CC(C)(C)OC(=O)Nc1cn2cc(Br)cc2cn1]

### Step 1. Synthesis of methyl 2-(bis(tert-butoxycarbonyl)amino)acrylate Int-20

To a solution of methyl (tert-butoxycarbonyl)serinate (3.00 g, 13.7 mmol) in CH₃CN (30 mL) was added DMAP (334 mg, 2.74 mmol) and Boc₂O (5.97 g, 27.4 mmol) at 0° C. The mixture was stirred at 60° C. for 12 hours. The reaction mixture was concentrated and the residue was diluted with H₂O (50 mL) and extracted with EtOAc (80 mL×3). The combined organic layers were washed with saturated aqueous NaHCO₃ (80 mL), brine (100 mL), dried over anhydrous Na₂SO₄, filtered and concentrated to give methyl 2-(bis(tert-butoxycarbonyl)amino)acrylate (4.00 g, yield: 97%) as yellow oil.

¹H NMR (400 MHz, CDCl₃) δ 1.48 (18H, s), 3.81 (3H, s), 5.66 (1H, s), 6.36 (1H, s).

### Step 2. Synthesis of methyl 2-(bis(tert-butoxycarbonyl)amino)-3-(4-bromo-2-formyl-1H-pyrrol-1-yl)propanoate

To a solution of methyl 2-(bis(tert-butoxycarbonyl)amino)acrylate (4.00 g, 13.3 mmol), 4-bromo-1H-pyrrole-2-carbaldehyde (2.31 g, 13.3 mmol) in CH₃CN (50 mL) was added K₂CO₃ (5.50 g, 39.8 mmol), then the mixture was stirred at 25° C. for 12 hours. The reaction mixture was filtered and the filtrate was concentrated. The residue was diluted with H₂O (50 mL) and extracted with EtOAc (80 mL×3). The combined organic layers were washed with brine (80 mL), dried over anhydrous Na₂SO₄, filtered and concentrated to give methyl 2-(bis(tert-butoxycarbonyl)amino)-3-(4-bromo-2-formyl-1H-pyrrol-1-yl)propanoate (5.00 g, yield: 79%) as a yellow solid.

### Step 3. Synthesis of methyl 7-bromo-3,4-dihydropyrrolo[1,2-a]pyrazine-3-carboxylate

To a solution of methyl 2-(bis(tert-butoxycarbonyl)amino)-3-(4-bromo-2-formyl-1H-pyrrol-1-yl)propanoate (5.00 g, 10.5 mmol) in DCM (25 mL) was added TFA (25 mL) at 0° C. The mixture was stirred at 20° C. for 12 hours. The reaction mixture was concentrated and the residue was basified with saturated aqueous NaHCO₃ to pH=8 and extracted with DCM (100 mL×2). The combined organic layers were washed with brine (100 mL), dried over anhydrous Na₂SO₄, filtered and concentrated to give methyl 7-bromo-3,4-dihydropyrrolo[1,2-a]pyrazine-3-carboxylate (2.50 g, yield: 92%) as a yellow solid.

¹H NMR (400 MHz, CDCl₃) δ 3.86 (3H, s), 4.52 (1H, dd, J=13.6, 5.6 Hz), 4.83 (1H, dd, J=13.6, 3.2 Hz), 5.15-5.26 (1H, m), 7.32-7.38 (2H, m), 8.88 (1H, s).

### Step 4. Synthesis of methyl 7-bromopyrrolo[1,2-a]pyrazine-3-carboxylate

To a solution of methyl 7-bromo-3,4-dihydropyrrolo[1,2-a]pyrazine-3-carboxylate (2.50 g, 9.72 mmol) in DCM (30 mL) was added MnO₂ (4.23 g, 48.6 mmol). The mixture was stirred at 40° C. for 1 hour. The reaction mixture was suspended in CH₃₀H (50 mL) and filtered. The filtrate was concentrated and the residue was purified by silica gel column (0% to 75% EtOAc in PE) to give methyl 7-bromopyrrolo[1,2-a]pyrazine-3-carboxylate (2.00 g, yield: 80%) as a brown solid. ¹H NMR (400 MHz, DMSO-d₆) δ 3.87 (3H, s), 7.15 (1H, s), 8.13 (1H, s), 8.87 (1H, s), 9.12 (1H, s).

### Step 5. Synthesis of 7-bromopyrrolo[1,2-a]pyrazine-3-carboxylic Acid

To a solution of methyl 7-bromopyrrolo[1,2-a]pyrazine-3-carboxylate (3.20 g, 12.5 mmol) in THF (20 mL) and H₂O (5 mL) was added LiOH·H₂O (2.11 g, 50.2 mmol). The mixture was stirred at 25° C. for 12 hours. The reaction mixture was concentrated and the residue was acidified with 1N aqueous HCl to pH=5 and filtered. The solid was washed with water (10 mL×2) and dried to give 7-bromopyrrolo[1,2-a]pyrazine-3-carboxylic acid (2.20 g, yield: 72%) as a brown solid.

### Step 6. Synthesis of 7-bromopyrrolo[1,2-a]pyrazine-3-carbonyl Azide

To a mixture of 7-bromopyrrolo[1,2-a]pyrazine-3-carboxylic acid (850 mg, 3.53 mmol) and TEA (714 mg, 7.05 mmol) in acetone (20 mL) was added isobutyl carbonochloridate (771 mg, 5.64 mmol) at 0° C. The mixture was stirred at 0° C. for 1 hour, then a solution of NaN₃ (573 mg, 8.82 mmol) in H₂O (5 mL) was added to the mixture and the reaction was stirred at 0° C. for another 1 hour. The reaction mixture was poured into ice-water (30 mL) and filtered. The solid was washed with water (10 mL×2) and dried to give 7-bromopyrrolo[1,2-a]pyrazine-3-carbonyl azide (800 mg, yield: 85%) as a black solid.

### Step 7. Synthesis of tert-butyl (7-bromopyrrolo[1,2-a]pyrazin-3-yl)carbamate

A mixture of 7-bromopyrrolo[1,2-a]pyrazine-3-carbonyl azide (800 mg, 3.01 mmol) in toluene (10 mL) and t-BuOH (50 mL) was stirred at 85° C. for 12 hours. The reaction mixture was concentrated and the residue was purified by silica gel column (0% to 15% EtOAc in PE) to give tert-butyl (7-bromopyrrolo[1,2-a]pyrazin-3-yl)carbamate (150 mg, yield: 16%) as a yellow solid.

¹H NMR (400 MHz, DMSO-d₆) δ 1.48 (9H, s), 6.90 (1H, s), 7.96 (1H, s), 8.58-8.66 (2H, m), 9.53 (1H, brs).

### Intermediate 31

### (1R,5S)-3-methyl-3-azabicyclo[3.1.0]hexane-6-carboxylic Acid

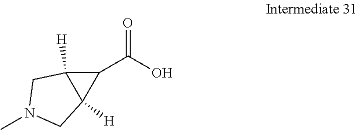

### Step 1. Synthesis of 3-(tert-butyl) 6-methyl (1R,5S)-3-azabicyclo[3.1.0]hexane-3,6-dicarboxylate

To a solution of (1R,5S)-3-(tert-butoxycarbonyl)-3-azabicyclo[3.1.0]hexane-6-carboxylic acid (1.00 g, 4.40 mmol) in MeOH (2 mL) and DCM (8 mL) was added TMSCHN₂ (4.4 mL, 8.80 mmol, 2M in hexane). The mixture was stirred at 25° C. for 4 hours and then concentrated. The residue was purified by Combi Flash (0% to 30% EtOAc in PE) to give 3-(tert-butyl) 6-methyl (1R,5S)-3-azabicyclo[3.1.0]hexane-3,6-dicarboxylate (1.00 g, yield: 94%) as colorless oil.

### Step 2. Synthesis of methyl (1R,5S)-3-azabicyclo[3.1.0]hexane-6-carboxylate

A mixture of 3-(tert-butyl) 6-methyl (1R,5S)-3-azabicyclo[3.1.0]hexane-3,6-dicarboxylate (500 mg, 2.07 mmol) in TFA (2 mL) and DCM (2 mL) was stirred at 25° C. for 3 hours. The reaction mixture was concentrated to give methyl (1R,5S)-3-

### Step 3. Synthesis of methyl (1R,5S)-3-methyl-3-azabicyclo[3.1.0]hexane-6-carboxylate

To a solution of methyl (1R,5S)-3-azabicyclo[3.1.0]hexane-6-carboxylate (500 mg, 3.54 mmol) in MeOH (10 mL) was added HOAc (213 mg, 3.54 mmol) until the pH=5 at 25° C. After the addition, 37% aqueous formaldehyde (1.44 g, 17.7 mmol) was added and the reaction was stirred at 25° C. for 0.5 hour. NaBH₃CN (668 mg, 10.6 mmol) was added and the resulting mixture was stirred at 25° C. for 1 hour. The mixture was concentrated and the residue was basified with saturated aqueous NaHCO₃ to pH=8 and extracted with DCM/MeOH (25 mL×2, 10/1). The combined organic layer was concentrated and the residue was acidified with 1N aqueous HCl to pH=2 and washed with EtOAc (25 mL). The aqueous layer was basified with 1N aqueous NaOH to pH=10 and extracted with DCM/MeOH (25 mL×2, 10/1). The combined organic layer was dried over anhydrous Na₂SO₄, filtered and concentrated. The residue was purified by Combi Flash (0% to 6% MeOH in DCM) to give methyl (1R,5S)-3-methyl-3-azabicyclo[3.1.0]hexane-6-carboxylate (360 mg, yield: 33%) as colorless oil.

### Step 4. Synthesis of (1R,5S)-3-methyl-3-azabicyclo[3.1.0]hexane-6-carboxylic Acid

A solution of methyl (1R,5S)-3-methyl-3-azabicyclo[3.1.0]hexane-6-carboxylate (250 mg, 1.61 mmol) in conc. HCl (1.3 mL) was stirred at 70° C. for 12 hours. The reaction mixture was concentrated to give (1R,5S)-3-methyl-3-azabicyclo[3.1.0]hexane-6-carboxylic acid (250 mg, yield: 87%, HCl salt) as a white solid.

¹H NMR (400 MHz, DMSO-d₆) δ1.98-2.07 (11, m), 2.15-2.19 (2H, m), 2.78 (3H, s), 3.30-3.35 (2H, m), 3.64-3.67 (2H, m).

### Intermediate 32

### 3-bromo-6-ethoxypyrazolo[1,5-a]pyridine

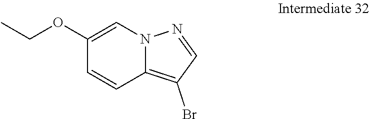

### Step 1. Synthesis of 6-(4,4,5,5-tetramethyl-1,3,2-dioxaborolan-2-yl)pyrazolo[1,5-a]pyridine

To a solution of 6-bromopyrazolo[1,5-a]pyridine (2.27 g, 11.5 mmol) and Bis-Pin (3.07 g, 12.1 mmol) in 1, 4-dioxane (40 mL) was added Pd(dppf)Cl₂ (422 mg, 0.576 mmol) and KOAc (3.39 g, 34.6 mmol) under N₂ atmosphere, the mixture was stirred at 100° C. for 16 hours under N₂ atmosphere to give black suspension. The reaction mixture was filtered through a pad of celite and the solid was washed with EtOAc (30 mL×3). The filtrate was concentrated and the residue was purified by flash silica gel chromatography (ISCO®; 12 g SepaFlash® Silica Flash Column, Eluent of 0˜8% Ethyl acetate/Petroleum ether gradient @40 mL/min) to give 6-(4,4,5,5-tetramethyl-1,3,2-dioxaborolan-2-yl)pyrazolo[1,5-a]pyridine (2.70 g, yield: 92%) as a green solid.

### Step 2. Synthesis of pyrazolo[1,5-a]pyridin-6-ol

To a solution of 6-(4,4,5,5-tetramethyl-1,3,2-dioxaborolan-2-yl)pyrazolo[1,5-a]pyridine (1.70 g, 6.96 mmol) in THF (40 mL) was added 2N aqueous NaOH (10.5 mL) dropwise at 0° C. and then 30% aqueous H₂O₂ (1.93 g, 17.0 mmol) was added dropwise at 0° C. The reaction mixture was stirred at 0° C. for 2.5 hours to give yellow solution. The reaction mixture was quenched with saturated aqueous Na₂SO₃ (20 mL) at 0° C. and stirred at 0° C. for 1 hour, then concentrated. The residue was acidified with 1N aqueous HCl to pH=5. The precipitate was filtered and dried then purified by flash silica gel chromatography (ISCO®; 4 g SepaFlash® Silica Flash Column, Eluent of ˜30% Ethyl acetate/Petroleum ether gradient @35 mL/min) to give pyrazolo[1,5-a]pyridin-6-ol (460 mg, yield: 49%) as a purple solid. ¹H NMR (400 MHz, DMSO-d₆) δ 6.42-6.51 (1H, m), 6.95 (1H, dd, J=9.2, 2.0 Hz), 7.55 (1H, d, J=9.6 Hz), 7.78 (1H, d, J=2.0 Hz), 8.04-8.13 (1H, m), 9.55 (1H, brs).

### Step 3. Synthesis of 6-ethoxypyrazolo[1,5-a]pyridine

To a solution of pyrazolo[1,5-a]pyridin-6-ol (140 mg, 1.04 mmol), K₂CO₃ (433 mg, 3.13 mmol) in DMAc (3 mL) was added EtI (244 mg, 1.57 mmol) and the reaction mixture was stirred at 60° C. for 2 hours to give gray suspension. The reaction mixture was diluted into H₂O (30 mL) and extracted with EtOAc (30 mL). The combined organic layer was washed with brine (30 mL), dried over anhydrous Na₂SO₄, filtered and concentrated. The residue was purified by flash silica gel chromatography (ISCO®; 4 g SepaFlash® Silica Flash Column, Eluent of ˜8% Ethyl acetate/Petroleum ether gradient @30 mL/min) to give 6-ethoxypyrazolo[1,5-a]pyridine (110 mg, yield: 64%) as a white solid.

### Step 4. Synthesis of 3-bromo-6-ethoxypyrazolo[1,5-a]pyridine

To a solution of 6-ethoxypyrazolo[1,5-a]pyridine (110 mg, 0.678 mmol) in DMF (2 mL) was added NBS (133 mg, 0.746 mmol) at 0° C. and the reaction mixture was stirred at 20° C. for 2 hours to give colorless solution. The reaction mixture was diluted with EtOAc (30 mL), washed with H₂O (20 mL), brine (20 mL) and dried over anhydrous Na₂SO₄, filtered and concentrated. The residue was purified by flash silica gel chromatography (ISCO®; 4 g SepaFlash® Silica Flash Column, Eluent of ˜6% Ethyl acetate/Petroleum ether gradient @35 mL/min) to give 3-bromo-6-ethoxypyrazolo[1,5-a]pyridine (150 mg, yield: 92%) as a white solid. ¹H NMR (400 MHz, CDCl₃) δ 1.47 (3H, t, J=6.8 Hz), 4.02 (2H, q, J=6.8 Hz), 7.01 (1H, dd, J=9.6, 1.6 Hz), 7.40 (1H, d, J=9.6 Hz), 7.82 (1H, s), 8.01 (1H, s).

### Intermediate 33

### 4-(4,4,5,5-tetramethyl-1,3,2-dioxaborolan-2-yl)pyridin-2-ol

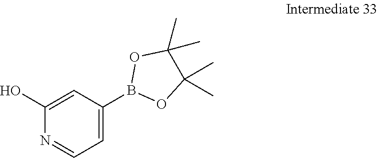 [SMILES: CC1(C)OB(c2ccnc(O)c2)OC1(C)C]

A mixture of 4-bromopyridin-2-ol (1.00 g, 5.75 mmol), Bis-Pin (1.61 g, 6.32 mmol), KOAc (846 mg, 8.62 mmol), PCy₃ (193 mg, 0.689 mmol) and Pd₂(dba)₃ (316 mg, 0.345 mmol) in 1,4-dioxane (20 mL) was degassed and purged with N₂ for 3 times, then the mixture was stirred at 85° C. for 3 hours under N₂ atmosphere. The reaction mixture was concentrated and the residue was diluted with water (100 mL), then extracted with EtOAc (100 mL×3). The combined organic layers were dried over anhydrous Na₂SO₄, filtered and concentrated. The residue was purified by Combi Flash (0% to 10% MeOH in DCM) to give 4-(4,4,5,5-tetramethyl-1,3,2-dioxaborolan-2-yl)pyridin-2-ol (1.20 g, yield: 94%) as a yellow solid.

### Intermediate 34

### 4-(azetidin-3-yl)-1-methylpiperidine

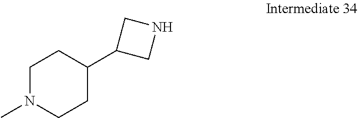

### Step 1. Synthesis of tert-butyl 3-(pyridin-4-yl)azetidine-1-carboxylate

To a solution of Zn powder (4.78 g, 73.2 mmol) in DMA (20 mL) was added 1, 2-dibromoethane (1.10 g, 5.85 mmol) and TMSCl (636 mg, 5.85 mmol) at 20° C. After stirring at 20° C. for 0.1 hour, a solution of tert-butyl 3-iodoazetidine-1-carboxylate (15.2 g, 53.7 mmol) in DMA (20 mL) was added to this solution at 20° C. and stirred at 20° C. for 0.5 hour, then the mixture was stirred at 50° C. for 2 hours. A solution of compound 4-iodopyridine (5.00 g, 24.4 mmol), Pd(dppf)Cl₂ (1.78 g, 2.44 mmol) and CuI (929 mg, 4.88 mmol) in DMA (60 mL) was added to the above solution under N₂ atmosphere. The mixture was stirred at 80° C. for 12 hours under N₂ atmosphere. The reaction mixture was filtered and the filtrate was concentrated. The residue was diluted with H₂O (100 mL) and extracted with EtOAc (100 mL×3). The combined organic layers were washed with H₂O (100 mL), brine (100 mL), dried over anhydrous Na₂SO₄, filtered and concentrated. The residue was purified by flash silica gel column (ISCO®; 40 g SepaFlash® Silica Flash Column, Eluent of 0˜80% EtOAc/PE gradient @50 mL/min) to give tert-butyl 3-(pyridin-4-yl)azetidine-1-carboxylate (5.10 g, yield: 84%) as a light brown solid.

### Step 2. Synthesis of 4-(1-(tert-butoxycarbonyl)azetidin-3-yl)-1-methylpyridin-1-ium

To a solution of tert-butyl 3-(pyridin-4-yl)azetidine-1-carboxylate (5.10 g, 21.8 mmol) in MeCN (60 mL) was added MeI (9.27 g, 65.3 mmol) and the reaction mixture was stirred at 80° C. for 5 hours. The reaction mixture was concentrated and the residue was purified by prep-HPLC (0.1% HCl as an additive), then lyophilized to give 4-(1-(tert-butoxycarbonyl)azetidin-3-yl)-1-methylpyridin-1-ium (5.10 g, yield: 62%) as a yellow solid.

### Step 3. Synthesis of tert-butyl 3-(1-methyl-1,2,3,6-tetrahydropyridin-4-yl)azetidine-1-carboxylate

To a solution of 4-(1-(tert-butoxycarbonyl)azetidin-3-yl)-1-methylpyridin-1-ium (3.00 g, 7.97 mmol) in EtOH (50 mL) was added NaBH₄ (1.59 g, 42.0 mmol) portion-wise at 0° C., the mixture was stirred at 20° C. for 3 hours. The reaction mixture was quenched with saturated aqueous NH₄Cl (100 mL) at 0° C., then extracted with EtOAc (100 mL×2). The combined organic layer was dried over anhydrous Na₂SO₄, filtered and concentrated. The residue was purified by flash silica gel column (ISCO®; 12 g SepaFlash® Silica Flash Column, Eluent of 4˜10% MeOH/DCM gradient @45 mL/min) to give tert-butyl 3-(1-methyl-1,2,3,6-tetrahydropyridin-4-yl)azetidine-1-carboxylate (900 mg, yield: 45%) as yellow oil. ¹H NMR (400 MHz, CDCl₃) δ 1.44 (9H, s), 2.20-2.26 (2H, m), 2.44 (3H, s), 2.66 (2H, t, J=5.6 Hz), 3.02-3.09 (2H, m), 3.12-3.22 (1H, m), 3.80-3.85 (2H, m), 3.98-4.05 (2H, m), 5.47-5.56 (1H, m).

### Step 4. Synthesis of tert-butyl 3-(1-methylpiperidin-4-yl)azetidine-1-carboxylate

A solution of tert-butyl 3-(1-methyl-1,2,3,6-tetrahydropyridin-4-yl)azetidine-1-carboxylate (900 mg, 3.57 mmol) and PtO₂ (500 mg, 2.20 mmol) in EtOH (20 mL) was degassed and purged with H₂ for three times, then the mixture was hydrogenated (15 psi) at 20° C. for 20 hours. The reaction mixture was filtered through a pad of celite and the solid was washed with EtOH (20 mL×3). The filtrate was concentrated to give tert-butyl 3-(1-methylpiperidin-4-yl)azetidine-1-carboxylate (900 mg, crude) as yellow gum, which was used for the next step without further purification.

### Step 5. Synthesis of 4-(azetidin-3-yl)-1-methylpiperidine

To a solution of tert-butyl 3-(1-methylpiperidin-4-yl)azetidine-1-carboxylate (400 mg, 1.57 mmol) in DCM (6 mL) was added TFA (3 mL) and the mixture was stirred at 20° C. for 3 hours. The reaction mixture was concentrated at −15° C. to give 4-(azetidin-3-yl)-1-methylpiperidine (1.00 g, crude, 2TFA salt) as yellow gum, which was used for the next step without further purification.

### Compounds of Formula (I)

### Example 1

### 3-(pyridin-4-yl)-1H-pyrrolo[2,3-c]pyridine

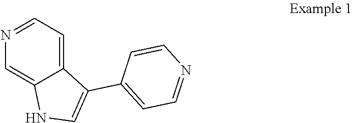 [SMILES: c1cc(-c2c[nH]c3cnccc23)ccn1]

### Step 1. Synthesis of 3-(pyridin-4-yl)-1-tosyl-1H-pyrrolo[2,3-c]pyridine

A mixture of Intermediate 1 (3.40 g, 8.54 mmol), 4-pyridylboronic acid (1.57 g, 12.8 mmol), K₂CO₃ (2.36 g, 17.1 mmol) and Pd(dppf)Cl₂ (625 mg, 0.854 mmol) in dioxane (40 mL) and H₂O (10 mL) was degassed and purged with N₂ for 3 times. Then the reaction mixture was stirred at 90° C. for 5 hours under N₂ atmosphere. The reaction mixture was diluted with water (20 mL) and extracted with EtOAc (25 mL×3). The combined organic layer was washed with brine (20 mL), dried over anhydrous Na₂SO₄, filtered and concentrated. The residue was purified by silica gel column (PE/EtOAc=1/1 to 0/1) to give 3-(pyridin-4-yl)-1-tosyl-1H-pyrrolo[2,3-c]pyridine (2.80 g, yield: 94%) as a brown solid. ¹H NMR (400 MHz, DMSO-d₆) δ 2.34 (3H, s), 7.44 (2H, d, J=8.4 Hz), 7.81-7.86 (2H, m), 8.00 (1H, d, J=5.6 Hz), 8.09 (2H, d, J=8.4 Hz), 8.50 (1H, d, J=5.2 Hz,), 8.64-8.68 (2H, m), 8.70 (1H, s), 9.31 (1H, s).

### Step 2. Synthesis of 3-(pyridin-4-yl)-1H-pyrrolo[2,3-c]pyridine

To a solution of 3-(pyridin-4-yl)-1-tosyl-1H-pyrrolo[2,3-c]pyridine (1.00 g, 2.86 mmol) in MeOH (4 mL) was added aqueous KOH (4.77 mL, 6 M) at 25° C. The mixture was stirred at 90° C. for 1 hour. The reaction mixture was diluted with water (20 mL) and extracted with EtOAc (25 mL×3). The combined organic layer was washed with brine (20 mL), dried over anhydrous Na₂SO₄, filtered and concentrated. The residue was purified by prep-HPLC (0.05% FA as an additive), then lyophilized to give the title compound (52.73 mg, yield: 9%) as a white solid. ¹H NMR (400 MHz, DMSO-d₆) δ 7.72-7.83 (2H, m). 1H), 7.97 (1H, dd, J=5.6, 0.8 Hz), 8.24 (1H, d, J=5.6 Hz), 8.32 (1H, s), 8.48-8.63 (2H, m), 8.85 (1H, s).

### Example 2

### 1-isopropyl-3-(pyridin-4-yl)-1H-pyrrolo[2,3-c]pyridine

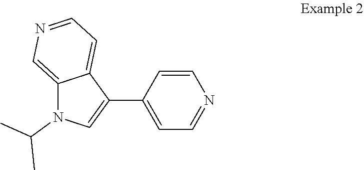 [SMILES: CC(C)n1cc(-c2ccncc2)c2ccncc21]

To a solution of Example 1 (200 mg, 1.02 mmol) and 2-propanol (92 mg, 1.54 mmol) in THF (3 mL) was added Ph₃P (537 mg, 2.05 mmol) and DEAD (357 mg, 2.05 mmol) at 0° C. The mixture was stirred at 25° C. for 12 hours. The mixture was concentrated and the residue was purified by prep-HPLC (0.05% FA as an additive) to give the title compound (59.60 mg, yield: 24%) as an off-white solid. ¹H NMR (400 MHz, CD₃OD) δ 1.66 (6H, d, J=6.8 Hz), 4.95-5.10 (1H, m), 7.76-7.88 (2H, m), 8.07 (1H, dd, J=5.6, 0.8 Hz), 8.27 (1H, d, J=5.6 Hz), 8.38 (1H, s), 8.49-8.58 (2H, m), 8.98 (1H, s).

### Example 3

### N-(4-(1H-pyrrolo[2,3-c]pyridin-3-yl)pyridin-2-yl)-1-isobutylpiperidine-4-carboxamide

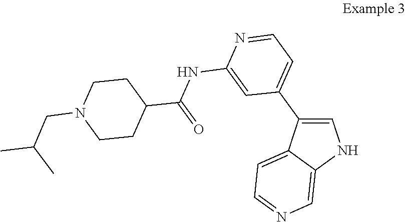 [SMILES: CC(C)CN1CCC(C(=O)Nc2cc(-c3c[nH]c4cnccc34)ccn2)CC1]

### Step 1. Synthesis of tert-butyl 4-((4-(1-tosyl-1H-pyrrolo[2,3-c]pyridin-3-yl)pyridin-2-yl)carbamoyl)piperidine-1-carboxylate

To a solution of Intermediate 2 (650 mg, 1.78 mmol) and 1-(tert-butoxycarbonyl)piperidine-4-carboxylic acid (491 mg, 2.14 mmol) in pyridine (10 mL) was added Et₃N (541 mg, 5.35 mmol), T₃P (2.27 g, 3.57 mmol, 50% purity in EtOAc) at 25° C. The mixture was stirred at 25° C. for 16 hours. The reaction mixture was quenched with H₂O (20 mL) at 25° C. and extracted with EtOAc (50 mL×3). The combined organic layer was washed with brine (20 mL), dried over anhydrous Na₂SO₄, filtered and concentrated. The crude product was triturated with EtOAc (10 mL) to give tert-butyl 4-((4-(1-tosyl-1H-pyrrolo[2,3-c]pyridin-3-yl)pyridin-2-yl)carbamoyl)piperidine-1-carboxylate (1.30 g, yield: 97%) as a white solid. ¹H NMR (400 MHz, DMSO-d₆) δ 1.41 (9H, s), 1.47-1.52 (2H, m), 1.75-1.85 (2H, m), 2.34 (3H, s), 2.65-2.85 (3H, m), 3.00-3.10 (2H, m), 7.44 (2H, d, J=8.0 Hz), 7.56 (1H, d, J=5.2 Hz), 7.91 (1H, d, J=4.8 Hz), 8.10 (2H, d, J=8.4 Hz), 8.40 (1H, d, J=5.2 Hz), 8.45-8.60 (2H, m), 8.64 (1H, s), 9.32 (1H, s), 10.66 (1H, brs).

### Step 2. Synthesis of N-(4-(1-tosyl-1H-pyrrolo[2,3-c]pyridin-3-yl)pyridin-2-yl)piperidine-4-carboxamide

To a solution of tert-butyl 4-((4-(1-tosyl-1H-pyrrolo[2,3-c]pyridin-3-yl)pyridin-2-yl)carbamoyl)piperidine-1-carboxylate (1.10 g, 1.91 mmol) in DCM (10 mL) was added TFA (10 mL). The mixture was stirred at 25° C. for 15 minutes. The reaction mixture was basified with saturated aqueous NaHCO₃ to pH=8 and extracted with EtOAc (50 mL×3). The combined organic layer was washed with brine (15 mL), dried over anhydrous Na₂SO₄, filtered and concentrated to give N-(4-(1-tosyl-1H-pyrrolo[2,3-c]pyridin-3-yl)pyridin-2-yl)piperidine-4-carboxamide (1.00 g, yield: 93%) as yellow oil.

### Step 3. Synthesis of 1-isobutyl-N-(4-(1-tosyl-1H-pyrrolo[2,3-c]pyridin-3-yl)pyridin-2-yl)piperidine-4-carboxamide

To a solution of N-(4-(1-tosyl-1H-pyrrolo[2,3-c]pyridin-3-yl)pyridin-2-yl)piperidine-4-carboxamide (800 mg, 1.68 mmol) in MeOH (10 mL) was added isobutyraldehyde (182 mg, 2.52 mmol) at 25° C. The mixture was stirred at 25° C. for 0.5 hour. Then NaBH₃CN (317 mg, 5.05 mmol) was added and the mixture was stirred at 25° C. for another 0.5 hour. The reaction mixture was quenched with H₂O (20 mL) and extracted with EtOAc (50 mL×3). The combined organic layer was washed with brine (20 mL), dried over anhydrous Na₂SO₄, filtered and concentrated. The residue was purified by silica gel column (DCM/MeOH=20/1 to 10/1) to give 1-isobutyl-N-(4-(1-tosyl-1H-pyrrolo[2,3-c]pyridin-3-yl)pyridin-2-yl)piperidine-4-carboxamide (800 mg, yield: 89%) as a yellow solid.

### Step 4. Synthesis of N-(4-(1H-pyrrolo[2,3-c]pyridin-3-yl)pyridin-2-yl)-1-isobutylpiperidine-4-carboxamide

A solution of 1-isobutyl-N-(4-(1-tosyl-1H-pyrrolo[2,3-c]pyridin-3-yl)pyridin-2-yl)piperidine-4-carboxamide (500 mg, 0.940 mmol) and Cs₂CO₃ (919 mg, 2.82 mmol) in dioxane (5 mL) and H₂O (5 mL) was stirred at 100° C. for 12 hours. The reaction mixture was quenched with H₂O (20 mL) and extracted with EtOAc (50 mL×3). The combined organic layer was washed with brine (20 mL), dried over anhydrous Na₂SO₄, filtered and concentrated. The residue was purified by prep-HPLC (Method A; 0.05% FA as an additive), then lyophilized to give the title compound (24.31 mg, yield: 6%) as a white solid.

¹H NMR (400 MHz, CD₃OD) δ 0.93 (6H, d, J=6.8 Hz), 1.75-1.95 (5H, m), 1.95-2.10 (2H, m), 2.10-2.15 (2H, m), 2.50-2.55 (1H, m), 2.95-3.05 (2H, m), 7.47 (1H, dd, J=5.6, 2.0 Hz), 8.05 (1H, dd, J=5.6, 0.8 Hz), 8.12 (1H, s), 8.23 (1H, d, J=5.6 Hz), 8.28 (1H, d, J=5.2 Hz), 8.57 (1H, s), 8.78 (1H, s).

The following compound was synthesized analogously to Example 3

| Example | | | |
|---|---|---|---|
| No. | Structure | Name | ¹H NMR (400 MHz) |
| 7 | 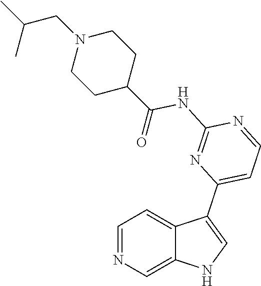 [SMILES: CC(C)CN1CCC(C(=O)Nc2nccc(-c3c[nH]c4cnccc34)n2)CC1] | N-(4-(1H- pyrrolo[2,3- c]pyridin-3- yl)pyrimidin-2-yl)-1- isobutylpiperidine-4- carboxamide | CD₃OD; δ 0.94 (6H, d, J = 6.8 Hz), 1.75-2.00 (5H, m), 1.95-2.10 (2H, m), 2.13-2.15 (2H, m), 2.55-2.70 (1H, m), 2.95-3.05 (2H, m), 7.54 (1H, d, J = 5.6 Hz), 8.25 (1H, d, J = 6.0 Hz), 8.45 (1H, s), 8.47 (1H, d, J = 5.2 Hz), 8.70-8.80 (2H, m). |

### Example 4

### 1-isobutyl-N-(4-(1-isopropyl-1H-pyrrolo[2,3-c]pyridin-3-yl)pyridin-2-yl)piperidine-4-carboxamide

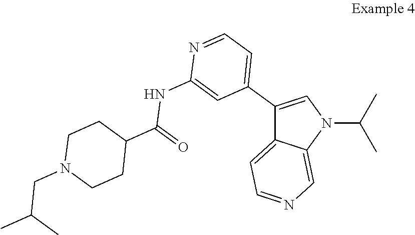 [SMILES: CC(C)CN1CCC(C(=O)Nc2cc(-c3cn(C(C)C)c4cnccc34)ccn2)CC1]

### Step 1. Synthesis of tert-butyl 4-((4-(1-isopropyl-1H-pyrrolo[2,3-c]pyridin-3-yl)pyridin-2-yl)carbamoyl)piperidine-1-carboxylate

To a solution of Intermediate 4 (300 mg, 1.19 mmol) and 1-(tert-butoxycarbonyl)piperidine-4-carboxylic acid (409 mg, 1.78 mmol) in pyridine (2 mL) was added Et₃N (361 mg, 3.57 mmol) and T₃P (1.51 g, 2.38 mmol) at 25° C. The mixture was stirred at 25° C. for 10 hours. The reaction mixture was diluted with water (20 mL) and extracted with EtOAc (50 mL×3). The combined organic layer was washed with brine (20 mL), dried over anhydrous Na₂SO₄, filtered and concentrated. The residue was purified by silica gel column (PE/EtOAc=1/1 to 0/1) to give tert-butyl 4-((4-(1-isopropyl-1H-pyrrolo[2,3-c]pyridin-3-yl)pyridin-2-yl)carbamoyl)piperidine-1-carboxylate (350 mg, yield: 64%) as a yellow solid.

### Step 2. Synthesis of N-(4-(1-isopropyl-H-pyrrolo[2,3-c]pyridin-3-yl)pyridin-2-yl)piperidine-4-carboxamide

To a solution of tert-butyl 4-((4-(1-isopropyl-1H-pyrrolo[2,3-c]pyridin-3-yl)pyridin-2-yl)carbamoyl)piperidine-1-carboxylate (350 mg, 0.755 mmol) in DCM (2 mL) was added TFA (2 mL). The mixture was stirred at 25° C. for 0.5 hour. The reaction mixture was basified with saturated aqueous Na₂CO₃ to pH=10 and extracted with EtOAc (50 mL×3). The combined organic layer was washed with brine (20 mL), dried over anhydrous Na₂SO₄, filtered and concentrated to give N-(4-(1-isopropyl-1H-pyrrolo[2,3-c]pyridin-3-yl)pyridin-2-yl)piperidine-4-carboxamide (200 mg, yield: 73%) as a yellow solid.

### Step 3. Synthesis of 1-isobutyl-N-(4-(1-isopropyl-1H-pyrrolo[2,3-c]pyridin-3-yl)pyridin-2-yl)piperidine-4-carboxamide

To a solution of N-(4-(1-isopropyl-1H-pyrrolo[2,3-c]pyridin-3-yl)pyridin-2-yl)piperidine-4-carboxamide (200 mg, 0.550 mmol) in MeOH (5 mL) was added isobutyraldehyde (60 mg, 0.83 mmol) at 25° C. The mixture was stirred at 25° C. for 0.5 hour. Then NaBH₃CN (104 mg, 1.65 mmol) was added and the mixture was stirred at 25° C. for another 0.5 hour. The reaction mixture was quenched with H₂O (20 mL) and extracted with EtOAc (50 mL×3). The combined organic layer was washed with brine (20 mL), dried over anhydrous Na₂SO₄, filtered and concentrated. The residue was purified by prep-HPLC (Method A; 0.05% FA as an additive) to give the title compound (24.89 mg, yield: 10%) as a white solid.

¹H NMR (400 MHz, CD₃OD) δ 0.96 (6H, d, J=6.4 Hz), 1.67 (6H, d, J=6.4 Hz), 1.84-1.98 (5H, m), 2.00-2.15 (2H, m), 2.15-2.20 (2H, m), 2.45-2.60 (1H, m), 3.00-3.10 (2H, m), 4.95-5.05 (1H, m), 7.51 (1H, dd, J=5.2, 1.6 Hz, 1H), 8.07 (1H, d, J=6.0 Hz), 8.25-8.35 (3H, m), 8.56 (1H, s), 8.94 (1H, s).

The following compounds were synthesized analogously to Example 4

| Example | | | |
|---|---|---|---|
| No. | Structure | Name | ¹H NMR (400 MHz) |
| 8 | 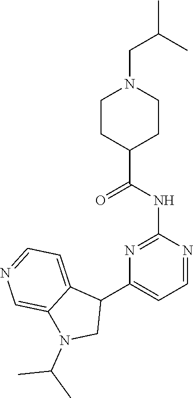 [SMILES: CC(C)CN1CCC(C(=O)Nc2nccc(C3CN(C(C)C)c4cnccc43)n2)CC1] | 1-isobutyl-N-(4-(1- isopropyl-1H-pyrrolo[2,3- c]pyridin-3-yl)pyrimidin- 2-yl)piperidine-4- carboxamide | CD₃OD; δ 0.96 (6H, d, J = 6.4 Hz), 1.68 (6H, d, J = 6.8 Hz), 1.85-2.00 (5H, m), 2.00-2.20 (4H, m), 2.60- 2.70 (1H, m), 3.00-3.10 (2H, m), 5.00-5.05 (1H, m), 7.59 (1H, d, J = 5.6 Hz), 8.31 (1H, d, J = 5.6 Hz), 8.49 (1H, d, J = 5.2 Hz), 8.63 (1H, s), 8.80 (1H, d, J = 5.6 Hz), 8.92 (1H, s). |
| 43 | 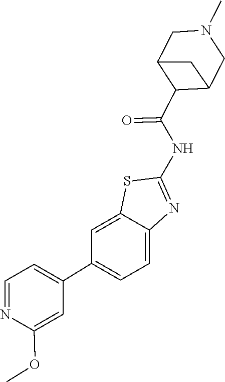 [SMILES: COc1cc(-c2ccc3nc(NC(=O)C4C5CC4CN(C)C5)sc3c2)ccn1] | N-(6-(2-methoxypyridin- 4-yl)benzo[d]thiazol-2- yl)-3-methyl-3- azabicyclo[3.1.1]heptane- 6-carboxamide | CD₃OD; δ 1.59-1.99 (1H, m), 2.62-2.84 (1H, m), 2.90-2.95 (2H, m), 3.00- 3.07 (4H, m), 3.50-3.60 (2H, m), 3.97-4.09 (2H, m), 4.35 (3H, s), 7.86-7.98 (3H, m), 8.07 (1H, dd, J = 8.8, 2.0 Hz), 8.37 (1H, d, J = 6.4 Hz), 8.62 (1H, d, J = 1.6 Hz). |
| 44 | 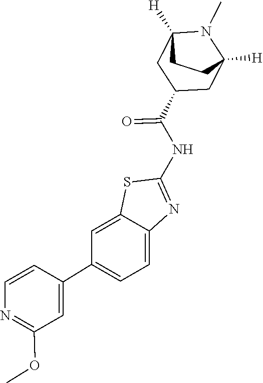 [SMILES: COc1cc(-c2ccc3nc(NC(=O)[C@H]4C[C@H]5CC[C@@H](C4)N5C)sc3c2)ccn1] | (1R,3s,5S)-N-(6-(2- methoxypyridin-4- yl)benzo[d]thiazol-2-yl)- 8-methyl-8- azabicyclo[3.2.1]octane- 3-carboxamide | CD₃OD; δ 1.85-1.98 (4H, m), 2.03-2.17 (2H, m), 2.18-2.31 (2H, m), 2.58 (3H, s), 2.91-3.08 (1H, m), 3.70-3.75 (2H, m), 3.86 (3H, s), 7.01 (1H, s), 7.20 (1H, dd, J = 5.2, 1.2 Hz), 7.64-7.77 (2H, m), 8.07 (1H, d, J = 5.2 Hz), 8.15 (1H, s), 8.46 (1H, brs). |
| 45 | 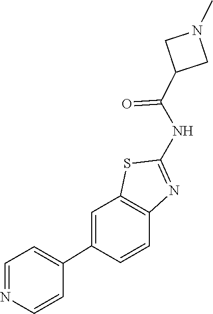 [SMILES: CN1CC(C(=O)Nc2nc3ccc(-c4ccncc4)cc3s2)C1] | 1-methyl-N-(6-(pyridin-4- yl)benzo[d]thiazol-2- yl)azetidine-3- carboxamide | DMSO-d₆; δ 2.49 (3H, s), 3.63-3.73 (3H, m), 3.75- 3.85 (2H, m), 7.73-7.82 (2H, m), 7.85-7.90 (2H, m), 8.53 (1H, d, J = 1.6 Hz), 8.60-8.70 (2H, m). |
| 46 | 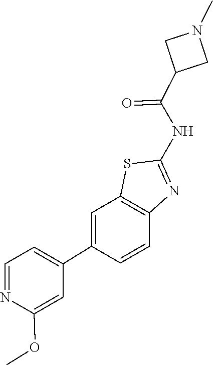 [SMILES: COc1cc(-c2ccc3nc(NC(=O)C4CN(C)C4)sc3c2)ccn1] | N-(6-(2-methoxypyridin- 4-yl)benzo[d]thiazol-2- yl)-1-methylazetidine-3- carboxamide | DMSO-d₆; δ 2.86 (3H, s), 3.82-4.01 (4H, m), 4.20- 4.40 (4H, m), 7.18 (1H, d, J = 0.8 Hz), 7.39 (1H, dd, J = 5.6, 1.6 Hz), 7.80-8.00 (2H, m), 8.24 (1H, s), 8.52 (1H, s), 12.71 (1H, brs). |
| 52 | 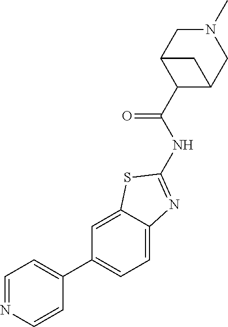 [SMILES: CN1CC2CC(C1)C2C(=O)Nc1nc2ccc(-c3ccncc3)cc2s1] | 3-methyl-N-(6-(pyridin-4- yl)benzo[d]thiazol-2-yl)- 3- azabicyclo[3.1.1]heptane- 6-carboxamide | CD₃OD; δ 1.85-1.95 (1H, m), 2.37-2.52 (1H, m), 2.90-2.95 (2H, m), 3.06 (3H, s), 3.45-3.55 (1H, m), 3.72-4.26 (4H, m), 7.95 (1H, d, J = 8.8 Hz), 8.03 (1H, d, J = 8.4 Hz), 8.15- 8.25 (2H, m), 8.56 (1H, s), 8.65-8.70 (2H, m). |
| 53 | 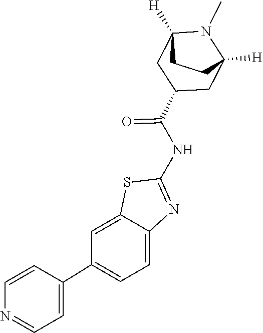 [SMILES: CN1[C@@H]2CC[C@H]1C[C@@H](C(=O)Nc1nc3ccc(-c4ccncc4)cc3s1)C2] | (1R,3s,5S)-8-methyl-N- (6-(pyridin-4- yl)benzo[d]thiazol-2-yl)- 8- azabicyclo[3.2.1]octane- 3-carboxamide | CD₃OD; δ 2.10-2.15 (4H, m), 2.21-2.33 (2H, m), 2.40-2.45 (2H, m), 2.82 (3H, s), 3.10-3.20 (1H, m), 4.00-4.05 (2H, m), 7.80- 7.85 (2H, m), 7.85-7.90 (2H, m), 8.36 (1H, s), 8.60- 8.65 (2H, m). |
| 61 | 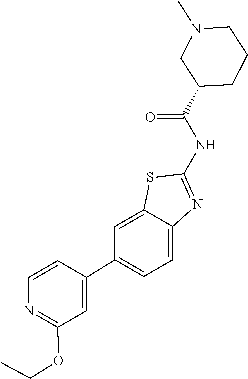 [SMILES: CCOc1cc(-c2ccc3nc(NC(=O)[C@H]4CCCN(C)C4)sc3c2)ccn1] | (S)-N-(6-(2- ethoxypyridin-4- yl)benzo[d]thiazol-2-yl)- 1-methylpiperidine-3- carboxamide | DMSO-d₆, δ 1.35 (3H, t, J = 6.8 Hz), 1.44-1.67 (2H, m), 1.72-2.01 (2H, m), 2.31- 2.44 (1H, m), 2.46 (3H, s), 2.82-3.00 (2H, m), 3.10- 3.15 (2H, m), 4.36 (2H, q, J = 6.8 Hz), 7.15 (1H, s), 7.36 (1H, dd, J = 7.2, 1.6 Hz), 7.80-7.90 (2H, m), 8.22 (1H, d, J = 5.6 Hz), 8.48 (1H, d, J = 1.2 Hz). |
| 62 | 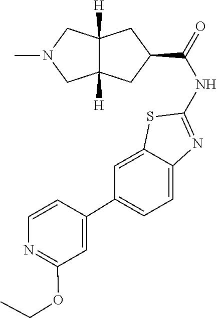 [SMILES: CCOc1cc(-c2ccc3nc(NC(=O)[C@H]4C[C@@H]5CN(C)C[C@@H]5C4)sc3c2)ccn1] | (3aR,5s,6aS)-N-(6-(2- ethoxypyridin-4- yl)benzo[d]thiazol-2-yl)- 2- methyloctahydrocyclo- penta[c]pyrrole-5- carboxamide | DMSO-d₆; δ 1.35 (3H, t, J = 7.2 Hz), 1.71-1.82 (2H, m), 1.83-1.95 (2H, m), 2.18- 2.29 (5H, m), 2.60-2.78 (4H, m), 3.15-3.29 (1H, m), 4.35 (2H, q, J = 7.2 Hz), 7.10-7.18 (1H, m), 7.36 (1H, dd, J = 5.6, 1.6 Hz), 7.77-7.88 (2H, m), 8.21 (1H, d, J = 5.6 Hz), 8.45 (1H, d, J = 1.2 Hz), 12.42 (1H, brs). |

### Example 5

### N-(1-isobutylpiperidin-4-yl)methyl)-4-(1H-pyrrolo[2,3-c]pyridin-3-yl)pyridin-2-amine

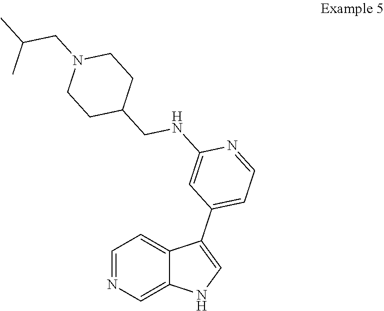 [SMILES: CC(C)CN1CCC(CNc2cc(-c3c[nH]c4cnccc34)ccn2)CC1]

### Step 1. Synthesis of tert-butyl 4-(((4-(J-tosyl-1H-pyrrolo[2,3-c]pyridin-3-yl)pyridin-2-yl)amino)methyl)piperidine-1-carboxylate

A mixture of Intermediate 5 (400 ing, 1.00 mmol), Int-6c (409 ins, 1.10 mmol), Pd(dppf)Cl₂ (73 ing, 0.10 mmol) and K₂CO₃ (278 ing, 2.01 mmol) in dioxane (10 mL) and H₂O (2 mL) was degassed and purged with N₂ for 3 times. Then the resulting reaction mixture was stirred at 90° C. for 3 hours under N₂ atmosphere. The reaction mixture was diluted with water (25 mL) and extracted with EtOAc (25 mL×3). The combined organic layer was washed with brine (25 mL), dried over anhydrous Na₂SO₄, filtered and concentrated. The residue was purified by silica gel column (PE/EtOAc=5/1 to 1/1) to give tert-butyl 4-(((4-(1-tosyl-1H-pyrrolo[2,3-c]pyridin-3-yl)pyridin-2-yl)amino)methyl)piperidine-1-carboxylate (300 mg, yield: 53%) as a white solid.

### Step 2. Synthesis of N-(piperidin-4-ylmethyl)-4-(1-tosyl-1H-pyrrolo[2,3-c]pyridin-3-yl)pyridin-2-amine

A solution of tert-butyl 4-(((4-(1-tosyl-1H-pyrrolo[2,3-c]pyridin-3-yl)pyridin-2-yl)amino)methyl)piperidine-1-carboxylate (300 mg, 0.534 mmol) in 4N HCl/MeOH (5 mL) was stirred at 25° C. for 2 hours. The reaction mixture was concentrated to give N-(piperidin-4-ylmethyl)-4-(1-tosyl-1H-pyrrolo[2,3-c]pyridin-3-yl)pyridin-2-amine (250 mg, yield: 94%, HCl salt) as a white solid.

### Step 3. Synthesis of N-((1-isobutylpiperidin-4-yl)methyl)-4-(1-tosyl-1H-pyrrolo[2,3-c]pyridin-3-yl)pyridin-2-amine

To a solution of N-(piperidin-4-ylmethyl)-4-(1-tosyl-1H-pyrrolo[2,3-c]pyridin-3-yl)pyridin-2-amine (250 mg, 0.502 mmol, HCl salt) in MeOH (2 mL) was added isobutyraldehyde (72 mg, 1.0 mmol) at 25° C. The mixture was stirred at 25° C. for 0.5 hour. Then NaBH₃CN (94 mg, 1.5 mmol) was added and the mixture was stirred at 25° C. for another 0.5 hour. The reaction mixture was quenched with H₂O (20 mL) and extracted with EtOAc (50 mL×3). The combined organic layer was washed with brine (20 mL), dried over anhydrous Na₂SO₄, filtered and concentrated to give N-((1-isobutylpiperidin-4-yl)methyl)-4-(1-tosyl-1H-pyrrolo[2,3-c]pyridin-3-yl)pyridin-2-amine (200 mg, yield: 77%) as yellow oil.

### Step 4. Synthesis of N-((1-isobutylpiperidin-4-yl)methyl)-4-(1H-pyrrolo[2,3-c]pyridin-3-yl)pyridin-2-amine

To a solution of N-((1-isobutylpiperidin-4-yl)methyl)-4-(1-tosyl-1H-pyrrolo[2,3-c]pyridin-3-yl)pyridin-2-amine (200 mg, 0.386 mmol) in MeOH (2 mL) and H₂O (2 mL) was added KOH (434 mg, 7.73 mmol) at 25° C. The mixture was stirred at 80° C. for 0.5 hour. The reaction mixture was diluted with H₂O (20 mL) and extracted with EtOAc (50 mL×3). The combined organic layer was washed with brine (20 mL), dried over anhydrous Na₂SO₄, filtered and concentrated. The residue was purified by prep-HPLC (Method A; 0.05% TFA as an additive), then lyophilized to give the title compound (18.93 mg, yield: 10%, TFA salt) as an off-white solid.

¹H NMR (400 MHz, D₂O) δ 0.85-0.95 (6H, m), 145-1.65 (2H, m), 1.95-2.15 (4H, m), 2.80-3.05 (4H, m), 3.25-3.40 (2H, m), 3.55-3.60 (2H, m), 7.15-7.25 (2H, m), 7.79 (1H, d, J=6.8 Hz), 8.22-8.32 (2H, m), 8.57 (1H, s), 9.04 (1H, s).

### Example 6

### N-((1-isobutylpiperidin-4-yl)methyl)-4-(l-isopropyl-1H-pyrrolo[2,3-c]pyridin-3-yl)pyridin-2-amine

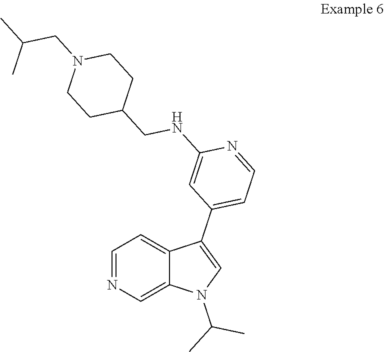 [SMILES: CC(C)CN1CCC(CNc2cc(-c3cn(C(C)C)c4cnccc34)ccn2)CC1]

### Step 1. Synthesis of tert-butyl 4-(((4-(1-isopropyl-1H-pyrrolo[2,3-c]pyridin-3-yl)pyridin-2-yl)amino)methyl)piperidine-1-carboxylate

A mixture of Intermediate 3 (500 mg, 1.75 mmol), Intermediate 6 (729 mg, crude), Pd(dppf)Cl₂ (128 mg, 0.175 mmol) and K₂CO₃ (483 mg, 3.50 mmol) in dioxane (10 mL) and H₂O (2 mL) was degassed and purged with N₂ for 3 times. Then the resulting reaction mixture was stirred at 100° C. for 3 hours under N₂ atmosphere. The reaction mixture was diluted with water (50 mL) and extracted with EtOAc (70 mL×3). The combined organic layer was washed with brine (25 mL), dried over anhydrous Na₂SO₄, filtered and concentrated. The residue was purified by silica gel column (PE/EtOAc=1/0 to 20/1) to give tert-butyl 4-(((4-(1-isopropyl-1H-pyrrolo[2,3-c]pyridin-3-yl)pyridin-2-yl)amino)methyl)piperidine-1-carboxylate (200 mg, yield: 25%) as yellow gum.

### Step 2. Synthesis of 4-(1-isopropyl-1H-pyrrolo[2,3-c]pyridin-3-yl)-N-(piperidin-4-ylmethyl)pyridin-2-amine

A solution of tert-butyl 4-(((4-(1-isopropyl-1H-pyrrolo[2,3-c]pyridin-3-yl)pyridin-2-yl)amino)methyl)piperidine-1-carboxylate (200 mg, 0.445 mmol) in 4N HCl/MeOH (5 mL) was stirred at 25° C. for 12 hours. The reaction mixture was concentrated to give 4-(1-isopropyl-1H-pyrrolo[2,3-c]pyridin-3-yl)-N-(piperidin-4-ylmethyl)pyridin-2-amine (170 mg, yield: 81%, HCl salt) as a yellow solid.

### Step 3. Synthesis of N-((1-isobutylpiperidin-4-yl)methyl)-4-(1-isopropyl-1H-pyrrolo[2,3-c]pyridin-3 yl)pyridin-2-amine

To a solution of 4-(1-isopropyl-1H-pyrrolo[2,3-c]pyridin-3-yl)-N-(piperidin-4-ylmethyl)pyridin-2-amine (170 mg, 0.360 mmol, HCl salt) in MeOH (2 mL) was added isobutyraldehyde (52 mg, 0.72 mmol) at 25° C. The mixture was stirred at 25° C. for 0.5 hour. Then NaBH₃CN (68 mg, 1.1 mmol) was added and the mixture was stirred at 25° C. for another 0.5 hour. The reaction mixture was quenched with 1120 (20 mL) and extracted with EtOAc (50 mL×3). The combined organic layer was washed with brine (20 mL), dried over anhydrous Na₂SO₄, filtered and concentrated. The residue was purified by prep-HPLC (0.05% TFA as an additive) to the title compound (53.75 mg, yield: 29%, TFA salt) as an off-white solid.

¹H NMR (400 MHz, D₂O) δ 0.85-0.95 (6H, m), 1.50-1.60 (8H, m), 1.95-2.15 (4H, m), 2.75-3.05 (4H, m), 3.00-3.30 (2H, m), 3.55-3.60 (2H, m), 4.90-5.02 (1H, m), 7.15-7.25 (2H, m), 7.77 (1H, d, J=6.8 Hz), 8.22-8.28 (2H, m), 8.70 (1H, s), 9.17 (1H, s).

The following compounds were synthesized analogously to Example 6

| Example | | | |
|---|---|---|---|
| No. | Structure | Name | ¹H NMR (400 MHz) |
| 17 | 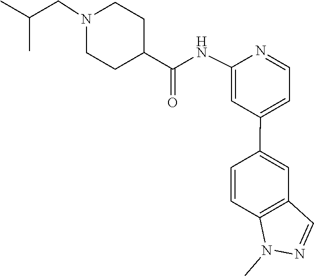 [SMILES: CC(C)CN1CCC(C(=O)Nc2cc(-c3ccc4c(cnn4C)c3)ccn2)CC1] | 1-isobutyl-N-(4-(1- methyl-1H-indazol-5- yl)pyridin-2-yl)piperidine- 4-carboxamide | CD₃OD; δ 0.94 (6H, d, J = 6.8 Hz), 1.79-1.90 (4H, m), 1.91-1.94 (1H, m), 2.00-2.05 (2H, m), 2.10-2.15 (2H, m), 2.46-2.55 (1H, m), 3.00-3.05 (2H, m), 4.11 (3H, s), 7.46 (1H, dd, J = 5.2, 1.6 Hz), 7.69 (1H, d, J = 8.8 Hz), 7.82 (1H, dd, J = 8.8, 1.6 Hz), 8.12 (1H, s), 8.16 (1H, s), 8.33 (1H, d, J = 5.2 Hz), 8.46 (1H, s). |
| 18 | 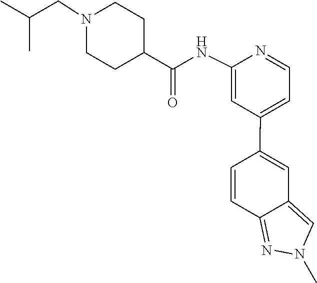 [SMILES: CC(C)CN1CCC(C(=O)Nc2cc(-c3ccc4nn(C)cc4c3)ccn2)CC1] | 1-isobutyl-N-(4-(2- methyl-2H-indazol-5- yl)pyridin-2-yl)piperidine- 4-carboxamide | CD₃OD; δ 0.93 (6H, d, J = 6.8 Hz), 1.75-1.95 (5H, m), 1.95-2.05 (2H, m), 2.10-2.20 (2H, m), 2.40-2.60 (1H, m), 2.95-3.05 (2H, m), 4.25 (3H, s), 7.44 (1H, dd, J = 5.2, 1.6 Hz), 7.60-7.75 (2H, m), 8.11 (1H, s), 8.25-8.35 (2H, m), 8.45 (1H, s). |
| 47 | 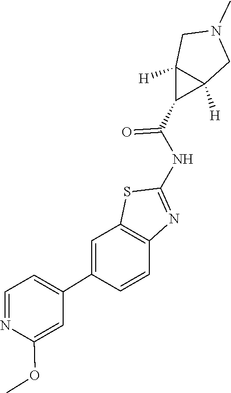 [SMILES: COc1cc(-c2ccc3nc(NC(=O)[C@H]4[C@@H]5CN(C)C[C@@H]54)sc3c2)ccn1] | (1R,5S,6r)-N-(6-(2- methoxypyridin-4- yl)benzo[d]thiazol-2-yl)- 3-methyl-3- azabicyclo[3.1.0]hexane- 6-carboxamide | CD₃OD; δ 2.20-2.30 (3H, m), 2.55-2.60 (3H, m), 2.85- 2.95 (2H, m), 3.40-3.50 (2H, m), 3.98 (3H, s), 7.14 (1H, s), 7.33 (1H, d, J = 5.2 Hz), 7.80 (1H, d, J = 8.4 Hz), 7.85 (1H, d, J = 8.8 Hz), 8.19 (1H, d, J = 5.6 Hz), 8.26 (1H, s), 8.34 (1H, s). |
| 54 | 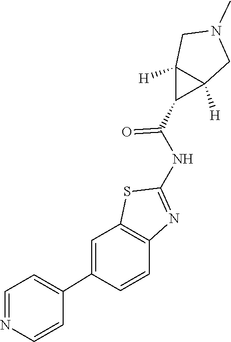 [SMILES: CN1C[C@@H]2[C@H](C1)[C@H]2C(=O)Nc1nc2ccc(-c3ccncc3)cc2s1] | (1R,5S,6r)-3-methyl-N-(6- (pyridin-4- yl)benzo[d]thiazol-2-yl)- 3- azabicyclo[3.1.0]hexane- 6-carboxamide | CD₃OD; δ 2.20-2.25 (1H, m), 2.30-2.35 (2H, m), 2.62- 2.68 (3H, m), 2.95-3.05 (2H, m), 3.38-3.50 (2H, m), 7.79- 7.83 (2H, m), 7.84-7.92 (2H, m), 8.30-8.35 (2H, m), 8.59- 8.65 (2H, m). |
| 70 | 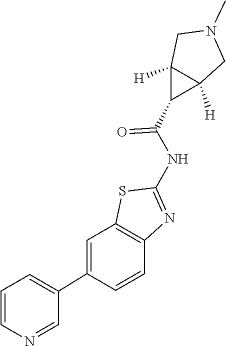 [SMILES: CN1C[C@@H]2[C@H](C1)[C@H]2C(=O)Nc1nc2ccc(-c3cccnc3)cc2s1] | (1R,5S)-3-methyl-N-(5- (pyridin-3-yl)thiazolo[5,4- b]pyridin-2-yl)-3- azabicyclo[3.1.0]hexane- 6-carboxamide | DMSO-d₆; δ 2.05-2.10 (2H, m), 2.25-2.35 (3H, m), 2.38- 2.47 (3H, m), 3.05-3.10 (2H, m), 7.54 (1H, dd, J = 8.0, 4.8 Hz), 8.14-8.17 (1H, m), 8.19-8.23 (1H, m), 8.44-8.52 (1H, m), 8.64 (1H, dd, J = 4.8, 1.6 Hz), 9.31 (1H, d, J = 2.0 Hz), 12.74 (1H, brs). |
| 101 | 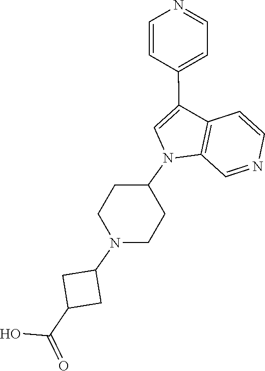 [SMILES: O=C(O)C1CC(N2CCC(n3cc(-c4ccncc4)c4ccncc43)CC2)C1] | 3-(4-(3-(pyridin-4-yl)-1H- pyrrolo[2,3-c]pyridin-1- yl)piperidin-1- yl)cyclobutane-1- carboxylic acid | DMSO-d₆; δ 1.93-2.45 (10H, m), 2.74-2.84 (1H, m), 2.87-3.01 (1H, m), 3.05-3.16 (2H, m), 4.65-4.81 (1H, m), 7.76-7.84 (2H, m), 8.01 (1H, d, J = 5.6 Hz), 8.30 (1H, d, J = 5.6 Hz), 8.52 (1H, s), 8.56- 8.61 (2H, m), 9.11 (1H, s). |

### Example 9

### N-((1-isobutylpiperidin-4-yl)methyl)-4-(1-isopropyl-1H-pyrrolo[2,3-c]pyridin-3-yl)pyrimidin-2-amine

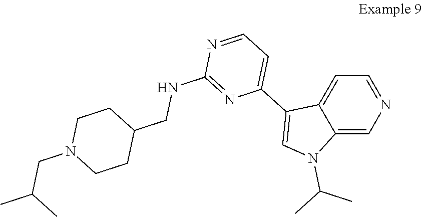 [SMILES: CC(C)CN1CCC(CNc2nccc(-c3cn(C(C)C)c4cnccc34)n2)CC1]

### Step 1. Synthesis of tert-butyl 4-(((4-(1-isopropyl-1H-pyrrolo[2,3-c]pyridin-3-yl)pyrimidin-2-yl)amino)methyl)piperidine-1-carboxylate

To a solution of Intermediate 9 (250 mg, 0.917 mmol) and tert-butyl 4-(aminomethyl)piperidine-1-carboxylate (589 mg, 2.75 mmol) in DMA (4 mL) was added K₂CO₃ (380 mg, 2.75 mmol) at 25° C. The mixture was stirred at 100° C. for 6 hours. The reaction mixture was diluted with water (20 mL) and extracted with EtOAc (50 mL×3). The combined organic layer was washed with brine (20 mL×3), dried over anhydrous Na₂SO₄, filtered and concentrated. The residue was purified by silica gel column (DCM/MeOH=I/O to 10/1) to give tert-butyl 4-(((4-(1-isopropyl-1H-pyrrolo[2,3-c]pyridin-3-yl)pyrimidin-2-yl)amino)methyl)piperidine-1-carboxylate (200 mg, yield: 38%) as a yellow solid.

¹H NMR (400 MHz, CDCl₃) δ 1.20-1.30 (2H, m), 1.46 (9H, s), 1.66 (6H, d, J=6.8 Hz), 1.80-1.90 (3H, m), 2.65-2.80 (2H, m), 341-3.50 (2H, m), 4.10-4.25 (2H, m), 4.80-4.90 (1H, m), 5.20-5.33 (1H, m), 6.89 (1H, d, J=5.2 Hz), 8.02 (1H, s), 8.22-8.28 (2H, m), 8.38 (1H, d, J=5.6 Hz), 8.89 (s, 1H, s).

### Step 2. Synthesis of 4-(1-isopropyl-1H-pyrrolo[2,3-c]pyridin-3-yl)-N-(piperidin-4-ylmethyl)pyrimidin-2-amine

A solution of tert-butyl 4-(((4-(1-isopropyl-1H-pyrrolo[2,3-c]pyridin-3-yl)pyrimidin-2-yl)amino)methyl)piperidine-1-carboxylate (200 mg, 0.444 mmol) in 4N HCl/MeOH (5 mL) was stirred at 25° C. for 1 hour. The reaction mixture was concentrated to give 4-(1-isopropyl-1H-pyrrolo[2,3-c]pyridin-3-yl)-N-(piperidin-4-ylmethyl)pyrimidin-2-amine (170 mg, yield: 99%, HCl salt) as a yellow solid.

### Step 3. Synthesis of N-((1-isobutylpiperidin-4-yl)methyl)-4-(1-isopropyl-1H-pyrrolo[2,3-c]pyridin-3-yl)pyrimidin-2-amine

To a solution of 4-(1-isopropyl-1H-pyrrolo[2,3-c]pyridin-3-yl)-N-(piperidin-4-ylmethyl)pyrimidin-2-amine (170 mg, 0.439 mmol, HCl salt) in MeOH (2 mL) was added isobutyraldehyde (63 mg, 0.88 mmol) at 25° C. The mixture was stirred at 25° C. for 0.5 hour. NaBH₃CN (83 mg, 1.3 mmol) was added to the reaction mixture at 25° C. The mixture was stirred at 25° C. for another 0.5 hour. The reaction mixture was quenched with H₂O (20 mL) and extracted with EtOAc (50 mL×3). The combined organic layer was washed with brine (20 mL), dried over anhydrous Na₂SO₄, filtered and concentrated. The residue was purified by prep-HPLC (0.1% HCl as an additive), then lyophilized to give the title compound (69.19 mg, yield: 35%, HCl salt) as an off-white solid.

¹H NMR (400 MHz, D₂O) δ 0.90 (6H, d, J=6.4 Hz), 1.50-1.75 (8H, m), 1.96-2.16 (4H, m), 2.75-2.90 (4H, m), 3.50-3.75 (4H, m), 4.95-5.05 (1H, m), 7.32 (1H, d, J=6.4 Hz), 8.08 (1H, d, J=6.8 Hz), 8.35 (1H, d, J=6.4 Hz), 8.75-8.85 (1H, m), 9.07 (s, 1H), 9.23 (s, 1H).

### Example 10

### 4-(2,3-dihydrobenzofuran-5-yl)-2-phenoxypyridine

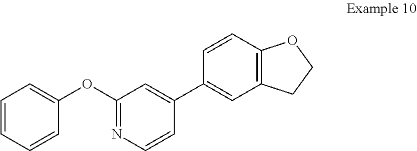 [SMILES: c1ccc(Oc2cc(-c3ccc4c(c3)CCO4)ccn2)cc1]

### Step 1. Synthesis of 4-bromo-2-phenoxypyridine

To a solution of phenol (267 mg, 2.84 mmol) in anhydrous DMF (8 mL) was added NaH (114 mg, 2.84 mmol, 60% dispersion in mineral oil) at 25° C. and stirred at 25° C. for 30 minutes. 4-bromo-2-fluoropyridine (500 mg, 2.84 mmol) in anhydrous DMF (1 mL) was added to the reaction mixture. Then the reaction was stirred at 80° C. for 16 hours. The reaction mixture was quenched with water (25 mL), then extracted with EtOAc (10 mL×4). The combined organic layer was washed with brine (20 mL), dried over anhydrous Na₂SO₄, filtered and concentrated. The residue was purified by flash silica gel chromatography (ISCO®; 12 g SepaFlash® Silica Flash Column, Eluent of 0˜30% Ethyl acetate/Petroleum ether gradient @30 m/min) to give 4-bromo-2-phenoxypyridine (545 mg, yield: 68%) as colorless oil.

¹H NMR (400 MHz, CDCl₃): δ 7.10 (1H, d, J=1.2 Hz). 7.12-7.17 (3H, m), 7.22-7.27 (1H, m), 7.39-7.46 (2H, m), 8.03 (1H, d, J=5.2 Hz).

### Step 2. Synthesis of 4-(2,3-dihydrobenzofuran-5-yl)-2-phenoxypyridine

A mixture of 4-bromo-2-phenoxypyridine (300 mg, 1.06 mmol), (2,3-dihydrobenzofuran-5-yl)boronic acid (210 mg, 1.28 mmol), Pd(PPh₃)₄ (122 mg, 0.106 mmol), Na₂CO₃ (340 mg, 3.21 mmol) in dioxane (1.6 mL) and H₂O (0.4 mL) was degassed and purged with N₂ for 3 times and then the mixture was stirred at 100° C. for 2 hours under N₂ atmosphere. The reaction mixture was filtered and the filtrate was diluted with water (10 mL), then extracted with EtOAc (10 mL×3). The combined organic layer was washed with brine (20 mL), dried over anhydrous Na₂SO₄, filtered and concentrated. The residue was purified by prep-HPLC (0.04% NH₃·H₂O as an additive) and lyophilized to give the title compound (220.81 mg, yield: 72%) as a white solid.

¹H NMR (400 MHz, DMSO-d₆): δ 3.25 (2H, t, J=8.8 Hz), 4.60 (2H, t, J=8.8 Hz), 6.89 (1H, d, J=8.4 Hz), 7.13-7.16 (2H, m), 7.18-7.24 (1H, m), 7.27 (1H, s), 7.35-7.46 (3H, m), 7.55-7.64 (1H, m), 7.73 (1H, s), 8.12 (1H, d, J=5.2 Hz).

The following compounds were synthesized analogously to Example 10

| Example | | | |
|---|---|---|---|
| No. | Structure | Name | ¹H NMR (400 MHz) |
| 35 | 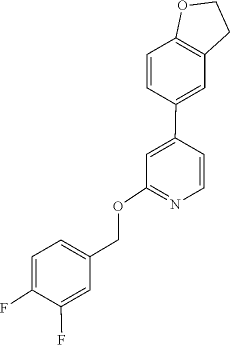 [SMILES: Fc1ccc(COc2cc(-c3ccc4c(c3)CCO4)ccn2)cc1F] | 2-((3,4- difluorobenzyl)oxy)-4- (2,3-dihydrobenzofuran- 5-yl)pyridine | CDCl₃; δ 3.28 (2H, t, J = 8.4 Hz), 4.64 (2H, t, J = 8.8 Hz), 5.37 (2H, s), 6.86 (1H, d, J = 8.0 Hz), 6.96 (1H, s), 7.07-7.21 (3H, m), 7.28- 7.35 (1H, m), 7.40 (1 H, dd, J = 8.4, 2.0 Hz), 7.48 (1H, s), 8.15 (1H, d, J = 5.2 Hz). |
| 36 | 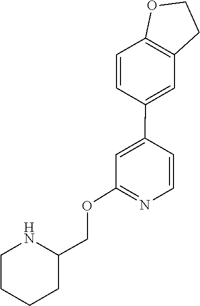 [SMILES: c1cc(-c2ccc3c(c2)CCO3)cc(OCC2CCCCN2)n1] | 4-(2,3- dihydrobenzofuran-5- yl)-2-(piperidin-2- ylmethoxy)pyridine | CD₃CN; δ 1.55-1.65 (1H, m), 1.78- 1.94 (5H, m), 2.88-3.04 (2H, m), 3.30-3.35 (2H, m), 3.41-3.45 (1H, m), 3.54-3.58 (1H, m), 4.65-4.75 (2H, m), 4.81-4.93 (2H, m), 6.95 (1H, d, J = 8.4 Hz), 7.58-7.62 (1H, m), 7.65 (1H, d, J = 6.4 Hz), 7.70- 7.76 (1H, m), 7.83 (1H, s), 8.24 (1H, d, J = 6.4 Hz, 1H), 9.71 (1H, brs), 9.96 (1H, brs). |
| 37 | 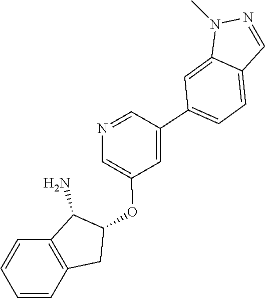 [SMILES: Cn1ncc2ccc(-c3cncc(O[C@@H]4Cc5ccccc5[C@@H]4N)c3)cc21] | (1S,2R)-2-((5-(1- methyl-1H-indazol-6- yl)pyridin-3-yl)oxy)- 2,3-dihydro-1H-inden-1- amine | DMSO-d₆; δ 3.21-3.34 (2H, m), 4.14 (3H, s), 5.09 (1H, t, J = 5.2 Hz), 5.55-5.65 (1H, m), 7.35-7.45 (3H, m), 7.55-7.75 (2H, m), 7.87- 7.98 (1H, m), 8.09-8.21 (2H, m), 8.46-9.00 (5H, m). |

### Example 11

### 4-(2,3-dihydrobenzofuran-5-yl)-2-(pyridin-3-yl)furo[2,3-c]pyridine

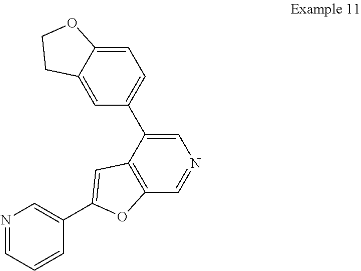 [SMILES: c1cncc(-c2cc3c(-c4ccc5c(c4)CCO5)cncc3o2)c1]

### Step 1. Synthesis of 3-bromo-5-(methoxymethoxy)pyridine

To a stirred solution of 5-bromopyridin-3-ol (5.00 g, 28.7 mmol) in anhydrous THF (50 mL) were successively added Et₃N (7.27 g, 71.8 mmol) and MOMCl (3.47 g, 43.1 mmol) at 0° C. The reaction mixture was then stirred at 20° C. for 3 hours to give a yellow mixture. Water (100 mL) was added to the reaction mixture and the reaction mixture was extracted with EtOAc (50 mL×3). The combined organic phase was washed with brine (100 mL), dried over anhydrous Na₂SO₄, filtered and concentrated to give 3-bromo-5-(methoxymethoxy)pyridine (6.20 g, yield: 99%) as yellow oil.

¹H NMR (400 MHz, CDCl₃) δ 3.49 (3H, s), 5.19 (2H, s), 7.56 (1H, t, J=2.4 Hz), 8.27-8.40 (2H, m).

### Step 2. Synthesis of 3-bromo-5-(methoxymethoxy)isonicotinaldehyde

To a solution of 3-bromo-5-(methoxymethoxy)pyridine (5.00 g, 22.9 mmol) in anhydrous THF (50 mL) was added LDA (12.6 mL, 25.2 mmol, 2 M in THF) dropwise at −70° C. under N₂ atmosphere. The reaction mixture was stirred at −70 for 1.5 hours. Then ethyl formate (3.40 g, 45.9 mmol) was added dropwise to the reaction mixture at −70° C. The reaction mixture was stirred at −70° C. for another 3 hours to give a brown mixture. Saturated aqueous NH₄Cl (150 mL) was added to quench the reaction mixture. The resulting mixture was extracted with EtOAc (100 mL×3). The combined organic phase was washed with brine (150 mL), dried over anhydrous Na₂SO₄, filtered and concentrated to give 3-bromo-5-(methoxymethoxy)isonicotinaldehyde (5.10 g, yield: 90%) as yellow oil.

¹H NMR (400 MHz, CDCl₃) δ 3.52 (3H, s), 5.32 (2H, s), 8.52 (1H, s), 8.59 (1H, s), 10.37 (1H, s).

### Step 3. Synthesis of 3-bromo-4-ethynyl-5-(methoxymethoxy)pyridine

A mixture of 3-bromo-5-(methoxymethoxy)isonicotinaldehyde (5.00 g, 20.3 mmol) and K₂CO₃ (5.62 g, 40.6 mmol) in MeOH (20 mL) was stirred at 20° C. for 0.5 hour. Then dimethyl (1-diazo-2-oxopropyl)phosphonate (6.25 g, 32.5 mmol) was added and the resulting reaction mixture was stirred at 20° C. for 2 hours to give a brown mixture. Water (150 mL) was added to the reaction mixture, then extracted with EtOAc (100 mL×3). The combined organic phase was washed with brine (100 mL), dried over anhydrous Na₂SO₄, filtered and concentrated. The residue was purified by flash silica gel chromatography (ISCO®; 40 g SepaFlash® Silica Flash Column, Eluent of 0˜30% Ethyl acetate/Petroleum ether gradient @40 mL/min) to give 3-bromo-4-ethynyl-5-(methoxymethoxy)pyridine (1.10 g, yield: 22%) as a yellow solid.

¹H NMR (400 MHz, CDCl₃) δ 3.54 (3H, s) 3.78 (1H, s), 5.31 (2H, s), 8.35-8.56 (2H, m).

### Step 4. Synthesis of 3-bromo-5-(methoxymethoxy)-4-(pyridin-3-ylethynyl)pyridine

A mixture of 3-bromo-4-ethynyl-5-(methoxymethoxy)pyridine (500 mg, 2.07 mmol), 3-iodopyridine (466 mg, 2.27 mmol), CuI (39 mg, 0.21 mmol), Pd(PPh₃)₄ (239 mg, 0.207 mmol) and Et₃N (627 mg, 6.20 mmol) in THF (20 mL) under N₂ was stirred at 20° C. for 16 hours to give a brown mixture. Water (50 mL) was added to the reaction mixture and extracted with EtOAc (30 mL×3). The combined organic phase was washed with brine (50 mL), dried over anhydrous Na₂SO₄, filtered and concentrated. The residue was purified by flash silica gel chromatography (ISCO®; 20 g SepaFlash® Silica Flash Column, Eluent of 0˜30% Ethyl acetate/Petroleum ether gradient @30 mL/min) to give 3-bromo-5-(methoxymethoxy)-4-(pyridin-3-ylethynyl)pyridine (310 mg, yield: 47%) as a yellow solid.

¹H NMR (400 MHz, CDCl₃) δ 3.57 (3H, s), 5.33 (2H, s), 7.31-7.37 (1H, m), 7.90 (1H, td, J=2.0, 8.0 Hz), 8.47 (1H, s), 8.49 (1H, s), 8.63 (1H, dd, J=5.2, 1.6 Hz), 8.85 (1H, d, J=1.6 Hz).

### Step 5. Synthesis of 5-bromo-4-(pyridin-3-ylethynyl)pyridin-3-ol

To a mixture of 3-bromo-5-(methoxymethoxy)-4-(pyridin-3-ylethynyl)pyridine (310 mg, 0.971 mmol) in THF (20 mL) was added 1N aqueous HCl (4.9 mL). The reaction mixture was stirred at 20° C. for 12 hours to give a yellow mixture. The reaction mixture was concentrated and dried to give 5-bromo-4-(pyridin-3-ylethynyl)pyridin-3-ol (320 mg, crude, HCl salt) as a yellow solid, which was directly used for the next step without further purification.

### Step 6. Synthesis of 4-bromo-2-(pyridin-3-yl)furo[2,3-c]pyridine

A mixture of 5-bromo-4-(pyridin-3-ylethynyl)pyridin-3-ol (320 mg, 1.03 mmol, HCl salt) and Cs₂CO₃ (1.34 g, 4.11 mmol) in MeCN (20 mL) was stirred at 60° C. for 12 hours to give a white mixture. Water (20 mL) was added to the reaction mixture and extracted with EtOAc (20 mL×3). The combined organic phase was washed with brine (30 mL), dried over anhydrous Na₂SO₄, filtered and concentrated. The residue was purified by flash silica gel chromatography (ISCO®; 20 g SepaFlash® Silica Flash Column, Eluent of 0˜50% Ethyl acetate/Petroleum ether gradient @25 mL/min) to give 4-bromo-2-(pyridin-3-yl)furo[2,3-c]pyridine (210 mg, yield: 74% for 2 steps) as a yellow solid.

¹H NMR (400 MHz, CDCl₃) δ 7.19 (1H, s), 7.46 (1H, dd, J=8.0, 5.2 Hz), 8.20 (1H, td, J=8.0, 2.0 Hz), 8.70 (1H, dd, J=4.8, 1.6 Hz), 8.55 (1H, s), 8.86 (1H, s), 9.19 (1H, d, J=2.0 Hz).

### Step 7. Synthesis of 4-(2,3-dihydrobenzofuran-5-yl)-2-(pyridin-3-yl)furo[2,3-c]pyridine

To a mixture of 4-bromo-2-(pyridin-3-yl)furo[2,3-c]pyridine (150 mg, 0.545 mmol), 2,3-dihydrobenzofuran-5-boronic acid (107 mg, 0.654 mmol) and Na₂CO₃ (116 mg, 1.09 mmol) in dioxane (4 mL) and H₂O (0.4 mL) was added Pd(PPh₃)₄ (63 mg, 0.055 mmol) under N₂ atmosphere. The mixture was stirred at 100° C. for 16 hours to give a brown mixture. Water (20 mL) was added to the reaction mixture and extracted with EtOAc (30 mL×3). The combined organic phase was washed with brine (50 mL), dried over anhydrous Na₂SO₄, filtered and concentrated. The residue was purified by flash silica gel chromatography (ISCO®; 25 g SepaFlash® Silica Flash Column, Eluent of 0˜100% Ethyl acetate/Petroleum ether gradient @30 mL/min) to give the title compound (130 mg, yield: 74%) as a yellow solid.

¹H NMR (400 MHz, CDCl₃) δ 3.35 (2H, t, J=8.8 Hz), 4.69 (2H, t, J=8.8 Hz), 6.97 (1H, d, J=8.4 Hz), 7.30 (1H, s), 7.41-7.47 (2H, m), 7.50 (1H, s), 8.21 (1H, d, J=8.0 Hz), 8.50 (1H, s), 8.67 (1H, d, J=4.0 Hz), 8.87 (1H, s), 9.17 (1H, d, J=2.0 Hz).

### Example 12

### N-(2-(3-methoxyphenyl)thiazolo[5,4-b]pyridin-6-yl)methanesulfonamide

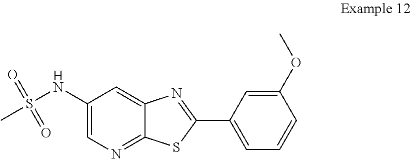 [SMILES: COc1cccc(-c2nc3cc(NS(C)(=O)=O)cnc3s2)c1]

### Step 1. Synthesis of 2-(3-methoxyphenyl)-6-nitrothiazolo[5,4-b]pyridine

A mixture of 2-chloro-3,5-dinitropyridine (590 mg, 2.90 mmol) and 3-methoxybenzothioamide (969 mg, 5.80 mmol) in sulfolane (10 mL) was degassed and purged with N₂ for 3 times, then the mixture was stirred at 110° C. for 2 hours under N₂ atmosphere. The reaction mixture was diluted with EtOAc (20 mL) and washed with water (15 mL×2), brine (15 mL), dried over anhydrous Na₂SO₄, filtered and concentrated. The residue was purified by Combi Flash (0% to 30% EtOAc in PE) to give 2-(3-methoxyphenyl)-6-nitrothiazolo[5,4-b]pyridine (600 mg, yield: 60%) as a yellow solid.

¹H NMR (400 MHz, DMSO-d₆) δ 3.90 (3H, s), 7.25-7.32 (1H, m), 7.57 (1H, t, J=8.0 Hz), 7.66-7.69 (1H, m), 7.75 (1H, d, J=7.6 Hz), 9.21 (1H, d, J=2.0 Hz), 9.43 (1H, d, J=2.4 Hz).

### Step 2. Synthesis of 2-(3-methoxyphenyl)thiazolo[5,4-b]pyridin-6-amine

To a solution of 2-(3-methoxyphenyl)-6-nitrothiazolo[5,4-b]pyridine (470 mg, 1.64 mmol) and NH₄Cl (350 mg, 6.54 mmol) in EtOH (6 mL) and H₂O (6 mL) was added Fe powder (117 mg, 2.09 mmol), then stirred at 75° C. for 1 hour. The reaction mixture was filtered and the filtrate was concentrated. The residue was purified by Combi Flash (0% to 100% EtOAc in PE) to give 2-(3-methoxyphenyl)thiazolo[5,4-b]pyridin-6-amine (420 mg, yield: 99%) as a light brown solid.

¹H NMR (400 MHz, CDCl₃) δ 3.91 (3H, s), 7.02-7.08 (1H, m), 7.40 (1H, t, J=8.0 Hz), 7.56 (1H, d, J=2.8 Hz), 7.59-7.64 (2H, m), 8.12 (1H, d, J=2.8 Hz).

### Step 3. Synthesis of N-(2-(3-methoxyphenyl)thiazolo[5,4-b]pyridin-6-yl)-N-(methylsulfonyl)methanesulfonamide

To a solution of 2-(3-methoxyphenyl)thiazolo[5,4-b]pyridin-6-amine (100 mg, 0.389 mmol) in anhydrous DCM (3 mL) was added Et₃N (79 mg, 0.78 mmol) and MsCl (53 mg, 0.47 mmol) at 0° C. The mixture was stirred at 25° C. for 1 hour. The reaction mixture was quenched with water (20 mL) and extracted with DCM (20 mL×2). The combined organic layer was washed with brine (20 mL), dried over anhydrous Na₂SO₄, filtered and concentrated to give N-(2-(3-methoxyphenyl)thiazolo[5,4-b]pyridin-6-yl)-N-(methylsulfonyl)methanesulfonamide (160 mg, crude) as a light yellow solid.

¹H NMR (400 MHz, DMSO-d₆) δ 3.35 (6H, s), 3.88 (3H, s), 7.23 (1H, dd, J=8.4, 2.4 Hz), 7.54 (1H, t, J=8.0H), 7.63-7.67 (1H, m), 7.71 (1H, d, J=2.8 Hz), 8.78-8.84 (2H, m).

### Step 4. Synthesis of N-(2-(3-methoxyphenyl)thiazolo[5,4-b]pyridin-6-yl)methanesulfonamide

To a solution of N-(2-(3-methoxyphenyl)thiazolo[5,4-b]pyridin-6-yl)-N-(methylsulfonyl)methanesulfonamide (160 mg, 0.387 mmol) in THF (2 mL) was added a solution of NaOH (110 mg, 2.75 mmol) in H₂O (1 mL) at 25° C. The mixture was stirred at 25° C. for 1 hour. The reaction mixture was diluted with water (25 mL) and extracted with EtOAc (20 mL×3). The combined organic layer was washed with brine (20 mL), dried over anhydrous Na₂SO₄, filtered and concentrated. The residue was purified by prep-HPLC (10 mM NH₄HCO₃ as an additive) and lyophilized to give the title compound (21.6 mg, yield: 16%) as a white solid.

¹H NMR (400 MHz, DMSO-d₆) δ 3.13 (3H, s), 3.89 (3H, s), 7.21 (1H, dd, J=8.4, 2.4 Hz), 7.52 (1H, t, J=8.0 Hz), 7.63-7.64 (1H, m), 7.68 (1H, d, J=8.0 Hz), 8.22 (1H, d, J=2.4 Hz), 8.48 (1H, d, J=2.4 Hz), 10.26 (1H, brs).

### Example 13

### N-(2-(3-hydroxyphenyl)thiazolo[5,4-b]pyridin-6-yl)methanesulfonamide

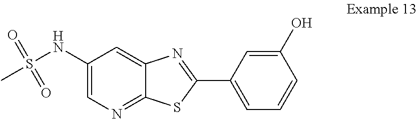 [SMILES: CS(=O)(=O)Nc1cnc2sc(-c3cccc(O)c3)nc2c1]

To a solution of Example 12 (300 mg, 0.894 mmol) in anhydrous DCM (5 mL) was added BBr₃ (448 mg, 1.79 mmol) at 0° C. The mixture was stirred at 0° C. for 1 hour. The reaction mixture was quenched with MeOH (1 mL) at 0° C., then concentrated. The residue was purified by prep-HPLC (0.04% NH₃H₂O and 10 mM NH₄HCO₃ as an additive) and lyophilized to give the title compound (29.22 mg, yield: 10%) as a white solid.

¹H NMR (400 MHz, DMSO-d₆) δ 3.04 (3H, s), 6.95-7.05 (1H, m), 7.39 (1H, t, J=8.0 Hz), 7.50-7.52 (2H, m), 8.12 (1H, d, J=2.4 Hz), 8.38 (1H, d, J=2.4 Hz), 9.96 (1H, brs).

### Example 14

### 1-methyl-N-(6-(pyridin-4-yl)benzo[d]thiazol-2-yl)piperidine-4-carboxamide

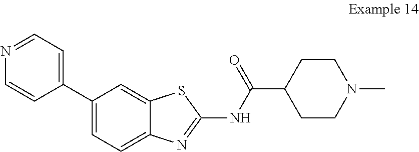 [SMILES: CN1CCC(C(=O)Nc2nc3ccc(-c4ccncc4)cc3s2)CC1]

To a solution of Intermediate 17 (100 mg, 0.440 mmol) in DMF (2 mL) was added 1-methylpiperidine-4-carboxylic acid (76 mg, 0.53 mmol), HATU (201 mg, 0.528 mmol) and DIPEA (114 mg, 0.880 mmol) at 25′C. Then the reaction mixture was stirred at 25° C. for 2 hours. The reaction mixture was diluted with water (10 mL) and extracted with EtOAc (10 mL×4). The combined organic layer was washed with brine (10 mL), dried over anhydrous Na₂SO₄, filtered and concentrated. The residue was purified by prep-HPLC (Method B; 0.05% NH₃·H₂O as an additive), then lyophilized to give the title compound (7.04 mg, yield: 4.4%) as a yellow solid.

¹H NMR (400 MHz, CDCl₃) δ 1.91-2.07 (6H, in), 2.31 (3H, s), 2.37-2.45 (1H, m), 2.92-3.00 (2H, m), 7.55-7.61 (2H, m), 7.73 (1H, d, J=8.4 Hz), 7.87 (1H, d, J=8.4 Hz), 8.09-8.148 (1H, m), 8.62-8.77 (2H, m), 9.43 (1H, brs).

The following compounds were synthesized analogously to Example 14

| Example | | | |
|---|---|---|---|
| No. | Structure | Name | ¹H NMR (400 MHz) |
| 23 | 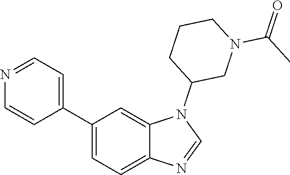 [SMILES: CC(=O)N1CCCC(n2cnc3ccc(-c4ccncc4)cc32)C1] | 1-(3-(6-(pyridin-4-yl)- 1H-benzo[d]imidazol-1- yl)piperidin-1-yl)ethan- 1-one | DMSO-d₆; δ 1.60-1.80 (1H, m), 1.84-1.93 (1H, m), 2.08 (3H, s), 2.18-2.31 (2H, m), 3.20-3.25 (2H, m), 3.55-3.60 (1H, m), 3.75-3.85 (1H, m), 4.60-4.65 (2H, m), 7.66 (1H, dd, J = 8.4, 1.6 Hz), 7.76-7.82 (3H, m), 8.13 (1H, s), 8.39 (1H, s), 8.61-8.67 (2H, m). |
| 38 | 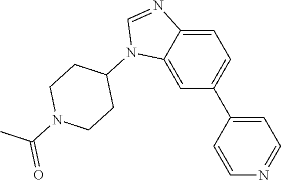 [SMILES: CC(=O)N1CCC(n2cnc3ccc(-c4ccncc4)cc32)CC1] | 1-(4-(6-(pyridin-4-yl)- 1H-benzo[d]imidazol-1- yl)piperidin-1-yl)ethan- 1-one | CD₃OD; δ 2.00-2.20 (2H, m), 2.22 (3H, s), 2.25-2.35 (2H, m), 2.85- 2.95 (1H, m), 3.40-3.50 (1H, m), 4.15-4.20 (1H, m), 4.80-4.85 (2H, m), 7.75 (1H, d, J = 8.8 Hz), 7.80- 7.90 (3H, m), 8.13 (1H, s), 8.44 (1H, s), 8.60-8.65 (2H, m). |
| 42 | 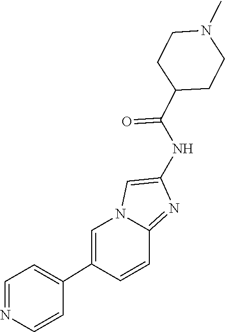 [SMILES: CN1CCC(C(=O)Nc2cn3cc(-c4ccncc4)ccc3n2)CC1] | 1-methyl-N-(6-(pyridin- 4-yl)imidazo[1,2- a]pyridin-2- yl)piperidine-4- carboxamide | DMSO-d₆; δ 1.59-1.79 (4H, m), 1.81-1.92 (2H, m), 2.16 (3H, s), 2.35-2.47 (1H, m), 2.80-2.85 (2H, m), 7.55 (1H, d, J = 9.2 Hz), 7.68 (1H, d, J = 9.2 Hz), 7.70-7.80 (2H, m), 8.15 (1H, s), 8.60-8.70 (2H, m), 9.15 (1H, s). |
| 58 | 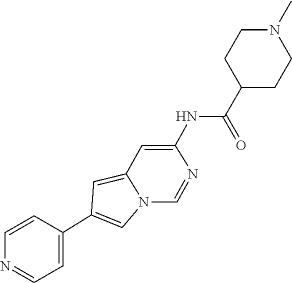 [SMILES: CN1CCC(C(=O)Nc2cc3cc(-c4ccncc4)cn3cn2)CC1] | 1-methyl-N-(6-(pyridin- 4-yl)pyrrolo[1,2- c]pyrimidin-3- yl)piperidine-4- carboxamide | DMSO-d₆; δ 1.59-1.79 (4H, m), 1.85-1.97 (2H, m), 2.19 (3H, s), 2.42-2.47 (1H, m), 2.80-2.90 (2H, m), 6.86 (1H, s), 7.69-7.75 (2H, m), 7.99 (1H, s), 8.16 (1H, s), 8.56-8.60 (2H, m), 9.05 (1H, s), 10.24 (1H, brs). |
| 64 | 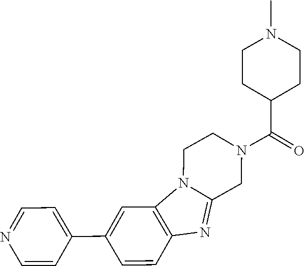 [SMILES: CN1CCC(C(=O)N2CCn3c(nc4ccc(-c5ccncc5)cc43)C2)CC1] | (1-methylpiperidin-4- yl)(7-(pyridin-4-yl)-3,4- dihydrobenzo[4,5]imidazo [1,2-a]pyrazin-2(1H)- yl)methanone | CD₃OD; δ 1.74-1.93 (4H, m), 2.10- 2.24 (2H, m), 2.30 (3H, s), 2.78- 2.90 (1H, m), 2.90-3.01 (2H, m), 4.17-4.25 (2H, m), 4.26-4.44 (2H, m), 5.01-5.14 (2H, m), 7.69-7.76 (2H, m), 7.79-7.83 (2H, m), 7.96 (1H, s), 8.56-8.61 (2H, m). |
| 65 | 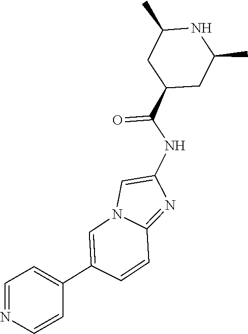 [SMILES: C[C@@H]1C[C@H](C(=O)Nc2cn3cc(-c4ccncc4)ccc3n2)C[C@H](C)N1] | (2R,4r,6S)-2,6- dimethyl-N-(6-(pyridin- 4-yl)imidazo[1,2- a]pyridin-2- yl)piperidine-4- carboxamide | DMSO-d₆; δ 1.26 (6H, d, J = 6.4 Hz, 6H), 1.50-1.65 (m, 2H, m), 1.94-2.10 (2H, m), 2.80-2.90 (1H, m), 3.20-3.30 (2H, m), 7.63 (1H, d, J = 9.6 Hz), 7.83 (1H, dd, J = 9.2, 1.6 Hz), 8.13-8.15 (2H, m), 8.20 (1H, s), 8.83-8.91 (2H, m), 9.37 (1H, d, J = 0.8 Hz), 11.02 (1H, brs). |
| 72 | 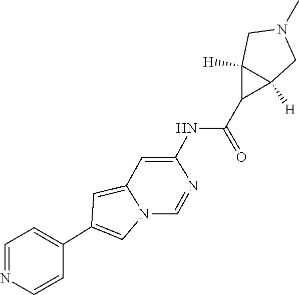 [SMILES: CN1C[C@@H]2C(C(=O)Nc3cc4cc(-c5ccncc5)cn4cn3)[C@@H]2C1] | (1R,5S)-3-methyl-N-(6- (pyridin-4- yl)pyrrolo[1,2- c]pyrimidin-3-yl)-3- azabicyclo[3.1.0]hexane- 6-carboxamide | CD₃OD; δ 2.02-2.04 (3H, m), 2.37 (3H, s), 2.50-2.55 (2H, m), 3.10- 3.15 (2H, m), 6.79 (1H, s), 7.70- 7.79 (2H, m), 7.95 (1H, s), 8.05- (1H, s), 8.50-8.55 (2H, m), 8.88 (1H, s). |
| 73 | 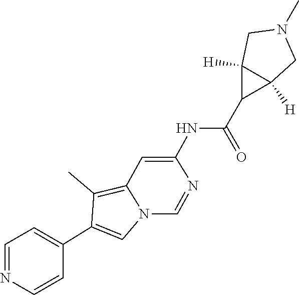 [SMILES: Cc1c(-c2ccncc2)cn2cnc(NC(=O)C3[C@H]4CN(C)C[C@@H]34)cc12] | (1R,5S)-3-methyl-N-(5- methyl-6-(pyridin-4- yl)pyrrolo[1,2- c]pyrimidin-3-yl)-3- azabicyclo[3.1.0]hexane- 6-carboxamide | CD₃OD; δ 2.03-2.08 (3H, m), 2.39 (3H, s), 2.40 (3H, s), 2.50-2.55 (2H, m), 3.10-3.15 (2H, m), 7.65-7.67 (2H, m), 7.80 (1H, s), 7.92 (1H, s), 8.56-8.59 (2H, m), 8.81 (1H, d, J = 1.6 Hz). |
| 74 | 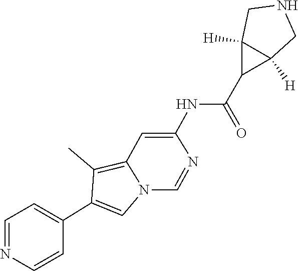 [SMILES: Cc1c(-c2ccncc2)cn2cnc(NC(=O)C3[C@H]4CNC[C@@H]34)cc12] | (1R,5S)-N-(5-methyl-6- (pyridin-4- yl)pyrrolo[1,2- c]pyrimidin-3-yl)-3- azabicyclo[3.1.0]hexane- 6-carboxamide | CD₃OD; δ 1.85-1.95 (1H, m), 2.30- 2.34 (2H, m), 2.36 (3H, s), 3.45- 3.55 (4H, m), 7.60-7.65 (2H, m), 7.77 (1H, s), 7.88 (1H, s), 8.45-8.60 (3H, m), 8.78 (1H, s). |

### Example 15

### 4-(3-cyclobutyl-1H-pyrrolo[2,3-b]pyridin-5-yl)benzamide

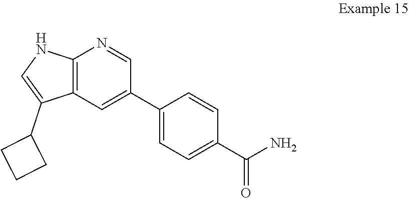 [SMILES: NC(=O)c1ccc(-c2cnc3[nH]cc(C4CCC4)c3c2)cc1]

To a solution of Intermediate 11 (100 mg, 0.398 mmol) and (4-carbamoylphenyl)boronic acid (85 mg, 0.52 mmol) in 1, 4-dioxane (2 mL) and H₂O (0.3 mL) was added Pd(PPh₃)₄ (46 mg, 0.040 mmol) and Na₂CO₃ (84 mg, 0.80 mmol) under N₂ atmosphere. The mixture was stirred at 100° C. for 16 hours under N₂ atmosphere. The reaction mixture was poured into water (15 mL) and extracted with EtOAc (15 mL×3). The combined organic layer was washed with brine (20 mL), dried over anhydrous Na₂SO₄, filtered and concentrated. The residue was purified by prep-HPLC (0.05% NH₃·H₂O as an additive) and lyophilized to give the title compound (12 mg, yield: 10%) as an off-white solid.

¹H NMR (400 MHz, DMSO-d₆) δ 1.84-2.210 (2H m), 2.13-2.27 (2H, m), 2.34-2.45 (2H, m), 3.67-3.82 (1H, m), 7.35 (1H, s), 7.39 (1H, brs), 7.81 (2H, d, J=8.4 Hz), 7.98 (2H, d, J=8.4 Hz), 8.03 (1H, brs), 8.21 (1H, d, J=2.0 Hz), 8.54 (1H, d, J=2.0 Hz), 11.47 (1H, brs).

The following compounds were synthesized analogously to Example 15

| Example | | | |
|---|---|---|---|
| No. | Structure | Name | ¹H NMR (400 MHz) |
| 16 | 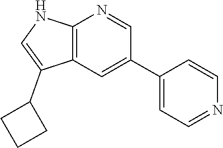 [SMILES: c1cc(-c2cnc3[nH]cc(C4CCC4)c3c2)ccn1] | 3-cyclobutyl-5-(pyridin-4- yl)-1H-pyrrolo[2,3- b]pyridine | CD₃OD; δ 1.93-2.04 (1H, m), 2.08-2.21 (1H, m), 2.21-2.33 (2H, m), 2.43-2.56 (2H, m), 3.76- 3.89 (1H, m), 7.26-7.32 (1H, m), 7.78-7.85 (2H, m), 8.14 (1H, s), 8.33 (1H, d, J = 2.0 Hz), 8.54- 8.65 (3H, m). |
| 30 | 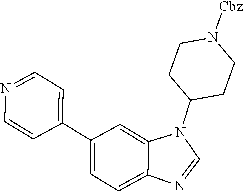 [SMILES: O=C(OCc1ccccc1)N1CCC(n2cnc3ccc(-c4ccncc4)cc32)CC1] | benzyl 4-(6-(pyridin-4-yl)- 1H-benzo[d]imidazol-1- yl)piperidine-1- carboxylate | CD₃OD; δ 2.10-2.20 (2H, m), 2.34-2.37 (2H, m), 3.15-3.21 (2H, m), 4.44-4.48 (2H, m), 4.95- 5.05 (1H, m), 5.19 (2H, s), 7.35- 7.45 (5H, m), 8.03 (1H, d, J = 8.4 Hz), 8.13 (1H, d, J = 8.4 Hz), 8.45-8.50 (2H, m), 8.59 (1H, s), 8.90-8.95 (2H, m), 9.25-9.30 (1H, m). |
| 48 | 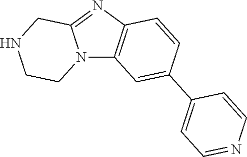 [SMILES: c1cc(-c2ccc3nc4n(c3c2)CCNC4)ccn1] | 7-(pyridin-4-yl)-1,2,3,4- tetrahydrobenzo[4,5]imidazo [1,2-a]pyrazine | DMSO-d₆; δ 3.81 (2H, t, J = 5.2 Hz), 4.57 (2H, t, J = 5.6 Hz), 4.68 (2H, s), 7.87 (1H, d, J = 8.63 Hz), 8.00 (1H, dd, J = 8.4, 2.0 Hz), 8.50-8.56 (3H, m), 8.97 (2H, d, J = 6.8 Hz), 10.47 (2H, brs). |
| 83 | 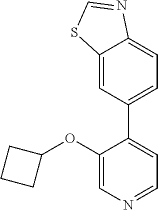 [SMILES: c1cc(-c2ccc3ncsc3c2)c(OC2CCC2)cn1] | 6-(3-cyclobutoxypyridin- 4-yl)benzo[d]thiazole | DMSO-d₆; δ 1.57-1.69 (1H, m), 1.72-1.82 (1H, m), 1.98-2.11 (2H, m), 2.40-2.46 (2H, m), 4.83- 4.91 (1H, m), 7.46 (1H, d, J = 4.8 Hz), 7.79 (1H, dd, J = 8.4, 1.6 Hz), 8.18 (1H, d, J = 3.6 Hz), 8.28-8.30 (2H, m), 8.42 (1H, d, J = 1.6 Hz), 9.46 (1H, s). |
| 86 | 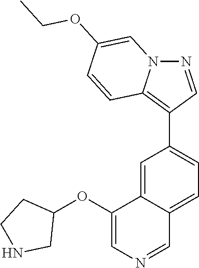 [SMILES: CCOc1ccc2c(-c3ccc4cncc(OC5CCNC5)c4c3)cnn2c1] | 6-(6-ethoxypyrazolo[1,5- a]pyridin-3-yl)-4- (pyrrolidin-3- yloxy)isoquinoline | DMSO-d₆; δ 1.39 (3H, t, J = 6.8 Hz), 2.28-2.43 (2H, m), 3.37-3.51 (2H, m), 3.52-3.64 (1H, m), 3.67- 3.79 (1H, m), 4.13 (2H, q, J = 6.8 Hz), 5.56-5.65 (1H, m), 7.35 (1H, dd, J = 9.6, 2.0 Hz), 8.22 (1H, d, J = 9.6 Hz), 8.37-8.44 (2H, m), 8.46-8.52 (1H, m), 8.52-8.59 (2H, m), 8.86 (1H, s), 9.43 (1H, s), 9.84 (1H, brs), 10.32 (1H, brs). |
| 87 | 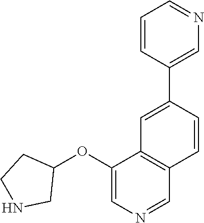 [SMILES: c1cncc(-c2ccc3cncc(OC4CCNC4)c3c2)c1] | 6-(pyridin-3-yl)-4- (pyrrolidin-3- yloxy)isoquinoline | DMSO-d₆; δ 2.32-2.39 (2H, m), 3.33-3.47 (2H, m), 3.48-3.60 (1H, m), 3.64-3.76 (1H, m), 5.61- 5.70 (1H, m), 7.92-8.01 (1H, m), 8.44 (1H, dd, J = 8.8, 1.6 Hz), 8.53 (1H, s), 8.59 (1H, d, J = 8.8 Hz), 8.88 (1H, dd, J = 5.2, 1.2 Hz), 8.92-9.03 (2H, m), 9.46-9.57 (2H, m), 9.74 (1H, brs), 10.33 (1H, brs). |

### Example 19

### 1-(piperidin-3-yl)-6-(pyridin-4-yl)-1H-benzo[d]imidazole

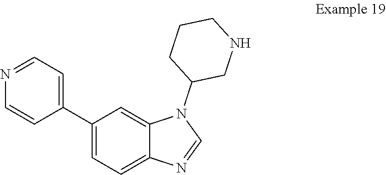 [SMILES: c1cc(-c2ccc3ncn(C4CCCNC4)c3c2)ccn1]

A solution of intermediate 12 (199 mg, 0.526 mmol) in 4N HCl/MeOH (5 mL) was stirred at 25° C. for 1 hour. The reaction mixture was concentrated to give the title compound (160 mg, yield: 97%, HCl salt) as a yellow solid.

¹H NMR (400 MHz, CDCl₃) δ 1.85-2.00 (1H, m), 2.05-2.15 (1H, m), 2.25-2.38 (2H, m), 2.85-3.05 (1H, m), 3.40-3.50 (2H, m), 3.70-3.75 (1H, m), 4.95-5.05 (1H, m), 7.92-7.98 (2H, m), 8.30-8.45 (3H, m), 8.81 (1H, s), 8.90-8.95 (2H, m), 9.25-9.45 (2H, m).

The following compound was synthesized analogously to Example 19

| Example | | | |
|---|---|---|---|
| No. | Structure | Name | ¹H NMR (400 MHz) |
| 31 | 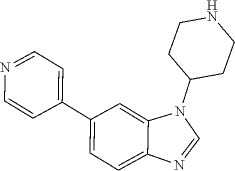 [SMILES: c1cc(-c2ccc3ncn(C4CCNCC4)c3c2)ccn1] | 1-(piperidin-4-yl)-6- (pyridin-4-yl)-1H- benzo[d]imidazole | CD₃OD; δ 2.09-2.28 (4H, m), 2.86- 2.99 (2H, m), 3.25-3.28 (2H, m), 4.65-4.75 (1H, m), 7.72-7.76 (1H, m), 7.83 (1H, d, J = 8.4 Hz), 7.85- 7.90 (2H, m), 8.16 (1H, s), 8.40 (1H, s), 8.60-8.65 (2H, m). |

### Example 20

### 1-(1-(methylsulfonyl)piperidin-3-yl)-6-(pyridin-4-yl)-1H-benzo[d]imidazole

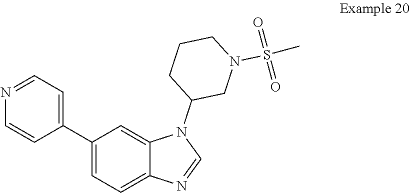 [SMILES: CS(=O)(=O)N1CCCC(n2cnc3ccc(-c4ccncc4)cc32)C1]

To a solution of Example 19 (170 mg, 0.540 mmol, HCl salt) and Et₃N (164 mg, 1.62 mmol) in DCM (5 mL) was added MsCl (74 mg, 0.65 mmol) at 0° C. The mixture was stirred at 0° C. for 1 hour. The reaction mixture was quenched with water (50 mL) and extracted with DCM (30 mL×3). The combined organic layer was washed with brine (30 mL×3), dried over anhydrous Na₂SO₄, filtered and concentrated. The residue was purified by prep-HPLC (0.05% NH₃·H₂O as an additive), then lyophilized to give the title compound (16.25 mg, yield: 8%) as a white solid.

¹H NMR (400 MHz, DMSO-d₆) δ 1.70-1.86 (1H, m), 1.85-2.00 (1H, m), 2.05-2.25 (2H, m), 2.90-3.00 (4H, m), 3.25-3.35 (1H, m), 3.55-3.65 (1H, m), 3.75-3.85 (1H, m), 4.80-4.90 (1H, m), 7.69 (1H, dd, J=8.4, 2.0 Hz), 7.76-7.89 (3H, m), 8.21 (1H, s), 8.50 (1H, s), 8.62-8.70 (2H, m).

The following compounds were synthesized analogously to Example 20

| Example | | | |
|---|---|---|---|
| No. | Structure | Name | ¹H NMR (400 MHz) |
| 32 | 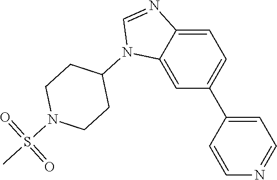 [SMILES: CS(=O)(=O)N1CCC(n2cnc3ccc(-c4ccncc4)cc32)CC1] | 1-(1- (methylsulfonyl)piperidin- 4-yl)-6-(pyridin-4-yl)-1H- benzo[d]imidazole | CD₃OD; δ 2.25-2.45 (4H, m), 2.98 (3H, s), 3.05-3.16 (2H, m), 4.00-4.06 (2H, m), 5.00-5.05 (1H, m), 7.95-8.10 (2H, m), 8.40- 8.55 (3H, m), 8.85-8.90 (2H, m), 8.95-9.20 (1H, m). |
| 33 | 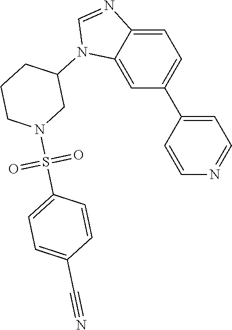 [SMILES: N#Cc1ccc(S(=O)(=O)N2CCCC(n3cnc4ccc(-c5ccncc5)cc43)C2)cc1] | 4-((3-(6-(pyridin-4-yl)- 1H-benzo[d]imidazol-1- yl)piperidin-1- yl)sulfonyl)benzonitrile | CDCl₃; δ 1.99-2.15 (3H, m), 2.27-2.47 (1H, m), 2.60-2.85 (2H, m), 3.79-3.94 (1H, m), 4.08- 4.24 (1H, m), 4.56-4.72 (1H, m), 7.58-7.63 (2H, m), 7.64 (1H, dd, J = 8.4, 1.6 Hz), 7.71 (1H, s), 7.84-7.92 (4H, m), 7.95 (1H, d, J = 8.8 Hz), 8.07 (1H, s), 8.69-8.77 (2H, m). |
| 39 | 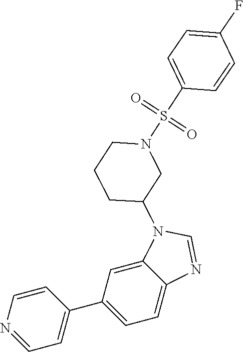 [SMILES: O=S(=O)(c1ccc(F)cc1)N1CCCC(n2cnc3ccc(-c4ccncc4)cc32)C1] | 1-(1-((4- fluorophenyl)sulfonyl) piperidin-3-yl)-6-(pyridin-4- yl)-1H-benzo[d]imidazole | CD₃OD; δ 1.85-2.03 (2H, m), 2.04-2.16 (1H, m), 2.02-2.23 (1H, m), 2.73-2.87 (1H, m), 2.95- 3.09 (1H, m), 3.35-3.38 (1H, m), 3.60-3.63 (1H, m), 3.90-3.92 (1H, m), 7.35-7.37 (2H, m), 7.71- 7.76 (1H, m), 7.80-7.85 (3H, m), 7.88-7.90 (2H, m), 8.04 (1H, s), 8.45 (1H, s), 8.60-8.70 (2H, m). |

### Example 21 and 22

### (S)-1-(1-(methylsulfonyl)piperidin-3-yl)-6-(pyridin-4-yl)-1H-benzo[d]imidazole (21) & (R)-1-(1-methylsulfonyl)piperidin-3-yl)-6-(pyridin-4-yl)-1H-benzo[d]imidazole (21)

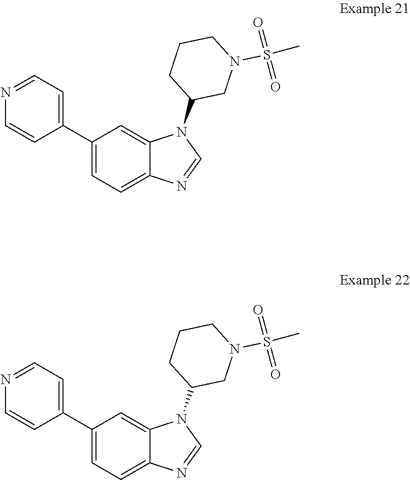

Example 20 (40 mg) was submitted for SFC separation (Column: DAICEL CHIRALCEL OD: 250 mm×30 mm, 10 um; Eluent of 35˜35% EtOH/0.1% NH₃H₂O) to give 2 fractions. The first fraction (peak 1) was concentrated and lyophilized to give Example 21 (12 mg, yield: 60%, ee=98.26%) as a white solid. The second fraction (peak 2) was concentrated and lyophilized to give Example 22 (17 mg, yield: 85%, ee=100%) as a white solid. Stereochemistry assigned randomly.

Example 21: ¹H NMR (400 MHz, DMSO-d₆) δ 1.80-2.04 (2H, m), 2.10-2.29 (2H, m), 3.03 (3H, s), 3.30-3.35 (1H, m), 3.38-3.40 (1H, m), 3.60-3.70 (1H, m), 3.80-3.90 (1H, m), 4.83-5.00 (1H, m), 7.74 (1H, dd, J=8.4, 2.0 Hz), 7.84-7.90 (3H, m), 8.25 (1H, s), 8.55 (1H, s), 8.69-8.72 (2H, m).

Example 22: ¹H NMR (400 MHz, DMSO-d₆) δ 1.74-1.98 (2H, m), 2.04-2.23 (2H, m), 2.97 (3H, s), 3.30-3.35 (2H, m), 3.55-3.60 (1H, m), 3.75-3.85 (1H, m), 4.83-5.00 (1H, m), 7.69 (1H, dd, J=8.4, 2.0 Hz), 7.78-7.84 (3H, m), 8.16 (1H, s), 8.50 (1H, s), 8.62-8.70 (2H, m).

### Example 24

### N-(4-fluorobenzyl)-6-(pyridin-4-yl)imidazo[1,2-a]pyridine-3-carboxamide

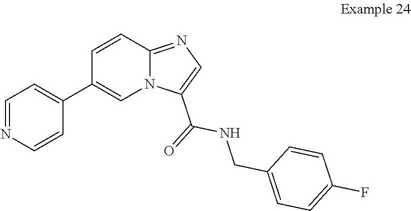 [SMILES: O=C(NCc1ccc(F)cc1)c1cnc2ccc(-c3ccncc3)cn12]

### Step 1. Synthesis of 6-bromo-N-(4-fluorobenzyl)imidazo[1,2-a]pyridine-3-carboxamide

To a solution of 6-bromoimidazo[1,2-a]pyridine-3-carboxylic acid (300 mg, 1.24 mmol), (4-fluorophenyl)methanamine (156 mg, 1.24 mmol) in DMF (4 mL) was added DIPEA (322 mg, 2.49 mmol) and HATU (568 mg, 1.49 mmol), then the mixture was stirred at 25° C. for 12 hours. The reaction mixture was diluted with H₂O (20 mL) and extracted with EtOAc (30 mL×3). The combined organic layers were washed with brine (50 mL), dried over anhydrous Na₂SO₄, filtered and concentrated. The residue was purified by prep-TLC (SiO₂, DCM/MeOH=10/1) to give 6-bromo-N-(4-fluorobenzyl)imidazo[1,2-a]pyridine-3-carboxamide (280 mg, yield: 65%) as yellow oil.

### Step 2. Synthesis of N-(4-fluorobenzyl)-6-(pyridin-4-yl)imidazo[1,2-a]pyridine-3-carboxamide

To a solution of 6-bromo-N-(4-fluorobenzyl)imidazo[1,2-a]pyridine-3-carboxamide (280 mg, 0.804 mmol), pyridin-4-ylboronic acid (119 mg, 0.965 mmol), Pd(dppf)Cl₂ (66 mg, 0.080 mmol) and Na₂CO₃ (256 mg, 2.41 mmol) in 1, 4-dioxane (4 mL) and H₂O (0.5 mL) was degassed and purged with N₂ for 3 times, and then the mixture was stirred at 90° C. for 6 hours under N₂ atmosphere. The reaction mixture was concentrated and the residue was diluted with H₂O (30 mL), then extracted with EtOAc (40 mL×3). The combined organic layers were washed with brine (80 mL), dried over anhydrous Na₂SO₄, filtered and concentrated. The residue was purified by prep-TLC (SiO₂, DCM/MeOH=10/1) to afford the title compound (28.76 mg, yield: 10%) as an off-white solid.

¹HNMR (400 MHz, CD₃OD) δ 4.60 (2H, s), 7.02-7.14 (2H, m), 7.37-7.48 (2H, m), 7.80-7.86 (3H, m), 7.94 (1H, dd, J=9.2, 1.6 Hz), 8.33 (1H, s), 8.62-8.71 (2H, m), 9.97 (1H, s).

The following compounds were synthesized analogously to Example 24

| Example | | | |
|---|---|---|---|
| No. | Structure | Name | ¹H NMR (400 MHz) |
| 25 | 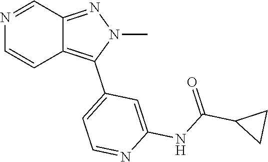 [SMILES: Cn1nc2cnccc2c1-c1ccnc(NC(=O)C2CC2)c1] | N-(4-(2-methyl-2H- pyrazolo[3,4-c]pyridin-3- yl)pyridin-2- yl)cyclopropane- carboxamide | DMSO-d₆; δ 0.82-0.89 (4H, m), 2.03-2.10 (1H, m), 4.33 (3H, s), 7.48 (1H, dd, J = 5.2, 1.6 Hz), 7.63 (1H, dd, J = 6.0 Hz), 8.19 (1H, d, J = 6.0 Hz), 8.39 (1H, s), 8.55 (1H, d, J = 4.8 Hz), 9.24 (1H, d, J = 1.2 Hz), 11.09 (1H, brs). |
| 34 | 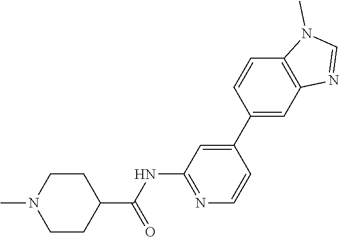 [SMILES: CN1CCC(C(=O)Nc2cc(-c3ccc4c(c3)ncn4C)ccn2)CC1] | 1-methyl-N-(4-(1- methyl-1H- benzo[d]imidazol-5- yl)pyridin-2- yl)piperidine-4- carboxamide | DMSO-d₆; δ 1.62-1.92 (6H, m), 2.16 (3H, s), 2.80-2.85 (2H, m), 3.93 (3H, s), 7.48-7.50 (1H, m), 7.58 (1H, d, J = 8.4 Hz), 7.77 (1H, d, J = 8.4 Hz), 7.95 (1H, s), 8.28 (1H, s), 8.37 (1H, d, J = 5.6 Hz), 8.51 (1H, s), 10.50 (1H, brs). |
| 51 | 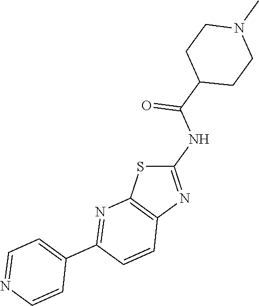 [SMILES: CN1CCC(C(=O)Nc2nc3ccc(-c4ccncc4)nc3s2)CC1] | 1-methyl-N-(5-(pyridin- 4-yl)thiazolo[5,4- b]pyridin-2- yl)piperidine-4- carboxamide | CD₃OD; δ 1.99-2.11 (2H, m), 2.12-2.21 (2H, m), 2.73 (3H, s), 2.80-2.90 (3H, m), 3.35-3.45 (2H, m), 8.09-8.23 (4H, m), 8.65-8.70 (2H, m). |
| 55 | 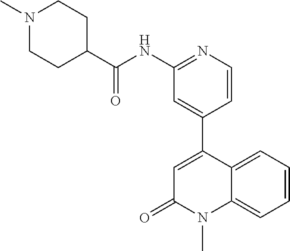 [SMILES: CN1CCC(C(=O)Nc2cc(-c3cc(=O)n(C)c4ccccc34)ccn2)CC1] | 1-methyl-N-(4-(1- methyl-2-oxo-1,2- dihydroquinolin-4- yl)pyridin-2- yl)piperidine-4- carboxamide | DMSO-d₆; δ 1.58-1.73 (2H, m), 1.75-1.85 (2H, m), 2.00-2.12 (2H, m), 2.26 (3H, s), 2.52-2.59 (1H, m), 2.90-2.95 (2H, m), 3.70 (3H, s), 6.58 (1H, s), 7.20-7.30 (2H, m), 7.42 (1H, d, J = 7.6 Hz), 7.62-7.74 (2H, m), 8.15-8.25 (2H, m), 8.47 (1H, d, J = 4.4 Hz), 10.73 (1H, brs). |
| 56 | 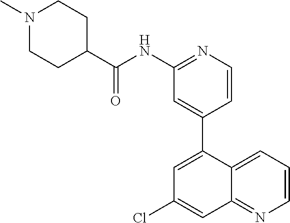 [SMILES: CN1CCC(C(=O)Nc2cc(-c3cc(Cl)cc4ncccc34)ccn2)CC1] | N-(4-(7-chloroquinolin- 5-yl)pyridin-2-yl)-1- methylpiperidine-4- carboxamide | DMSO-d₆; δ 1.56-1.71 (2H, m), 1.72-1.83 (2H, m), 1.90-2.00 (2H, m), 2.21 (s, 3H), 2.52-2.55 (1H, m), 2.85-2.88 (2H, m), 7.28 (1H, dd, J = 5.2, 1.6 Hz), 7.57-7.71 (2H, m), 8.17-8.25 (4H, m), 8.48 (1H, d, J = 5.2 Hz), 9.01 (1H, dd, J = 4.0, 1.6 Hz), 10.73 (1H, brs). |
| 57 | 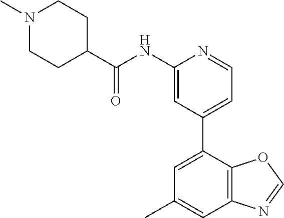 [SMILES: Cc1cc(-c2ccnc(NC(=O)C3CCN(C)CC3)c2)c2ocnc2c1] | 1-methyl-N-(4-(5- methylbenzo[d]oxazol-7- yl)pyridin-2- yl)piperidine-4- carboxamide | CD₃OD; δ 1.88-2.00 (4H, m), 2.11-2.20 (2H, m), 2.32 (3H, s), 2.49-2.57 (1H, m), 2.58 (3H, s), 2.94-3.06 (2H, m), 7.64-7.70 (3H, m), 8.39-8.46 (1H, m), 8.56 (1H, s), 8.72 (1H, s). |
| 59 | 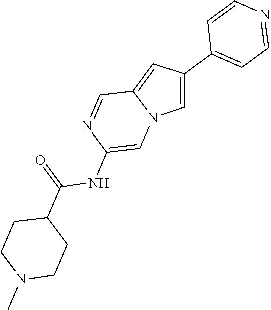 [SMILES: CN1CCC(C(=O)Nc2cn3cc(-c4ccncc4)cc3cn2)CC1] | 1-methyl-N-(7-(pyridin- 4-yl)pyrrolo[1,2- a]pyrazin-3- yl)piperidine-4- carboxamide | DMSO-d₆; δ 1.70-1.82 (4H, m), 2.01-2.10 (3H, m), 2.29 (3H, s), 2.90-3.01 (2H, m), 7.39 (1H, s), 7.72-7.81 (2H, m), 8.48 (1H, s), 8.55-8.67 (2H, m), 8.81 (1H, s), 9.02 (1H, s), 10.33 (1H, br s). |
| 60 | 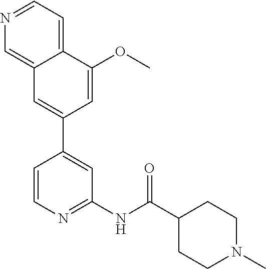 [SMILES: COc1cc(-c2ccnc(NC(=O)C3CCN(C)CC3)c2)cc2cnccc12] | N-(4-(5- methoxyisoquinolin-7- yl)pyridin-2-yl)-1- methylpiperidine-4- carboxamide | DMSO-d₆; δ 1.64-1.73 (2H, m), 1.74-1.82 (2H, m), 1.83-1.91 (2H, m), 2.17 (3H, s), 2.43-2.46 (1H, m), 2.77-2.86 (2H, m), 4.13 (3H, s), 7.46-7.50 (1H, m), 7.61 (1H, d, J = 4.8 Hz), 7.98 (1H, d, J = 6.0 Hz), 8.06 (1H, s), 8.45 (1H, d, J = 4.8 Hz), 8.55-8.62 (2H, m), 9.40- 9.47 (1H, m), 10.59 (1H, brs). |
| 63 | 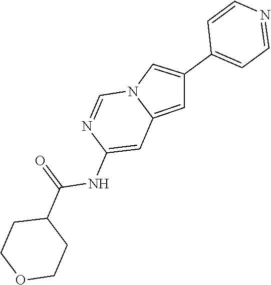 [SMILES: O=C(Nc1cc2cc(-c3ccncc3)cn2cn1)C1CCOCC1] | N-(6-(pyridin-4- yl)pyrrolo[1,2- c]pyrimidin-3- yl)tetrahydro-2H-pyran- 4-carboxamide | DMSO-d₆; δ 1.57-1.77 (4H, m), 2.70-2.80 (1H, m), 3.40-3.50 (2H, m), 3.83-3.97 (2H, m), 6.87 (1H, s), 7.70-7.77 (2H, m), 7.99 (1H, s), 8.13-8.20 (1H, m), 8.54-8.61 (2H, m), 9.05 (1H, s), 10.27 (1H, brs). |
| 67 | 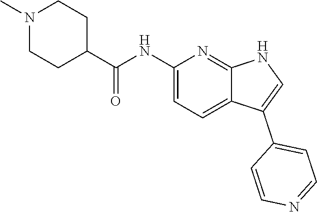 [SMILES: CN1CCC(C(=O)Nc2ccc3c(-c4ccncc4)c[nH]c3n2)CC1] | 1-methyl-N-(3-(pyridin- 4-yl)-1H-pyrrolo[2,3- b]pyridin-6- yl)piperidine-4- carboxamide | DMSO-d₆; δ 1.60-1.70 (2H, m), 1.72-1.79 (2H, m), 1.80-1.91 (2H, m), 2.16 (3H, s), 2.40-2.45 (1H, m), 2.75-2.85 (2H, m), 7.70-7.80 (2H, m), 8.01 (1H, d, J = 8.8 Hz, 8.09 (1H, s), 8.40 (1H, d, J = 8.4 Hz), 8.50-8.55 (2H, m), 10.31 (1H, brs), 11.96 (1H, brs). |
| 71 | 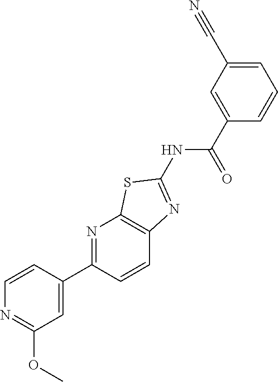 [SMILES: COc1cc(-c2ccc3nc(NC(=O)c4cccc(C#N)c4)sc3n2)ccn1] | 3-cyano-N-(5-(2- methoxypyridin-4- yl)thiazolo[5,4-b]pyridin- 2-yl)benzamide | DMSO-d₆; δ 3.92 (3H, s), 7.53 (1H, s), 7.74 (1H, d, J = 5.6 Hz), 7.80 (1H, t, J = 7.6 Hz), 8.14 (1H, d, J = 8.0 Hz), 8.20-8.27 (2H, m), 8.30 (1H, d, J = 5.2 Hz), 8.41 (1H, d, J = 8.4 Hz), 8.59 (1H, s), 13.33 (1H, brs). |
| 93 | 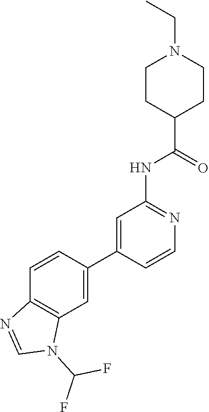 [SMILES: CCN1CCC(C(=O)Nc2cc(-c3ccc4ncn(C(F)F)c4c3)ccn2)CC1] | N-(4-(1- (difluoromethyl)-1H- benzo[d]imidazol-6- yl)pyridin-2-yl)-1- ethylpiperidine-4- carboxamide | DMSO-d₆; δ 0.99 (3H, t, J = 7.2 Hz), 1.57-1.69 (2H, m), 1.73-1.90 (4H, m), 2.32 (2H, q, J = 7.2 Hz), 2.52-2.53 (1H, m), 2.86-2.97 (2H, m), 7.47 (1H, dd, J = 5.2, 2.0 Hz), 7.73 (1H, dd, J = 8.4, 1.6 Hz), 7.92 (1H, d, J = 8.4 Hz), 8.02-8.36 (2H, m), 8.39 (1H, d, J = 4.8 Hz), 8.49 (1H, d, J = 1.2 Hz), 8.72 (1H, s), 10.55 (1H, brs). |
| 94 | 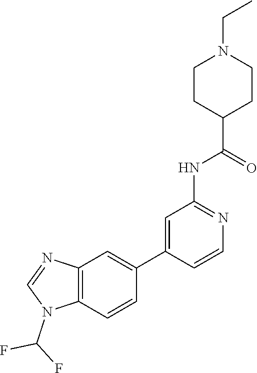 [SMILES: CCN1CCC(C(=O)Nc2cc(-c3ccc4c(c3)ncn4C(F)F)ccn2)CC1] | N-(4-(1- (difluoromethyl)-1H- benzo[d]imidazol-5- yl)pyridin-2-yl)-1- ethylpiperidine-4- carboxamide | DMSO-d₆; δ 0.99 (3H, t, J = 7.2 Hz), 1.56-1.71 (2H, m), 1.73-1.91 (4H, m), 2.31 (2H, q, J = 7.2 Hz), 2.52-2.55 (1H, m), 2.86-2.96 (2H, m), 7.50 (1H, dd, J = 5.2, 1.6 Hz), 7.78 (1H, dd, J = 8.4, 1.6 Hz), 7.85-7.91 (1H, m), 7.97-8.31 (2H, m), 8.37 (1H, d, J = 5.6 Hz), 8.49 (1H, d, J = 0.8 Hz), 8.69 (1H, s), 10.51 (1H, brs). |

### Example 26

### N-(4-(2-(2-methoxyethyl)-2H-pyrazolo[3,4-c]pyridin-3-yl)pyridin-2-yl)cyclopropanecarboxamide

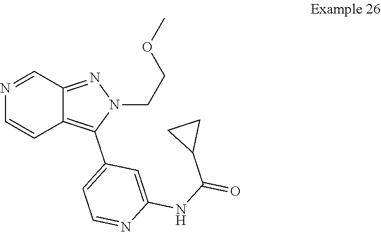 [SMILES: COCCn1nc2cnccc2c1-c1ccnc(NC(=O)C2CC2)c1]

### Step 1. Synthesis of 2-(2-methoxyethyl)-2H-pyrazolo[3,4-c]pyridine

To a solution of NaH (470 mg, 11.7 mmol, 60% dispersion in mineral oil) in DMF (5 mL) was added 2H-pyrazolo[3,4-c]pyridine (1.40 g, 11.7 mmol) in DMF (3 mL) dropwise at 0° C., the mixture was stirred at 0° C. for 1 hour. A solution of compound 2 (1.80 g, 12.9 mmol) in DMF (3 mL) was added dropwise to the mixture, the mixture was stirred at 20° C. for 16 hours. The reaction mixture was quenched with water (15 mL) and extracted with EtOAc (15 mL×3), the combined organic layer was washed with brine (20 mL), dried over anhydrous Na₂SO₄, filtered and concentrated. The residue was purified by flash silica gel chromatography (Eluent of 0˜100% Ethyl acetate/Petroleum ether gradient) to give 2-(2-methoxyethyl)-2H-pyrazolo[3,4-c]pyridine (330 mg, yield: 16%) as a yellow solid.

¹H NMR (400 MHz, CDCl₃) δ 3.35 (3H, s), 3.91 (2H, t, J=5.2 Hz), 4.66 (2H, t, J=5.2 Hz), 7.52 (1H, dd, J=6.0, 1.2 Hz), 8.08 (1H, s), 8.17 (1H, d, J=6.0 Hz), 9.27 (1H, s).

### Step 2. Synthesis of 3-bromo-2-(2-methoxyethyl)-2H-pyrazolo[3,4-c]pyridine

To a solution of 2-(2-methoxyethyl)-2H-pyrazolo[3,4-c]pyridine (180 mg, 1.02 mmol) in DMF (2 mL) was added NBS (180 mg, 1.02 mmol), the mixture was stirred at 20° C. for 2 hours, then stirred at 80° C. for 14 hours. The reaction mixture was concentrated and the residue was purified by flash silica gel chromatography (Eluent of 0˜80% Ethyl acetate/Petroleum ether gradient) to give 3-bromo-2-(2-methoxyethyl)-2H-pyrazolo[3,4-c]pyridine (23 mg, yield: 9%) as a light yellow solid.

¹H NMR (400 MHz, CDCl₃) δ 3.35 (3H, s), 3.97 (2H, t, J=5.6 Hz), 4.73 (2H, t, J=5.6 Hz), 7.39 (1H, dd, J=6.0, 1.2 Hz), 8.21 (1H, d, J=6.0 Hz), 9.24 (1H, s).

### Step 3. Synthesis of N-(4-(2-(2-methoxyethyl)-2H-pyrazolo[3,4-c]pyridin-3-yl)pyridin-2-yl)cyclopropanecarboxamide

To a solution of 3-bromo-2-(2-methoxyethyl)-2H-pyrazolo[3,4-c]pyridine (23 mg, 0.089 mmol) and N-(4-(4,4,5,5-tetramethyl-1,3,2-dioxaborolan-2-yl)pyridin-2-yl)cyclopropanecarboxamide (25-c; 38 mg, 0.13 mmol) in 1, 4-dioxane (2 mL) and H₂O (0.5 mL) was added Pd(dppf)Cl₂ (13 mg, 0.018 mol) and Na₂CO₃ (19 mg, 0.18 mmol) under N₂ atmosphere. The mixture was stirred at 90° C. for 16 hours under N₂ atmosphere. The reaction mixture was concentrated and the residue was purified by prep-HPLC (0.04% NH₃H₂O+10 mM NH₄HCO₃ as an additive) and lyophilized to give the title compound (8 mg, yield: 28%) as a white solid.

¹H NMR (400 MHz, CDCl₃) δ 0.91-0.99 (2H, m), 1.11-1.18 (2H, m), 1.61-1.65 (1H, m), 3.28 (3H, s), 4.02 (2H, t, J=5.2 Hz), 4.71 (2H, t, J=5.6 Hz), 7.37 (1H, dd, J=4.8, 1.6 Hz), 7.52 (1H, dd, J=6.0, 1.2 Hz), 8.22 (1H, d, J=6.0 Hz), 8.27 (1H, brs), 8.44-8.51 (2H, m), 9.30 (1H, s).

### Example 27

### (1H-indazol-4-yl)(6-(pyridin-4-yl)imidazo[1,2-a]pyridin-3-yl)methanol

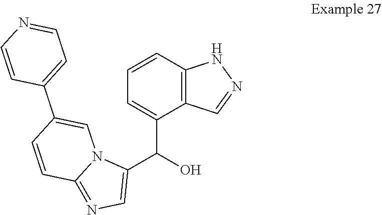 [SMILES: OC(c1cccc2[nH]ncc12)c1cnc2ccc(-c3ccncc3)cn12]

### Step 1. Synthesis of (6-(pyridin-4-yl)imidazo[1,2-a]pyridin-3-yl)(1-(tetrahydro-2H-pyran-2-yl)-1H-indazol-4-yl)methanol

To a solution of Intermediate 14 (300 mg, 1.34 mmol) in anhydrous THF (10 mL) was added n-BuLi (0.64 mL, 1.60 mmol, 2.5 M in hexane) dropwise at −70° C. under N₂ atmosphere, then the reaction mixture was stirred at −70° C. for 30 minutes. Intermediate 13 (491 mg, 1.75 mmol) was added to this solution at −70° C. and stirred for another 2.5 hours under N₂ atmosphere. The reaction mixture was quenched with water (30 mL) and extracted with EtOAc (30 mL×3). The combined organic layer was washed with brine (30 mL), dried over anhydrous Na₂SO₄, filtered and concentrated to give (6-(pyridin-4-yl)imidazo[1,2-a]pyridin-3-yl)(1-(tetrahydro-2H-pyran-2-yl)-1H-indazol-4-yl)methanol (300 mg, yield: 24%) as a yellow solid.

### Step 2. Synthesis of (1H-indazol-4-yl)(6-(pyridin-4-yl)imidazo[1,2-a]pyridin-3-yl)methanol

To a solution of (6-(pyridin-4-yl)imidazo[1,2-a]pyridin-3-yl)(1-(tetrahydro-2H-pyran-2-yl)-1H-indazol-4-yl)methanol (20 mg, 0.047 mmol) in anhydrous DCM (1 mL) was added TFA (1 mL) and the reaction mixture was stirred at 25° C. for 1 hour. The reaction mixture was basified with saturated aqueous NaHCO₃ to pH=8 and extracted with DCM (15 mL×3). The combined organic layer was concentrated and the residue was purified by prep-HPLC (0.05% NH₃H₂O+10 mM NH₄HCO₃ as an additive), then lyophilized to give the title compound (0.92 mg, yield: 5%) as a white solid.

¹H NMR (400 MHz, MeOD) δ 6.98 (1H, s), 7.20-7.27 (1H, m), 7.41-7.51 (1H, m), 7.58 (1H, d, J=8.8 Hz), 8.02 (1H, d, J=8.4 Hz), 8.14 (1H, s), 8.22 (1H, d, J=9.2 Hz), 8.45-8.50 (2H, m), 8.54 (1H, dd, J=8.0, 1.6 Hz), 9.00-9.05 (2H, m), 9.59 (1H, s).

### Example 28

### 4-((6-(pyridin-4-yl)imidazo[1,2-a]pyridin-3-yl)methyl)-1H-indazole

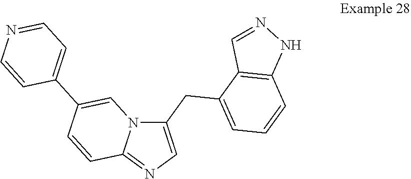 [SMILES: c1cc(Cc2cnc3ccc(-c4ccncc4)cn23)c2cn[nH]c2c1]

A solution of (6-(pyridin-4-yl)imidazo[1,2-a]pyridin-3-yl)(1-(tetrahydro-2H-pyran-2-yl)-1H-indazol-4-yl)methanol (from Example 27; 200 mg, 0.470 mmol) and Et₃SiH (7.28 g, 62.0 mmol) in anhydrous DCM (2 mL) and TFA (15.4 g, 135 mmol) was stirred at 25° C. for 12 hours. The reaction mixture was concentrated and the residue was diluted basified with saturated aqueous NaHCO₃ to pH=8, then extracted with DCM (20 mL×3). The combined organic layer was washed with brine (20 mL), dried over anhydrous Na₂SO₄, filtered and concentrated. The residue was purified by prep-HPLC (0.04% NH₃H₂O+10 mM NH₄HCO₃ as an additive) and lyophilized to give the title compound (20 mg, yield: 12%) as a yellow solid.

¹H NMR (400 MHz, MeOD) δ 4.79 (2H, s), 7.10 (1H, t, J=7.2 Hz), 7.47 (1H, t, J=6.0 Hz), 7.68 (1H, d, J=8.4 Hz), 7.69 (1H, s), 7.70-7.80 (5H, m), 8.58-8.63 (2H, m), 8.80 (1H, s).

### Example 29

### 3-(3-(1H-benzo[d]imidazol-2-yl)-1H-indazol-5-yl)benzonitrile

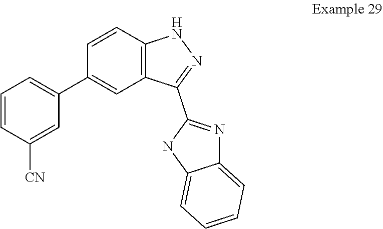

### Step 1. Synthesis of 2-(3-formyl-1H-indazol-5-yl)benzonitrile

A mixture of 5-bromo-1H-indazole-3-carbaldehyde (100 mg, 0.444 mmol), (3-cyanophenyl)boronic acid (98 mg, 0.67 mmol), Pd(dppf)Cl₂ (65 mg, 0.089 mmol) and Na₂CO₃ (141 mg, 1.33 mmol) in dioxane (4 mL) and water (1 mL) was degassed and purged with N₂ for 3 times, and then the mixture was stirred at 90° C. for 16 hours under N₂ atmosphere. The reaction mixture was filtered and the filtrate was concentrated. The residue was purified by flash silica gel chromatography (ISCO®; 4 g SepaFlash® Silica Flash Column, Eluent of 0˜52 Ethyl acetate/Petroleum ether gradient @20 mL/min) to give 2-(3-formyl-1H-indazol-5-yl)benzonitrile (20 mg, yield: 18%) as a brown solid.

¹H NMR (400 MHz, CDCl₃) δ 7.50-7.55 (1H, m), 7.59-7.67 (3H, m), 7.80-7.92 (2H, m), 8.46 (1H, s), 10.26 (1H, s), 10.51 (1H, brs).

### Step 2. Synthesis of 3-(3-(1H-benzo[d]imidazol-2-yl)-1H-indazol-5-yl)benzonitrile

A solution of 2-(3-formyl-1H-indazol-5-yl)benzonitrile (20 mg, 0.081 mmol) and benzene-1, 2-diamine (26 mg, 0.24 mmol) in THF (5 mL) containing 2N aqueous HCl (0.08 mL) was stirred at 60° C. for 30 minutes. Then NaHSO₃ (8 mg, 0.08 mmol) was added to the reaction mixture and the resulting mixture was stirred at 60° C. for another 16 hours. The reaction mixture filtered and the filtrate was concentrated. The residue was purified by prep-HPLC (Method A; 0.05% TFA as an additive) and lyophilized to give the title compound (11 mg, yield: 40%, TFA salt) as a white solid.

¹H NMR (400 MHz, CD₃OD) δ 7.58-7.64 (2H, m), 7.71-7.76 (1H, m), 7.78-7.82 (1H, m), 7.84-7.92 (3H, m), 7.94-7.98 (1H, m), 8.16 (1H, d, J=8.0 Hz), 8.25 (1H, s), 8.75 (1H, s).

### Example 40

### 1-(1-(methylsulfonyl)azepan-3-yl)-6-(pyridin-4-yl)-1H-benzo[d]imidazole

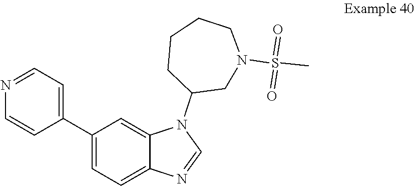 [SMILES: CS(=O)(=O)N1CCCCC(n2cnc3ccc(-c4ccncc4)cc32)C1]

### Step 1. Synthesis of tert-butyl 3-((5-bromo-2-nitrophenyl)amino)azepane-1-carboxylate

A mixture of 4-bromo-2-fluoro-1-nitrobenzene (840 mg, 3.82 mmol), tert-butyl 3-aminoazepane-1-carboxylate (900 mg, 4.20 mmol), Na₂CO₃ (2.02 g, 19.1 mmol) in DMF (15 mL) was stirred at 80° C. for 1 hour under N₂ atmosphere. The reaction mixture was diluted with water (100 mL) and extracted with EtOAc (50 mL×3). The combined organic layer was washed with brine (100 mL×3), dried over anhydrous Na₂SO₄, filtered and concentrated. The residue was purified by flash silica gel chromatography (ISCO®; 20 g SepaFlash® Silica Flash Column, Eluent of 0˜15% Ethyl acetate/Petroleum ether gradient @25 mL/min) to give tert-butyl 3-((5-bromo-2-nitrophenyl)amino)azepane-1-carboxylate (1.20 g, yield: 76%) as yellow oil.

¹H NMR (400 MHz, CDCl₃) δ 1.46 (9H, s), 1.58-1.90 (5H, m), 2.01-2.05 (1H, m), 3.16-3.38 (2H, m), 3.46-3.71 (1H, m), 3.75-3.89 (2H, m), 6.66-6.81 (1H, m), 7.96-8.07 (1H, m), 8.10-8.31 (1H, m).

### Step 2. Synthesis of tert-butyl 3-((2-amino-5-bromophenyl)amino)azepane-1-carboxylate

To a solution of tert-butyl 3-((5-bromo-2-nitrophenyl)amino)azepane-1-carboxylate (1.20 g, 2.90 mmol), reductive Fe powder (1.29 g, 23.2 mmol) and NH₄Cl (1.08 g, 20.3 mmol) in EtOH (20 mL) and H₂O (5 mL) was stirred at 75° C. for 1 hour. The reaction was filtered and the solid was washed with hot EtOH (20 mL×3). The filtrate was concentrated and the residue was diluted with EtOAc (100 mL) and washed with water (60 mL×3), dried over anhydrous Na₂SO₄, filtered and concentrated to give tert-butyl 3-((2-amino-5-bromophenyl)amino)azepane-1-carboxylate (1.00 g, yield: 90%) as black brown gum, which was directly used for the next step without further purification.

### Step 3. Synthesis of tert-butyl 3-(6-bromo-1H-benzo[d]imidazol-1-yl)azepane-1-carboxylate

A mixture of tert-butyl 3-((2-amino-5-bromophenyl)amino)azepane-1-carboxylate (1.00 g, 2.60 mmol) and PPTS (100 mg, 0.398 mmol) in trimethoxymethane (10.2 g, 96.1 mmol) was stirred at 90° C. for 16 hours under N₂ atmosphere. The reaction mixture was concentrated. The residue was diluted with EtOAc (50 mL), washed with water (10 mL×3), dried over anhydrous Na₂SO₄ and concentrated to give tert-butyl 3-(6-bromo-1H-benzo[d]imidazol-1-yl)azepane-1-carboxylate (800 mg, 2.03 mmol, 77.97% yield) as yellow gum.

¹H NMR (400 MHz, CDCl₃) δ 1.37-1.58 (9H, m), 1.59-1.93 (2H, m), 1.97-2.12 (2H, m), 2.13-2.31 (2H, m), 3.40-3.77 (3H, m), 3.84-4.09 (1H, m), 4.60-4.70 (1H, m), 7.44 (1H, d, J=8.4 Hz), 7.61 (1H, t, J=8.4 Hz), 7.83-8.05 (1H, m), 8.36 (1H, d, J=8.4 Hz).

### Step 4. Synthesis of tert-butyl 3-(6-(pyridin-4-yl)-1H-benzo[d]imidazol-1-yl)azepane-1-carboxylate

A mixture of tert-butyl 3-(6-bromo-1H-benzo[d]imidazol-1-yl)azepane-1-carboxylate (400 mg, 1.01 mmol), 4-pyridylboronic acid (187 mg, 1.52 mmol), Pd(dppf)Cl₂ (148 mg, 0.203 mmol) and Na₂CO₃ (323 mg, 3.04 mmol) in dioxane (8 mL) and water (2 mL) was degassed and purged with N₂ for 3 times. Then the reaction mixture was stirred at 90° C. for 16 hours under N₂ atmosphere. The reaction mixture was filtered and the filtrate was concentrated. The residue was purified by flash silica gel chromatography (ISCO®; 12 g SepaFlash® Silica Flash Column, Eluent of 0˜10% MeOH/DCM gradient @30 mL/min) to give tert-butyl 3-(6-(pyridin-4-yl)-1H-benzo[d]imidazol-1-yl)azepane-1-carboxylate (300 mg, yield: 75%) as yellow gum.

### Step 5. Synthesis of 1-(azepan-3-yl)-6-(pyridin-4-yl)-1H-benzo[d]imidazole

A solution of tert-butyl 3-(6-(pyridin-4-yl)-1H-benzo[d]imidazol-1-yl)azepane-1-carboxylate (300 mg, 0.764 mmol) in 4N HCl/EtOAc (5 mL) was stirred at 25° C. for 2 hours. The reaction mixture was concentrated to give 1-(azepan-3-yl)-6-(pyridin-4-yl)-1H-benzo[d]imidazole (200 mg, yield: 80%, HCl salt) as a yellow solid.

### Step 6. Synthesis of 1-(1-(methylsulfonyl)azepan-3-yl)-6-(pyridin-4-yl)-1H-benzo[d]imidazole

To a solution of 1-(azepan-3-yl)-6-(pyridin-4-yl)-1H-benzo[d]imidazole (100 mg, 0.342 mmol) and Et₃N (104 mg, 1.03 mmol) in DCM (2 mL) was added MsCl (43 mg, 0.38 mmol) at 0° C. and the reaction mixture was stirred at 0° C. for 1 hour. The reaction mixture was quenched with water (25 mL) then extracted with DCM (30 mL×3). The combined organic layer was washed with saturated brine (30 mL×3), dried over Na₂SO₄, filtered and concentrated. The residue was purified by prep-HPLC (Method C; 0.225% FA as an additive), then lyophilized to give the title compound (6.7 mg, yield: 5%) as a white solid.

¹H NMR (400 MHz, CD₃OD) δ 1.71-2.00 (2H, m), 2.05-2.23 (2H, m), 2.28-2.46 (2H, m), 2.93 (3H, s), 3.42-3.49 (1H, m), 3.51-3.63 (2H, m), 3.90-3.95 (1H, m), 4.80-4.85 (1H, m), 7.74-7.79 (1H, m), 7.83 (1H, d, J=8.4 Hz), 7.85-7.90 (2H, m), 8.22 (1H, s), 8.46 (1H, s), 8.60-8.65 (2H, m).

### Example 41

### 8-((2-hydroxyethyl)(methyl)amino)-2-(pyridin-4-yl)-4H-chromen-4-one

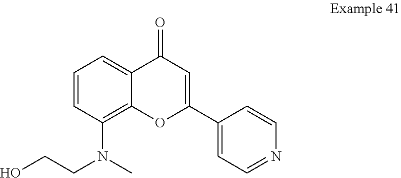 [SMILES: CN(CCO)c1cccc2c(=O)cc(-c3ccncc3)oc12]

### Step 1. Synthesis of 1-(3-bromo-2-hydroxyphenyl)-3-(pyridin-4-yl)propane-1,3-dione

To a solution of LiHMDS (14 mL, 14.0 mmol, 1 M in THF,) in THF (5 mL) was added a solution of 1-(3-bromo-2-hydroxyphenyl)ethan-1-one (1.00 g, 4.65 mmol) in THF (5 mL) dropwise at −78° C. under N₂ atmosphere. The mixture was stirred at −78° C. for 0.5 hour and then at 0° C. for 0.5 hour. A solution of methyl isonicotinate (844 mg, 5.58 mmol) in THF (5 mL) was added to the above mixture at −78° C. under N₂ atmosphere. The mixture was stirred at −78° C. for 1 hour and then at 20° C. for 2 hours. The reaction mixture was quenched with saturated aqueous NH₄Cl (30 mL) and extracted with EtOAc (30 mL×3). The combined organic layer was washed with brine (40 mL), dried over anhydrous Na₂SO₄, filtered and concentrated to give 1-(3-bromo-2-hydroxyphenyl)-3-(pyridin-4-yl)propane-1,3-dione (1.60 g, crude) as a yellow solid, which was used directly for the next step without purification.

¹H NMR (400 MHz, DMSO-d₆) δ 6.54-6.64 (1H, m), 6.65-6.75 (1H, m), 7.57 (1H, d, J=7.6 Hz), 7.78-7.88 (2H, m), 7.95 (1H, d, J=7.6 Hz), 8.60-8.70 (2H, m).

### Step 2. Synthesis of 8-bromo-2-(pyridin-4-yl)-4H-chromen-4-one

To a solution of 1-(3-bromo-2-hydroxyphenyl)-3-(pyridin-4-yl)propane-1,3-dione (1.00 g, 3.12 mmol) in DCE (10 mL) was added Tf₂O (2.64 g, 9.37 mmol), the mixture was stirred at 50° C. for 2 hours. The reaction mixture was basified with 2N aqueous NaOH to pH=8 and diluted with H₂O (40 mL), then extracted with EtOAc (40 mL×3). The combined organic layer was washed with brine (50 mL), dried over anhydrous Na₂SO₄, filtered and concentrated. The residue was purified by flash silica gel chromatography (ISCO®; 12 g SepaFlash® Silica Flash Column, Eluent of 0˜68% Ethyl acetate/Petroleum ether gradient @25 mL/min) to give 8-bromo-2-(pyridin-4-yl)-4H-chromen-4-one (270 mg, yield: 30% for two steps) as a brown solid.

### Step 3. Synthesis of 8-((2-((tert-butyldimethylsilyl)oxy)ethyl)(methyl)amino)-2-(pyridin-4-yl)-4H-chromen-4-one

To a solution of 8-bromo-2-(pyridin-4-yl)-4H-chromen-4-one (270 mg, 0.894 mmol), 2-((tert-butyldimethylsilyl)oxy)-N-methylethan-1-amine (508 mg, 2.68 mmol), Pd₂(dba)₃ (82 mg, 0.089 mmol) and XPhos (85 mg, 0.10 mmol) in 1, 4-dioxane (10 mL) was added Cs₂CO₃ (582 mg, 1.79 mmol) under N₂ atmosphere, The mixture was stirred at 85° C. for 16 hours under N₂ atmosphere. The reaction mixture was poured into water (30 mL) and extracted with EtOAc (30 mL×3). The combined organic layer was washed with brine (50 mL), dried over anhydrous Na₂SO₄, filtered and concentrated. The residue was purified by flash silica gel chromatography (ISCO®; 12 g SepaFlash® Silica Flash Column, Eluent of 0˜70% Ethyl acetate/Petroleum ether gradient @20 mL/min) to give 8-((2-((tert-butyldimethylsilyl)oxy)ethyl)(methyl) amino)-2-(pyridin-4-yl)-4H-chromen-4-one (110 mg, yield: 30%) as a brown solid.

¹H NMR (400 MHz, CDCl₃) δ −0.03 (6H, s), 0.81 (9H, s), 3.07 (3H, s), 3.54 (2H, t, J=6.0 Hz), 3.82 (2H, t, J=5.6 Hz), 6.89-6.92 (1H, m), 7.29-7.38 (2H, m), 7.77-7.84 (3H, m), 8.80-8.87 (2H, m).

### Step 4. Synthesis of 8-((2-hydroxyethyl)(methyl)amino)-2-(pyridin-4-yl)-4H-chromen-4-one

A solution of 8-((2-((tert-butyldimethylsilyl)oxy)ethyl)(methyl) amino)-2-(pyridin-4-yl)-4H-chromen-4-one (110 mg, 0.268 mmol) and 6N aqueous HCl (0.22 mL) in MeOH (10 mL) was stirred at 20° C. for 0.5 hour. The reaction mixture was basified with saturated aqueous Na₂CO₃ to pH=9 and diluted into water (30 mL), then extracted with DCM (25 mL×3). The combined organic layer was washed with brine (24 mL), dried over anhydrous Na₂SO₄, filtered and concentrated. The residue was purified by prep-TLC (DCM/MeOH=10/1) to give the title compound (40 mg, yield: 49%) as a yellow solid.

¹H NMR (400 MHz, CDCl₃) δ 2.23-2.33 (1H, m), 3.02 (3H, s), 3.45 (2H, t, J=5.2 Hz), 3.83-3.92 (2H, m), 6.93 (1H, s), 7.35-7.45 (2H, m), 7.81-7.88 (2H, m), 7.91 (1H, dd, J=7.6, 2.0 Hz), 8.80-8.86 (2H, m).

### Example 68

### (1R,5S,6r)-3-methyl-N-(5-(pyridin-4-yl)thiazolo[5,4-b]pyridin-2-yl)-3-azabicyclo[3.1.0]hexane-6-carboxamide

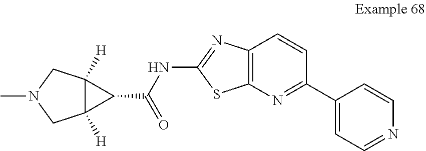 [SMILES: CN1C[C@@H]2[C@H](C1)[C@H]2C(=O)Nc1nc2ccc(-c3ccncc3)nc2s1]

### Step 1. Synthesis of tert-butyl (1R 5S,6r)-6-((5-bromothiazolo[5,4-b]pyridin-2-yl)carbamoyl)-3-azabicyclo[3.1.0]hexane-3-carboxylate

A mixture of 5-bromothiazolo[5,4-b]pyridin-2-amine (Int-17a; 200 mg, 0.869 mmol), compound 2 (198 mg, 0.869 mmol), HATU (562 mg, 1.48 mmol), DIPEA (225 mg, 1.74 mmol) in DMF (1 mL) was stirred at 50° C. for 16 hours. The reaction mixture was diluted with water (60 mL) and extracted with EtOAc (20 mL×3). The combined organic layer was washed with brine (20 mL×3), dried over anhydrous Na₂SO₄, filtered and concentrated. The residue was purified by flash silica gel chromatography (ISCO®; 4 g SepaFlash® Silica Flash Column, Eluent of 0˜30% EA/PE ether gradient @25 mL/min) to give tert-butyl (1R,5S,6r)-6-((5-bromothiazolo[5,4-b]pyridin-2-yl)carbamoyl)-3-azabicyclo[3.1.0]hexane-3-carboxylate (380 mg, yield: 50%) as a yellow solid.

### Step 2. Synthesis of tert-butyl (1R,5S,6r)-6-((5-(pyridin-4-yl)thiazolo[5,4-b]pyridin-2-yl)carbamoyl)-3-azabicyclo[3.1.0]hexane-3-carboxylate

A mixture of tert-butyl (1R,5S,6r)-6-((5-bromothiazolo[5,4-b]pyridin-2-yl)carbamoyl)-3-azabicyclo[3.1.0]hexane-3-carboxylate (380 mg, 0.865 mmol), 4-pyridylboronic acid (159 mg, 1.30 mmol), Pd(dppf)Cl₂ (63 mg, 0.087 mmol) and Na₂CO₃ (275 mg, 2.59 mmol) in dioxane (4 mL) and H₂O (1 mL) was degassed and purged with N₂ for 3 times. Then the mixture was stirred at 90° C. for 2 hours under N₂ atmosphere. The reaction mixture was filtered and the filtrate was concentrated. The residue was purified by flash silica gel chromatography (ISCO®; 12 g SepaFlash® Silica Flash Column, Eluent of 0˜10% MeOH/DCM @25 mL/min). The crude product was triturated with MeOH (2 mL) to give tert-butyl (1R,5S,6r)-6-((5-(pyridin-4-yl)thiazolo[5,4-b]pyridin-2-yl)carbamoyl)-3-azabicyclo[3.1.0]hexane-3-carboxylate (50 mg, yield: 13%) as a yellow solid.

### Step 3. Synthesis of (1R,5S,6r)-N-(5-(pyridin-4-yl)thiazolo[5,4-b]pyridin-2-yl)-3-azabicyclo[3.1.0]hexane-6-carboxamide

A solution of tert-butyl (1R,5S,6r)-6-((5-(pyridin-4-yl)thiazolo[5,4-b]pyridin-2-yl)carbamoyl)-3-azabicyclo[3.1.0]hexane-3-carboxylate (50 mg, 0.11 mmol) in TFA (1 mL) and DCM (1 mL) was stirred at 25° C. for 1 hour. The reaction mixture was concentrated to give (1R,5S,6r)-N-(5-(pyridin-4-yl)thiazolo[5,4-b]pyridin-2-yl)-3-azabicyclo[3.1.0]hexane-6-carboxamide (50 mg, crude, TFA salt) was obtained as yellow gum, which was used for the next step without further purification.

### Step 4. Synthesis of (1R,5S,6r)-3-methyl-N-(5-(pyridin-4-yl)thiazolo[5,4-b]pyridin-2-yl)-3-azabicyclo[3.1.0]hexane-6-carboxamide

To a solution of (1R,5S,6r)-N-(5-(pyridin-4-yl)thiazolo[5,4-b]pyridin-2-yl)-3-azabicyclo[3.1.0]hexane-6-carboxamide (40 mg, 0.12 mmol) in MeOH (2 mL) was added DIPEA to adjust the pH=5, then 37% aqueous HCHO (48 mg, 0.59 mmol) was added to the reaction mixture and stirred at 25° C. for 30 minutes. NaBH₃CN (22 mg, 0.36 mmol) was added and the resulting reaction mixture was stirred at 25° C. for another 2 hours. The reaction mixture was concentrated and the residue was purified by prep-HPLC (0.225% FA as an additive), then lyophilized to give the title compound (15 mg, yield: 35% for 2 steps, FA salt) as a white solid.

¹H NMR (400 MHz, DMSO-d₆) δ 2.05-2.10 (2H, m), 2.31 (3H, s), 2.35-2.40 (1H, m), 2.40-2.46 (2H, m), 3.05-3.10 (2H, m), 8.06-8.12 (2H, m), 8.19-8.25 (2H, m), 8.65-8.71 (2H, m), 12.75 (1H, brs).

The following compound was synthesized analogously to Example 68

| Example | | | |
|---|---|---|---|
| No. | Structure | Name | ¹H NMR (400 MHz) |
| 69 | 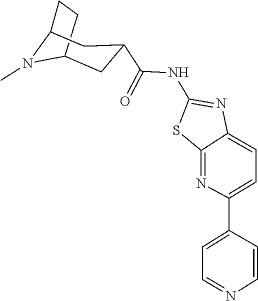 [SMILES: CN1C2CCC1CC(C(=O)Nc1nc3ccc(-c4ccncc4)nc3s1)C2] | 8-methyl-N-(5-(pyridin-4- yl)thiazolo[5,4-b]pyridin- 2-yl)-8- azabicyclo[3.2.1]octane-3- carboxamide | DMSO-d₆; δ 1.55-1.63 (4H, m), 1.76-1.86 (2H, m), 1.93-2.02 (2H, m), 2.19 (3H, s), 2.79-2.90 (1H, m), 3.11-3.18 (2H, m), 8.06- 8.20 (4H, m), 8.65-8.71 (2H, m). |

### Example 75

### 4-(6-(1-methyl-6-oxo-1,6-dihydropyridin-3-yl)pyrazolo[1,5-a]pyridin-3-yl)pyridine 1-oxide

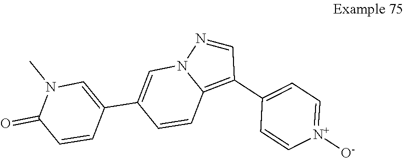 [SMILES: Cn1cc(-c2ccc3c(-c4cc[n+]([O-])cc4)cnn3c2)ccc1=O]

### Step 1. Synthesis of 6-bromo-3-(pyridin-4-yl)pyrazolo[1,5-a]pyridine

A mixture of 6-bromo-3-iodopyrazolo[1,5-a]pyridine (200 mg, 0.619 mmol), pyridin-4-ylboronic acid (99 mg, 0.81 mmol), Pd(dppf)Cl₂ (45 mg, 0.062 mmol) and K₂CO₃ (171 mg, 1.24 mmol) in dioxane (4 mL) and H₂O (0.5 mL) was degassed and purged with N₂ for 3 times. Then the mixture was stirred at 90° C. for 12 hours under N₂ atmosphere. The reaction mixture was dissolved in MeOH (20 mL) and filtered. The filtrate was concentrated and the residue was purified by Combi Flash (0% to 25% EtOAc in PE) to give 6-bromo-3-(pyridin-4-yl)pyrazolo[1,5-a]pyridine (50 mg, yield: 29%) as a yellow solid.

¹H NMR (400 MHz, DMSO-d₆) δ 7.57 (1H, dd, J=9.6, 1.6 Hz), 7.68-7.78 (2H, m), 8.13 (1H, d, J=9.2 Hz), 8.57-8.62 (2H, m), 8.65 (1H, s), 9.23 (1H, d, J=1.2 Hz).

### Step 2. Synthesis of 4-(6-bromopyrazolo[1,5-a]pyridin-3-yl)pyridine 1-oxide

To a solution of 6-bromo-3-(pyridin-4-yl)pyrazolo[1,5-a]pyridine (110 mg, 0.401 mmol) in DCM (3 mL) was added m-CPBA (244 mg, 1.20 mmol, 85% purity) at 0° C. The mixture was stirred at 25° C. for 16 hours to give a yellow mixture. The reaction mixture was diluted with saturated aqueous Na₂CO₃ (10 mL) and extracted with DCM (20 mL×3). The combined organic layer was washed with brine (15 mL), dried over anhydrous Na₂SO₄, filtered and concentrated to give 4-(6-bromopyrazolo[1,5-a]pyridin-3-yl)pyridine 1-oxide (105 mg, yield: 90%) as a yellow solid.

### Step 3. Synthesis of 4-(6-(1-methyl-6-oxo-1,6-dihydropyridin-3-yl)pyrazolo[1,5-a]pyridin-3-yl)pyridine 1-oxide

A mixture of 4-(6-bromopyrazolo[1,5-a]pyridin-3-yl)pyridine 1-oxide (105 mg, 0.362 mmol), 1-methyl-5-(4,4,5,5-tetramethyl-1,3,2-dioxaborolan-2-yl)pyridin-2(1H)-one (94 mg, 0.40 mmol), Pd(dppf)Cl₂ (26 mg, 0.036 mmol) and Na₂CO₃ (77 mg, 0.72 mmol) in dioxane (4 mL) and 8.2 (0.5 mL) was degassed and purged with N₂ for 3 times. Then the mixture was stirred at 90° C. for 2 hours under N₂ atmosphere. The reaction mixture was dissolved in MeOH (20 mL) and filtered. The filtrate was concentrated and the residue was titurated with MeOH (3 mL), then filtered. The solid was washed with H₂O (1 mL×3), then lyophilized to give the title compound (9.5 mg, yield: 7.9%) as a yellow solid.

¹H NMR (400 MHz, CD₃OD) δ 3.69 (3H, s), 6.71 (1H, d, J=9.6 Hz), 7.72 (1H, dd, J=9.2, 1.2 Hz), 7.85-7.95 (2H, m), 7.97 (1H, dd, J=9.6, 2.8 Hz), 8.10-8.19 (2H, m), 8.30-8.38 (2H, m), 8.49 (1H, s), 8.90 (1H, s).

The following compounds were synthesized analogously to Example 75

| Example | | | |
|---|---|---|---|
| No. | Structure | Name | ¹H NMR (400 MHz) |
| 79 | 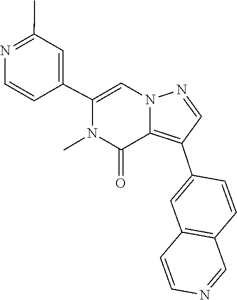 [SMILES: Cc1cc(-c2cn3ncc(-c4ccc5cnccc5c4)c3c(=O)n2C)ccn1] | 3-(isoquinolin-6-yl)-5- methyl-6-(2- methylpyridin-4-yl) pyrazolo[1,5-a]pyrazin- 4(5H)-one | DMSO-d₆; δ 2.58 (3H, s), 3.28 (3H, s), 7.46 (1H, dd, J = 5.2, 1.2 Hz), 7.52 (1H, s), 7.84 (1H, d, J = 6.0 Hz), 7.95 (1H, s), 8.15-8.23 (2H, m), 8.45-8.49 (2H, m), 8.53 (1H, d, J = 6.0 Hz), 8.62 (1H, d, J = 5.2 Hz), 9.33 (1H, s). |
| 80 | 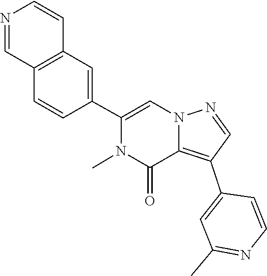 [SMILES: Cc1cc(-c2cnn3cc(-c4ccc5cnccc5c4)n(C)c(=O)c23)ccn1] | 6-(isoquinolin-6-yl)-5- methyl-3-(2- methylpyridin-4- yl)pyrazolo[1,5-a]pyrazin- 4(5H)-one | DMSO-d₆; δ 2.53 (3H, s), 3.29 (3H, s), 7.80 (1H, dd, J = 5.2, 1.2 Hz), 7.85 (1H, s), 7.90 (1H, dd, J = 8.4, 1.6 Hz), 7.95 (1H, d, J = 5.6 Hz), 7.99 (1H, s), 8.23 (1H, s), 8.29 (1H, d, J = 8.4 Hz), 8.46 (1H, s), 8.48 (1H, d, J = 5.2 Hz), 8.63 (1H, d, J = 5.6 Hz), 9.44 (1H, s). |
| 84 | 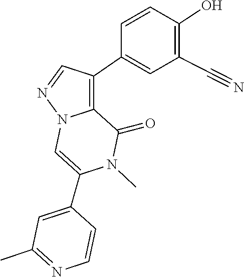 [SMILES: Cc1cc(-c2cn3ncc(-c4ccc(O)c(C#N)c4)c3c(=O)n2C)ccn1] | 2-hydroxy-5-(5-methyl-6- (2-methylpyridin-4-yl)-4- oxo-4,5- dihydropyrazolo[1,5- a]pyrazin-3- yl)benzonitrile | DMSO-d₆; δ 2.56 (3H, s), 3.25 (3H, s), 7.07 (1H, d, J = 8.8 Hz), 7.43 (1H, d, J = 4.8 Hz), 7.49 (1H, s), 7.86 (1H, s), 8.01 (1H, dd, J = 8.8, 2.4 Hz), 8.18 (1H, d, J = 2.4 Hz), 8.27 (1H, s), 8.60 (1H, d, J = 4.8 Hz), 11.24 (1H, brs). |

### Example 76

### 3-methyl-6-(pyridin-4-yl)-2-((tetrahydro-2H-pyran-4-yl)ethynyl)imidazo[1,2-a]pyridine

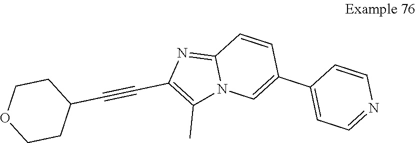 [SMILES: Cc1c(C#CC2CCOCC2)nc2ccc(-c3ccncc3)cn12]

### Step 1. Synthesis of 2-bromo-6-chloro-3-methylimidazo[1,2-a]pyridine

A mixture of but-2-ynoic acid (660 mg, 7.85 mmol), 5-chloropyridin-2-amine (3.03 g, 23.5 mmol) and CuBr₂ (1.75 g, 7.85 mmol) in CH₃CN (30 mL) was stirred at 90° C. for 16 hours under 02 atmosphere (15 psi). The reaction mixture was filtered, the solid was washed with CH₃CN (10 mL) and the filtrate was concentrated. The residue was purified by Combi Flash (0% to 20% EtOAc in PE) to give 2-bromo-6-chloro-3-methylimidazo[1,2-a]pyridine (190 mg, yield: 9%) as a white solid.

### Step 2. Synthesis of 6-chloro-3-methyl-2-((tetrahydro-2H-pyran-4-yl)ethynyl)imidazo[1,2-a]pyridine

A mixture of 2-bromo-6-chloro-3-methylimidazo[1,2-a]pyridine (110 mg, 0.448 mmol), 4-ethynyltetrahydro-2H-pyran (99 mg, 0.90 mmol), Pd(PPh₃)₂Cl₂ (31 mg, 0.045 mmol) and CuI (17 mg, 0.0896 mmol) in Et₃N (3 mL) was degassed and purged with N₂ for 3 times, the mixture was stirred at 90° C. for 3 hours under N₂ atmosphere. The reaction mixture was concentrated and the residue was purified by Combi Flash (0% to 50% EtOAc in PE) to give 6-chloro-3-methyl-2-((tetrahydro-2H-pyran-4-yl)ethynyl)imidazo[1,2-a]pyridine (90 mg, yield: 54%) as a light brown solid.

### Step 3. Synthesis of 3-methyl-6-(pyridin-4-yl)-2-((tetrahydro-2H-pyran-4-yl)ethynyl)imidazo[1,2-a]pyridine

A mixture of 6-chloro-3-methyl-2-((tetrahydro-2H-pyran-4-yl)ethynyl)imidazo[1,2-a]pyridine (70 mg, 0.25 mmol), pyridin-4-ylboronic acid (47 mg, 0.38 mmol), Xphos-Pd-G₃ (22 mg, 0.025 mmol) and K₂CO₃ (70 mg, 0.51 mmol) in dioxane (3 mL) and H₂O (0.5 mL) was degassed and purged with N₂ for 3 times, and then the mixture was stirred at 100° C. for 5 hours. The reaction mixture was filtered and the filtrate was concentrated. The residue was purified by Combi Flash (0% to 100% EtOAc in PE), then further purified by prep-HPLC (0.225% FA as an additive) and lyophilized to give the title compound (11.57 mg, yield: 14%) as an off-white solid.

¹H NMR (400 MHz, DMSO-d₆) δ 1.58-1.71 (2H, m), 1.83-1.94 (2H, m), 2.58 (3H, s), 2.92-3.05 (1H, m), 3.48 (2H, m), 3.77-3.88 (2H, m), 7.60 (1H, d, J=9.6 Hz), 7.72 (1H, dd, J=9.2, 1.6 Hz), 7.82-7.88 (2H, m), 8.65-8.68 (2H, m), 8.71 (1H, s).

The following compounds were synthesized analogously to Example 76

| Example | | | |
|---|---|---|---|
| No. | Structure | Name | ¹H NMR (400 MHz) |
| 95 | 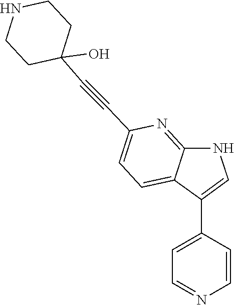 [SMILES: OC1(C#Cc2ccc3c(-c4ccncc4)c[nH]c3n2)CCNCC1] | 4-((3-(pyridin-4-yl)-1H- pyrrolo[2,3-b]pyridin-6- yl)ethynyl)piperidin-4-ol | DMSO-d₆; δ 1.52-1.68 (2H, m), 1.80-1.90 (2H, m), 2.63-2.77 (2H, m), 2.85-2.95 (2H, m), 5.63 (1H, brs), 7.30 (1H, d, J = 8.4 Hz), 7.71-7.79 (2H, m), 8.31 (1H, s), 8.41 (1H, d, J = 8.4 Hz), 8.52- 8.59 (2 H, m). |
| 103 | 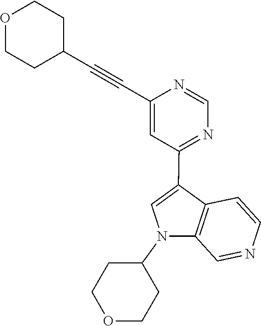 [SMILES: C(#CC1CCOCC1)c1cc(-c2cn(C3CCOCC3)c3cnccc23)ncn1] | 1-(tetrahydro-2H-pyran-4- yl)-3-(6-((tetrahydro-2H- pyran-4- yl)ethynyl)pyrimidin-4- yl)-1H-pyrrolo[2,3- c]pyridine | DMSO-d₆; δ 1.61-1.72 (2H, m), 1.85-1.95 (2H, m), 2.02-2.17 (4H, m), 2.98-3.06 (1H, m), 3.44- 3.52 (2H, m), 3.57-3.65 (2H, m), 3.80-3.88 (2H, m), 4.00-4.05 (2H, m), 4.85-4.98 (1H, m), 8.08 (1H, d, J = 1.2 Hz), 8.31 (1H, d, J = 5.6 Hz), 8.41 (1H, d, J = 5.6 Hz), 8.94 (1H, s), 9.05 (1H, d, J = 1.6 Hz), 9.12 (1H, s). |

### Example 77

### ethyl 4-7-(pyridin-3-yl)-5,6,7,8-tetrahydroimidazo[1,2-a]pyrazin-3-yl)benzoate

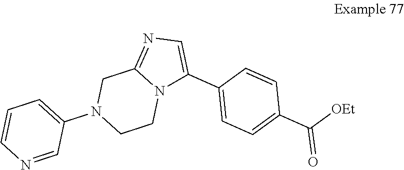 [SMILES: CCOC(=O)c1ccc(-c2cnc3n2CCN(c2cccnc2)C3)cc1]

### Step 1. Synthesis of 7-(pyridin-3-yl)-5,6,7,8-tetrahydroimidazo[1,2-a]pyrazine

A mixture of 5,6,7,8-tetrahydroimidazo[1,2-a]pyrazine (1.00 g, 8.12 mmol), 3-iodopyridine (2.50 g, 12.2 mmol), Pd₂(dba)₃ (744 mg, 0.812 mmol), RuPhos (758 mg, 1.62 mmol) and Cs₂CO₃ (5.29 g, 16.2 mmol) in 1, 4-dioxane (25 mL) was degassed and purged with N₂ for 3 times and then stirred at 90° C. for 16 hours under N₂ atmosphere. The reaction mixture was concentrated and the residue was purified by silica gel column (DCM/MeOH=10/1) to afford 7-(pyridin-3-yl)-5,6,7,8-tetrahydroimidazo[1,2-a]pyrazine (1.30 g, yield: 80%) as yellow oil.

¹H NMR (400 MHz, DMSO-d₆) δ 3.77 (2H, t, J=5.6 Hz), 4.10 (2H, t, J=5.6 Hz), 4.45 (2H, s), 6.89 (1H, s), 7.12 (1H, s), 7.26 (1H, dd, J=8.4, 4.4 Hz), 7.43-7.48 (1H, m), 8.04 (1H, dd, J=4.4, 1.2 Hz), 8.42 (1H, d, J=2.8 Hz).

### Step 2. Synthesis of 3-bromo-7-(pyridin-3-yl)-5,6,7,8-tetrahydroimidazo[1,2-a]pyrazine

A mixture of 7-(pyridin-3-yl)-5,6,7,8-tetrahydroimidazo[1,2-a]pyrazine (1.30 g, 6.49 mmol) and NBS (1.44 g, 8.12 mmol) in DMF (15 mL) was stirred at 25° C. for 16 hours. The reaction mixture was concentrated and the residue was purified by silica gel column (DCM/MeOH=10/1) to afford 7-(pyridin-3-yl)-5,6,7,8-tetrahydroimidazo[1,2-a]pyrazine (600 mg, yield: 33%) as a yellow solid.

¹H NMR (400 MHz, DMSO-d₆) δ 3.83 (2H, t, J=5.6 Hz), 3.96 (2H, t, J=5.6 Hz), 4.47 (2H, s), 7.01 (1H, s), 7.28 (1H, dd, J=8.4, 4.8 Hz), 7.46-7.53 (1H, m), 8.06 (1H, d, J=3.6 Hz), 8.44 (1H, d, J=2.8 Hz).

### Step 3. Synthesis of ethyl 4-(7-(pyridin-3-yl)-5,6,7,8-tetrahydroimidazo[1,2-a]pyrazin-3-yl)benzoate

A mixture of compound 7-(pyridin-3-yl)-5,6,7,8-tetrahydroimidazo[1,2-a]pyrazine (200 mg, 0.717 mmol), (4-(ethoxycarbonyl)phenyl)boronic acid (208 mg, 1.07 mmol), Pd(dppf)Cl₂ (52 mg, 0.072 mmol) and Na₂CO₃ (152 mg, 1.43 mmol) in 1, 4-dioxane (3 mL) and H₂O (0.6 mL) was bubbled with N₂ for 6 minutes and then stirred at 90° C. for 16 hours. The reaction mixture was concentrated and the residue was purified by silica gel column (DCM/MeOH=10/1) to afford the title compound (200 mg, yield: 64%) as a yellow solid.

### Example 78

### 4-(7-(pyridin-3-yl)-5,6,7,8-tetrahydroimidazo[1,2-a]pyrazin-3-yl)benzoic Acid

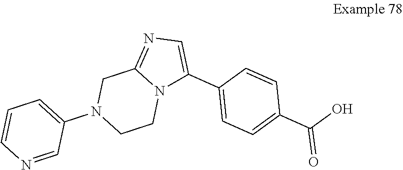 [SMILES: O=C(O)c1ccc(-c2cnc3n2CCN(c2cccnc2)C3)cc1]

### Example 78

A mixture of Example 77 (130 mg, 0.297 mmol) and LiOH·H₂O (37 mg, 0.89 mmol) in THF (1.5 mL), MeOH (1.5 mL) and H₂O (1.5 mL) was stirred at 25° C. for 16 hours. The reaction mixture was concentrated and purified by prep-HPLC (Method C; 0.225% FA as an additive), then lyophilized to afford the title compound (52.99 mg, yield: 48%, FA salt) as a white solid.

¹H NMR (400 MHz, DMSO-d₆) δ 3.78 (2H, t, J=5.2 Hz), 4.24 (2H, t, J=5.2 Hz), 4.57 (2H, s), 7.25-7.33 (2H, m), 7.45-7.53 (1H, m), 7.65 (2H, d, J=8.4 Hz), 7.90-8.00 (2H, m), 8.06 (1H, d, J=4.8 Hz), 8.16 (1H, s), 8.46 (1H, d, J=2.8 Hz).

### Example 81

### 4-(6-oxo-7-(tetrahydro-2H-pyran-4-yl)-5,6,7,8-tetrahydroimidazo[1,2-a]pyrazin-3-yl)benzoic Acid

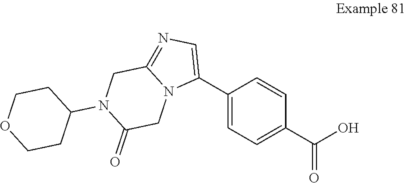 [SMILES: O=C(O)c1ccc(-c2cnc3n2CC(=O)N(C2CCOCC2)C3)cc1]

### Step 1. Synthesis of ethyl 2-(2-formyl-1H-imidazol-1-yl)acetate

A mixture of 1H-imidazole-2-carbaldehyde (5.00 g, 52.0 mmol, ethyl 2-bromoacetate (13.0 g, 78.1 mmol), K₂CO₃ (10.8 g, 78.1 mmol) in DMF (30 mL) was stirred at 25° C. for 16 hours. The reaction mixture was concentrated and the residue was diluted with water (100 mL), then extracted with DCM (60 mL×2). The combined organic layer was dried over anhydrous Na₂SO₄, filtered and concentrated. The residue was purified by flash silica gel column (MeOH/DCM=1/50) to give ethyl 2-(2-formyl-1H-imidazol-1-yl)acetate (4.30 g, yield: 45%) as yellow oil.

### Step 2. Synthesis of 7-(tetrahydro-2H-pyran-4-yl)-7,8-dihydroimidazo[1,2-a]pyrazin-6(5H)-one

To a solution of 2-(2-formyl-1H-imidazol-1-yl)acetate (1.00 g, 5.49 mmol), tetrahydropyran-4-amine (611 mg, 6.04 mmol) and HOAc (659 mg, 11.0 mmol) in MeOH (50 mL) was stirred at 25° C. for 0.5 hour. The mixture was cooled to 0° C. and NaBH(OAc)₃ (3.49 g, 16.5 mmol) was added. The mixture was stirred at 25° C. for another 3.5 hours. The reaction mixture was concentrated and the residue was dissolved in HOAc (20 mL), then stirred at 70° C. for 16 hours. The reaction mixture was concentrated and the residue was basified with saturated aqueous NaHCO₃ to pH=8, then extracted with DCM (50 ml×2). The combined organic layer was dried over anhydrous Na₂SO₄, filtered and concentrated. The residue was purified by flash silica gel column (MeOH/DCM=1/20) to afford 7-(tetrahydro-2H-pyran-4-yl)-7,8-dihydroimidazo[1,2-a]pyrazin-6(5H)-one (350 mg, yield: 30%) as a white solid.

### Step 3. Synthesis of 3-bromo-7-(tetrahydro-2H-pyran-4-yl)-7,8-dihydroimidazo[1,2-a]pyrazin-6(5H)-one

A solution of -(tetrahydro-2H-pyran-4-yl)-7,8-dihydroimidazo[1,2-a]pyrazin-6(5H)-one (237 mg, 1.07 mmol) and NBS (153 mg, 0.860 mmol) in DMF (6 mL) was stirred at 0° C. for 2 hours. The reaction mixture was concentrated and the residue was purified by flash silica gel column (DCM/MeOH=10/1) to afford Synthesis of 3-bromo-7-(tetrahydro-2H-pyran-4-yl)-7,8-dihydroimidazo[1,2-a]pyrazin-6(5H)-one (195 mg, yield: 61%) as a white solid.

¹H NMR (400 MHz, DMSO-d₆) δ 1.45-1.55 (2H, m), 1.80-1.90 (2H, m), 3.40-3.50 (2H, m), 3.90-3.95 (2H, m), 4.51 (s, 2H), 4.53 (2H, s), 4.55-4.61 (1H, m), 7.08 (1H, s).

### Step 4. Synthesis of tert-butyl 4-(6-oxo-7-(tetrahydro-2H-pyran-4-yl)-5,6,7,8-tetrahydroimidazo[1,2-a]pyrazin-3-yl)benzoate

A mixture of 3-bromo-7-(tetrahydro-2H-pyran-4-yl)-7,8-dihydroimidazo[1,2-a]pyrazin-6(5H)-one (90 mg, 0.30 mmol), (4-(tert-butoxycarbonyl)phenyl)boronic acid (80 mg, 0.36 mmol), Pd(dppf)Cl₂ (22 mg, 0.030 mmol), Na₂CO₃ (64 mg, 0.60 mmol) in dioxane (3 mL) and H₂O (0.5 mL) was degassed and purged with N₂ for 3 times, then the mixture was stirred at 90° C. for 12 hours under N₂ atmosphere. The reaction mixture was concentrated and the residue was purified by flash silica gel column (DCM/MeOH=10/1) to afford tert-butyl 4-(6-oxo-7-(tetrahydro-2H-pyran-4-yl)-5,6,7,8-tetrahydroimidazo[1,2-a]pyrazin-3-yl)benzoate (88 mg, yield: 55%) as a white solid.

### Step 5. Synthesis of 4-(6-oxo-7-(tetrahydro-2H-pyran-4-yl)-5,6,7,8-tetrahydroimidazo[1,2-a]pyrazin-3-yl)benzoic Acid

A solution of tert-butyl 4-(6-oxo-7-(tetrahydro-2H-pyran-4-yl)-5,6,7,8-tetrahydroimidazo[1,2-a]pyrazin-3-yl)benzoate (88 mg, 0.17 mmol) in DCM (3 mL) and TFA (3 mL) was stirred at 20° C. for 3 hours. The reaction was concentrated and the residue was purified by prep-HPLC (0.05% HCl as an additive), then lyophilized to afford the title compound (45.78 mg, yield: 73%, HCl salt) as a white solid.

¹H NMR (400 MHz, DMSO-d₆) δ 1.50-155 (2H, m), 1.82-1.95 (2H, m), 2.50-2.55 (2H, m), 3.90-4.00 (2H, m), 4.54-4.60 (1H, m), 4.70 (2H, s), 4.90 (2H, s), 7.68-7.74 (3H, m), 8.04 (2H, d, J=8.4 Hz), 13.12 (1H, brs)

### Example 82

### 4-(7-(4-methoxybenzyl)-6-oxo-5,6,7,8-tetrahydroimidazo[1,2-a]pyrazin-3-yl)benzonitrile

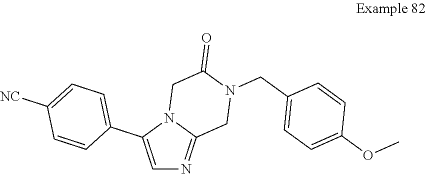 [SMILES: COc1ccc(CN2Cc3ncc(-c4ccc(C#N)cc4)n3CC2=O)cc1]

### Step 1. Synthesis of ethyl 2-(2-formyl-1H-imidazol-1-yl)acetate

A mixture of 1H-imidazole-2-carbaldehyde (3.00 g, 31.2 mmol), ethyl-2-bromoacetate (5.74 g, 34.3 mmol) and K₂CO₃ (4.75 g, 34.3 mmol) in DMF (30 mL) was stirred at 25° C. for 4 hours. The reaction was filtered and the filtrate was concentrated. The residue was purified by Combi Flash (0% to 60% EtOAc in PE) to give ethyl 2-(2-formyl-1H-imidazol-1-yl)acetate (1.50 g, yield: 26%) as colorless oil.

### Step 2. Synthesis of ethyl 2-(2-formyl-1H-imidazol-1-yl)acetate

To a solution of ethyl 2-(2-formyl-1H-imidazol-1-yl)acetate (1.39 g, 7.62 mmol), (4-methoxyphenyl)methanamine (0.95 g, 6.93 mmol) and HOAc (832 mg, 13.9 mmol) in MeOH (40 mL) was stirred at 25° C. for 4 hours. Then the mixture was cooled to 0° C. and NaBH₄ (786 mg, 20.8 mmol) was added. The mixture was stirred at 25° C. for 12 hours. The reaction mixture was quenched with saturated aqueous NaHCO₃ (50 mL) and concentrated. The residue was diluted with water (50 mL) and extracted with EtOAc (50 mL×3). The combined organic layer was washed with brine (50 mL), dried over anhydrous Na₂SO₄ and concentrated. The residue was purified silica gel column (0 to 80% EtOAc in PE) to give 7-(4-methoxybenzyl)-7,8-dihydroimidazo[1,2-a]pyrazin-6(5H)-one (470 mg, yield: 26%) as a yellow solid.

### Step 3. Synthesis of 3-bromo-7-(4-methoxybenzyl)-7,8-dihydroimidazo[1,2-a]pyrazin-6(5H)-one

A mixture of 7-(4-methoxybenzyl)-7,8-dihydroimidazo[1,2-a]pyrazin-6(5H)-one (50 mg, 0.19 mmol) and NBS (38 mg, 0.21 mmol) in DMF (2 mL) was stirred at 25° C. for 2 hours under N₂ atmosphere. The reaction mixture was concentrated and the residue was purified by Combi Flash (0% to 80% EtOAc in PE) to give 3-bromo-7-(4-methoxybenzyl)-7,8-dihydroimidazo[1,2-a]pyrazin-6(5H)-one (30 mg, yield: 46%) as a white solid.

### Step 4. Synthesis of 4-(7-(4-methoxybenzyl)-6-oxo-5,6,7,8-tetrahydroimidazo[1,2-a]pyrazin-3-yl)benzonitrile

A mixture of 3-bromo-7-(4-methoxybenzyl)-7,8-dihydroimidazo[1,2-a]pyrazin-6(5H)-one (30 mg, 0.089 mmol), (4-cyanophenyl)boronic acid (20 mg, 0.13 mmol), Pd(dppf)Cl₂ (7 mg, 0.01 mmol) and Na₂CO₃ (19 mg, 0.18 mmol) in 1,4-dioxane (5 mL) and H₂O (1 mL) was degassed and purged with N₂ for 3 times, then the mixture was stirred at 90° C. for 12 hours under N₂ atmosphere. The reaction mixture was filtered and the filtrate was concentrated. The residue was purified by prep-HPLC (0.225% FA as an additive) and lyophilized to give the title compound (7 mg, yield: 22%) as a white solid.

¹H NMR (400 MHz, CD₃OD) δ 3.80 (3H, s), 4.59 (2H, s), 4.62 (1H, s), 4.74 (2H, s), 4.86-4.87 (1H, m), 6.94 (2H, d, J=8.8 Hz), 7.28-7.37 (3H, m), 7.72 (2H, d, J=8.4 Hz), 7.85 (2H, d, J=8.0 Hz).

### Example 85

### 6-(((1-methylpiperidin-4-yl)oxy)methyl)-4-propoxy-3-(pyridin-4-yl)-1H-pyrrolo[2,3-b]pyridine

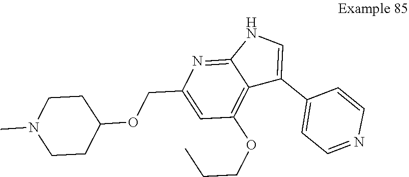 [SMILES: CCCOc1cc(COC2CCN(C)CC2)nc2[nH]cc(-c3ccncc3)c12]

### Step 1. Synthesis of 6-chloro-3-iodo-4-propoxy-1-((2-(trimethylsilyl)ethoxy)methyl)-1H-pyrrolo[2,3-b]pyridine

To a solution of 4,6-dichloro-3-iodo-1-((2-(trimethylsilyl)ethoxy)methyl)-1H-pyrrolo[2,3-b]pyridine (1.00 g, 2.26 mmol) and propan-1-ol (407 mg, 6.78 mmol) in anhydrous DMF (10 mL) was added NaH (136 mg, 3.39 mmol, 60% dispersion in mineral oil) at 20° C. Then the reaction mixture was stirred at 20° C. for 14.5 hours. The reaction mixture turned into yellow suspension from yellow solution. The reaction mixture was quenched with H₂O (50 mL) and extracted with EtOAc (60 mL×3). The combined organic layers were washed with brine (50 mL), dried over anhydrous Na₂SO₄, filtered and concentrated. The residue was purified by flash silica gel chromatography (ISCO®; 20 g SepaFlash® Silica Flash Column, Eluent of 0˜10% Ethyl acetate/Petroleum ether gradient @30 mL/min) to give 6-chloro-3-iodo-4-propoxy-1-((2-(trimethylsilyl)ethoxy)methyl)-1H-pyrrolo[2,3-b]pyridine (900 mg, yield: 85%) as a white solid.

¹H NMR (400 MHz, CDCl₃) δ −0.04 (9H, s), 0.90-0.95 (2H, m), 1.19 (3H, t, J=7.2 Hz), 1.92-1.99 (2H, m), 3.50-3.56 (2H, m), 4.10 (2H, t, J=6.4 Hz), 5.55 (2H, s), 6.55 (1H, s), 7.27 (1H, s).

### Step 2. Synthesis of 6-chloro-4-propoxy-3-(pyridin-4-yl)-1-((2-(trimethylsilyl)ethoxy)methyl)-1H-pyrrolo[2,3-b]pyridine

A mixture of 6-chloro-3-iodo-4-propoxy-1-((2-(trimethylsilyl)ethoxy)methyl)-1H-pyrrolo[2,3-b]pyridine (900 mg, 1.93 mmol), 4-pyridylboronic acid (284 mg, 2.31 mmol), Pd(dppf)Cl₂ (141 mg, 0.192 mmol) and Na₂CO₃ (613 mg, 5.78 mmol,) in dioxane (10 mL) and H₂O (1 mL) was degassed and purged with N₂ for 3 times, and then the mixture was stirred at 90° C. for 1.5 hours under N₂ atmosphere. The reaction mixture was concentrated and the residue was purified by flash silica gel chromatography (ISCO®; 12 g SepaFlash® Silica Flash Column, Eluent of 0˜20% Ethyl acetate/Petroleum ether gradient @25 mL/min) to give 6-chloro-4-propoxy-3-(pyridin-4-yl)-1-((2-(trimethylsilyl)ethoxy)methyl)-1H-pyrrolo[2,3-b]pyridine (200 mg, yield: 23%) as brown gum.

### Step 3. Synthesis of 1-methyl-4-((tributylstannyl)methoxy)piperidine (85-g)

To a solution of 1-methylpiperidin-4-ol (1.00 g, 8.68 mmol) and anhydrous THF (10 mL) was added NaH (347 mg, 8.68 mmol, 60% dispersion in mineral oil) at 0° C. Then the reaction mixture was stirred at 25° C. for 30 minutes. A solution of tributyl(iodomethyl) stannane (2.49 g, 5.79 mmol) in anhydrous DMF (18 mL) was added to the above reaction mixture at 0° C. The reaction mixture was stirred at 25° C. for another 1.5 hours. The reaction mixture was quenched with H₂O (25 mL) and extracted with DCM/MeOH (50 mL×2, 10/1). The combined organic layers were washed with brine (20 mL×5), dried over anhydrous Na₂SO₄, filtered and concentrated. The residue was purified by flash silica gel chromatography (ISCO®; 12 g SepaFlash® Silica Flash Column, Eluent of 0˜30% Ethyl acetate/Petroleum ether gradient @40 mL/min) to give 1-methyl-4-((tributylstannyl)methoxy)piperidine (1.10 g, yield: 45%) as yellow oil.

### Step 4. Synthesis of 6-(((1-methylpiperidin-4-yl)oxy)methyl)-4-propoxy-3-(pyridin-4-yl)-1-((2-(trimethylsilyl)ethoxy)methyl)-1H-pyrrolo[2,3-b]pyridine

A mixture of 6-chloro-4-propoxy-3-(pyridin-4-yl)-1-((2-(trimethylsilyl)ethoxy)methyl)-1H-pyrrolo[2,3-b]pyridine (50 mg, 0.12 mmol), 1-methyl-4-((tributylstannyl)methoxy)piperidine (75 mg, 0.18 mmol), Pd(OAc)₂ (3 mg, 0.01 mmol) and XPhos (18 mg, 0.036 mmol) in anhydrous DMF (2 mL) was degassed and purged with N₂ for 3 times, and then the mixture was stirred at 110° C. for 12 hours under N₂ atmosphere. The reaction mixture was quenched by addition saturated aqueous KF (20 mL) and extracted with EtOAc (25 mL×2). The combined organic layers were washed with brine (20 mL×2), dried over anhydrous Na₂SO₄, filtered and concentrated. The residue was purified by Combi Flash (0% to 15% MeOH in DCM) to give 6-(((1-methylpiperidin-4-yl)oxy)methyl)-4-propoxy-3-(pyridin-4-yl)-1-((2-(trimethylsilyl)ethoxy)methyl)-1H-pyrrolo[2,3-b]pyridine (40 mg, yield: 33%) as a yellow solid.

### Step 5. Synthesis of 6-(((1-methylpiperidin-4-yl)oxy)methyl)-4-propoxy-3-(pyridin-4-yl)-1H-pyrrolo[2,3-b]pyridine

A mixture of 6-(((1-methylpiperidin-4-yl)oxy)methyl)-4-propoxy-3-(pyridin-4-yl)-1-((2-(trimethylsilyl)ethoxy)methyl)-1H-pyrrolo[2,3-b]pyridine (40 mg, 0.78 mmol) in TFA (2 mL) and DCM (2 mL) was stirred at 25° C. for 1 hour. The mixture was concentrated and the residue was stirred in NH₃·H₂O (2 mL) and MeOH (2 mL) at 25° C. for another 1 hour. The reaction mixture was concentrated and the residue was purified by perp-HPLC (Method C; 0.225% FA as an additive), then lyophilized to give the title compound (10 mg, yield: 34%, FA salt) as a white solid.

¹H NMR (400 MHz, DMSO-d₆) δ 0.92 (3H, t, J=7.2 Hz), 1.51-1.63 (2H, m), 1.72-1.82 (2H, m), 1.84-1.92 (2H, m), 2.11-2.19 (2H, m), 2.21 (3H, s), 2.45-2.50 (1H, m), 2.67-2.74 (2H, m), 4.12 (2H, t, J=6.4 Hz), 4.56 (2H, s), 6.78 (1H, s), 7.65-7.69 (2H, m), 7.72 (1H, d, J=2.8 Hz), 8.42-8.47 (2H, m), 11.99 (1H, brs).

### Example 88

### 4-(2-((1-methylpyrrolidin-3-yl)oxy)-5H-pyrrolo[2,3-b]pyrazin-7-yl)pyridin-2(1H)-one

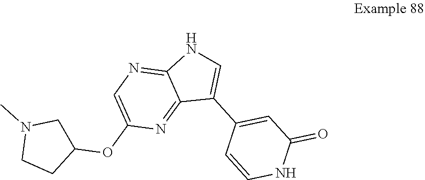 [SMILES: CN1CCC(Oc2cnc3[nH]cc(-c4cc[nH]c(=O)c4)c3n2)C1]

### Step 1. Synthesis of 2-bromo-5-((2-(trimethylsilyl)ethoxy)methyl)-5H-pyrrolo[2,3-b]pyrazine

To a solution of 2-bromo-5H-pyrrolo[2,3-b]pyrazine (5.00 g, 25.3 mmol) in THF (250 mL) was added NaH (2.00 g, 50.5 mmol, 60% dispersion in mineral oil) at 0° C. SEM-Cl (8.40 g, 50.5 mmol) was added dropwise at 0° C. The resulting mixture was stirred at 20° C. for 12 hours. The reaction mixture was quenched with H₂O (50 mL) and extracted EtOAc (100 mL×3). The combined organic layers were dried over anhydrous Na₂SO₄, filtered and concentrated and the residue was purified by Combi Flash (0% to 30% EtOAc in PE) to give 2-bromo-5-((2-(trimethylsilyl)ethoxy)methyl)-5H-pyrrolo[2,3-b]pyrazine (4.50 g, yield: 54%) as yellow oil.

### Step 2. Synthesis of tert-butyl 3-((5-((2-(trimethylsilyl)ethoxy)methyl)-5H-pyrrolo[2,3-b]pyrazin-2-yl)oxy)pyrrolidine-1-carboxylate

A mixture of 2-bromo-5-((2-(trimethylsilyl)ethoxy)methyl)-5H-pyrrolo[2,3-b]pyrazine (4.50 g, 13.7 mmol), tert-butyl 3-hydroxypyrrolidine-1-carboxylate (5.13 g, 27.4 mmol), Pd₂(dba)₃ (628 mg, 0.685 mmol), dppf (456 mg, 0.822 mmol) and Cs₂CO₃ (8.93 g, 27.4 mmol) in toluene (100 mL) was degassed and purged with N₂ for 3 times, then the mixture was stirred at 100° C. for 12 hours under N₂ atmosphere. The reaction mixture was diluted with H₂O (50 mL) and extracted with EtOAc (100 mL×3). The combined organic layers were dried over anhydrous Na₂SO₄, filtered and concentrated. The residue was purified by Combi Flash (0% to 30% EtOAc in PE) to give tert-butyl 3-((5-((2-(trimethylsilyl)ethoxy)methyl)-5H-pyrrolo[2,3-b]pyrazin-2-yl)oxy)pyrrolidine-1-carboxylate (4.40 g, yield: 52%) as yellow oil.

### Step 3. Synthesis of tert-butyl 3-((7-bromo-5-((2-(trimethylsilyl)ethoxy)methyl)-5H-pyrrolo[2,3-b]pyrazin-2-yl)oxy)pyrrolidine-1-carboxylate

To a solution of tert-butyl 3-((5-((2-(trimethylsilyl)ethoxy)methyl)-5H-pyrrolo[2,3-b]pyrazin-2-yl)oxy)pyrrolidine-1-carboxylate (4.40 g, 10.1 mmol) in DMF (50 mL) was added NBS (2.16 g, 12.2 mmol) and stirred at 20° C. for 2 hours. The reaction mixture was concentrated and the residue was purified by prep-HPLC (0.225% FA as an additive), then lyophilized to give tert-butyl 3-((7-bromo-5-((2-(trimethylsilyl)ethoxy)methyl)-5H-pyrrolo[2,3-b]pyrazin-2-yl)oxy)pyrrolidine-1-carboxylate (1.50 g, yield: 29%) as yellow oil.

### Step 4. Synthesis of tert-butyl 3-((7-(2-oxo-1,2-dihydropyridin-4-yl)-5-((2-(trimethylsilyl)ethoxy)methyl)-5H-pyrrolo[2,3-b]pyrazin-2-yl)oxy)pyrrolidine-1-carboxylate

A mixture of tert-butyl 3-((7-bromo-5-((2-(trimethylsilyl)ethoxy)methyl)-5H-pyrrolo[2,3-b]pyrazin-2-yl)oxy)pyrrolidine-1-carboxylate (500 mg, 0.974 mmol), Intermediate 33 (431 mg, 1.95 mmol), XPhos-Pd-G₃ (83 mg, 0.10 mmol) and Na₂CO₃ (206 mg, 1.95 mmol) in 1,4-dioxane (10 mL) and H₂O (1 mL) was degassed and purged with N₂ for 3 times, then the mixture was stirred at 80° C. for 12 hours under N₂ atmosphere. The reaction mixture was diluted with H₂O (30 mL) and extracted EtOAc (50 mL×3). The combined organic layers were dried over anhydrous Na₂SO₄, filtered and concentrated. The residue was purified by Combi Flash (0% to 20% EtOAc in PE) to give tert-butyl 3-((7-(2-oxo-1,2-dihydropyridin-4-yl)-5-((2-(trimethylsilyl)ethoxy)methyl)-5H-pyrrolo[2,3-b]pyrazin-2-yl)oxy)pyrrolidine-1-carboxylate (120 mg, yield: 23%) as yellow oil.

### Step 5. Synthesis of 4-(2-(pyrrolidin-3-yloxy)-5H-pyrrolo[2,3-b]pyrazin-7-yl)pyridin-2(1H)-one

A mixture of tert-butyl 3-((7-(2-oxo-1,2-dihydropyridin-4-yl)-5-((2-(trimethylsilyl)ethoxy)methyl)-5H-pyrrolo[2,3-b]pyrazin-2-yl)oxy)pyrrolidine-1-carboxylate (120 mg, 0.227 mmol) in DCM (4 mL) and TFA (2 mL) was stirred at 20° C. for 2 hours. The reaction mixture was concentrated and the residue was dissolved in NH₃·H₂O (2 mL) and MeOH (2 mL) and stirred at 20° C. for another 2 hours. The reaction mixture was concentrated to give 4-(2-(pyrrolidin-3-yloxy)-5H-pyrrolo[2,3-b]pyrazin-7-yl)pyridin-2(1H)-one (100 mg, crude) as a white solid.

### Step 6. Synthesis of 4-(2-((1-methylpyrrolidin-3-yl)oxy)-5H-pyrrolo[2,3-b]pyrazin-7-yl)pyridin-2(1H)-one

To a solution of 4-(2-(pyrrolidin-3-yloxy)-5H-pyrrolo[2,3-b]pyrazin-7-yl)pyridin-2(1H)-one (100 mg, 0.336 mmol) and HOAc (21 mg, 0.34 mmol) in MeOH (4 mL) was added 37% aqueous formaldehyde (137 mg, 1.68 mmol) at 20° C. and stirred for 0.5 hour. NaBH₃CN (64 mg, 1.0 mmol) was added to the reaction mixture and stirred at 20° C. for another 1 hour. The reaction mixture was concentrated and the residue was purified by prep-HPLC (0.225% FA as an additive), then lyophilized to give the title compound (19.04 mg, yield: 16%, FA salt) as a white solid.

¹H NMR (400 MHz, DMSO-d₆) δ 1.88-1.96 (1H, m), 2.30 (3H, s), 2.40-2.45 (2H, m), 2.70-2.76 (2H, m), 2.90-3.00 (1H, m), 5.40-5.50 (1H, m), 6.91 (1H, d, J=6.8 Hz), 7.34-7.37 (2H, m), 7.96 (1H, s), 8.41 (1H, s), 11.25 (1H, brs), 12.42 (1H, brs).

### Example 89

### 4-(3-(1-methylpiperidin-4-yl)azetidin-1-yl)-7-(pyridin-4-yl)-1,2-dihydro-3H-pyrrolo[3,4-c]pyridin-3-one

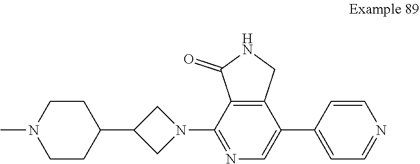 [SMILES: CN1CCC(C2CN(c3ncc(-c4ccncc4)c4c3C(=O)NC4)C2)CC1]

### Step 1. Synthesis of 4,6-dichloro-N-(prop-2-yn-1-yl)pyrimidine-5-carboxamide

To a solution of 4,6-dichloropyrimidine-5-carboxylic acid (8.96 g, 46.4 mmol) and DMF (851 mg, 11.6 mmol) in DCM (90 mL) was added oxalyl chloride (11.8 g, 92.8 mmol) at 0° C. and the mixture was stirred at 20° C. for 1 hour under N₂ atmosphere. The reaction mixture was concentrated and the residue was dissolved in DCM (100 mL), which was added dropwise to a solution of Et₃N (23.5 g, 232 mmol) and propargylamine (2.81 g, 51.1 mmol) in DCM (90 mL) at −78° C. The mixture was stirred at −78° C. for 1 hour under N₂ atmosphere. The reaction mixture was diluted with H₂O (100 mL) and extracted with DCM (100 mL×3). The combined organic layer was washed with brine (150 mL), dried over anhydrous Na₂SO₄, filtered and concentrated. The residue was purified by flash silica gel chromatography (ISCO®; 120 g SepaFlash® Silica Flash Column, Eluent of 0˜25% EtOAc/PE gradient @70 mL/min) to give 4,6-dichloro-N-(prop-2-yn-1-yl)pyrimidine-5-carboxamide (9.64 g, yield: 86%) as a light yellow solid.

### Step 2. Synthesis of 4-chloro-1,2-dihydro-3H-pyrrolo[3,4-c]pyridin-3-one

A mixture of 4,6-dichloro-N-(prop-2-yn-1-yl)pyrimidine-5-carboxamide (200 mg, 0.869 mmol) in nitrobenzene (2 mL) was bubbled with N₂ for 6 minutes. The mixture was microwaved at 250° C. for 2.5 minutes. Five batches were run, combined, and purified by flash silica gel chromatography (ISCO®; 40 g SepaFlash® Silica Flash Column, Eluent of ˜100% EtOAc/PE gradient @50 m/min) to give 4-chloro-1,2-dihydro-3H-pyrrolo[3,4-c]pyridin-3-one (270 mg, yield: 37%) as a yellow solid.

¹H NMR (400 MHz, DMSO-d₆) δ 4.43 (2H, s), 7.68 (1H, d, J=5.2 Hz), 8.53 (1H, d, J=4.8 Hz), 8.88 (1H, brs).

### Step 3. Synthesis of 4-(3-(1-methylpiperidin-4-yl)azetidin-1-yl)-1,2-dihydro-3H-pyrrolo[3,4-c]pyridin-3-one

A mixture of 4-chloro-1,2-dihydro-3H-pyrrolo[3,4-c]pyridin-3-one (130 mg, 0.771 mmol), Intermediate 34 (1.00 g, crude, 2TFA salt) and K₂CO₃ (1.28 g, 9.25 mmol) in CH₃CN (5 mL) was stirred at 90° C. for 16 hours. The reaction mixture was filtered through a pad of celite and the solid was washed with CH₃CN (10 mL×2). The filtrate was concentrated and the residue was purified by flash silica gel column (ISCO®; 12 g SepaFlash® Silica Flash Column, Eluent of 10˜12% MeOH (1.5% NH₃·H₂O as an additive)/DCM gradient @40 m/min), then further purified by prep-HPLC (0.1% NH₄HCO₃ as an additive) and lyophilized to give 4-(3-(1-methylpiperidin-4-yl)azetidin-1-yl)-1,2-dihydro-3H-pyrrolo[3,4-c]pyridin-3-one (80 mg, yield: 34%) as a yellow solid.

### Step 4. Synthesis of 7-bromo-4-(3-(1-methylpiperidin-4-yl)azetidin-1-yl)-1,2-dihydro-3H-pyrrolo[3,4-c]pyridin-3-one

A solution of 4-(3-(1-methylpiperidin-4-yl)azetidin-1-yl)-1,2-dihydro-3H-pyrrolo[3,4-c]pyridin-3-one (93 mg, 0.32 mmol) and NBS (69 mg, 0.39 mmol) in DMF (10 mL) was stirred at 0° C. for 2 hours, then stirred at 20° C. for 14 hours. The reaction mixture was quenched with saturated aqueous Na₂SO₃ (1 mL) and concentrated. The residue was diluted with H₂O (20 mL) and extracted with DCM: MeOH (30 mL×2, 10/1). The combined organic layer was dried over anhydrous Na₂SO₄, filtered and concentrated. The residue was purified by prep-TLC (DCM/MeOH=10/1, 1% NH₃·H₂O as an additive) to give 7-bromo-4-(3-(1-methylpiperidin-4-yl)azetidin-1-yl)-1,2-dihydro-3H-pyrrolo[3,4-c]pyridin-3-one (40 mg, yield: 33%) as a yellow solid.

### Step 5. Synthesis of 4-(3-(1-methylpiperidin-4-yl)azetidin-1-yl)-7-(pyridin-4-yl)-1,2-dihydro-3H-pyrrolo[3,4-c]pyridin-3-one

A mixture of 7-bromo-4-(3-(1-methylpiperidin-4-yl)azetidin-1-yl)-1,2-dihydro-3H-pyrrolo[3,4-c]pyridin-3-one (57 mg, 0.16 mmol), 4-pyridylboronic acid (38 mg, 0.31 mmol), Xphos-Pd-G₃ (26 mg, 0.031 mmol) and Na₂CO₃ (50 mg, 0.47 mmol) in 1, 4-dioxane (3 mL) and H₂O (0.6 mL) was degassed and purged with N₂ for 3 times. The reaction mixture was stirred at 90° C. for 16 hours under N₂ atmosphere. The reaction mixture was concentrated and the residue was purified by flash silica gel column (ISCO®; 4 g SepaFlash® Silica Flash Column, Eluent of ˜14% MeOH/DCM gradient @35 mL/min), then purified by prep-HPLC (0.1% NH₄OAc as an additive) and lyophilized to give the title compound (6 mg, yield: 10%, HOAc salt) as a yellow solid.

¹H NMR (400 MHz, CD₃OD) δ 1.34-1.47 (2H, m), 1.71-1.82 (1H, m), 1.91-2.00 (5H, m), 2.48-2.59 (1H, m), 2.62-2.74 (5H, m), 3.32-3.35 (2H, m), 4.09-4.18 (2H, m), 4.42-4.52 (2H, m), 4.61 (2H, s), 7.57-7.61 (2H, m), 8.37 (1H, s), 8.57-8.62 (2H, m).

### Example 90

### 3-(2-(piperidin-4-ylethynyl)piperidin-4-yl)-1-(tetrahydro-2H-pyran-4-yl)-1H-pyrrolo[2,3-c]pyridine

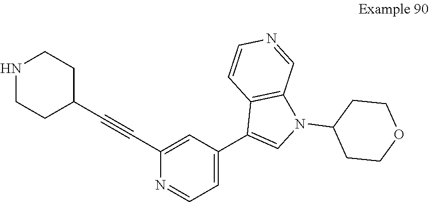 [SMILES: C(#CC1CCNCC1)c1cc(-c2cn(C3CCOCC3)c3cnccc23)ccn1]

### Step 1. Synthesis of tert-butyl 4-((4-(1-(tetrahydro-2H-pyran-4-yl)-1H-pyrrolo[2,3-c]pyridin-3-yl)pyridin-2-yl)ethynyl)piperidine-1-carboxylate

A mixture of Intermediate 25 (350 mg, 1.12 mmol), tert-butyl 4-ethynylpiperidine-1-carboxylate (350 mg, 1.67 mmol) Pd(CH₃CN)₂Cl₂ (29 mg, 0.11 mmol), XPhos (106 mg, 0.223 mmol) and Cs₂CO₃ (1.09 g, 3.35 mmol) in CH₃CN (15 mL) was degassed and purged with N₂ for 3 times, then the mixture was stirred at 80° C. for 12 hours under N₂ atmosphere. The reaction mixture was filtered and the filtrate was concentrated. The residue was purified by flash silica gel chromatography (MeOH/DCM=1/20) to afford tert-butyl 4-((4-(1-(tetrahydro-2H-pyran-4-yl)-1H-pyrrolo[2,3-c]pyridin-3-yl)pyridin-2-yl)ethynyl)piperidine-1-carboxylate (287 mg, yield: 28%) as yellow gum.

### Step 2. Synthesis of 3-(2-(piperidin-4-ylethynyl)pyridin-4-yl)-1-(tetrahydro-2H-pyran-4-yl)-1H-pyrrolo[2,3-c]pyridine

A solution of tert-butyl 4-((4-(1-(tetrahydro-2H-pyran-4-yl)-1H-pyrrolo[2,3-c]pyridin-3-yl)pyridin-2-yl)ethynyl)piperidine-1-carboxylate (55 mg, 0.11 mmol) in DCM (4 mL) and TFA (2 mL) was stirred at 20° C. for 1.5 hours. The reaction mixture was concentrated and the residue was purified by prep-HPLC (0.225% FA as an additive), then lyophilized to afford the title compound (10.78 mg, yield: 24%, FA salt) as a light yellow solid.

¹H NMR (400 MHz, CD₃OD) δ 1.97-2.06 (2H, m), 2.09-2.15 (2H, m), 2.17-2.30 (4H, m), 3.13-3.25 (3H, m), 3.42-3.52 (2H, m), 3.70-3.75 (2H, m), 4.10-4.17 (2H, m), 4.90-4.95 (1H, m), 7.74-7.82 (1H, m), 7.90 (1H, s), 8.02 (1H, d, J=5.6 Hz), 8.29 (1H, d, J=6.0 Hz), 8.41 (1H, s), 8.50 (1H, d, J=5.2 Hz), 9.05 (1H, s).

The following compound was synthesized analogously to Example 90

| Example | | | |
|---|---|---|---|
| No. | Structure | Name | ¹H NMR (400 MHz) |
| 97 | 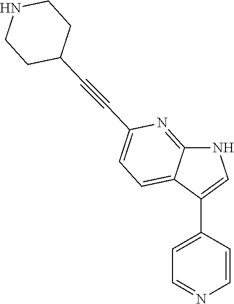 [SMILES: C(#CC1CCNCC1)c1ccc2c(-c3ccncc3)c[nH]c2n1] | 6-(piperidin-4-ylethynyl)- 3-(pyridin-4-yl)-1H- pyrrolo[2,3-b]pyridine | DMSO-d₆; 1.69-1.83 (2H, m), 1.95-2.05 (2H, m), 2.81-3.03 (3H, m), 3.10-3.20 (2H, m), 7.30 (1H, d, J = 8.0 Hz), 7.70-7.80 (2H, m), 8.30 (1H, s), 8.35-8.45 (2H, m), 8.50-8.60 (2H, m). |

### Example 91

### 3-(2-((1-methylpiperidin-4-yl)ethynyl)pyridin-4-yl)-1-(tetrahydro-2H-pyran-4-yl)-1H-pyrrolo[2,3-c]pyridine

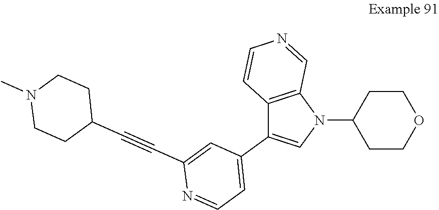 [SMILES: CN1CCC(C#Cc2cc(-c3cn(C4CCOCC4)c4cnccc34)ccn2)CC1]

To a solution of Example 90 (680 mg, 1.76 mmol) in MeOH (8 mL) was added DIPEA to adjust to pH=8, then HOAc was added to adjust pH=5, 37% aqueous HCHO (810 mg, 8.80 mmol) was added to the reaction mixture and stirred at 20° C. for 0.5 hour. NaBH₃CN (332 mg, 5.28 mmol) was added to the reaction mixture and stirred at 20° C. for another 2 hours. The reaction mixture was diluted with 112 (20 mL) and extracted with DCM (20 mL×3). The combined organic layers were dried over anhydrous Na₂SO₄, filtered and concentrated. The residue was purified by prep-HPLC (0.05% NH₃·H₂O as an additive) and lyophilized to afford the title compound (16.92 mg, yield: 2%) as a white solid.

¹H NMR (400 MHz, DMSO-d₆) δ 1.60-1.70 (2H, m), 1.84-1.91 (2H, m), 1.98-2.19 (9H, m), 2.58-2.70 (3H, m), 3.55-3.65 (2H, m), 3.99-4.10 (2H, m), 4.84-4.98 (1H, m), 7.71-7.79 (2H, m), 7.83 (1H, s), 7.95 (1H, d, J=5.6 Hz), 8.28 (1H, d, J=5.6 Hz), 8.49 (1H, d, J=5.2 Hz), 8.61 (1H, s), 9.11 (1H, s).

The following compounds were synthesized analogously to Example 90

| Example | | | |
|---|---|---|---|
| No. | Structure | Name | ¹H NMR (400 MHz) |
| 49 | 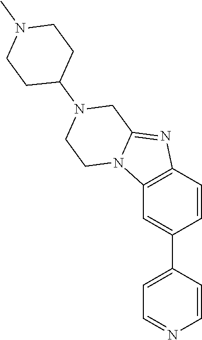 [SMILES: CN1CCC(N2CCn3c(nc4ccc(-c5ccncc5)cc43)C2)CC1] | 2-(1-methylpiperidin-4- yl)-7-(pyridin-4-yl)- 1,2,3,4- tetrahydrobenzo[4,5] imidazo[1,2-a]pyrazine | CD₃OD; δ 1.64-1.78 (2H, m), 1.94-2.03 (2H, m), 2.06-2.18 (2H, m), 2.30 (3H, s), 2.58-2.69 (1H, m), 2.94-3.02 (2H, m), 3.20 (2H, t, J = 5.6 Hz), 4.06 (2H, s), 4.24 (2H, t, J = 5.2 Hz), 7.66-7.73 (2H, m), 7.78-7.82 (2H, m), 7.90- 7.93 (1H, m), 8.55-8.60 (2H, m). |
| 50 | 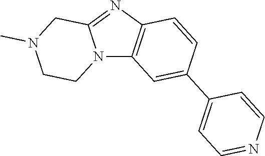 [SMILES: CN1CCn2c(nc3ccc(-c4ccncc4)cc32)C1] | 2-methyl-7-(pyridin-4-yl)- 1,2,3,4- tetrahydrobenzo[4,5] imidazo[1,2-a]pyrazine | CD₃OD; δ 2.59 (3H, s), 3.09 (2H, t, J = 5.6 Hz), 3.90 (2H, s), 4.29 (2H, t, J = 5.6 Hz), 7.68-7.73 (2H, m), 7.78-7.82 (2H, m), 7.91-7.94 (1H, m), 8.55-8.60 (2H, m). |
| 66 | 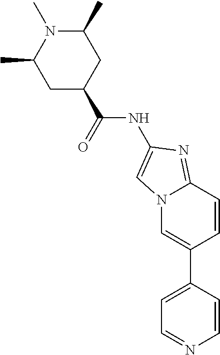 [SMILES: C[C@@H]1C[C@H](C(=O)Nc2cn3cc(-c4ccncc4)ccc3n2)C[C@H](C)N1C] | (2R,4r,6S)-1,2,6- trimethyl-N-(6-(pyridin-4- yl)imidazo[1,2-a]pyridin- 2-yl)piperidine-4- carboxamide | CD₃OD; δ 1.40-1.50 (6H, m), 1.83-2.33 (5H, m), 2.70-2.75 (1H, m), 2.98 (3H, s), 3.70-3.75 (1H, m), 7.69 (1H, dd, J = 9.2, 4.0 Hz), 7.80-7.98 (1H, m), 8.22-8.92 (5H, m), 9.25 (1H, d, J = 4.8 Hz). |
| 96 | 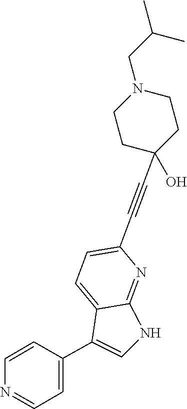 [SMILES: CC(C)CN1CCC(O)(C#Cc2ccc3c(-c4ccncc4)c[nH]c3n2)CC1] | 1-isobutyl-4-((3-(pyridin- 4-yl)-1H-pyrrolo[2,3- b]pyridin-6- yl)ethynyl)piperidin-4-ol | DMSO-d₆; δ 0.86 (6H, d, J = 6.8 Hz), 1.75-1.80 (3H, m), 1.85-1.95 (2H, m), 2.05-2.10 (2H, m), 2.22- 2.40 (2H, m), 2.56-2.72 (2H, m), 5.65 (1H, brs), 7.30 (1H, d, J = 8.4 Hz), 7.72-7.79 (2H, m), 8.31 (1H, d, J = 2.8 Hz), 8.41 (1H, d, J = 8.4 Hz), 8.51-8.58 (2H, m), 12.30 (1H, brs). |
| 98 | 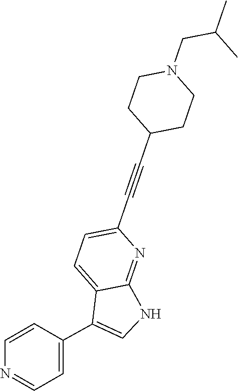 [SMILES: CC(C)CN1CCC(C#Cc2ccc3c(-c4ccncc4)c[nH]c3n2)CC1] | 6-((1-isobutylpiperidin-4- yl)ethynyl)-3-(pyridin-4- yl)-1H-pyrrolo[2,3- b]pyridine | DMSO-d₆; δ 0.85 (6H, d, J = 6.4 Hz), 1.59-1.70 (2H, m), 1.71-1.82 (1H, m), 1.84-1.93 (2H, m), 2.00- 2.05 (2H, m), 2.08-2.12 (2H, m), 2.45-2.50 (1H, m), 2.65-2.68 (2H, m), 7.26 (1H, d, J = 8.4 Hz), 7.72-7.77 (2H, m), 8.29 (1H, s), 8.38 (1H, d, J = 8.4 Hz), 8.52- 8.57 (2H, m). |

### Example 92

### 1-(tetrahydro-2H-pyran-4-yl)-3-(2-((tetrahydro-2H-pyran-4-yl)ethynyl)pyridin-4-yl)-1H-pyrrolo[2,3-c]pyridine

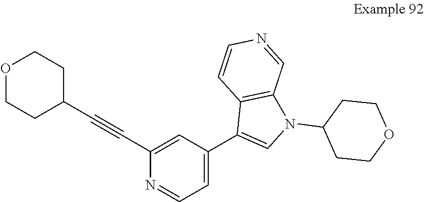 [SMILES: C(#CC1CCOCC1)c1cc(-c2cn(C3CCOCC3)c3cnccc23)ccn1]

A mixture of Intermediate 25 (240 mg, 0.765 mmol), 4-ethynyltetrahydropyran (421 mg, 3.82 mmol), Cs₂CO₃ (748 mg, 2.29 mmol), Pd(CH₃CN)₂Cl₂ (20 mg, 0.076 mmol) and XPhos (73 mg, 0.15 mmol) in CH₃CN (15 mL) was degassed and purged with N₂ for 3 times at 0° C., then the mixture was stirred at 80° C. for 16 hours under N₂ atmosphere. The reaction mixture was concentrated and the residue was diluted with H₂O (20 mL), then extracted with DCM (20 mL×3). The combined organic layer was dried over anhydrous Na₂SO₄, filtered and concentrated. The residue was purified by flash silica gel column (MeOH/DCM=1/20), then further purified by prep-HPLC (0.05% NH₃·H₂O as an additive) and lyophilized to afford the title compound (149.12 mg, yield: 50%) as an off-white solid.

¹H NMR (400 MHz, DMSO-d₆) δ 1.60-1.70 (2H, m), 1.85-1.91 (2H, m), 1.98-2.04 (2H, m), 2.09-2.19 (2H, m), 2.90-3.00 (1H, m), 3.42-3.50 (2H, m), 3.55-3.65 (2H, m), 3.79-3.88 (2H, m), 4.00-4.05 (2H, m), 4.85-4.95 (1H, m), 7.74-7.78 (1H, m), 7.85 (1H, s), 7.96 (1H, m), 8.28 (1H, d, J=5.6 Hz), 8.50 (1H, d, J=5.2 Hz), 8.62 (1H, s), 9.11 (1H, s).

### Example 99

### 4-(3-cyclopropyl-2-((1-isobutylpiperidin-4-yl)ethynyl)-3H-imidazo[4,5-b]pyridin-5-yl)pyridin-2-amine

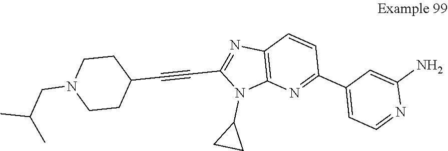 [SMILES: CC(C)CN1CCC(C#Cc2nc3ccc(-c4ccnc(N)c4)nc3n2C2CC2)CC1]

### Step 1. Synthesis of 6-chloro-N-cyclopropyl-3-nitropyridin-2-amine

A mixture of 2,6-dichloro-3-nitropyridine (10.0 g, 51.8 mmol) and cyclopropanamine (5.92 g, 103 mmol) in toluene (80 mL) was stirred at 25° C. for 3 hours under N₂ atmosphere. The reaction mixture was diluted with water (180 mL) and extracted with EtOAc (180 mL×3). The combined organic layers were dried over anhydrous Na₂SO₄, filtered and concentrated to afford 6-chloro-N-cyclopropyl-3-nitropyridin-2-amine (10.0 g, yield: 90%) as a yellow solid.

¹H NMR (400 MHz, DMSO-d₆) δ 0.67-0.73 (2H, m), 0.78-0.84 (2H, m), 2.92-3.03 (1H, m), 6.84 (1H, d, J=8.4 Hz), 8.32-8.48 (2H, m).

### Step 2. Synthesis of 6-chloro-N²-cyclopropylpyridine-2,3-diamine

To a solution of 6-chloro-N-cyclopropyl-3-nitropyridin-2-amine (10.0 g, 46.8 mmol) in EtOH (50 mL) and H₂O (50 mL) was added Fe powder (13.1 g, 234 mmol) and NH₄Cl (12.5 g, 234 mmol) with stirring. The mixture was stirred at 80° C. for 1 hour. The reaction mixture was filtered and the filtrate was concentrated. The residue was purified by silica gel column (PE/EtOAc=5/1) to afford 6-chloro-N²-cyclopropylpyridine-2,3-diamine (5.40 g, yield: 48%) as brown oil.

¹H NMR (400 MHz, DMSO-d₆) δ 0.37-0.43 (2H, m), 0.64-0.71 (2H, m), 2.62-2.78 (1H, m), 4.77 (2H, brs), 6.09 (1H, d, J=2.4 Hz), 6.38 (1H, d, J=8.0 Hz), 6.66 (1H, d, J=7.6 Hz).

### Step 3. Synthesis of 5-chloro-3-cyclopropyl-1,3-dihydro-2H-imidazo[4,5-b]pyridin-2-one

To a solution of triphosgene (8.28 g, 27.9 mmol) and Et₃N (14 mL) in THF (50 mL) was added 6-chloro-N²-cyclopropylpyridine-2,3-diamine (4.70 g, 25.0 mmol) at −10° C. and the mixture was stirred at −10° C. for 0.5 hour. The reaction mixture was basified with saturated aqueous Na₂CO₃ to pH=8 and extracted with EtOAc (200 mL×3). The combined organic layer was dried over anhydrous Na₂SO₄, filtered and concentrated. The residue was purified by silica gel column (PE/EtOAc=3/1) to afford 5-chloro-3-cyclopropyl-1,3-dihydro-2H-imidazo[4,5-b]pyridin-2-one (1.75 g, yield: 33%) as an off-white solid.

¹H NMR (400 MHz, DMSO-d₆) δ 0.95-1.00 (4H, m), 2.86-2.93 (1H, m), 7.05 (1H, d, J=7.6 Hz), 7.29 (1H, d, J=8.0 Hz), 11.17 (1H, brs).

### Step 4. Synthesis of 2-bromo-5-chloro-3-cyclopropyl-3H-imidazo[4,5-b]pyridine

A mixture of 5-chloro-3-cyclopropyl-1,3-dihydro-2H-imidazo[4,5-b]pyridin-2-one (1.00 g, 4.77 mmol), POBr₃ (6.84 g, 23.8 mmol) in toluene (13 mL) was stirred at 80° C. for 16 hours under N₂ atmosphere. The reaction solution was basified with saturated aqueous Na₂CO₃ to pH=8 and extracted with DCM (50 mL×3). The combined organic layer was dried over anhydrous Na₂SO₄, filtered and concentrated. The residue was purified by silica gel column (PE/EtOAc=3/1) to afford 2-bromo-5-chloro-3-cyclopropyl-3H-imidazo[4,5-b]pyridine (510 mg, yield: 39%) as a light yellow solid.

### Step 5. Synthesis of tert-butyl 4-((5-chloro-3-cyclopropyl-3H-imidazo[4,5-b]pyridin-2-yl)ethynyl)piperidine-1-carboxylate

A mixture of 2-bromo-5-chloro-3-cyclopropyl-3H-imidazo[4,5-b]pyridine (770 mg, 2.83 mmol), tert-butyl 4-ethynylpiperidine-1-carboxylate (1.18 g, 5.65 mmol), Pd(PPh₃)₂Cl₂ (198 mg, 0.283 mmol), Et₃N (2 mL) and CuI (108 mg, 0.566 mmol) in THF (10 mL) was degassed and purged with N₂ for 3 times, then the mixture was stirred at 25° C. for 12 hours under N₂ atmosphere. The reaction mixture was concentrated and the residue was purified by silica gel column (PE/EtOAc=5/1) to afford tert-butyl 4-((5-chloro-3-cyclopropyl-3H-imidazo[4,5-b]pyridin-2-yl)ethynyl)piperidine-1-carboxylate (950 mg, yield: 84%) as a brown solid.

¹H NMR (400 MHz, DMSO-d₆) δ 1.10-1.20 (4H, m), 1.40 (9H, s), 1.56-1.66 (2H, m), 1.86-1.96 (2H, m), 3.03-3.11 (1H, m), 3.17-3.24 (2H, m), 3.38-3.46 (1H, m), 3.63-3.73 (2H, m), 7.36 (1H, d, J=8.4 Hz), 8.06 (1H, d, J=8.4 Hz).

### Step 6. Synthesis of tert-butyl 4-((5-(2-((tert-butoxycarbonyl)amino)pyridin-4-yl)-3-cyclopropyl-3H-imidazo[4,5-b]pyridin-2-yl)ethynyl)piperidine-1-carboxylate

A mixture of tert-butyl 4-((5-chloro-3-cyclopropyl-3H-imidazo[4,5-b]pyridin-2-yl)ethynyl)piperidine-1-carboxylate (200 mg, 0.498 mmol), tert-butyl (4-(4,4,5,5-tetramethyl-1,3,2-dioxaborolan-2-yl)pyridin-2-yl)carbamate (192 mg, 0.599 mmol), Xphos-Pd-G₃ (42 mg, 0.49 mmol) and Na₂CO₃ (106 mg, 0.998 mmol) in 1, 4-dioxane (3 mL) and H₂O (0.4 mL) was degassed and purged with N₂ for 3 times, then the mixture was stirred at 100° C. for 2 hours under N₂ atmosphere. The reaction mixture was concentrated and the residue was purified by silica gel column (PE/EtOAc=1/1) to afford tert-butyl 4-((5-(2-((tert-butoxycarbonyl)amino)pyridin-4-yl)-3-cyclopropyl-3H-imidazo[4,5-b]pyridin-2-yl)ethynyl)piperidine-1-carboxylate (250 mg, yield: 72%) as a white solid.

¹H NMR (400 MHz, DMSO-d₆) δ 1.21-1.27 (2H, m), 1.31-1.36 (2H, m), 1.42 (9H, s), 1.51 (9H, s), 1.59-1.70 (2H, m), 1.89-1.98 (2H, m), 3.06-3.15 (1H, m), 3.18-3.25 (2H, m), 3.48-3.57 (1H, m), 3.65-3.75 (2H, m), 7.75 (1H, dd, J=5.2, 1.6 Hz), 7.93 (1H, d, J=8.4 Hz), 8.15 (1H, d, J=8.4 Hz), 8.37 (1H, d, J=5.2 Hz), 8.57 (1H, s), 9.85 (1H, brs).

### Step 7. Synthesis of 4-(3-cyclopropyl-2-(piperidin-4-ylethynyl)-3H-imidazo[4,5-b]pyridin-5-yl)pyridin-2-amine

To a solution of tert-butyl 4-((5-(2-((tert-butoxycarbonyl)amino)pyridin-4-yl)-3-cyclopropyl-3H-imidazo[4,5-b]pyridin-2-yl)ethynyl)piperidine-1-carboxylate (250 mg, 0.447 mmol) in DCM (4 mL) was added TFA (1 mL). The mixture was stirred at 25° C. for 2 hours. The reaction mixture was concentrated to afford 4-(3-cyclopropyl-2-(piperidin-4-ylethynyl)-3H-imidazo[4,5-b]pyridin-5-yl)pyridin-2-amine (420 mg, crude) as brown oil.

### Step 8. Synthesis of 4-(3-cyclopropyl-2-((1-isobutylpiperidin-4-yl)ethynyl)-3H-imidazo[4,5-b]pyridin-5-yl)pyridin-2-amine

To a solution of 4-(3-cyclopropyl-2-(piperidin-4-ylethynyl)-3H-imidazo[4,5-b]pyridin-5-yl)pyridin-2-amine (200 mg, 0.558 mmol) in MeOH (5 mL) was added DIPEA (258 mg, 2.00 mmol) and the mixture was stirred at 25° C. for 10 minutes. Then HOAc (183 mg, 3.04 mmol) and isobutyraldehyde (36 mg, 0.50 mmol) were added and stirred at 25° C. for 20 minutes. NaBH₃CN (53 mg, 0.84 mmol) was added and the reaction mixture was stirred at 25° C. for another 30 minutes. The reaction mixture was concentrated and the residue was purified by prep-HPLC (0.05% NH₄HCO₃ as an additive), then lyophilized to afford the title compound (18.17 mg, yield: 8%) as a yellow solid.

¹H NMR (400 MHz, DMSO-d₆) δ 0.86 (6H, d, J=6.4 Hz), 1.20-1.27 (2H, m), 1.28-1.35 (2H, m), 1.68-1.81 (3H, m), 1.91-2.00 (2H, m), 2.04 (2H, d, J=7.6 Hz), 2.20-2.30 (2H, m), 2.65-2.75 (2H, m), 2.85-2.90 (1H, m), 3.45-3.53 (1H, m), 6.05 (2H, brs), 7.17-7.20 (2H, m), 7.82 (1H, d, J=8.4 Hz), 8.00-8.04 (1H, m), 8.09 (1H, d, J=8.8 Hz).

### Example 100

### 3-(2-((1-isobutylpiperidin-4-yl)ethynyl)pyridin-4-yl)-1-(tetrahydro-2H-pyran-4-yl)-1H-pyrrolo[2,3-c]pyridine

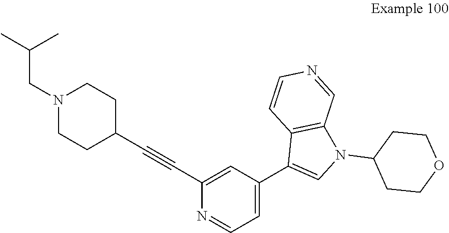 [SMILES: CC(C)CN1CCC(C#Cc2cc(-c3cn(C4CCOCC4)c4cnccc34)ccn2)CC1]

### Step 1. Synthesis of tert-butyl 4-((4-(1-(tetrahydro-2H-pyran-4-yl)-1H-pyrrolo[2,3-c]pyridin-3-yl)pyridin-2-yl)ethynyl)piperidine-1-carboxylate

A mixture of Intermediate 25 (220 mg, 0.701 mmol), tert-butyl 4-ethynylpiperidine-1-carboxylate (220 mg, 1.05 mmol), Pd(CH₃CN)₂Cl₂ (18 mg, 0.07 mmol), XPhos (67 mg, 0.14 mmol) and Cs₂CO₃ (685 mg, 2.10 mmol) in CH₃CN (4 mL) was degassed and purged with N₂ for 3 times, then the mixture was stirred at 80° C. for 14 hours under N₂ atmosphere. The reaction mixture was concentrated and the residue was purified by silica gel column (PE/EtOAc=0/1) to afford tert-butyl 4-((4-(1-(tetrahydro-2H-pyran-4-yl)-1H-pyrrolo[2,3-c]pyridin-3-yl)pyridin-2-yl)ethynyl)piperidine-1-carboxylate (220 mg, yield: 64%) as a brown solid.

¹H NMR (400 MHz, DMSO-d₆) δ 1.41 (9H, s), 1.84-1.90 (2H, m), 2.00-2.07 (3H, m), 2.07-2.20 (3H, m), 2.86-2.99 (2H, m), 3.08-3.21 (3H, m), 3.57-3.63 (2H, m), 3.66-3.73 (2H, m), 4.85-4.95 (1H, m), 7.74-7.79 (1H, m), 7.85 (1H, s), 7.96 (1H, d, J=5.6 Hz), 8.28 (1H, d, J=5.6 Hz), 8.50 (1H, d, J=5.2 Hz), 8.60 (1H, s), 9.11 (1H, s).

### Step 2. Synthesis of 3-(2-(piperidin-4-ylethynyl)pyridin-4-yl)-1-(tetrahydro-2H-pyran-4-yl)-1H-pyrrolo[2,3-c]pyridine

A solution of tert-butyl 4-((4-(1-(tetrahydro-2H-pyran-4-yl)-1H-pyrrolo[2,3-c]pyridin-3-yl)pyridin-2-yl)ethynyl)piperidine-1-carboxylate (220 mg, 0.452 mmol) in DCM (4 mL) and TFA (1 mL) was stirred at 25° C. for 1 hour. The reaction mixture was concentrated to afford 3-(2-(piperidin-4-ylethynyl)pyridin-4-yl)-1-(tetrahydro-2H-pyran-4-yl)-1H-pyrrolo[2,3-c]pyridine (380 mg, crude, TFA salt) as brown gum.

### Step 3. Synthesis of 3-(2-((1-isobutylpiperidin-4-yl)ethynyl)pyridin-4-yl)-1-(tetrahydro-2H-pyran-4-yl)-1H-pyrrolo[2,3-c]pyridine

To a solution of 3-(2-(piperidin-4-ylethynyl)pyridin-4-yl)-1-(tetrahydro-2H-pyran-4-yl)-1H-pyrrolo[2,3-c]pyridine (380 mg, 0.983 mmol) in MeOH (4 mL) was added DIPEA (381 mg, 2.95 mmol) at 25° C. and stirred for 0.5 hour, then HOAc (177 mg, 2.95 mmol), isobutyraldehyde (71 mg, 0.98 mmol) and NaBH₃CN (74 mg, 1.2 mmol) were added. The resulting reaction mixture was stirred at 25° C. for another 0.5 hour. The mixture was concentrated and the residue was purified by prep-HPLC (0.05% NH₃·H₂O as an additive), then lyophilized to afford the title compound (25.31 mg, yield: 6%) as a white solid.

¹H NMR (400 MHz, DMSO-d₆) δ 0.86 (6H, d, J=6.4 Hz), 1.61-1.71 (2H, m), 1.72-1.81 (1H, m), 1.86-1.96 (2H, m), 1.98-2.05 (4H, m), 2.07-2.20 (4H, m), 2.65-2.70 (3H, m), 3.56-3.65 (2H, m), 4.00-4.05 (2H, m), 4.85-4.95 (1H, m), 7.75 (1H, d, J=5.2 Hz), 7.83 (1H, s), 7.95 (1H, d, J=5.6 Hz), 8.28 (1H, d, J=5.6 Hz), 8.49 (1H, d, J=5.2 Hz), 8.61 (1H, s), 9.12 (1H, s).

### Example 102

### 1-methyl-N-(5-(1-methyl-1H-benzo[d]imidazol-6-yl)thiazol-2-yl)piperidine-4-carboxamide

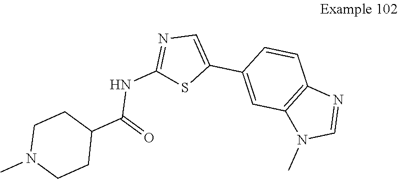 [SMILES: CN1CCC(C(=O)Nc2ncc(-c3ccc4ncn(C)c4c3)s2)CC1]

A mixture of 1-methyl-N-(thiazol-2-yl)piperidine-4-carboxamide (200 mg, 0.888 mmol), 6-bromo-1-methyl-1H-benzo[d]imidazole (206 mg, 0.976 mmol), t-Bu₃PHBF₄ (52 mg, 0.18 mmol), Pd(OAc)₂ (20 mg, 0.09 mmol) and Cs₂CO₃ (578 mg, 1.78 mmol) in DMF (2 mL) was degassed and purged with N₂ for 3 times, then the mixture was stirred at 120° C. for 3 hours under N₂ atmosphere. The mixture was concentrated and the residue was purified by silica gel column (DCM/MeOH=10/1), then further purified by prep-HPLC (0.05% NH₃·H₂O as an additive) and lyophilized to afford the title compound (79.61 mg, yield: 25%) as a white solid.

¹H NMR (400 MHz, DMSO-d₆) δ 1.60-1.71 (2H, m), 1.75-1.81 (2H, m), 1.82-1.90 (2H, m), 2.15 (3H, s), 2.40-2.47 (1H, m), 2.75-2.85 (2H, m), 3.87 (3H, s), 7.47 (1H, dd, J=8.4, 1.6 Hz), 7.66 (1H, d, J=8.4 Hz), 7.79 (1H, s), 7.85 (1H, s), 8.19 (1H, s), 11.98 (1H, brs).

### Example 105

### 1-methyl-N-(6-(2-methylpyridin-4-yl)pyrrolo[1,2-c]pyrimidin-3-yl)piperidine-4-carboxamide

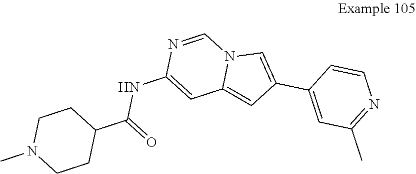 [SMILES: Cc1cc(-c2cc3cc(NC(=O)C4CCN(C)CC4)ncn3c2)ccn1]

### Step 1: Synthesis of tert-Butyl (6-(2-methylpyridin-4-yl)pyrrolo[1,2-c]pyrimidin-3-yl)carbamate

A mixture of tert-butyl (6-bromopyrrolo[1,2-c]pyrimidin-3-yl)carbamate (220 mg, 0.70 mmol.), (2-methylpyridin-4-yl)boronic acid (116 mg, 0.85 mmol), Pd(dppf)Cl₂ (51 mg, 0.07 mmol) and K₂CO₃ (198 mg, 1.41 mmol) in DMA (6 mL) and H₂O (1 mL) was degassed and purged with N₂ 3×. The reaction was stirred at 90° C. for 30 minutes. The reaction mixture was diluted with EtOAc (10 mL) and washed sequentially with H₂O (2×10 mL) and brine (1×10 mL), dried over anhydrous Na₂SO₄, filtered and concentrated in vacuo. Purification by flash chromatography on silica gel (0% to 100% EtOAc/Hexanes) afforded 112 mg (49%) of tert-Butyl (6-(2-methylpyridin-4-yl)pyrrolo[1,2-c]pyrimidin-3-yl)carbamate as an off-white solid. ES-MS [M+1]⁺: 325.3.

### Step 2: Synthesis of 6-(2-Methylpyridin-4-yl)pyrrolo[1,2-c]pyrimidin-3-amine

A solution of tert-Butyl (6-(2-methylpyridin-4-yl)pyrrolo[1,2-c]pyrimidin-3-yl)carbamate (80 mg, 0.25 mmol) in DCM (1 mL) and TFA (1 mL) was stirred at 20° C. for 1 hour. Saturated aqueous NaHCO₃ was added to pH=8 and the reaction extracted with DCM (3×5 mL). The combined organic layers were washed with brine (10 mL), dried over anhydrous Na₂SO₄, filtered and concentrated in vacuo to afford 52 mg (93%) of 6-(2-Methylpyridin-4-yl)pyrrolo[1,2-c]pyrimidin-3-amine as a brown solid which was used without further purification. ES-MS [M+1]⁺: 225.1.

### Step 3: Synthesis of 1-Methyl-N-(6-(2-methylpyridin-4-yl)pyrrolo[1,2-c]pyrimidin-3-yl)piperidine-4-carboxamide

6-(2-Methylpyridin-4-yl)pyrrolo[1,2-c]pyrimidin-3-amine (25 mg, 0.11 mmol,), 1-methylpiperidine-4-carboxylic acid hydrochloride (24 mg, 0.13 mmol), and EDCI·HCl (43 mg, 0.22 mmol) were dissolved in pyridine (2.7 mL) and heated to 90° C. for 1 hour under inert atmosphere. Saturated aqueous NaHCO₃ (5 mL) was added and the reaction extracted with 3:1 CHCl₃:IPA (3×5 mL). The combined organic layers were passed through a phase separator and concentrated in vacuo. The resulting residue was dissolved in DMSO (1.5 mL) and purified by prep HPLC (5-40% CH₃CN/0.05% aqueous NH₄OH over 10 min). Fractions containing the desired product were concentrated to afford 12.9 mg (33%) of 1-Methyl-N-(6-(2-methylpyridin-4-yl)pyrrolo[1,2-c]pyrimidin-3-yl)piperidine-4-carboxamide as a yellow oil. ES-MS [M+1]⁺: 350.2. ¹H NMR (400 MHz, CD₃OD) δ 8.85-8.80 (m, 1H), 8.36 (dd, J=5.4, 0.8 Hz, 1H), 8.01-7.95 (m, 2H), 7.64-7.59 (m, 1H), 7.56-7.50 (m, 1H), 6.75 (t, J=1.2 Hz, 1H), 2.95 (dt, J=12.0, 3.5 Hz, 2H), 2.56 (s, 3H), 2.43 (m, 1H), 2.29 (s, 3H), 2.15-2.04 (m, 2H), 1.87 (td, J=10.7, 9.3, 3.5 Hz, 4H).

### Example 106

### 2,2,5,5-tetramethyl-N-(6-(2-methylpyridin-4-yl)imidazo[1,2-a]pyridin-2-yl)tetrahydrofuran-3-carboxamide

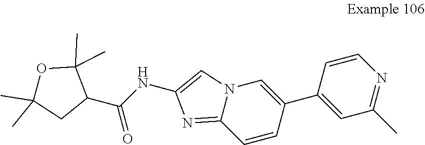

### Step 1: Synthesis of N-(6-bromoimidazo[1,2-a]pyridin-2-yl)-2,2,5,5-tetramethyloxolane-3-carboxamide

HATU (134.48 mg, 0.35 mmol) was added to 2,2,5,5-tetramethyloxolane-3-carboxylic acid (60.9 mg, 0.35 mmol) and 2-amino-6-bromoimidazo[1,2-a]pyridine (50 mg, 0.24 mmol) in DMA (1 mL) at 0° C. then stirred at RT for 30 min. The solution was purified by semi-prep HPLC (2-60% MeCN in 0.05% NH₄OH (aq) over 10 min) to give N-(6-bromoimidazo[1,2-a]pyridin-2-yl)-2,2,5,5-tetramethyloxolane-3-carboxamide (8 mg, 9% yield) as a tan glass. ES-MS [M+1]⁺: 366.1.

### Step 2: Synthesis of 2,2,5,5-tetramethyl-N-[6-(2-methylpyridin-4-yl)imidazo[1,2-a]pyridin-2-yl]oxolane-3-carboxamide

N-(6-Bromoimidazo[1,2-a]pyridin-2-yl)-2,2,5,5-tetramethyloxolane-3-carboxamide (8 mg, 0.02 mmol), 2-picoline-4-boronic acid pinacol ester (32 mg, 0.14 mmol), dichloro[1,1′-bis(diphenylphosphino)ferrocene]palladium dichloromethane adduct (4 mg, 0.005 mmol) and potassium carbonate (34 mg, 0.24 mmol) in DMA (0.5 mL) and water (0.1 mL) was stirred at 90° C. for 2 hr. The solution was purified by prep HPLC (2-60% MeCN in 0.05% NH₄OH (aq) over 12 min) to give 2,2,5,5-tetramethyl-N-[6-(2-methylpyridin-4-yl)imidazo[1,2-a]pyridin-2-yl]oxolane-3-carboxamide (2.5 mg, 7% yield) as a colorless glass. ES-MS [M+1]⁺: 379.5.

### Example 107

### 8-(1-methyl-1H-pyrazol-4-yl)-2-phenylpyrazolo[1,5-a]quinazolin-5(4H)-one

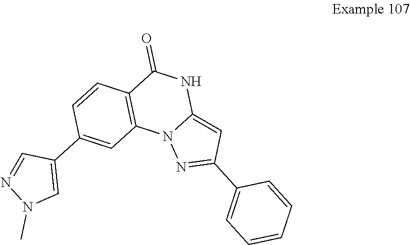 [SMILES: Cn1cc(-c2ccc3c(=O)[nH]c4cc(-c5ccccc5)nn4c3c2)cn1]

### Step 1: Synthesis of 8-Bromo-2-phenylpyrazolo[1,5-a]quinazolin-5(4H)-one

To a stirring suspension of 4-bromo-2-hydrazineylbenzoic acid hydrochloride (30 mg, 0.11 mmol) in acetic acid (0.5 mL) was added 2-oxo-2-phenylacetyl cyanide (16 mg, 0.11 mmol). The reaction was irradiated in a microwave at 150° C. for 10 minutes. Water was added and the resulting precipitate was collect by vacuum filtration, washing with additional water. Precipitate was dried under vacuum to afford 35 mg of 8-Bromo-2-phenylpyrazolo[1,5-a]quinazolin-5(4H)-one as an off-white solid, which was used without further purification. ES-MS [M+1]⁺: 340.0/342.1.

### Step 2: Synthesis of 8-(1-Methyl-H-pyrazol-4-yl)-2-phenylpyrazolo[1,5-a]quinazolin-5(4H)-one

A mixture of 8-Bromo-2-phenylpyrazolo[1,5-a]quinazolin-5(4H)-one (35 mg, 0.10 mmol), 1-methyl-4-(4,4,5,5-tetramethyl-1,3,2-dioxaborolan-2-yl)-1H-pyrazole (25 mg, 0.12 mmol), Cs₂CO₃ (67 mg, 0.21 mmol.) and Pd(dppf)Cl₂ (8 mg, 0.01 mmol) in 1,4-dioxane (1 mL) and water (0.1 mL) was degassed and purged with N₂ 3×. The reaction mixture was stirred at 100° C. for 1 hour. The reaction mixture was concentrated in vacuo and the resulting residue dissolved in DMSO (1.5 mL) and purified by prep HPLC (20-60% CH₃CN/0.1% aqueous TFA over 10 min). Fractions containing the product were basified with saturated aqueous NaHCO₃ solution and extracted with 3:1 CHCl₃:IPA (3×). Combined organic extracts were filtered through a phase separator and concentrated to afford 8-(1-Methyl-1H-pyrazol-4-yl)-2-phenylpyrazolo[1,5-a]quinazolin-5(4H)-one as an off-white solid. ES-MS [M+1]⁺: 342.0. ¹H NMR (400 MHz, CDCl₃) δ 9.62 (s, 1H), 8.33 (d, J=1.6 Hz, 1H), 8.27 (d, J=8.2 Hz, 1H), 8.03-7.91 (m, 3H), 7.89 (s, 1H), 7.56-7.38 (m, 4H), 6.30 (s, 1H), 4.01 (s, 3H).

### Intermediates of Formula (II)

### Intermediate 1

### 6-chloro-3-iodo-1-tosyl-1H-pyrrolo[2,3-b]pyridine

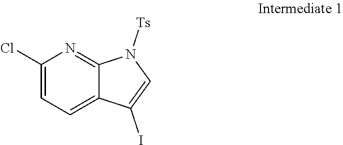 [SMILES: Cc1ccc(S(=O)(=O)n2cc(I)c3ccc(Cl)nc32)cc1]

### Step 1. Synthesis of 6-chloro-3-iodo-1H-pyrrolo[2,3-b]pyridine

To a solution of 6-chloro-1H-pyrrolo[2,3-b]pyridine (5.00 g, 32.8 mmol) in anhydrous DMF (65 mL) was added NIS (8.85 g, 39.3 mmol) at 20-25° C. Then the reaction mixture was stirred at 20-25° C. for 16 hours. The reaction mixture turned into red solution from colorless. To the reaction mixture was added saturated aqueous Na₂SO₃ (200 mL), then filtered. The solid was dissolved in EtOAc (300 mL), then washed with water (100 mL×2), brine (100 mL), dried over anhydrous Na₂SO₄ and concentrated. The crude product was triturated with PE/EtOAc (100 mL, 5/1) to give 6-chloro-3-iodo-1H-pyrrolo[2,3-b]pyridine (9.00 g, yield: 98%) as a yellow solid.

### Step 2. Synthesis of 6-chloro-3-iodo-1-tosyl-1H-pyrrolo[2,3-b]pyridine

To a solution of 6-chloro-3-iodo-1H-pyrrolo[2,3-b]pyridine (7.50 g, 26.9 mmol) in anhydrous THF (150 mL) was added NaH (1.62 g, 40.4 mmol, 60% dispersion in mineral oil) portion-wise at 0° C. After the addition, the reaction mixture was stirred at 0° C. for 1 hour. Then TsCl (6.67 g, 35.0 mmol) was added to the reaction mixture and the resulting reaction mixture was stirred at 10-15° C. for 15 hours. The reaction mixture turned into yellow suspension from yellow solution. The reaction mixture was poured into cold water (50 mL), then extracted with EtOAc (50 mL×3). The combined organic layer was washed with brine (50 mL), dried over anhydrous Na₂SO₄ and concentrated. The crude product was triturated with PE/EtOAc (20 mL, 5/1) to give 6-chloro-3-iodo-1-tosyl-1H-pyrrolo[2,3-b]pyridine (11.0 g, yield: 94%) as a yellow solid.

### Intermediate 2

### 2-(1-methyl-6-oxo-1,6-dihydropyridin-3-yl)acetic Acid

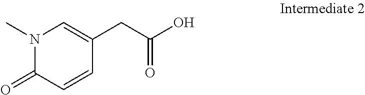

### Step 1. Synthesis of methyl 2-(1-methyl-6-oxo-1,6-dihydropyridin-3-yl)acetate

A mixture of 5-bromo-1-methylpyridin-2(1H)-one (1.00 g, 5.32 mmol), (t-Bu₃P)₂Pd (272 mg, 0.532 mmol) and ZnF₂ (1.65 g, 16.0 mmol) in DMF (10 mL) was degassed and purged with N₂ for 3 times. Then tert-butyl((1-methoxyvinyl)oxy)dimethylsilane (2.00 g, 10.6 mmol) was added to the reaction mixture and stirred at 80° C. for 16 hours. The reaction mixture was filtered and the filtrate was concentrated. The residue was purified by flash silica gel column (EtOAc to DCM/MeOH=10/1) to give methyl 2-(1-methyl-6-oxo-1,6-dihydropyridin-3-yl)acetate (800 mg, yield: 83%) as a yellow oil.

### Step 2. Synthesis of 2-(1-methyl-6-oxo-1,6-dihydropyridin-3-yl)acetic Acid

To a solution of methyl 2-(1-methyl-6-oxo-1,6-dihydropyridin-3-yl)acetate (690 mg, 3.81 mmol) in 1, 4-dioxane (10 mL) was added 4N aqueous HCl (14 mL) and the reaction mixture was stirred at 90° C. for 16 hours. The reaction mixture was concentrated to give 2-(1-methyl-6-oxo-1,6-dihydropyridin-3-yl)acetic acid (1.00 g, crude) as yellow gum.

### Intermediate 3

### 6-chloro-3-iodo-1-((2-(trimethylsilyl)ethoxy)methyl)-1H-pyrrolo[2,3-b]pyridine

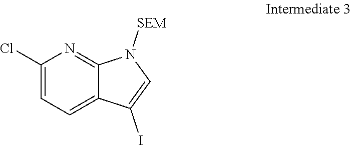 [SMILES: C[Si](C)(C)CCOCn1cc(I)c2ccc(Cl)nc21]

### Step 1. Synthesis of 6-chloro-3-iodo-1H-pyrrolo[2,3-b]pyridine

To a solution of 6-chloro-1H-pyrrolo[2,3-b]pyridine (1.00 g, 6.55 mmol) in DMF (10 mL) was added NIS (1.77 g, 7.86 mmol) and the mixture was stirred at 20° C. for 1 hour. The reaction mixture was poured into H₂O (50 mL) and the precipitate was filtered and dried to give 6-chloro-3-iodo-1H-pyrrolo[2,3-b]pyridine (1.60 g, yield: 87%) as a yellow solid.

¹H NMR (400 MHz, DMSO-d₆) δ 7.20 (1H, d, J=8.4 Hz), 7.70-7.75 (2H, m), 12.31 (1H, brs).

### Step 2. Synthesis of 6-chloro-3-iodo-1-((2-(trimethylsilyl)ethoxy)methyl)-1H-pyrrolo[2,3-b]pyridine

To a solution of 6-chloro-3-iodo-1H-pyrrolo[2,3-b]pyridine (8.79 g, 31.6 mmol) in DMF (100 mL) was added NaH (1.89 g, 47.4 mmol, 60% dispersion in mineral oil) at 0° C. and the mixture was stirred at 0° C. for 0.5 hour. Then SEM-C1 (6.31 g, 37.9 mmol) was added dropwise to the mixture at 0° C. and the mixture was stirred at 20° C. for 1.5 hours. The reaction mixture was quenched by addition H₂O (150 mL) and then extracted with EtOAc (200 mL×3). The combined organic layer was washed with brine (200 mL), dried over anhydrous Na₂SO₄, filtered and concentrated. The residue was purified by flash silica gel column (PE/EtOAc=10/1) to give 6-chloro-3-iodo-1-((2-(trimethylsilyl)ethoxy)methyl)-1H-pyrrolo[2,3-b]pyridine (6.20 g, yield: 48%) as yellow gum.

¹H NMR (400 MHz, CDCl₃) δ −0.04 (9H, s), 0.91-0.95 (2H, m), 3.50-3.60 (2H, m), 5.62 (2H, s), 7.18 (1H, d, J=8.0 Hz), 7.44 (1H, s), 7.67 (1H, d, J=8.0 Hz).

### Intermediate 4

### 1-methyl-4-((tributylstannyl)methoxy)piperidine

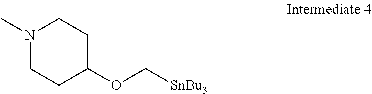

To a mixture of 1-methylpiperidin-4-ol (1.00 g, 8.68 mmol) in THF (10 mL) was added NaH (348 mg, 8.68 mmol, 60% dispersion in mineral oil) at 0° C., then the reaction mixture was stirred at 25° C. for 30 minutes. A solution of tributyl(iodomethyl)stannane (2.49 g, 5.79 mmol) in DMF (18 mL) was added dropwise to the reaction mixture at 0° C. and stirred at 25° C. for 1.5 hours. The reaction mixture was quenched by addition H₂O (50 mL) and extracted with MeOH/DCM (50 mL×2, 1/10). The combined organic layers were washed with brine (100 mL×2), dried over anhydrous Na₂SO₄, filtered and concentrated. The residue was purified by Combi Flash (0% to 30% MeOH in DCM) to give 1-methyl-4-((tributylstannyl)methoxy)piperidine (1.10 g, yield: 45%) as yellow oil.

### Intermediate 5

### 3-iodo-5-nitro-4-propoxy-1-tosyl-1H-pyrrolo[2,3-b]pyridine

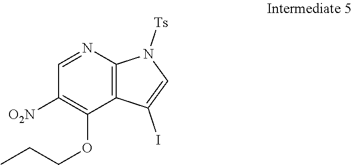 [SMILES: CCCOc1c([N+](=O)[O-])cnc2c1c(I)cn2S(=O)(=O)c1ccc(C)cc1]

### Step 1. Synthesis of 1-((4-bromophenyl)sulfonyl)-4-chloro-5-nitro-1H-pyrrolo[2,3-b]pyridine

To a solution of 1-((4-bromophenyl)sulfonyl)-4-chloro-1H-pyrrolo[2,3-b]pyridine (20.0 g, 68.3 mmol) in DCM (300 mL) was added a solution of n-Bu₄NNO₃ (41.6 g, 137 mmol) in DCM (100 mL) dropwise at 0° C. After the addition, TFAA (28.7 g, 137 mmol) was added slowly at 0° C. The mixture was stirred at 0° C. for 30 minutes, then at 25° C. for another 12.5 hours. The reaction mixture was quenched with H₂O (100 mL) and extracted with DCM (200 mL×3). The combined organic layers were washed with brine (200 mL), dried over anhydrous Na₂SO₄, filtered and concentrated. The residue was purified by column chromatography (SiO₂, DCM/EtOAc=1/0 to 10/1) to give 1-((4-bromophenyl)sulfonyl)-4-chloro-5-nitro-1H-pyrrolo[2,3-b]pyridine (15.0 g, yield: 65%) as a light yellow solid.

### Step 2. Synthesis of 5-nitro-4-propoxy-1H-pyrrolo[2,3-b]pyridine

To n-PrOH (150 mL) was added NaH (8.88 g, 222 mmol, 60% dispersion in mineral oil) portion-wise at 25° C. under N₂ atmosphere. The mixture was stirred at 25° C. for 30 minutes. 1-((4-bromophenyl)sulfonyl)-4-chloro-5-nitro-1H-pyrrolo[2,3-b]pyridine (15.0 g, 44.4 mmol) was added to the reaction mixture and stirred at 80° C. for another 4.5 hours. The reaction mixture was quenched with saturated aqueous NH₄Cl (100 mL) and extracted with EtOAc (200 mL×3). The combined organic layers were washed with brine (300 mL), dried over anhydrous Na₂SO₄, filtered and concentrated to give 5-nitro-4-propoxy-1H-pyrrolo[2,3-b]pyridine (9.00 g, crude) as a brown solid.

### Step 3. Synthesis of 3-iodo-5-nitro-4-propoxy-1H-pyrrolo[2,3-b]pyridine

To a solution of 5-nitro-4-propoxy-1H-pyrrolo[2,3-b]pyridine (9.00 g, 40.7 mmol) in DMF (50 mL) was added a solution of NIS (9.15 g, 40.7 mmol) in dry DMF (50 mL) at 25° C. The resulting reaction mixture was stirred at 25° C. for 5 minutes. The reaction mixture was diluted with H₂O (250 mL) and extracted with EtOAc (200 mL×3). The combined organic layers were washed with brine (100 mL×3), dried over anhydrous Na₂SO₄, filtered and concentrated to give 3-iodo-5-nitro-4-propoxy-1H-pyrrolo[2,3-b]pyridine (14.0 g, yield: 99%) as yellow oil.

### Step 4. Synthesis of 3-iodo-5-nitro-4-propoxy-1-tosyl-1H-pyrrolo[2,3-b]pyridine

To a solution of 3-iodo-5-nitro-4-propoxy-1H-pyrrolo[2,3-b]pyridine (14.0 g, 40.3 mmol) in DCM (150 mL) was added DIPEA (15.6 g, 121 mmol), TsCl (5.69 g, 80.7 mmol) and DMAP (493 mg, 4.03 mmol) at 25° C. The mixture was stirred at 25° C. for 5 minutes. The reaction mixture was concentrated and the residue was purified by column chromatography (SiO₂, Petroleum ether/Ethyl acetate=10/1 to 5/1) to give 3-iodo-5-nitro-4-propoxy-1-tosyl-1H-pyrrolo[2,3-b]pyridine (13.0 g, yield: 64%) as a yellow solid.

### Intermediate 6

### N-(6-bromobenzo[d]thiazol-2-yl)-1-methylpiperidine-4-carboxamide

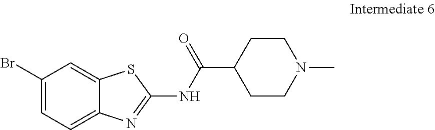 [SMILES: CN1CCC(C(=O)Nc2nc3ccc(Br)cc3s2)CC1]

To a solution of 6-bromobenzo[d]thiazol-2-amine (1.00 g, 4.36 mmol), 1-methylpiperidine-4-carboxylic acid (937 mg, 6.55 mmol), HATU (2.82 g, 7.42 mmol) and DIPEA (1.13 g, 8.73 mmol) in anhydrous DMF (20 mL) was stirred at 40° C. for 16 hours. The reaction mixture was poured into water (100 mL) and EtOAc (25 mL). The precipitate was collected by filtration and dried under vacuum to give N-(6-bromobenzo[d]thiazol-2-yl)-1-methylpiperidine-4-carboxamide (1.00 g, yield: 65%) as a yellow solid.

¹H NMR (400 MHz, CD₃OD) δ1.83-1.93 (2H, m), 1.95-2.00 (2H, m), 2.10-2.20 (2H, m), 2.33 (3H, s), 2.50-2.64 (1H, m), 2.93-3.04 (2H, m), 7.55 (1H, dd, J=8.8, 2.0 Hz), 7.64 (1H, d, J=8.8 Hz), 8.05 (1H, d, J=1.6 Hz).

### Intermediate 7

### 4-(tetrahydro-2H-pyran-4-yl)aniline

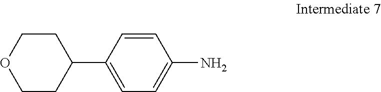 [SMILES: Nc1ccc(C2CCOCC2)cc1]

### Step 1. Synthesis of 4-(4-nitrophenyl)-3,6-dihydro-2H-pyran

A mixture of 1-bromo-4-nitrobenzene (2.00 g, 9.90 mmol) 2-(3,6-dihydro-2H-pyran-4-yl)-4,4,5,5-tetramethyl-1,3,2-dioxaborolane (2.50 g, 11.9 mmol) Pd(dppf)Cl₂ (724 mg, 0.990 mmol) and Na₂CO₃ (3.15 g, 29.7 mmol) in dioxane (40 mL) and H₂O (10 mL) was degassed and purged with N₂ for 3 times. Then the resulting reaction mixture was stirred at 90° C. for 16 hours under N₂ atmosphere. The reaction mixture was diluted with water (50 mL) and EtOAc (50 mL), then filtered through a pad of celite and the solid was washed with EtOAc (20 mL×3). The filtrate was separated and the aqueous layer was extracted with EtOAc (25 mL×3). The combined organic layer was washed with brine (50 mL), dried over anhydrous Na₂SO₄ and concentrated. The residue was purified by Combi Flash (SiO₂, 10% to 20% EtOAc in PE) to give 4-(4-nitrophenyl)-3,6-dihydro-2H-pyran (1.54 g, yield: 76%) as a yellow solid.

¹H NMR (400 MHz, CDCl₃) δ 2.43-2.52 (2H, m), 3.89 (2H, t, J=5.6 Hz), 4.25-4.35 (2H, m), 6.25-6.30 (1H, m), 7.39-7.52 (2H, m), 8.06-8.19 (2H, m).

### Step 2. Synthesis of 4-(tetrahydro-2H-pyran-4-yl)aniline

To a solution of 4-(4-nitrophenyl)-3,6-dihydro-2H-pyran (1.54 g, 7.50 mmol) in MeOH (30 mL) was added 10% Pd/C (300 mg) under N₂ atmosphere. Then the reaction mixture was hydrogenated (45 psi) at 25° C. for 16 hours under H₂ atmosphere. The reaction mixture was filtered through a pad of celite. The solid was washed with MeOH (10 mL×3) and the filtrate was concentrated. The residue was purified by Combi Flash (SiO₂, 20% to 40% EtOAc in PE) to give 4-(tetrahydro-2H-pyran-4-yl)aniline (1.00 g, yield: 75%) as a white solid.

¹H NMR (400 MHz, CDCl₃) δ 1.60-1.75 (4H, m), 2.55-2.65 (1H, m), 3.40-3.47 (2H, m), 3.51 (2H, brs), 3.95-4.05 (2H, m), 6.58 (2H, d, J=8.4 Hz), 6.95 (2H, d, J=8.0 Hz).

### Intermediate 8

### 3-iodo-4-propoxy-1-tosyl-1H-pyrrolo[2,3-b]pyridine

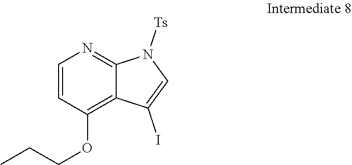 [SMILES: CCCOc1ccnc2c1c(I)cn2S(=O)(=O)c1ccc(C)cc1]

### Step 1. Synthesis of 4-propoxy-1H-pyrrolo[2,3-b]pyridine

To a solution of PPh₃ (2.15 g, 8.20 mmol) in dry THF (5 mL) was added DEAD (1.30 g, 7.46 mmol) dropwise. The above mixture was added to a solution of 1H-pyrrolo[2,3-b]pyridin-4-ol (500 mg, 3.73 mmol) and n-PrOH (269 mg, 4.47 mmol) in dry THF (5 mL). The resulting reaction mixture was stirred at 25° C. for 0.5 hour. The mixture was concentrated and the residue was purified by column chromatography (SiO₂, Petroleum ether/Ethyl acetate=5/1 to 1/1) to give 4-propoxy-1H-pyrrolo[2,3-b]pyridine (220 mg, yield: 33%) as a white solid.

¹H NMR (400 MHz, CDCl₃) δ 1.12 (3H, t, J=7.6 Hz), 1.85-1.95 (2H, m), 4.18 (2H, t, J=6.4 Hz), 6.54 (1H, d, J=5.6 Hz), 6.61 (1H, d, J=3.6 Hz), 7.19 (1H, d, J=3.6 Hz), 8.18 (1H, d, J=5.6 Hz), 9.76 (1H, brs).

### Step 2. Synthesis of 3-iodo-4-propoxy-1H-pyrrolo[2,3-b]pyridine

A solution of I₂ (1.73 g, 6.81 mmol) in dry DMF (5 mL) was added to a solution of 4-propoxy-1H-pyrrolo[2,3-b]pyridine (1.00 g, 5.67 mmol) and KOH (796 mg, 14.2 mmol) in dry DMF (5 mL) at 25° C. The resulting reaction mixture was stirred at 25° C. for 0.5 hour. The reaction mixture was diluted with H₂O (50 mL) and extracted with EtOAc (50 mL×3). The combined organic layers were washed with brine (20 mL×3), dried over anhydrous Na₂SO₄, filtered and concentrated. The residue was purified by column chromatography (SiO₂, Petroleum ether/Ethyl acetate=5/1 to 1/1) to give 3-iodo-4-propoxy-1H-pyrrolo[2,3-b]pyridine (1.50 g, yield: 87%) as a white solid.

### Step 3. Synthesis of 3-iodo-4-propoxy-1-tosyl-1H-pyrrolo[2,3-b]pyridine

To a solution of 3-iodo-4-propoxy-1H-pyrrolo[2,3-b]pyridine (1.50 g, 4.97 mmol) and DIPEA (1.93 g, 14.9 mmol) in DCM (20 mL) was added TsCl (700 mg, 9.93 mmol) and DMAP (61 mg, 0.50 mmol). The mixture was stirred at 25° C. for 0.5 hour. The mixture was concentrated and the residue was purified by column chromatography (SiO₂, Petroleum ether/Ethyl acetate=10/1 to 5/1) to give 3-iodo-4-propoxy-1-tosyl-1H-pyrrolo[2,3-b]pyridine (1.60 g, yield: 71%) as a white solid.

### Intermediate 9

### 5-bromo-4-ethyl-3,3-dimethyl-1,3-dihydro-2H-pyrrolo[2,3-b]pyridin-2-one

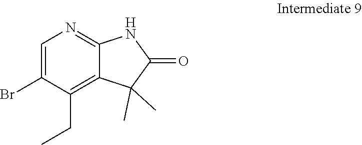

### Step 1. Synthesis of 4-chloro-3,3-dimethyl-1,3-dihydro-2H-pyrrolo[2,3-b]pyridin-2-one

To a mixture of 4-chloro-1,3-dihydro-2H-pyrrolo[2,3-b]pyridin-2-one (1.00 g, 5.93 mmol) in THF (20 mL) was added LiHMDS (11.9 mL, 11.9 mmol, 1 M in THF) at −78° C. under N₂ atmosphere. The reaction mixture was stirred at −78° C. for 1 hour, then MeI (1.52 g, 10.7 mmol) was added and stirred at −78° C. for 0.5 hour. The reaction was allowed to stir at 20-25° C. for another 1.5 hours. The mixture was poured into saturated aqueous NH₄Cl (50 mL) and extracted with EtOAc (50 mL×3). The combined organic layer was washed with brine (50 mL), dried over anhydrous Na₂SO₄, filtered and concentrated. The residue was purified by silica gel column (PE/EtOAc=1/1) to afford 4-chloro-3,3-dimethyl-1,3-dihydro-2H-pyrrolo[2,3-b]pyridin-2-one (400 mg, yield: 34%) as a red solid.

¹H NMR (400 MHz, DMSO-d₆) δ 1.40 (6H, s), 7.06 (1H, d, J=6.0 Hz), 8.06 (1H, d, J=6.0 Hz), 11.31 (1H, brs).

### Step 2. Synthesis of 3,3-dimethyl-4-vinyl-1,3-dihydro-2H-pyrrolo[2,3-b]pyridin-2-one

A mixture of 4-chloro-3,3-dimethyl-1,3-dihydro-2H-pyrrolo[2,3-b]pyridin-2-one (400 mg, 2.03 mmol), potassium vinyltrifluoroborate (551 mg, 4.11 mmol), Ruphos (100 mg, 0.214 mmol), Ruphos-Pd-G₂ (80 mg, 0.10 mmol) and K₂CO₃ (840 mg, 6.08 mmol) in dioxane (10 mL) and H₂O (2 mL) was degassed and purged with N₂ for 3 times. Then the resulting reaction mixture was stirred at 110° C. for 16 hours under N₂ atmosphere. The reaction mixture was diluted with EtOAc (50 mL), then washed with brine (50 mL), dried over anhydrous Na₂SO₄, filtered and concentrated. The residue was purified by silica gel column (35% EtOAc in PE) to afford 3,3-dimethyl-4-vinyl-1,3-dihydro-2H-pyrrolo[2,3-b]pyridin-2-one (300 mg, yield: 78%) as a yellow solid.

¹H NMR (400 MHz, DMSO-d₆) δ 1.30 (6H, s), 5.61 (1H, d, J=11.6 Hz), 6.09 (1H, d, J=18.0 Hz), 7.00 (1H, dd, J=18.0, 11.6 Hz), 7.19 (1H, d, J=5.6 Hz), 8.02 (1H, d, J=5.6 Hz), 11.05 (1H, brs).

### Step 3. Synthesis of 4-ethyl-3,3-dimethyl-1,3-dihydro-2H-pyrrolo[2,3-b]pyridin-2-one

To a solution of 3,3-dimethyl-4-vinyl-1,3-dihydro-2H-pyrrolo[2,3-b]pyridin-2-one (300 mg, 1.59 mmol) in MeOH (10 mL) was added 10% Pd/C (150 mg) under N₂ atmosphere. The reaction mixture was degassed and purged with H₂ for 3 times, then hydrogenated (15 psi) at 20-25° C. for 1 hour. The reaction mixture was filtered and the filtrate was concentrated to give 4-ethyl-3,3-dimethyl-1,3-dihydro-2H-pyrrolo[2,3-b]pyridin-2-one (300 mg, yield: 99%) as a black solid.

### Step 4. Synthesis of 5-bromo-4-ethyl-3,3-dimethyl-1,3-dihydro-2H-pyrrolo[2,3-b]pyridin-2-one

To a solution of 4-ethyl-3,3-dimethyl-1,3-dihydro-2H-pyrrolo[2,3-b]pyridin-2-one (300 mg, 1.58 mmol) in DMF (5 mL) was added NBS (420 mg, 2.36 mmol) at 20-25° C. The reaction mixture was stirred at 20-25° C. for 1 hour. The mixture was concentrated under reduced pressure and the residue was purified by silica gel column (PE/EtOAc=2/1) to afford 5-bromo-4-ethyl-3,3-dimethyl-1,3-dihydro-2H-pyrrolo[2,3-b]pyridin-2-one (400 mg, yield: 94%) as a yellow solid.

¹H NMR (400 MHz, DMSO-d₆) δ 1.17 (3H, t, J=7.2 Hz), 1.37 (6H, s), 2.67-2.81 (2H, m), 8.20 (1H, s), 11.18 (1H, brs).

### Intermediate 10

### tert-butyl (3-amino-3-(3-chlorophenyl)propyl)carbamate

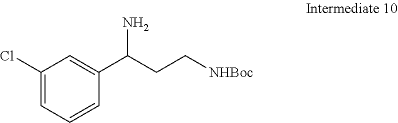 [SMILES: CC(C)(C)OC(=O)NCCC(N)c1cccc(Cl)c1]

### Step 1. Synthesis of 3-amino-J-(3-chlorophenyl)propan-1-ol

To a stirred suspension of LAH (1.27 g, 33.4 mmol) in dry THF (40 mL) was added a solution of 3-(3-chlorophenyl)-3-oxopropanenitrile (2.00 g, 11.1 mmol) in dry THF (40 mL) dropwise at 0° C. under N₂ atmosphere. Then the mixture was stirred at 25° C. for 12 hours. The reaction mixture was quenched with H₂O (20 mL) carefully at 0° C., then 10% aqueous NaOH (20 mL) and H₂O (20 mL) were added at 0° C. The mixture was filtered and the filtrate was extracted with EtOAc (50 mL×3). The combined organic layer was washed with brine (60 mL), dried over anhydrous Na₂SO₄, filtered and concentrated to give 3-amino-1-(3-chlorophenyl)propan-1-ol (1.60 g, crude) as black brown oil.

### Step 2. Synthesis of tert-butyl (3-(3-chlorophenyl)-3-hydroxypropyl)carbamate

To a solution of 3-amino-1-(3-chlorophenyl)propan-1-ol (1.60 g, crude) and Et₃N (1.74 g, 17.2 mmol) in DCM (16 mL) was added Boc₂O (2.82 g, 12.9 mmol) at 0° C. The resulting reaction mixture was stirred at 25° C. for 6 hours. The reaction mixture was diluted with H₂O (40 mL) and extracted with DCM (30 mL×2). The combined organic layer was washed with brine (20 mL), dried over anhydrous Na₂SO₄, filtered and concentrated. The residue was purified by flash silica gel chromatography (ISCO®; 20 g SepaFlash® Silica Flash Column, Eluent of 0˜15% Ethyl acetate/Petroleum ether gradient @36 mL/min) to give tert-butyl (3-(3-chlorophenyl)-3-hydroxypropyl)carbamate (2.00 g, yield: 49% for 2 steps) as a yellow gum.

¹H NMR (400 MHz, CDCl₃) δ 1.49 (9H, s), 1.75-1.90 (2H, m), 3.15-3.25 (1H, m), 3.50-3.70 (2H, m), 4.70-4.80 (1H, m), 4.90-4.95 (1H, m), 6.20-6.25 (3H, m), 7.38 (1H, s).

### Step 3. Synthesis of 2-(((3-((tert-butoxycarbonyl)amino)-1-(3-chlorophenyl)propyl)-l2-azaneyl)carbonyl)benzoic Acid

A mixture of tert-butyl (3-(3-chlorophenyl)-3-hydroxypropyl)carbamate (600 mg, 2.10 mmol), compound Int-10d (340 mg, 2.31 mmol) and PPh₃ (716 mg, 2.73 mmol) in anhydrous THF (6 mL) was added DEAD (475 mg, 2.73 mmol) dropwise at 0° C. Then the mixture was stirred at 20° C. for 1 hour under N₂ atmosphere. The reaction mixture was diluted with H₂O (20 mL) and extracted with EtOAc (20 mL×3). The combined organic layer was washed with brine (20 mL), dried over anhydrous Na₂SO₄, filtered and concentrated. The residue was purified by flash silica gel chromatography (ISCO®; 12 g SepaFlash® Silica Flash Column, Eluent of 0˜25% Ethyl acetate/Petroleum ether gradient @36 mL/min) to give 2-(((3-((tert-butoxycarbonyl)amino)-1-(3-chlorophenyl)propyl)-l2-azaneyl)carbonyl)benzoic acid (800 mg, yield: 64%) as a yellow gum.

### Step 4. Synthesis of tert-butyl (3-amino-3-(3-chlorophenyl)propyl)carbamate

To a solution of 2-(((3-((tert-butoxycarbonyl)amino)-1-(3-chlorophenyl)propyl)-l2-azaneyl)carbonyl)benzoic acid (800 mg, 1.93 mmol) in MeOH (8 mL) and THF (8 mL) was added NH₂NH₂·H₂O (0.75 mL, 715 mmol) at 25° C. Then the mixture was stirred at 55° C. for 3 hours under N₂ atmosphere. The reaction mixture was concentrated, then diluted with H₂O (10 mL) and extracted with ethyl acetate (10 mL×3). The combined organic layer was washed with water (10 mL×3) and brine (10 mL), dried over anhydrous Na₂SO₄, filtered and concentrated. The residue was purified by flash silica gel chromatography (ISCO®; 4 g SepaFlash® Silica Flash Column, Eluent of 0˜8% DCM/MeOH @30 mL/min) to give tert-butyl (3-amino-3-(3-chlorophenyl)propyl)carbamate (400 mg, yield: 44%) as yellow gum.

### Intermediate 11

### 2-methylquinazoline-7-carboxylic Acid

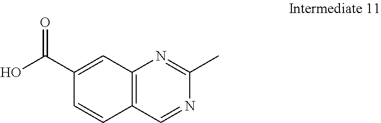

### Step 1. Synthesis of methyl 2-methylquinazoline-7-carboxylate

A mixture of 7-bromo-2-methylquinazoline (200 mg, 0.897 mmol), Pd(OAc)₂ (40 mg, 0.18 mmol), dppp (74 mg, 0.18 mmol) and Et₃N (907 mg, 8.97 mmol) in MeOH (2 mL) and DMSO (2 mL) was degassed and purged with CO for 3 times. Then the resulting reaction mixture was stirred at 75° C. for 16 hours under CO atmosphere (45 psi). The reaction mixture was concentrated. The residue was diluted with H₂O (10 mL) and extracted with EtOAc (10 mL×3). The combined organic layer was washed with brine (10 mL), dried over anhydrous Na₂SO₄, filtered and concentrated. The residue was purified by flash silica gel chromatography (ISCO®; 4 g SepaFlash® Silica Flash Column, Eluent of 0˜45% Ethyl acetate/Petroleum ether gradient @20 mL/min) to give methyl 2-methylquinazoline-7-carboxylate (150 mg, yield: 79%) as a yellow solid.

¹H NMR (400 MHz, CDCl₃) δ 2.97 (3H, s). 4.02 (3H, s), 7.97 (1H, d, J=8.4 Hz), 8.20 (1H, dd, J=8.4, 1.6 Hz), 8.68 (1H, s), 9.42 (1H, s).

### Step 2. Synthesis of 2-methylquinazoline-7-carboxylic Acid

A solution of methyl 2-methylquinazoline-7-carboxylate (150 mg, 0.742 mmol) and LiOH·H₂O (156 mg, 3.71 mmol) in MeOH (1 mL), THF (4 mL) and H₂O (1 mL) was stirred at 25° C. for 1 hour. The reaction mixture was diluted with H₂O (10 mL) and washed with EtOAc (10 mL). The aqueous phase was acidified with 1N aqueous HCl to pH=3 and extracted with EtOAc (10 mL×3). The combined organic layer was washed with brine (20 mL), dried over anhydrous Na₂SO₄, filtered and concentrated to give 2-methylquinazoline-7-carboxylic acid (120 mg, yield: 69%) as a yellow solid.

¹H NMR (400 MHz, DMSO-d₆) δ 2.81 (3H, s), 8.13 (1H, d, J=8.4 Hz), 8.18 (1H, d, J=8.4 Hz), 8.39 (1H, s), 9.61 (1H, s).

### Intermediate 12

### 3-phenoxypyrrolidine

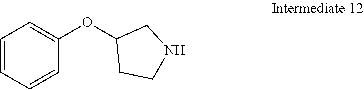 [SMILES: c1ccc(OC2CCNC2)cc1]

### Step 1. Synthesis of tert-butyl 3-phenoxypyrrolidine-1-carboxylate

A mixture of tert-butyl 3-hydroxypyrrolidine-1-carboxylate (2.00 g, 10.7 mmol), phenyl methanesulfonate (1.84 g, 10.7 mmol) and t-BuONa (1.03 g, 10.7 mmol) in CH₃CN (10 mL) was degassed and purged with N₂ for 3 times, and then the mixture was stirred at 80° C. for 16 hours under N₂ atmosphere. The reaction mixture was filtered and the filtrate was concentrated. The residue was purified by flash silica gel chromatography (ISCO®; 20 g SepaFlash® Silica Flash Column, Eluent of 0˜30% Ethyl acetate/Petroleum ether gradient @40 mL/min) to give tert-butyl 3-phenoxypyrrolidine-1-carboxylate (2.00 g, yield: 35%) as colorless oil.

### Step 2. Synthesis of 3-phenoxypyrrolidine

A solution of tert-butyl 3-phenoxypyrrolidine-1-carboxylate (2.00 g, 7.59 mmol) in 4N HCl/MeOH (20 mL) was stirred at 25° C. for 16 hours. The reaction mixture was concentrated and the residue was dissolved in water (20 mL), then washed with EtOAc (20 mL×2). The aqueous layer was basified with 2N aqueous NaOH to pH=12 and extracted with DCM (30 mL×3). The combined organic layer was washed with brine (25 mL), dried over anhydrous Na₂SO₄, filtered and concentrated to give 3-phenoxypyrrolidine (500 mg, yield: 40%) as yellow oil.

### Intermediate 13

### 5-bromo-3-iodo-1-tosyl-1H-pyrrolo[2,3-b]pyridine

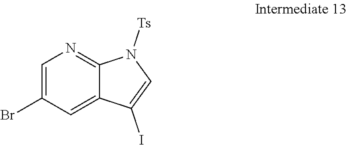 [SMILES: Cc1ccc(S(=O)(=O)n2cc(I)c3cc(Br)cnc32)cc1]

To a solution of 5-bromo-3-iodo-1H-pyrrolo[2,3-b]pyridine (11.0 g, 34.1 mmol) in THF (100 mL) was added NaH (1.77 g, 44.3 mmol, 60% dispersion in mineral oil) in portions at 0° C. After the addition, the mixture was stirred at 0° C. for 30 minutes. TsCl (9.74 g, 51.1 mmol) was added to the reaction mixture and the resulting mixture was stirred at 25° C. for 16 hours. The reaction mixture was quenched with H₂O (100 mL) and extracted with EtOAc/THF (100 mL×3, 2/1). The combined organic layer was washed with brine (50 mL×3), dried over anhydrous Na₂SO₄, filtered and concentrated. The crude product was triturated with MeOH (30 mL) to give 5-bromo-3-iodo-1-tosyl-1H-pyrrolo[2,3-b]pyridine (15.4 g, yield: 94%) as a white solid.

¹H NMR (400 MHz, DMSO-d₆) δ 2.34 (s, 3H), 7.43 (2H, d, J=8.4 Hz), 7.96-8.06 (3H, m), 8.18-8.24 (1H, m), 8.51 (1H, d, J=2.4 Hz).

### Compounds of Formula (II)

### Example 1

### methyl 3-(6-(1-methylpiperidine-4-carboxamido)-1H-pyrrolo[2,3-b]pyridin-3-yl)cyclobutane-1-carboxylate

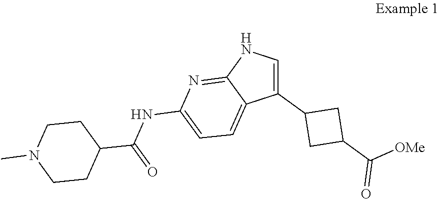 [SMILES: COC(=O)C1CC(c2c[nH]c3nc(NC(=O)C4CCN(C)CC4)ccc23)C1]

### Step 1. Synthesis of ethyl 3-(6-chloro-1-tosyl-H-pyrrolo[2,3-b]pyridin-3-yl)-3-hydroxycyclobutane-1-carboxylate

To a solution of compound Int-1 (5.00 g, 11.6 mmol) in anhydrous THF (75 mL) was added i-PrMgCl (6.9 mL, 2M in Et₂O) dropwise at −10° C. After the addition, the reaction mixture was stirred at −10° C. for 1 hour. Ethyl 3-oxocyclobutanecarboxylate (3.29 g, 23.1 mmol) was added to the reaction mixture at 0° C. Then the resulting reaction mixture was stirred at 0° C. for 2 hours. The reaction mixture turned into yellow solution from colorless solution. The reaction mixture was quenched with 1N aqueous HCl (100 mL), then extracted with EtOAc (100 mL×3). The combined organic layer was washed with brine (100 mL), dried over anhydrous Na₂SO₄ and concentrated. The residue was purified by Combi Flash (SiO₂, 10% to 30% EtOAc in PE) to give ethyl 3-(6-chloro-1-tosyl-1H-pyrrolo[2,3-b]pyridin-3-yl)-3-hydroxycyclobutane-1-carboxylate (4.90 g, yield: 88%) as colorless gum.

### Step 2. Synthesis of ethyl 3-(6-chloro-1-tosyl-1H-pyrrolo[2,3-b]pyridin-3-yl)cyclobutane-J-carboxylate

To a solution of ethyl 3-(6-chloro-1-tosyl-1H-pyrrolo[2,3-b]pyridin-3-yl)-3-hydroxycyclobutane-1-carboxylate (4.90 g, 10.9 mmol) in TFA (15 mL) and DCM (30 mL) was added Et₃SiH (15 mL) dropwise at 15-20° C. After the addition, the reaction mixture was stirred at 15-20° C. for 16 hours. The reaction mixture turned into yellow solution from colorless solution. The reaction mixture was concentrated and the residue was basified with saturated aqueous NaHCO₃ to pH=8, then extracted with EtOAc (100 mL×3). The combined organic layer was washed with brine (100 mL), dried over anhydrous Na₂SO₄ and concentrated. The residue was purified by Combi Flash (SiO₂, 10% to 30% EtOAc in PE), then triturated with PE/EtOAc (25 mL, 10/1) to give ethyl 3-(6-chloro-1-tosyl-1H-pyrrolo[2,3-b]pyridin-3-yl)cyclobutane-1-carboxylate (2.50 g, yield: 53%) as a white solid.

¹H NMR (400 MHz, CDCl₃) δ 1.22 (3H, d, J=7.2 Hz), 2.26-2.48 (5H, m), 2.51-2.72 (2H, m), 3.05-3.18 (1H, m), 3.38-3.75 (1H, m), 4.11 (2H, q, J=7.2 Hz), 7.09 (1H, dd, J=8.4, 4.0 Hz), 7.20-7.25 (2H, m), 7.40-7.45 (1H, m), 7.60-7.80 (1H, m), 8.00-8.10 (2H, m).

### Step 3. Synthesis of ethyl 3-(6-amino-1-tosyl-1H-pyrrolo[2,3-b]pyridin-3-yl)cyclobutane-1-carboxylate

A mixture of ethyl 3-(6-chloro-1-tosyl-1H-pyrrolo[2,3-b]pyridin-3-yl)cyclobutane-1-carboxylate (1.00 g, 2.31 mmol), BocNH₂ (406 mg, 3.46 mmol), Pd₂(dba)₃ (212 mg, 0.231 mmol), Brettphos (248 mg, 0.462 mmol) and Cs₂CO₃ (2.26 g, 6.93 mmol) in anhydrous dioxane (15 mL) was degassed and purged with N₂ for 3 times. Then the reaction mixture was stirred at 90° C. for 16 hours under N₂ atmosphere. The reaction mixture turned into yellow suspension from black suspension. The reaction mixture was diluted with water (50 mL), then extracted with EtOAc (50 mL×3). The combined organic layer was washed with brine (50 mL), dried over anhydrous Na₂SO₄ and concentrated to give intermediate (1.19 g, crude), which was dissolved in TFA (10 mL) and DCM (10 mL) and stirred at 15-20° C. for 2 hours. The reaction mixture turned into yellow brown solution. The reaction mixture was concentrated and the residue was basified with saturated aqueous NaHCO₃ to pH=8, then extracted with EtOAc (50 mL×3). The combined organic layer was washed with brine (50 mL), dried over anhydrous Na₂SO₄ and concentrated. The residue was purified by Combi Flash (SiO₂, 10% to 50% EtOAc in PE) to give ethyl 3-(6-amino-1-tosyl-1H-pyrrolo[2,3-b]pyridin-3-yl)cyclobutane-1-carboxylate (700 mg, yield: 71%) as a yellow gum.

### Step 4. Synthesis of methyl 3-(6-amino-1H-pyrrolo[2,3-b]pyridin-3-yl)cyclobutane-1-carboxylate

To a suspension of ethyl 3-(6-amino-1-tosyl-1H-pyrrolo[2,3-b]pyridin-3-yl)cyclobutane-1-carboxylate (450 mg, 1.09 mmol) in MeOH (10 mL) and H₂O (5 mL) was added KOH (305 mg, 5.44 mmol), then the reaction mixture was stirred at 60° C. for 16 hours. The reaction mixture turned into yellow brown solution from yellow suspension. The reaction mixture was concentrated and the residue was acidified with 1N aqueous HCl to pH=3. The solution was concentrated and used for the next step. The residue was suspended in MeOH (2.5 mL) and DCM (10 mL), then TMSCHN₂ (2.2 mL, 2M in hexane) was added to the reaction mixture at 15-20° C. The reaction mixture was stirred at 15-20° C. for 1 hour. The reaction mixture turned into yellow solution from brown. The reaction mixture was concentrated and the residue was diluted with water (25 mL), then extracted with EtOAc (25 mL×3). The combined organic layer was washed with brine (25 mL), dried over anhydrous Na₂SO₄ and concentrated. The residue was purified by Combi Flash (SiO₂, 20% to 50% EtOAc in PE) to give methyl 3-(6-amino-1H-pyrrolo[2,3-b]pyridin-3-yl)cyclobutane-1-carboxylate (200 mg, yield: 63%) as a yellow gum.

### Step 5. Synthesis of methyl 3-(6-(1-methylpiperidine-4-carboxamido)-1H-pyrrolo[2,3-b]pyridin-3-yl)cyclobutane-1-carboxylate

A mixture of methyl 3-(6-amino-1H-pyrrolo[2,3-b]pyridin-3-yl)cyclobutane-1-carboxylate (200 mg, 0.815 mmol), 1-methylpiperidine-4-carboxylic acid (234 mg, 1.63 mmol) and EDCI (469 mg, 2.45 mmol) in pyridine (3 mL) was stirred at 90° C. for 16 hours. The reaction mixture turned into brown solution from yellow. The reaction mixture was concentrated and the residue was diluted with water (25 mL), then extracted with EtOAc/THF (20 mL×3, 1/1). The combined organic layer was washed with brine (20 mL), dried over anhydrous Na₂SO₄ and concentrated. The crude product was triturated with CH₃CN (5 mL) to give the title compound (130 mg, yield: 41%) as an off-white solid. Among, 40 mg of it was purified by prep-HPLC (Method C; 0.225% FA as an additive), then lyophilized to give Example 1 (12.4 mg, FA salt) as a white solid.

¹H NMR (400 MHz, DMSO-d₆) δ 1.64-1.87 (4H, m), 2.12-2.20 (2H, m), 2.29-2.46 (5H, m), 2.54-2.65 (2H, m), 2.95-3.00 (2H, m), 3.12-3.30 (1H, m), 3.60-3.65 (5H, m), 7.14-7.15 (1H, m), 7.80-8.00 (2H, m), 8.28 (1H, s), 10.22 (1H, brs), 11.15 (1H, brs).

The following compounds were synthesized analogously to Example 1

| Example | | | |
|---|---|---|---|
| No. | Structure | Name | ¹H NMR (400 MHz) |
| 10 | 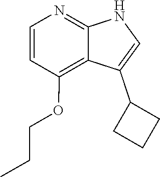 [SMILES: CCCOc1ccnc2[nH]cc(C3CCC3)c12] | 3-cyclobutyl-4- propoxy-1H- pyrrolo[2,3- b]pyridine | CD₃OD; δ 1.14 (3H, t, J = 7.2 Hz), 1.81-2.07 (4H, m), 2.09-2.22 (2H, m), 2.35-2.45 (2H, m), 3.85- 3.95 (1H, m), 4.12 (2H, t, J = 6.4 Hz), 6.56 (1H, d, J = 5.6 Hz), 6.96 (1H, s), 7.97 (1H, d, J = 5.6 Hz). |
| 17 | 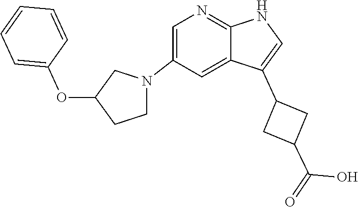 [SMILES: O=C(O)C1CC(c2c[nH]c3ncc(N4CCC(Oc5ccccc5)C4)cc23)C1] | 3-(5-(3- phenoxypyrrolidin- 1-yl)-1H- pyrrolo[2,3- b]pyridin-3- yl)cyclobutane-1- carboxylic acid | DMSO-d₆; δ 2.10-2.20 (1H, m), 2.30-2.45 (4H, m), 2.53-2.64 (2H, m), 2.96-3.20 (1H, m), 3.41- 3.52 (3H, m), 3.68-3.79 (1H, m), 5.15-5.20 (1H, m), 6.92-7.00 (3H, m), 7.12-7.27 (1H, m), 7.28- 7.35 (3H, m), 7.75-7.80 (1H, m), 10.90-10.95 (1H, m). |

### Example 2

### 3-(6-(1-methylpiperidine-4-carboxamido)-1H-pyrrolo[2,3-b]pyridin-3-yl)cyclobutane-1-carboxylic Acid

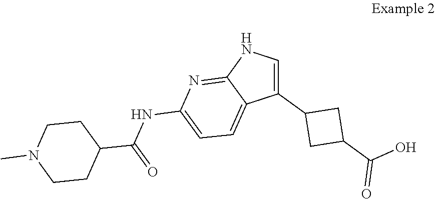 [SMILES: CN1CCC(C(=O)Nc2ccc3c(C4CC(C(=O)O)C4)c[nH]c3n2)CC1]

To a solution of Example 1 (90 mg, 0.24 mmol) in THF (1 mL), MeOH (1 mL) and H₂O (0.5 mL) was added LiOH·H₂O (102 mg, 2.43 mmol) at 20-25° C. Then the reaction mixture was stirred at 20-25° C. for 3 hr. The reaction mixture turned into brown solution from suspension. The reaction mixture was acidified with FA to pH=3 then concentrated. The residue was purified by prep-HPLC (0.225% FA as an additive) then lyophilized to give the title compound (54.14 mg, yield: 62%, FA salt) as a white solid.

¹H NMR (400 MHz, DMSO-d₆) δ 1.61-1.84 (4H, m), 2.02-2.10 (2H, m), 2.27 (3H, s), 2.30-2.40 (2H, m), 2.53-2.63 (2H, m), 2.90-2.95 (2H, m), 3.03-3.17 (1H, m), 3.66-3.69 (2H, m), 7.10-7.25 (1H, m), 7.80-7.95 (2H, m), 8.27 (1H, s), 10.19 (1H, brs), 11.12 (1H, brs).

The following compound was synthesized analogously to Example 2

| Example | | | |
|---|---|---|---|
| No. | Structure | Name | ¹H NMR (400 MHz) |
| 21 | 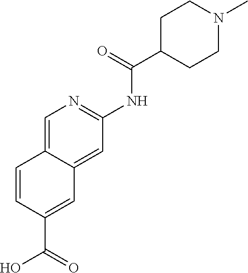 [SMILES: CN1CCC(C(=O)Nc2cc3cc(C(=O)O)ccc3cn2)CC1] | 3-(1-methylpiperidine-4- carboxamido)isoquinoline- 6-carboxylic acid | DMSO-d₆; δ 1.74-1.91 (4H, m), 2.20-2.30 (2H, m), 2.38 (3H, s), 2.60-2.65 (1H, m), 3.00-3.05 (2H, m), 7.95 (1H, dd, J = 8.4, 1.2 Hz), 8.08 (1H, d, J = 8.4 Hz), 8.41 (1H, s), 8.58 (1H, s), 9.20 (1H, s), 10.67 (1H, brs). |

### Example 3

### 2-(1-methyl-6-oxo-1,6-dihydropyridin-3-yl)-N-(3-(trifluoromethyl)-1H-pyrrolo[2,3-b]pyridin-6-yl)acetamide

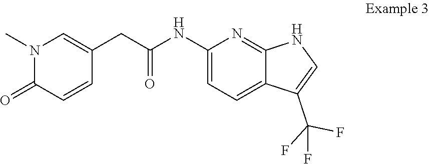 [SMILES: Cn1cc(CC(=O)Nc2ccc3c(C(F)(F)F)c[nH]c3n2)ccc1=O]

### Step 1. Synthesis of 6-chloro-3-(trifluoromethyl)-1-((2-(trimethylsilyl)ethoxy)methyl)-1H-pyrrolo[2,3-b]pyridine

A mixture of compound Int-3 (2.00 g, 4.89 mmol), FSO₂CF₂CO₂Me (2.82 g, 14.7 mmol) and CuI (2.80 g, 14.7 mmol) in DMF (20 mL) was degassed and purged with N₂ for 3 times at 0° C., and then the mixture was stirred at 100° C. for 10 hours under N₂ atmosphere. The reaction mixture was quenched by addition H₂O (20 mL), then basified with saturated aqueous NaHCO₃ to pH=8 and extracted with EtOAc (60 mL×3). The combined organic layer was washed with brine (50 mL), dried over anhydrous Na₂SO₄, filtered and concentrated. The residue was purified by flash silica gel column (PE/EtOAc=10/1) to give 6-chloro-3-(trifluoromethyl)-1-((2-(trimethylsilyl)ethoxy)methyl)-1H-pyrrolo[2,3-b]pyridine (1.50 g, yield: 87%) as a yellow gum.

¹H NMR (400 MHz, CDCl₃) δ −0.02-0.01 (9H, m), 0.94-1.01 (2H, m), 3.55-3.65 (2H, m), 5.70 (2H, s), 7.29-7.30 (1H, m), 7.72 (1H, d, J=1.2 Hz), 8.02 (1H, d, J=8.0 Hz).

### Step 2. Synthesis of tert-butyl (3-(trifluoromethyl)-1-((2-(trimethylsilyl)ethoxy)methyl)-1H-pyrrolo[2,3-b]pyridin-6-yl)carbamate

A mixture of 6-chloro-3-(trifluoromethyl)-1-((2-(trimethylsilyl)ethoxy)methyl)-1H-pyrrolo[2,3-b]pyridine (2.45 g, 6.98 mmol), Pd₂(dba)₃ (639 mg, 0.698 mmol), XPhos (666 mg, 1.40 mmol), Cs₂CO₃ (6.83 g, 20.9 mmol) and BocNH₂ (3.27 g, 27.9 mmol) in 1, 4-dioxane (30 mL) was degassed and purged with N₂ for 3 times, then the mixture was stirred at 90° C. for 16 hours under N₂ atmosphere. The reaction mixture was filtered and the filtrate was concentrated. The residue was purified by flash silica gel column (PE/EtOAc=10/1) to give tert-butyl (3-(trifluoromethyl)-1-((2-(trimethylsilyl)ethoxy)methyl)-1H-pyrrolo[2,3-b]pyridin-6-yl)carbamate (3.10 g, yield: 97%) as a brown gum.

¹H NMR (400 MHz, CDCl₃) δ −0.05-−0.02 (9H, m), 0.88-0.95 (2H, m), 1.55 (9H, s), 3.51-3.59 (2H, m), 5.56 (2H, s), 7.24 (1H, brs), 7.52 (1H, d, J=1.2 Hz), 7.93 (1H, d, J=8.4 Hz), 8.00 (1H, d, J=8.8 Hz).

### Step 3. Synthesis of 3-(trifluoromethyl)-1H-pyrrolo[2,3-b]pyridin-6-amine

A solution of tert-butyl (3-(trifluoromethyl)-1-((2-(trimethylsilyl)ethoxy)methyl)-1H-pyrrolo[2,3-b]pyridin-6-yl)carbamate (2.00 g, 4.63 mmol) in DCM (80 mL) was added TFA (40 mL) and stirred at 20° C. for 2.5 hours. The reaction mixture was concentrated and the residue was dissolved in MeOH (80 mL) and 28% aqueous NH₃·H₂O (40 mL), then stirred at 20° C. for another 0.5 hour. The reaction mixture was concentrated and the residue was diluted with water (50 mL), then extracted with DCM/MeOH (100 mL×3, 10/1). The combined organic layer was washed with brine (100 mL), dried over anhydrous Na₂SO₄ filtered and concentrated to afford 3-(trifluoromethyl)-1H-pyrrolo[2,3-b]pyridin-6-amine (782 mg, yield: 84%) as a yellow solid.

### Step 4. Synthesis of 2-(1-methyl-6-oxo-1,6-dihydropyridin-3-yl)-N-(3-(trifluoromethyl)-1H-pyrrolo[2,3-b]pyridin-6-yl)acetamide

A mixture of 3-(trifluoromethyl)-1H-pyrrolo[2,3-b]pyridin-6-amine (300 mg, 1.49 mmol), compound Int-2 (748 mg, 4.48 mmol) and EDCI (1.14 g, 5.97 mmol) in pyridine (6 mL) was stirred at 90° C. for 1.5 hours. The reaction mixture was concentrated and the residue was diluted with H₂O (50 mL), then basified with saturated aqueous Na₂CO₃ to pH=8, then extracted with DCM/MeOH (60 mL×3, 10/1). The combined organic layer was washed with brine (50 mL), dried over anhydrous Na₂SO₄, filtered and concentrated. The residue was purified by prep-HPLC (0.225% FA as an additive) and lyophilized to afford the title compound (122.06 mg, yield: 22%) as a red solid.

¹H NMR (400 MHz, DMSO-d₆) δ 3.41 (3H, s), 3.50 (2H, s), 6.36 (1H, d, J=9.2 Hz), 7.41 (1H, dd, J=9.6, 2.8 Hz), 7.60 (1H, d, J=2.4 Hz), 7.79-8.21 (3H, m), 10.63 (1H, brs), 12.26 (1H, brs).

The following compounds were synthesized analogously to Example 3

| Example | | | |
|---|---|---|---|
| No. | Structure | Name | ¹H NMR (400 MHz) |
| 4 | 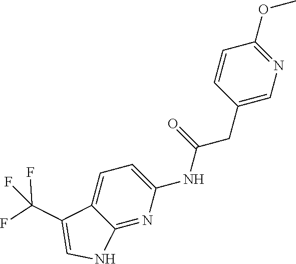 [SMILES: COc1ccc(CC(=O)Nc2ccc3c(C(F)(F)F)c[nH]c3n2)cn1] | 2-(6-methoxypyridin-3-yl)- N-(3-(trifluoromethyl)-1H- pyrrolo[2,3-b]pyridin-6- yl)acetamide | DMSO-d₆; δ 3.70 (2H, s), 3.82 (3H, s), 6.79 (1H, d, J = 8.4 Hz), 7.68 (1H, dd, J = 8.8, 2.4 Hz), 7.95- 8.00 (3H, m), 8.11 (1H, d, J = 2.0 Hz), 10.69 (1H, brs), 12.26 (1H, brs). |
| 9 | 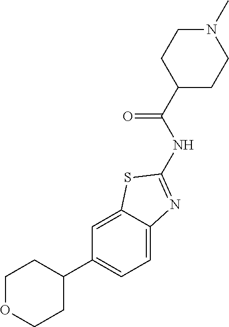 [SMILES: CN1CCC(C(=O)Nc2nc3ccc(C4CCOCC4)cc3s2)CC1] | 1-methyl-N-(6-(tetrahydro- 2H-pyran-4- yl)benzo[d]thiazol-2- yl)piperidine-4-carboxamide | DMSO-d₆; δ 1.60- 1.80 (6H, m), 1.80- 1.90 (2H, m), 2.05- 2.15 (2H, m), 2.28 (3H, s), 2.53-2.63 (1H, m), 2.80-3.10 (3H, m), 3.38-3.52 (2H, m), 3.90-4.00 (2H, m), 7.33 (1H, dd, J = 8.4, 1.6 Hz), 7.66 (1H, d, J = 8.4 Hz), 7.85 (1H, s), 8.22 (1H, s), 12.26 (1H, brs). |
| 13 | 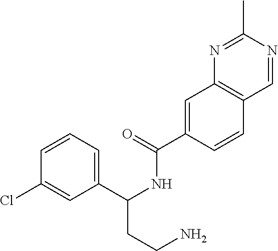 [SMILES: Cc1ncc2ccc(C(=O)NC(CCN)c3cccc(Cl)c3)cc2n1] | N-(3-amino-1-(3- chlorophenyl)propyl)-2- methylquinazoline-7- carboxamide | D₂O; δ 2.20-2.40 (2H, m), 2.62 (3H, s), 2.93- 3.18 (2H, m), 5.05- 5.10 (1H, m), 7.22- 7.36 (3H, m), 7.42 (1H, s), 7.59 (1H, d, J = 8.4 Hz), 7.70-7.75 (2H, m), 8.40 (1H, s), 9.08 (1H, s). |
| 16 | 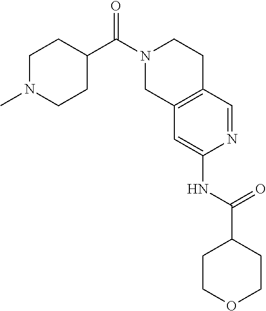 [SMILES: CN1CCC(C(=O)N2CCc3cnc(NC(=O)C4CCOCC4)cc3C2)CC1] | N-(6-(1-methylpiperidine-4- carbonyl)-5,6,7,8-tetrahydro- 2,6-naphthyridin-3- yl)tetrahydro-2H-pyran-4- carboxamide | CD₃OD; δ 1.74-1.92 (8H, m), 2.12-2.25 (2H, m), 2.31 (3H, s), 2.66-2.89 (3H, m), 2.90-3.02 (3H, m), 3.46-3.56 (2H, m), 3.79-3.91 (2H, m), 3.97-4.09 (2H, m), 4.53-4.65 (2H, m), 7.90-7.99 (1H, m), 8.14 (1H, s). |
| 23 | 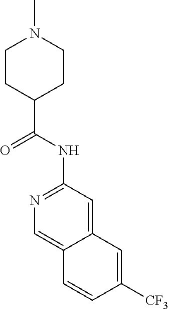 [SMILES: CN1CCC(C(=O)Nc2cc3cc(C(F)(F)F)ccc3cn2)CC1] | 1-methyl-N-(6- (trifluoromethyl)isoquinolin- 3-yl)piperidine-4- carboxamide | DMSO-d₆; δ 1.62- 1.74 (2H, m), 1.75- 1.82 (2H, m), 1.83- 1.94 (2H, m), 2.16 (3H, s), 2.52-2.55 (1H, m), 2.77-2.88 (2H, m), 7.75 (1H, dd, J = 8.8, 1.2 Hz), 8.28 (1H, d, J = 8.4 Hz), 8.39 (1H, s), 8.67 (1H, s), 9.30 (1H, s), 10.71 (1H, brs). |
| 25 | 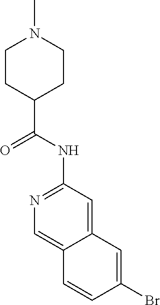 [SMILES: CN1CCC(C(=O)Nc2cc3cc(Br)ccc3cn2)CC1] | N-(6-bromoisoquinolin-3-yl)- 1-methylpiperidine-4- carboxamide | DMSO-d₆; δ 1.64- 1.72 (2H, m), 1.73- 1.80 (2H, m), 1.83- 1.92 (2H, m), 2.10- 2.12 (1H, m), 2.16 (3H, s), 2.80-2.85 (2H, m), 7.63 (1H, dd, J = 8.4, 1.6 Hz), 7.99 (1H, d, J = 8.8 Hz), 8.18 (1H, d, J = 1.2 Hz), 8.45 (1H, s), 9.14 (1H, s), 10.61 (1H, brs). |
| 26 | 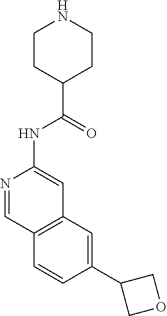 [SMILES: O=C(Nc1cc2cc(C3COC3)ccc2cn1)C1CCNCC1] | N-(6-(oxetan-3- yl)isoquinolin-3- yl)piperidine-4-carboxamide | DMSO-d₆; 1.69- 1.72 (2H, m), 1.85- 1.88 (2H, m), 2.61- 2.84 (3H, m), 3.16- 3.19 (2H, m), 4.41- 4.66 (1H, m), 4.70- 4.75 (2H, m), 4.95- 5.05 (2H, m), 7.61 (1H, dd, J = 8.4, 1.2 Hz), 7.81 (1H, s), 8.06 (1H, d, J = 8.4 Hz), 8.37 (1H, s), 8.45 (1H, s), 9.10 (1H, s), 10.59 (1H, brs). |

### Example 5

### N-(6-(3,6-dihydro-2H-pyran-4-yl)benzo[d]thiazol-2-yl)-1-methylpiperidine-4-carboxamide

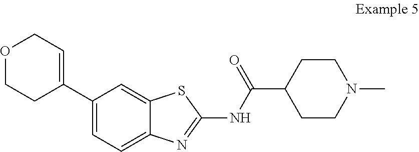 [SMILES: CN1CCC(C(=O)Nc2nc3ccc(C4=CCOCC4)cc3s2)CC1]

A mixture of compound Int-6 (400 mg, 1.13 mmol), 2-(3,6-dihydro-2H-pyran-4-yl)-4,4,5,5-tetramethyl-1,3,2-dioxaborolane (500 mg, 2.38 mmol), Pd(dppf)Cl₂ (165 mg, 0.226 mmol) and Na₂CO₃ (239 mg, 2.26 mmol) in dioxane (6 mL) and water (1.5 mL) was degassed and purged with N₂ for 3 times. Then the reaction mixture was stirred at 130° C. for 1 hour under microwave irradiation. The reaction mixture was filtered and the filtrate was concentrated. The residue was purified by flash silica gel chromatography (ISCO®; 4 g SepaFlash® Silica Flash Column, Eluent of 0˜10% MeOH/DCM ether gradient @20 mL/min), then further purified by prep-HPLC (Method C; 0.225% FA as an additive) and lyophilized to give the title compound (21.21 mg, yield: 18%, FA salt) as a white solid.

¹H NMR (400 MHz, CD₃OD) δ 2.03-2.09 (2H, m), 2.12-2.23 (2H, m), 2.55-2.65 (2H, m), 2.78 (3H, s), 2.90-2.95 (3H, m), 3.42-3.52 (2H, m), 3.97 (2H, t, J=4.4 Hz), 4.30-4.35 (2H, m), 6.25-6.30 (1H, m), 7.58 (1H, dd, J=8.8, 2.0 Hz), 7.71 (1H, d, J=8.4 Hz), 7.94 (1H, d, J=1.6 Hz), 8.46 (1H, s).

### Example 6

### 1-methyl-N-(6-morpholinobenzo[d]thiazol-2-yl)piperidine-4-carboxamide

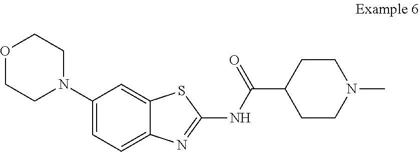 [SMILES: CN1CCC(C(=O)Nc2nc3ccc(N4CCOCC4)cc3s2)CC1]

A mixture of compound Int-6 (200 mg, 0.565 mmol), morpholine (59 mg, 0.68 mmol), Pd₂(dba)₃ (52 mg, 0.056 mmol), Ruphos (26 mg, 0.056 mmol) and Cs₂CO₃ (552 mg, 1.69 mmol) and in anhydrous DMF (5 mL) was degassed and purged with N₂ for 3 times. Then the reaction mixture was stirred at 120° C. for 16 hours under N₂ atmosphere. The reaction mixture was diluted with water (30 mL) and extracted with EtOAc (15 mL×3). The combined organic layer was washed with brine (20 mL×3), dried over anhydrous Na₂SO₄, filtered and concentrated. The residue was purified by prep-HPLC (Method C; 0.225% FA as an additive), then lyophilized to give the title compound (12 mg, yield: 24%, FA salt) as a white solid.

¹H NMR (400 MHz, CD₃OD) δ 2.03-2.10 (2H, m), 2.12-2.28 (2H, m), 2.80 (3H, s), 2.90-2.95 (3H, m), 3.17-3.21 (4H, m), 3.45-3.50 (2H, m), 3.84-3.93 (4H, m), 7.19 (1H, dd, J=8.8, 2.4 Hz), 7.44 (1H, d, J=2.4 Hz), 7.64 (1H, d, J=8.8 Hz), 8.46 (1H, s).

The following compounds were synthesized analogously to Example 6

| Example | | | |
|---|---|---|---|
| No. | Structure | Name | ¹H NMR (400 MHz) |
| 22 | 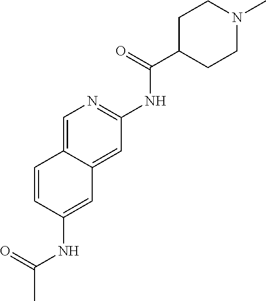 [SMILES: CC(=O)Nc1ccc2cnc(NC(=O)C3CCN(C)CC3)cc2c1] | N-(6- acetamidoisoquinolin- 3-yl)-1- methylpiperidine-4- carboxamide | DMSO-d₆; δ 1.65-1.77 (2H, m), 1.77-1.85 (2H, m), 2.03- 2.11 (2H, m), 2.12 (3H, s), 2.28 (3H, s), 2.53-2.60 (1H, m), 2.90-2.95 (2H, m), 7.57 (1H, dd, J = 9.2, 2.0 Hz), 7.95 (1H, d, J = 8.8 Hz), 8.17 (1H, s), 8.32 (1H, s), 8.96 (1H, s), 10.33 (1H, brs), 10.50 (1H, brs). |
| 24 | 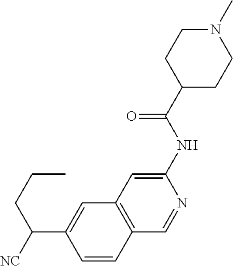 [SMILES: CCCC(C#N)c1ccc2cnc(NC(=O)C3CCN(C)CC3)cc2c1] | N-(6-(1- cyanobutyl)isoquinolin- 3-yl)-1- methylpiperidine-4- carboxamide | DMSO-d₆; δ 0.93 (3H, t, J = 7.2 Hz), 1.36-1.49 (2H, m), 1.65-1.76 (2H, m), 1.79-1.86 (2H, m), 1.87-2.00 (2H, m), 2.02-2.13 (2H, m), 2.30-2.40 (3H, m), 2.54-2.62 (1H, m), 2.90-3.00 (2H, m), 4.40-4.48 (1H, m), 7.54 (1H, dd, J = 8.4, 1.2 Hz), 7.86 (1H, s), 8.10 (1H, d, J = 8.4 Hz), 8.17-8.25 (1H, m), 8.48 (1H, s), 9.15 (1H, s), 10.62 (1H, brs). |

### Example 7

### 6-((1-methylpiperidin-4-y)oxy)methyl)-4-propoxy-3-(trifluoromethyl)-1H-pyrrolo[2,3-b]pyridine

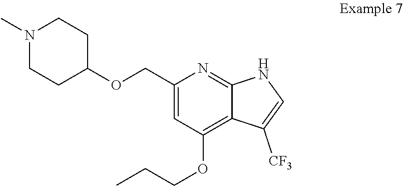 [SMILES: CCCOc1cc(COC2CCN(C)CC2)nc2[nH]cc(C(F)(F)F)c12]

### Step 1. Synthesis of 6-(((1-methylpiperidin-4-yl)oxy)methyl)-4-propoxy-3-(trifluoromethyl)-1-((2-(trimethylsilyl)ethoxy)methyl)-1H-pyrrolo[2,3-b]pyridine

A mixture of 6-chloro-4-propoxy-3-(trifluoromethyl)-1-((2-(trimethylsilyl)ethoxy)methyl)-1H-pyrrolo[2,3-b]pyridine (180 mg, 0.440 mmol), compound Int-4 (276 mg, 0.660 mmol), Pd(OAc)₂ (10 mg, 0.05 mmol), Xphos (63 mg, 0.13 mmol) in DMF (10 mL) was degassed and purged with N₂ for 3 times, then the mixture was stirred at 90° C. for 12 hours under N₂ atmosphere. The reaction mixture was quenched with saturated aqueous KF (20 mL) and extracted with EtOAc (25 mL×2). The combined organic layers were washed with brine (25 mL×2), dried over anhydrous Na₂SO₄, filtered and concentrated. The residue was purified by Combi Flash (0% to 30% MeOH in DCM) to give 6-(((1-methylpiperidin-4-yl)oxy)methyl)-4-propoxy-3-(trifluoromethyl)-1-((2-(trimethylsilyl)ethoxy)methyl)-1H-pyrrolo[2,3-b]pyridine (130 mg, yield: 59%) as yellow oil.

### Step 2. Synthesis of 6-(((1-methylpiperidin-4-yl)oxy)methyl)-4-propoxy-3-(trifluoromethyl)-1H-pyrrolo[2,3-b]pyridine

A mixture of 6-(((1-methylpiperidin-4-yl)oxy)methyl)-4-propoxy-3-(trifluoromethyl)-1-((2-(trimethylsilyl)ethoxy)methyl)-1H-pyrrolo[2,3-b]pyridine (130 mg, 0.259 mmol) in TFA (2 mL) was stirred at 25° C. for 1 hour. Then the reaction mixture was concentrated and the residue was dissolved in 28% aqueous NH₃·H₂O (2 mL) and MeOH (2 mL) and stirred at 25° C. for another 1 hour. The reaction mixture was concentrated and the residue was purified by prep-HPLC (Method C; 0.225% FA as an additive), then lyophilized to give the title compound (67 mg, yield: 70%, FA salt) as a white solid.

¹H NMR (400 MHz, DMSO-d₆) δ 1.04 (3H, t, J=7.4 Hz), 1.50-1.64 (2H, m), 1.76-1.84 (2H, m), 1.86-1.95 (2H, m), 2.10-2.15 (2H, m), 2.20 (3H, s), 2.50-2.55 (1H, m), 2.65-2.75 (2H, m), 4.15 (2H, t, J=6.2 Hz), 4.58 (2H, s), 6.84 (1H, s), 7.86 (1H, s), 8.18 (1H, s), 12.23 (1H, brs).

### Example 8

### 5-isopropoxy-3-isopropyl-4-propoxy-1H-pyrrolo[2,3-b]pyridine

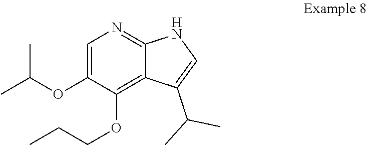 [SMILES: CCCOc1c(OC(C)C)cnc2[nH]cc(C(C)C)c12]

### Step 1. Synthesis of 5-nitro-3-(prop-1-en-2-yl)-4-propoxy-1-tosyl-1H-pyrrolo[2,3-b]pyridine

A mixture of compound Int-5 (13.0 g, 25.9 mmol), potassium trifluoro(prop-1-en-2-yl)borate (4.99 g, 33.7 mmol), Pd(dppf)Cl₂ (949 mg, 1.30 mmol) and Cs₂CO₃ (12.7 g, 38.9 mmol) in dioxane (130 mL) and H₂O (30 mL) was degassed and purged with N₂ for 3 times then at 25° C. and then stirred at 80° C. for 3 hours under N₂ atmosphere. The reaction mixture was diluted with H₂O (200 mL) and extracted with EtOAc (100 mL×3). The combined organic layers were washed with brine (100 mL), dried over anhydrous Na₂SO₄, filtered and concentrated. The residue was purified by column chromatography (SiO₂, Petroleum ether/Ethyl acetate=20/1 to 10/1) to give 5-nitro-3-(prop-1-en-2-yl)-4-propoxy-1-tosyl-1H-pyrrolo[2,3-b]pyridine (8.50 g, yield: 79%) as a yellow oil.

¹H NMR (400 MHz, CDCl₃) δ 0.89 (3H, t, J=7.6 Hz), 1.61-1.75 (2H, m), 2.08 (3H, s), 2.34 (3H, s), 3.95 (2H, t, J=6.4 Hz), 4.98-5.37 (2H, m), 7.26 (2H, d, J=8.4 Hz), 7.60 (1H, s), 8.03 (2H, d, J=8.4 Hz), 8.76 (1H, s).

### Step 2. Synthesis of 3-isopropyl-4-propoxy-1-tosyl-1H-pyrrolo[2,3-b]pyridin-5-amine

To a solution of 5-nitro-3-(prop-1-en-2-yl)-4-propoxy-1-tosyl-1H-pyrrolo[2,3-b]pyridine (8.00 g, 19.3 mmol) in MeOH (50 mL) was added 5% Pd/C (7 g) under N₂ atmosphere. The reaction mixture was degassed and purged with H₂ for several times. The mixture was hydrogenated (40 psi) at 50° C. for 4 hours. The reaction mixture was filtered and the filtrate was concentrated. The residue was purified by column chromatography (SiO₂, Petroleum ether/Ethyl acetate=10/1 to 1/1) to give 3-isopropyl-4-propoxy-1-tosyl-1H-pyrrolo[2,3-b]pyridin-5-amine (7.00 g, yield: 94%) as a light yellow oil.

¹H NMR (400 MHz, CDCl₃) δ 1.09 (3H, t, J=7.2 Hz), 1.32 (6H, d, J=6.8 Hz), 1.80-1.90 (2H, m), 2.38 (3H, s), 3.10-3.20 (1H, m), 3.58 (2H, brs), 3.89 (2H, t, J=6.8 Hz), 7.25 (2H, d, J=8.4 Hz), 7.31 (1H, s), 7.96 (s, 1H), 8.01 (2H, d, J=8.4 Hz).

### Step 3. Synthesis of 5-bromo-3-isopropyl-4-propoxy-1-tosyl-1H-pyrrolo[2,3-b]pyridine

To a solution of 3-isopropyl-4-propoxy-1-tosyl-1H-pyrrolo[2,3-b]pyridin-5-amine (4.00 g, 10.3 mmol) in THF (80 mL) was added tert-butyl nitrite (2.13 g, 20.7 mmol) and CuBr₂ (5.76 g, 25.8 mmol). The mixture was stirred at 50° C. for 1 hour. The reaction mixture was quenched with H₂O (100 mL) and extracted with EtOAc (100 mL×3). The combined organic layers were washed with brine (100 mL), dried over anhydrous Na₂SO₄, filtered and concentrated. The residue was purified by column chromatography (SiO₂, Petroleum ether/Ethyl acetate=20/1 to 5/1) to give 5-bromo-3-isopropyl-4-propoxy-1-tosyl-1H-pyrrolo[2,3-b]pyridine (1.60 g, yield: 34%) as a yellow solid.

¹H NMR (400 MHz, CDCl₃) δ 1.06 (3H, t, J=7.6 Hz), 1.29 (6H, d, J=6.8 Hz), 1.80-1.95 (2H, m), 2.36 (3H, s), 2.34-2.39 (1H, m), 4.06 (2H, t, J=6.4 Hz), 7.22-7.26 (2H, m), 7.36 (1H, s), 8.02 (2H, d, J=8.4 Hz), 8.38 (1H, s).

### Step 4. Synthesis of 3-isopropyl-4-propoxy-5-(4,4,5,5-tetramethyl-1,3,2-dioxaborolan-2-yl)-1-tosyl-1H-pyrrolo[2,3-b]pyridine

A mixture of 5-bromo-3-isopropyl-4-propoxy-1-tosyl-1H-pyrrolo[2,3-b]pyridine (2.00 g, 4.43 mmol), Bis-Pin (5.63 g, 22.2 mmol), Pd(dppf)Cl₂ (324 mg, 0.443 mmol) and KOAc (1.30 g, 13.3 mmol) in anhydrous dioxane (20 mL) was degassed and purged with N₂ for 3 times. Then the resulting reaction mixture was stirred at 110° C. for 36 hours under N₂ atmosphere. The reaction mixture was quenched with H₂O (50 mL) and extracted with EtOAc (50 mL×3). The combined organic layers were washed with brine (50 mL), dried over anhydrous Na₂SO₄, filtered and concentrated. The residue was purified by column chromatography (SiO₂, Petroleum ether/Ethyl acetate=10/1 to 5/1) to give 3-isopropyl-4-propoxy-5-(4,4,5,5-tetramethyl-1,3,2-dioxaborolan-2-yl)-1-tosyl-1H-pyrrolo[2,3-b]pyridine (630 mg, yield: 29%) as a white solid.

### Step 5. Synthesis of 3-isopropyl-4-propoxy-1-tosyl-1H-pyrrolo[2,3-b]pyridin-5-ol

To a solution of 3-isopropyl-4-propoxy-5-(4,4,5,5-tetramethyl-1,3,2-dioxaborolan-2-yl)-1-tosyl-1H-pyrrolo[2,3-b]pyridine (800 mg, 1.61 mmol) in EtOAc (15 mL) was added 30% aqueous H₂O₂ (3.1 mL, 32.1 mmol) at 25° C. The mixture was stirred at 25° C. for 12 hours. The reaction mixture was quenched with saturated aqueous Na₂SO₃ (50 mL) and extracted with EtOAc (50 mL×3). The combined organic layers were washed with brine (50 mL), dried over anhydrous Na₂SO₄, filtered and concentrated. The residue was purified by column chromatography (SiO₂, Petroleum ether/Ethyl acetate=5/1 to 1/1) to give 3-isopropyl-4-propoxy-1-tosyl-1H-pyrrolo[2,3-b]pyridin-5-ol (500 mg, yield: 80%) as a white solid.

¹H NMR (400 MHz, CDCl₃) δ 1.06 (3H, t, J=7.6 Hz), 1.31 (6H, d, J=6.4 Hz), 1.80-1.90 (2H, m), 2.38 (3H, s), 3.10-3.20 (1H, m), 4.04 (2H, t, J=6.8 Hz), 5.03 (1H, brs), 7.22-7.25 (2H, m), 7.36 (1H, s), 8.02 (2H, d, J=8.4 Hz), 8.11 (1H, s).

### Step 6. Synthesis of 5-isopropoxy-3-isopropyl-4-propoxy-1-tosyl-1H-pyrrolo[2,3-b]pyridine

To a solution of 3-isopropyl-4-propoxy-1-tosyl-1H-pyrrolo[2,3-b]pyridin-5-ol (500 mg, 1.29 mmol) in DMF (10 mL) was added K₂CO₃ (356 mg, 2.57 mmol) and 2-iodopropane (1.09 g, 6.44 mmol). The mixture was stirred at 70° C. for 2 hours. The reaction mixture was quenched with H₂O (25 mL) and extracted with EtOAc (25 mL×3). The combined organic layers were washed with brine (20 mL×3), dried over anhydrous Na₂SO₄, filtered and concentrated. The residue was purified by column chromatography (SiO₂, Petroleum ether/Ethyl acetate=10/1 to 5/1) to give 5-isopropoxy-3-isopropyl-4-propoxy-1-tosyl-1H-pyrrolo[2,3-b]pyridine (300 mg, yield: 53%) as a colorless oil.

### Step 7. Synthesis of 5-isopropoxy-3-isopropyl-4-propoxy-1H-pyrrolo[2,3-b]pyridine

To a solution of 5-isopropoxy-3-isopropyl-4-propoxy-1-tosyl-1H-pyrrolo[2,3-b]pyridine (300 mg, 0.697 mmol) in THF (2 mL) and MeOH (1 mL) was added 6N aqueous KOH (1.2 mL). The mixture was stirred at 50° C. for 2 hours. The reaction mixture was diluted with H₂O (25 mL) and extracted with EtOAc (30 mL×3). The combined organic layers were washed with brine (25 mL), dried over anhydrous Na₂SO₄, filtered and concentrated. The residue was purified by prep-HPLC (10 nm NH₄HCO₃ as an additive) and lyophilized to give the title compound (112.31 mg, yield: 57%) as a white solid.

¹H NMR (400 MHz, CD₃OD) δ 1.09 (3H, t, J=7.2 Hz), 1.25-1.35 (12H, m), 1.80-1.90 (2H, m), 3.35-3.40 (1H, m), 4.29-4.35 (2H, m), 4.37-4.42 (1H, m), 7.00 (1H, s), 7.85 (1H, s).

### Example 11

### 5-(cyclopentyloxy)-4-ethyl-3,3-dimethyl-1,3-dihydro-2H-pyrrolo[2,3-b]pyridin-2-one

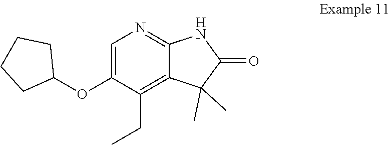 [SMILES: CCc1c(OC2CCCC2)cnc2c1C(C)(C)C(=O)N2]

### Step 1. Synthesis of 4-ethyl-3,3-dimethyl-5-(4,4,5,5-tetramethyl-1,3,2-dioxaborolan-2-yl)-1,3-dihydro-2H-pyrrolo[2,3-b]pyridin-2-one

A mixture of compound Int-9 (480 mg, 1.78 mmol), Bis-Pin (1.35 g, 5.33 mmol), Pd(dppf)Cl₂ (144 mg, 0.176 mmol) and KOAc (528 mg, 5.38 mmol) in dioxane (15 mL) was degassed and purged with N₂ for 3 times. The mixture was stirred at 110° C. for 16 hours under N₂ atmosphere. The mixture was poured into water (30 mL) and extracted with EtOAc (30 mL×3). The combined organic layer was washed with brine (30 mL×2), dried over anhydrous Na₂SO₄, filtered and concentrated. The residue was purified by silica gel column (PE/EtOAc=1/1) to afford 4-ethyl-3,3-dimethyl-5-(4,4,5,5-tetramethyl-1,3,2-dioxaborolan-2-yl)-1,3-dihydro-2H-pyrrolo[2,3-b]pyridin-2-one (240 mg, yield: 43%) as a yellow solid.

### Step 2. Synthesis of 4-ethyl-5-hydroxy-3,3-dimethyl-1,3-dihydro-2H-pyrrolo[2,3-b]pyridin-2-one

To a mixture of 4-ethyl-3,3-dimethyl-5-(4,4,5,5-tetramethyl-1,3,2-dioxaborolan-2-yl)-1,3-dihydro-2H-pyrrolo[2,3-b]pyridin-2-one (240 mg, 0.759 mmol) in EtOAc (3 mL) was added 30% aqueous H₂O₂ (1 mL, 10.4 mmol) dropwise at 20-25° C. The reaction mixture was stirred at 20-25° C. for 2 hours. The reaction mixture was quenched with saturated aqueous Na₂SO₃ (2 mL) at 0° C. and extracted with EtOAc (15 mL×3). The combined organic layer was concentrated to give 4-ethyl-5-hydroxy-3,3-dimethyl-1,3-dihydro-2H-pyrrolo[2,3-b]pyridin-2-one (80 mg, yield: 51%) as a white solid.

¹H NMR (400 MHz, DMSO-d₆) δ 1.13 (3H, t, J=7.6 Hz), 1.32 (6H, s), 2.60 (2H, q, J=7.6 Hz), 7.56 (1H, s), 9.28 (1H, brs), 10.61 (1H, brs).

### Step 3. Synthesis of 5-(cyclopentyloxy)-4-ethyl-3,3-dimethyl-1,3-dihydro-2H-pyrrolo[2,3-b]pyridin-2-one

A mixture of 4-ethyl-5-hydroxy-3,3-dimethyl-1,3-dihydro-2H-pyrrolo[2,3-b]pyridin-2-one (20 mg, 0.097 mmol), bromocyclopentane (86 mg, 0.58 mmol) and K₂CO₃ (40 mg, 0.29 mmol) in DMF (2 mL) was stirred at 80° C. for 2 hours. The reaction mixture was concentrated and the residue was purified by prep-TLC (PE/EtOAc=1/1), then further purified by prep-HPLC (0.05% NH₃H₂O +10 mM NH₄HCO₃ as an additive) and lyophilized to afford the title compound (2 mg, yield: 8%) as a white solid.

¹H NMR (400 MHz, CD₃OD) δ 1.22 (3H, t, J=7.6 Hz), 1.47 (6H, s), 1.67-2.04 (8H, m), 2.73 (2H, q, J=7.6 Hz), 4.85-4.90 (1H, m), 7.67 (1H, s).

### Example 12

### 6-(benzo[d][1,3]dioxol-4-yl)-7H-pyrrolo[2,3-c]pyridazine

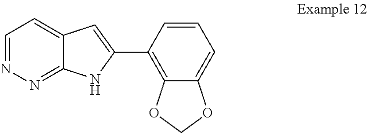 [SMILES: c1cc2c(c(-c3cc4ccnnc4[nH]3)c1)OCO2]

### Step 1. Synthesis of 4-ethynylbenzo[d][1,3]dioxole

To a mixture of benzo[d][1,3]dioxole-4-carbaldehyde (1.00 g, 6.66 mmol) and methyl 2-diazo-2-(dimethoxyphosphoryl)acetate (2.00 g, 10.4 mmol) in MeOH (50 mL) was added K₂CO₃ (2.80 g, 20.3 mmol) at 25° C. The mixture was stirred at 25° C. for 16 hours. The mixture was filtered and the filtrate was concentrated. The residue was purified by silica gel chromatography (DCM as the eluent) to afford 4-ethynylbenzo[d][1,3]dioxole (800 mg, yield: 82%) as yellow oil.

¹H NMR (400 MHz, CDCl₃) δ 3.20 (1H, s), 5.95 (2H, s), 6.70 (1H, d, J=7.6 Hz), 6.72-6.75 (1H, m), 6.85 (1H, dd, J=7.6, 1.6 Hz).

### Step 2. Synthesis of 4-(benzo[d][1,3]dioxol-4-ylethynyl)-6-chloropyridazin-3-amine

A mixture of 4-ethynylbenzo[d][1,3]dioxole (2.00 g, 13.7 mmol), 4-bromo-6-chloropyridazin-3-amine (2.85 g, 13.7 mmol), (PPh₃)₄Pd (500 mg, 0.433 mmol) and CuI (261 mg, 1.37 mmol) in Et₃N (100 mL) was degassed and purged with N₂ for 3 times. Then the reaction mixture was stirred at 65° C. for 1 hour under N₂ atmosphere. The mixture was filtered and the filtrate was concentrated. The residue was purified by silica gel chromatography (ISCO®; 40 g SepaFlash® Silica Flash Column, Eluent of 100% Ethyl acetate/Petroleum ether gradient @50 mL/min) to give 4-(benzo[d][1,3]dioxol-4-ylethynyl)-6-chloropyridazin-3-amine (3.50 g, yield: 93%) as a yellow solid.

### Step 3. Synthesis of 6-(benzo[d][1,3]dioxol-4-yl)-7H-pyrrolo[2,3-c]pyridazine

To a solution of 4-(benzo[d][1,3]dioxol-4-ylethynyl)-6-chloropyridazin-3-amine (1.20 g, 4.38 mmol) in NMP (30 mL) was added NaH (1.23 g, 30.7 mmol, 60% dispersion in mineral oil) at 25° C. under N₂ atmosphere. The mixture was stirred at 100° C. for 48 hours. The reaction mixture was quenched with water (100 mL) and extracted with EtOAc (100 mL×3). The combined organic layer was washed with brine (100 mL×3), dried over anhydrous Na₂SO₄, filtered and concentrated. The crude product was triturated with MeOH/EtOAc (10 mL, 3/1) to give the title compound (1.00 g, yield: 90%) as a yellow solid. Among 200 mg of it was further purified by prep-HPLC (0.05% NH₃H₂O+10 mM NH₄HCO₃ as an additive), then lyophilized to give Example 12 (12 mg) as a yellow solid and delivered.

¹H NMR (400 MHz, DMSO-d₆) δ 6.24 (2H, s), 7.00-7.07 (3H, m), 7.58-7.65 (1H, m), 7.83 (1H, d, J=5.6 Hz), 8.85 (1H, d, J=5.6 Hz).

### Example 14

### 6-(3,5-difluorophenoxy)-4-(1-methyl-1H-pyrazol-4-yl)isoquinoline

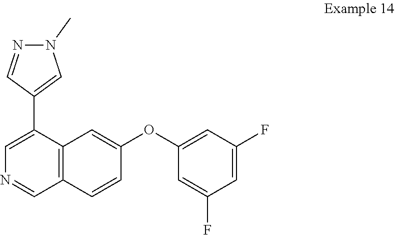 [SMILES: Cn1cc(-c2cncc3ccc(Oc4cc(F)cc(F)c4)cc23)cn1]

### Step 1. Synthesis of 6-(3,5-difluorophenoxy)-4-iodoisoquinoline

To a solution of 6-bromo-4-iodoisoquinoline (600 mg, 1.80 mmol) and 3,5-difluorophenol (234 mg, 1.80 mmol) in DMSO (10 mL) was added CuI (34 mg, 0.18 mmol), K₃PO₄ (763 mg, 3.59 mmol) and 2-picolinic acid (44 mg, 0.36 mmol) under N₂ atmosphere. The mixture was stirred at 130° C. for 16 hours under N₂ atmosphere. The reaction mixture was poured into water (30 mL) and extracted with EtOAc (30 mL×3). The combined organic layer was washed with brine (40 mL), dried over anhydrous Na₂SO₄, filtered and concentrated to give the residue. The residue was purified by flash silica gel chromatography (Eluent of 0˜12% Ethylacetate/Petroleum ether gradient) to give a mixture of 6-(3,5-difluorophenoxy)-4-iodoisoquinoline and 6-bromo-4-(3,5-difluorophenoxy)isoquinoline (220 mg) as a light yellow solid.

### Step 2. Synthesis of 6-(3,5-difluorophenoxy)-4-(1-methyl-1H-pyrazol-4-yl)isoquinoline

To a solution of 6-bromo-4-(3,5-difluorophenoxy)isoquinoline and 6-(3,5-difluorophenoxy)-4-iodoisoquinoline (220 mg), (1-methyl-1H-pyrazol-4-yl)boronic acid (75 mg, 0.60 mmol) in 1, 4-dioxane (4 mL) and H₂O (1 mL) was added Na₂CO₃ (63 mg, 0.60 mmol) and Pd(dppf)Cl₂ (22 mg, 0.030 mmol) under N₂ atmosphere The mixture was stirred at 100° C. for 16 hours under N₂ atmosphere. The reaction mixture was diluted with H₂O (30 mL) and extracted with EtOAc (40 mL×3). The combined organic layer was washed with brine (50 mL), dried over Na₂SO₄, filtered and concentrated to give a residue. The residue was purified by flash silica gel chromatography (ISCO®; 12 g SepaFlash® Silica Flash Column, Eluent of 0˜69% Ethyl acetate/Petroleum ether gradient @23 mL/min) and then further purified by prep-TLC (DCM/MeOH=20/1) to give the title compound (27 mg, yield: 26%) as a yellow gum.

¹H NMR (400 MHz, CDCl₃) δ 4.02 (3H, s), 6.53-6.66 (3H, m), 7.37 (1H, dd, J=8.8, 2.4 Hz), 7.61 (1H, s), 7.69 (1H, d, J=2.4 Hz), 7.72 (1H, s), 8.07 (1H, d, J=8.8 Hz), 8.51 (1H, s), 9.17 (1H, s).

The following compound was synthesized analogously to Example 14

| Example | | | |
|---|---|---|---|
| No. | Structure | Name | ¹H NMR (400 MHz) |
| 15 | 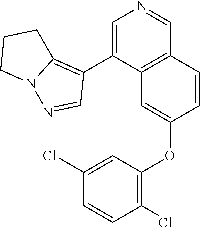 [SMILES: Clc1ccc(Cl)c(Oc2ccc3cncc(-c4cnn5c4CCC5)c3c2)c1] | 6-(2,5- dichlorophenoxy)-4- (5,6-dihydro-4H- pyrrolo[1,2- b]pyrazol-3- yl)isoquinoline | CDCl₃; δ 2.59-2.69 (2H, m), 2.86 (2H, t, J = 6.8 Hz), 4.23 (2H, t, J = 7.2 Hz), 7.11 (1H, d, J = 2.0 Hz), 7.18 (1H, dd, J = 8.4, 2.4 Hz), 7.30-7.33 (1H, m), 7.39 (1H, dd, J = 8.8, 2.0 Hz), 7.45 (1H, d, J = 8.8 Hz), 7.67 (1H, s) 8.06 (1H, d, J = 9.2 Hz), 8.45 (1H, s), 9.14 (1H, s). |

### Example 18

### 1-methyl-N-(6-(methylsulfonyl)isoquinolin-3-yl)piperidine-4-carboxamide

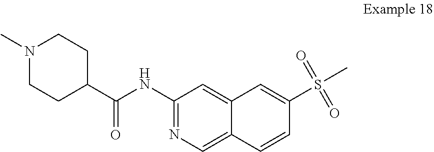

A mixture of Example 25 (300 mg, 0.861 mmol), MeSO₂Na (132 mg, 1.29 mmol), CuI (33 mg, 0.17 mmol), NaOH (14 mg, 0.34 mmol) and L-proline (40 mg, 0.34 mmol) in DMSO (6 mL) was bubbled with N₂ for 6 minutes and the reaction mixture was stirred at 115° C. for 5 hours under N₂ atmosphere. The reaction mixture was filtered and the filtrate was purified by prep-HPLC (0.05% NH₃H₂O+10 mM NH₄HCO₃ as an additive), then lyophilized to give the title compound (194 mg, yield: 65%) as a white solid.

¹H NMR (400 MHz, DMSO-d₆) δ 1.62-1.74 (2H, m), 1.74-1.93 (4H, m), 2.16 (3H, s), 2.55-2.60 (1H, m), 2.77-2.86 (2H, m), 3.34 (3H, s), 7.93 (1H, dd, J=8.4, 1.6 Hz), 8.30 (1H, d, J=8.8 Hz), 8.50 (1H, s), 8.67 (1H, s), 9.32 (1H, s), 10.73 (1H, brs).

### Example 19

### N-(6-(cyanomethyl)isoquinolin-3-yl)-1-methylpiperidine-4-carboxamide

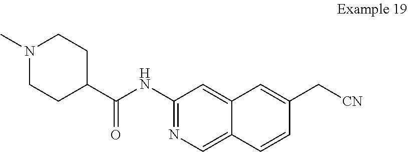 [SMILES: CN1CCC(C(=O)Nc2cc3cc(CC#N)ccc3cn2)CC1]

A solution of Example 25 (150 mg, 0.431 mmol), 4-(4,4,5,5-tetramethyl-1,3,2-dioxaborolan-2-yl)isoxazole (126 mg, 0.646 mmol), 1N aqueous KF (0.86 mL) and Pd(dppf)Cl₂ (63 mg, 0.086 mmol) in DMSO (4.5 mL) was bubbled with N₂ for 6 minutes and the reaction mixture was stirred at 130° C. for 16 hours to give brown solution. The reaction mixture was concentrated and the residue was purified by flash silica gel chromatography (ISCO®; 20 g SepaFlash® Silica Flash Column, Eluent of 8˜20% MeOH/DCM (3% Et₃N as an additive) gradient @35 mL/min), then further purified by prep-HPLC (Method C; 0.225% FA as an additive) and lyophilized to give the title compound (55 mg, yield: 35%, FA salt) as a yellow solid.

¹H NMR (400 MHz, DMSO-d₆) δ 1.63-1.75 (2H, m), 1.76-1.84 (2H, m), 1.93-2.04 (2H, m), 2.23 (3H, s), 2.53-2.58 (1H, m), 2.83-2.93 (2H, m), 4.25 (2H, s), 7.47 (1H, dd, J=8.4, 1.6 Hz), 7.82 (1H, s), 8.08 (1H, d, J=8.8 Hz), 8.19 (1H, s), 8.46 (1H, s), 9.13 (1H, s), 10.59 (1H, brs).

### Example 20

### 2-(3-(1-methylpiperidine-4-carboxamido)isoquinolin-6-yl)acetic Acid

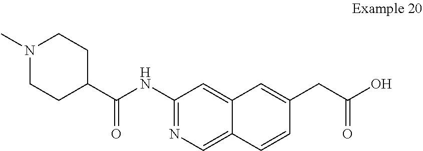 [SMILES: CN1CCC(C(=O)Nc2cc3cc(CC(=O)O)ccc3cn2)CC1]

### Step 1. Synthesis of methyl 2-(3-(1-methylpiperidine-4-carboxamido)isoquinolin-6-yl)acetate

A mixture of Example 25 (200 mg, 0.574 mmol), (t-Bu₃P)₂Pd (44 mg, 0.086 mmol) and ZnF₂ (178 mg, 1.72 mmol) in DMF (6 mL) was degassed and purged with N₂ for 3 times, then compound 20-a (325 mg, 1.72 mmol) was added to the reaction mixture. The mixture was stirred at 80° C. for 16 hours under N₂ atmosphere to give yellow suspension. The reaction mixture was concentrated and the residue was purified by flash silica gel chromatography (ISCO®; 12 g SepaFlash® Silica Flash Column, Eluent of ˜10% MeOH/DCM (3% Et₃N as an additive) gradient @35 mL/min) to give methyl 2-(3-(1-methylpiperidine-4-carboxamido)isoquinolin-6-yl)acetate (130 mg, yield: 48%) as a yellow solid.

### Step 2. Synthesis of 2-(3-(1-methylpiperidine-4-carboxamido)isoquinolin-6-yl)acetic Acid

To a solution of methyl 2-(3-(1-methylpiperidine-4-carboxamido)isoquinolin-6-yl)acetate (130 mg, 0.278 mmol) in H₂O (3 mL) and THF (3 mL) was added LiOH·H₂O (58 mg, 1.4 mmol) and the reaction mixture was stirred at 20° C. for 2 hours to give yellow solution. The reaction mixture was acidified with FA to pH=5 and concentrated. The residue was purified by prep-HPLC (0.225% FA as an additive) and lyophilized to give the title compound (39 mg, yield: 42%) as an off-white solid.

¹H NMR (400 MHz, DMSO-d₆) δ 1.61-1.82 (4H, m), 1.85-1.97 (2H, m), 2.19 (3H, s), 2.53-2.56 (1H, m), 2.78-2.88 (2H, m), 3.76 (2H, s), 7.42 (1H, dd, J=8.4, 1.6 Hz), 7.70 (1H, s), 7.97 (1H, d, J=8.4 Hz), 8.40 (1H, s), 9.07 (1H, s), 10.50 (1H, brs).

### Example 27

### 1-ethyl-N-(6-(oxetan-3-yl)isoquinolin-3-yl)piperidine-4-carboxamide

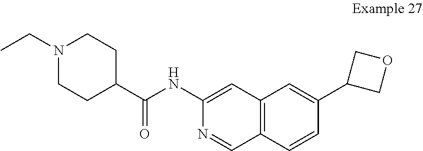 [SMILES: CCN1CCC(C(=O)Nc2cc3cc(C4COC4)ccc3cn2)CC1]

A mixture of benzyl 4-((6-(oxetan-3-yl)isoquinolin-3-yl)carbamoyl)piperidine-1-carboxylate (see Example 26; 100 mg, 0.224 mmol) and 10% Pd/C (100 mg) in absolute EtOH (3 mL) was degassed and purged with H₂ for 3 times. The reaction mixture was hydrogenated (15 psi) at 20° C. for 16 hours. The reaction mixture was filtered through a pad of celite and the solid was washed with EtOH (5 mL×2). The filtrate was concentrated and the residue was purified by prep-HPLC (Method C; 0.225% FA as an additive), then lyophilized to give the title compound (7 mg, yield: 7%, 74% LCMS purity, FA salt) as a white solid.

¹H NMR (400 MHz, CD₃OD) δ 1.33 (3H, t, J=7.6 Hz), 1.96-2.25 (4H, m), 2.75-3.00 (3H, m), 3.10 (2H, q, J=7.2 Hz), 3.54-3.58 (2H, m), 4.45-4.50 (1H, m), 4.80-4.90 (2H, m), 5.15-5.19 (2H, m), 7.68 (1H, dd, J=8.4, 1.2 Hz), 7.79 (1H, s), 8.04 (1H, d, J=8.8 Hz), 8.53 (1H, s), 9.03 (1H, s).

### Example 76

### 1-(6-(6-methylpyridazin-4-yl)imidazo[1,2-a]pyridin-2-yl)imidazolidin-2-one

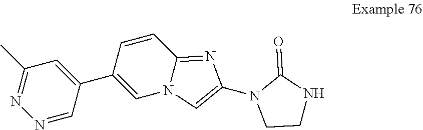 [SMILES: Cc1cc(-c2ccc3nc(N4CCNC4=O)cn3c2)cnn1]

### Step 1: Synthesis of 6-(6-methylpyridazin-4-yl)imidazo[1,2-a]pyridin-2-amine

3-Methylpyridazine-5-boronic acid pinacol ester (1.56 g, 7.07 mmol) was added to a solution of 6-bromoimidazo[1,2-a]pyridin-2-amine (1.0 g, 4.72 mmol), Pd(DPPF)Cl₂ (242. mg, 0.33 mmol), and potassium carbonate (991 mg, 7.07 mmol) in DMA (12 mL) and Water (2 mL). The mixture was reacted in a microwave reactor at 160° C. for 30 min. The crude was purified using prep HPLC (0-35% ACN/0.05% aqueous NH₄OH over 10 min) to give 6-(6-methylpyridazin-4-yl)imidazo[1,2-a]pyridin-2-amine as a brown solid (196 mg, 10% yield). ES-MS [M+1]⁺: 222.6.

### Step 2: Synthesis of 1-(6-(6-methylpyridazin-4-yl)imidazo[1,2-a]pyridin-2-yl)imidazolidin-2-one

2-chloroethyl isocyanate (9.4 mg, 0.09 mmol), 6-(6-methylpyridazin-4-yl)imidazo[1,2-a]pyridin-2-amine (25 mg, 0.11 mmol), N,N-diisopropylethylamine (48 uL, 0.2 mmol) were combined in DMA (1 mL) and reacted in a microwave reactor at 150° C. for 20 min. The crude product syringe was purified by prep HPLC (0-35% ACN/0.05% aqueous NH₄OH over 10 min). Fractions containing the desired product were concentrated to give 1-(6-(6-methylpyridazin-4-yl)imidazo[1,2-a]pyridin-2-yl)imidazolidin-2-one as a yellow solid (4.2 mg, 13% yield). ES-MS [M+1] 295.2. NMR. ¹H NMR (400 MHz, DMSO) δ 9.57 (dd, J=2.0, 0.9 Hz, 1H), 9.39 (d, J=2.3 Hz, 1H), 7.85-7.79 (m, 2H), 7.48 (dd, J=9.1, 0.8 Hz, 1H), 6.17 (s, 2H), 4.39 (t, J=9.1 Hz, 2H), 3.99 (t, J=9.1 Hz, 2H), 2.69 (s, 3H).

Examples 28-75 were synthesized analogously to the examples described above.

| Example No. | Structure |
|---|---|
| 28 | 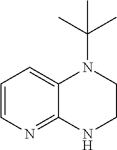 |
| 29 | 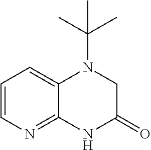 |
| 30 | 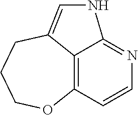 [SMILES: c1cc2c3c(c[nH]c3n1)CCCO2] |
| 31 | 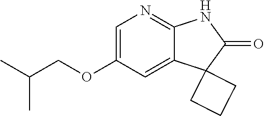 [SMILES: CC(C)COc1cnc2c(c1)C1(CCC1)C(=O)N2] |
| 32 | 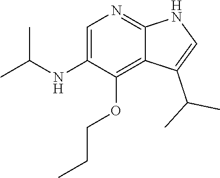 [SMILES: CCCOc1c(NC(C)C)cnc2[nH]cc(C(C)C)c12] |
| 33 | 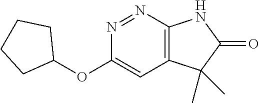 [SMILES: CC1(C)C(=O)Nc2nnc(OC3CCCC3)cc21] |
| 34 | 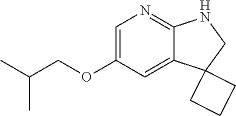 [SMILES: CC(C)COc1cnc2c(c1)C1(CCC1)CN2] |
| 35 | 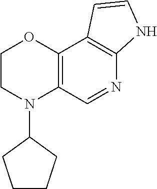 [SMILES: c1cc2c3c(cnc2[nH]1)N(C1CCCC1)CCO3] |
| 36 | 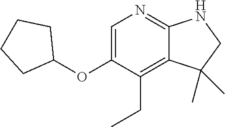 [SMILES: CCc1c(OC2CCCC2)cnc2c1C(C)(C)CN2] |
| 37 | 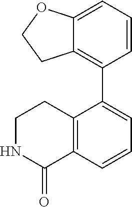 [SMILES: O=C1NCCc2c1cccc2-c1cccc2c1CCO2] |
| 38 | 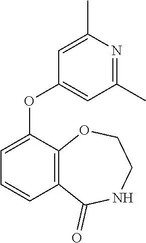 [SMILES: Cc1cc(Oc2cccc3c2OCCNC3=O)cc(C)n1] |
| 39 | 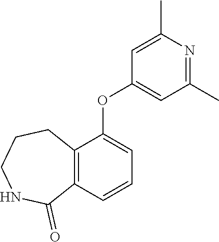 [SMILES: Cc1cc(Oc2cccc3c2CCCNC3=O)cc(C)n1] |
| 40 | 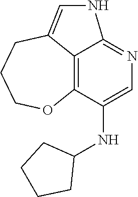 [SMILES: c1nc2[nH]cc3c2c(c1NC1CCCC1)OCCC3] |
| 41 | 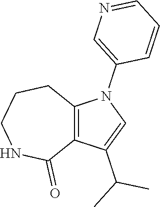 [SMILES: CC(C)c1cn(-c2cccnc2)c2c1C(=O)NCCC2] |
| 42 | 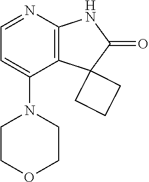 [SMILES: O=C1Nc2nccc(N3CCOCC3)c2C12CCC2] |
| 43 | 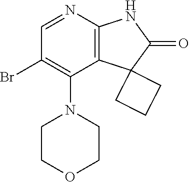 [SMILES: O=C1Nc2ncc(Br)c(N3CCOCC3)c2C12CCC2] |
| 44 | 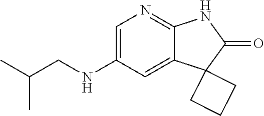 [SMILES: CC(C)CNc1cnc2c(c1)C1(CCC1)C(=O)N2] |
| 45 | 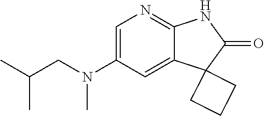 [SMILES: CC(C)CN(C)c1cnc2c(c1)C1(CCC1)C(=O)N2] |
| 46 | 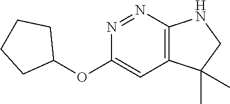 [SMILES: CC1(C)CNc2nnc(OC3CCCC3)cc21] |
| 47 | 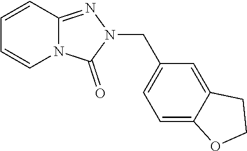 [SMILES: O=c1n(Cc2ccc3c(c2)CCO3)nc2ccccn12] |
| 48 | 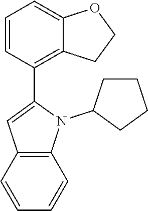 [SMILES: c1cc2c(c(-c3cc4ccccc4n3C3CCCC3)c1)CCO2] |
| 49 | 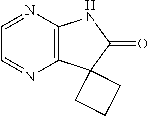 [SMILES: O=C1Nc2nccnc2C12CCC2] |
| 50 | 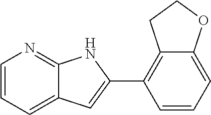 [SMILES: c1cc2c(c(-c3cc4cccnc4[nH]3)c1)CCO2] |
| 51 | 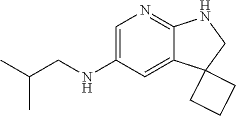 [SMILES: CC(C)CNc1cnc2c(c1)C1(CCC1)CN2] |
| 52 | 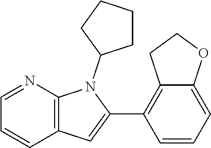 [SMILES: c1cc2c(c(-c3cc4cccnc4n3C3CCCC3)c1)CCO2] |
| 53 | 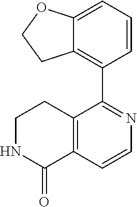 [SMILES: O=C1NCCc2c1ccnc2-c1cccc2c1CCO2] |
| 54 | 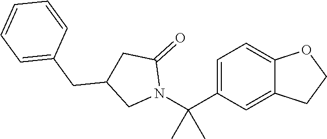 [SMILES: CC(C)(c1ccc2c(c1)CCO2)N1CC(Cc2ccccc2)CC1=O] |
| 55 | 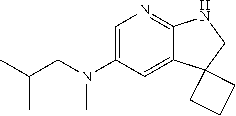 [SMILES: CC(C)CN(C)c1cnc2c(c1)C1(CCC1)CN2] |
| 56 | 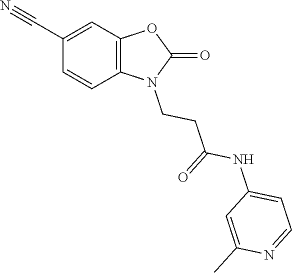 [SMILES: Cc1cc(NC(=O)CCn2c(=O)oc3cc(C#N)ccc32)ccn1] |
| 57 | 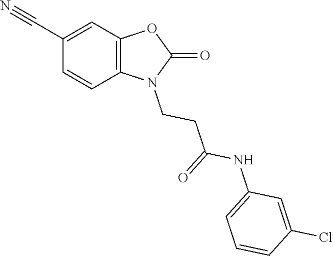 [SMILES: N#Cc1ccc2c(c1)oc(=O)n2CCC(=O)Nc1cccc(Cl)c1] |
| 58 | 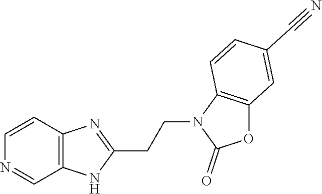 [SMILES: N#Cc1ccc2c(c1)oc(=O)n2CCc1nc2ccncc2[nH]1] |
| 59 | 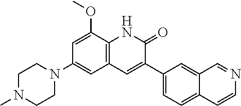 [SMILES: COc1cc(N2CCN(C)CC2)cc2cc(-c3ccc4ccncc4c3)c(=O)[nH]c12] |
| 60 | 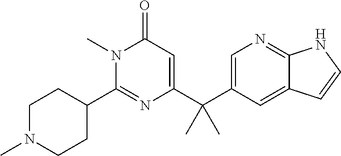 [SMILES: CN1CCC(c2nc(C(C)(C)c3cnc4[nH]ccc4c3)cc(=O)n2C)CC1] |
| 61 | 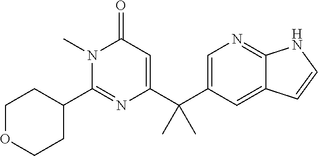 [SMILES: Cn1c(C2CCOCC2)nc(C(C)(C)c2cnc3[nH]ccc3c2)cc1=O] |
| 62 | 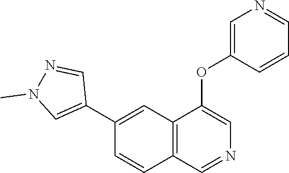 [SMILES: Cn1cc(-c2ccc3cncc(Oc4cccnc4)c3c2)cn1] |
| 63 | 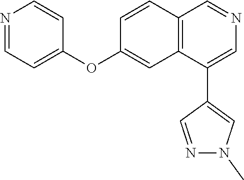 [SMILES: Cn1cc(-c2cncc3ccc(Oc4ccncc4)cc23)cn1] |
| 64 | 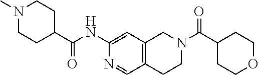 [SMILES: CN1CCC(C(=O)Nc2cc3c(cn2)CCN(C(=O)C2CCOCC2)C3)CC1] |
| 65 | 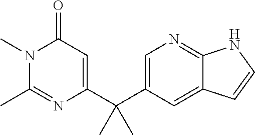 [SMILES: Cc1nc(C(C)(C)c2cnc3[nH]ccc3c2)cc(=O)n1C] |
| 66 | 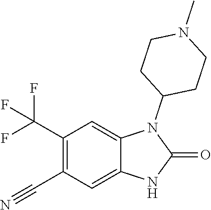 [SMILES: CN1CCC(n2c(=O)[nH]c3cc(C#N)c(C(F)(F)F)cc32)CC1] |
| 67 | 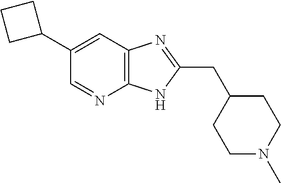 [SMILES: CN1CCC(Cc2nc3cc(C4CCC4)cnc3[nH]2)CC1] |
| 68 | 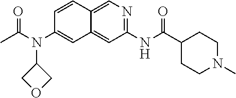 [SMILES: CC(=O)N(c1ccc2cnc(NC(=O)C3CCN(C)CC3)cc2c1)C1COC1] |
| 69 | 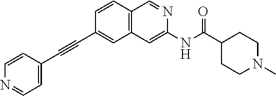 [SMILES: CN1CCC(C(=O)Nc2cc3cc(C#Cc4ccncc4)ccc3cn2)CC1] |
| 70 | 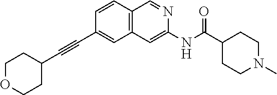 [SMILES: CN1CCC(C(=O)Nc2cc3cc(C#CC4CCOCC4)ccc3cn2)CC1] |
| 71 | 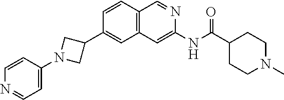 [SMILES: CN1CCC(C(=O)Nc2cc3cc(C4CN(c5ccncc5)C4)ccc3cn2)CC1] |
| 72 | 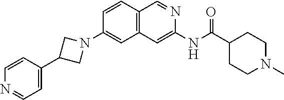 [SMILES: CN1CCC(C(=O)Nc2cc3cc(N4CC(c5ccncc5)C4)ccc3cn2)CC1] |
| 73 | 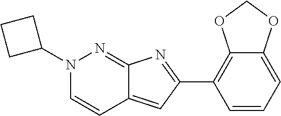 [SMILES: c1cc2c(c(-c3cc4ccn(C5CCC5)nc-4n3)c1)OCO2] |
| 74 | 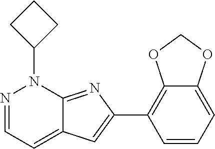 [SMILES: c1cc2c(c(-c3cc4ccnn(C5CCC5)c-4n3)c1)OCO2] |
| 75 | 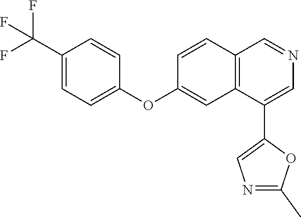 [SMILES: Cc1ncc(-c2cncc3ccc(Oc4ccc(C(F)(F)F)cc4)cc23)o1] |

Compounds of Formula (I) and (II) and related analogs and their associated LC-MS data are shown in the Table below. These compounds were prepared according to procedures analogous to the procedures above, with modifications where appropriate that are within the purview of one skilled in the art. Liquid Chromatography-Mass Spectrometry (LCMS) was taken on a quadruple Mass Spectrometer on Waters QDa/Acquity I-Class LCMS (Column: Phenomenex Kinetex EVO C18 (1.0×50 mm, 1.7 um)) operating in ESI (+) ionization mode. Flow Rate: 0.4 mL/min, Acquire Time: 1.5 min, Wavelength: UV215 & 254, Oven Temp.: 55° C.

TABLE 3

| | LCMS | |
|---|---|---|
| | Retention | m/z |
| Compound Structure | Time (min) | (M + H) |
| 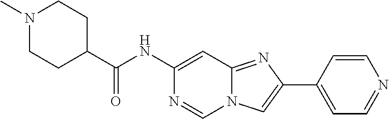 [SMILES: CN1CCC(C(=O)Nc2cc3nc(-c4ccncc4)cn3cn2)CC1] | 0.489 | 337.3 |
| 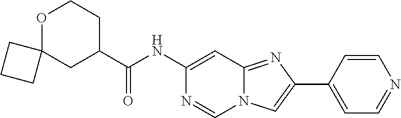 [SMILES: O=C(Nc1cc2nc(-c3ccncc3)cn2cn1)C1CCOC2(CCC2)C1] | 0.63 | 364.4 |
| 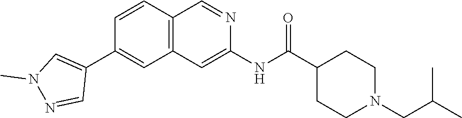 [SMILES: CC(C)CN1CCC(C(=O)Nc2cc3cc(-c4cnn(C)c4)ccc3cn2)CC1] | 0.47 | 392.2 |
| 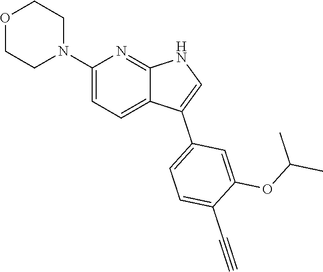 [SMILES: C#Cc1ccc(-c2c[nH]c3nc(N4CCOCC4)ccc23)cc1OC(C)C] | 0.740 | 363.3 |
| 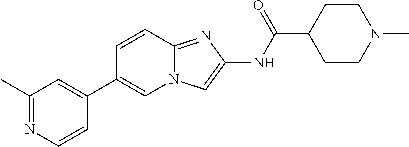 | 0.624 | 350.4 |
| 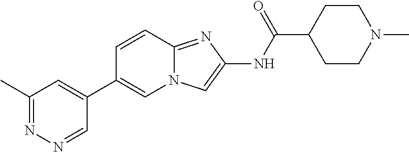 | 0.38 | 351.3 |
| 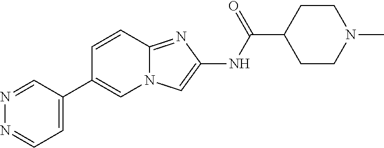 [SMILES: CN1CCC(C(=O)Nc2cn3cc(-c4ccnnc4)ccc3n2)CC1] | 0.398 | 337.2 |
| 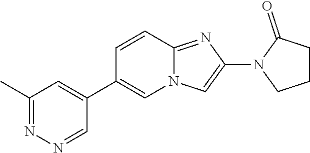 [SMILES: Cc1cc(-c2ccc3nc(N4CCCC4=O)cn3c2)cnn1] | 0.368 | 294.2 |
| 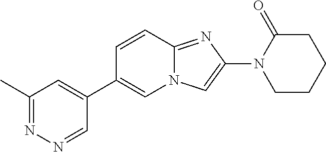 [SMILES: Cc1cc(-c2ccc3nc(N4CCCCC4=O)cn3c2)cnn1] | 0.547 | 308.2 |
| 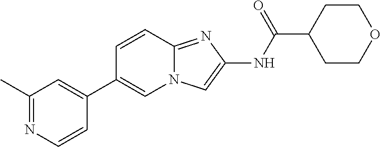 [SMILES: Cc1cc(-c2ccc3nc(NC(=O)C4CCOCC4)cn3c2)ccn1] | 0.485 | 338.3 |
| 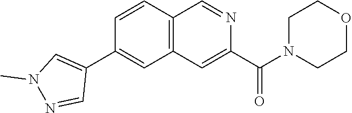 [SMILES: Cn1cc(-c2ccc3cnc(C(=O)N4CCOCC4)cc3c2)cn1] | 0.473 | 323.0 |
| 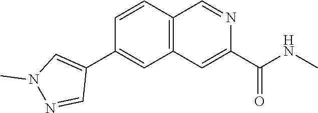 | 0.499 | 267.0 |
| 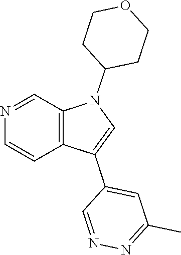 [SMILES: Cc1cc(-c2cn(C3CCOCC3)c3cnccc23)cnn1] | 0.539 | 295.1 |
| 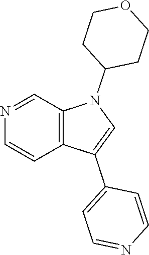 [SMILES: c1cc(-c2cn(C3CCOCC3)c3cnccc23)ccn1] | 0.650 | 280.1 |
| 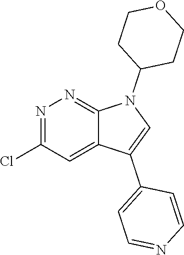 [SMILES: Clc1cc2c(-c3ccncc3)cn(C3CCOCC3)c2nn1] | 0.715 | 315.1 |
| 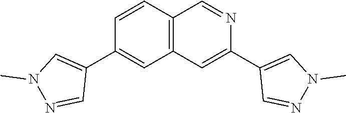 | 0.427 | 290.2 |
| 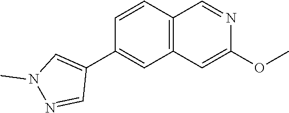 | 0.568 | 240.1 |
| 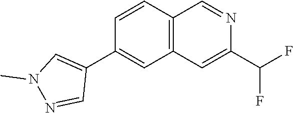 [SMILES: Cn1cc(-c2ccc3cnc(C(F)F)cc3c2)cn1] | 0.548 | 260.1 |
| 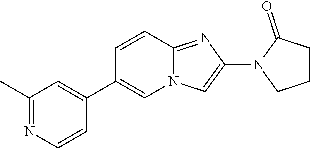 [SMILES: Cc1cc(-c2ccc3nc(N4CCCC4=O)cn3c2)ccn1] | 0.523 | 293.1 |
| 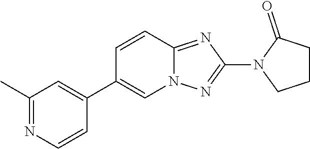 [SMILES: Cc1cc(-c2ccc3nc(N4CCCC4=O)nn3c2)ccn1] | 0.47 | 249.1 |
| 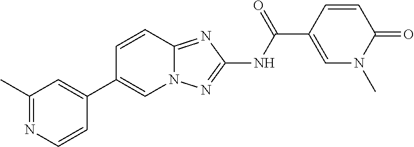 [SMILES: Cc1cc(-c2ccc3nc(NC(=O)c4ccc(=O)n(C)c4)nn3c2)ccn1] | 0.355 | 361.3 |
| 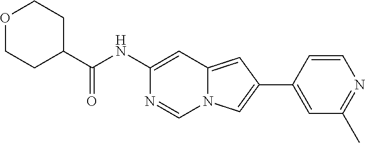 [SMILES: Cc1cc(-c2cc3cc(NC(=O)C4CCOCC4)ncn3c2)ccn1] | 0.556 | 337.1 |
| 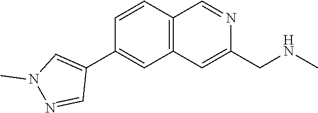 | 0.439 | 253.1 |
| 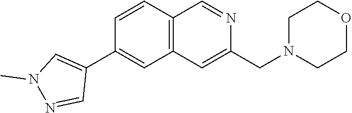 [SMILES: Cn1cc(-c2ccc3cnc(CN4CCOCC4)cc3c2)cn1] | 0.469 | 309.0 |
| 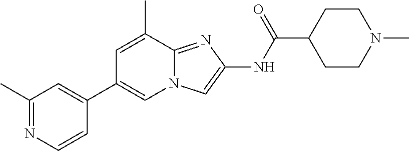 | 0.45 | 364.4 |
| 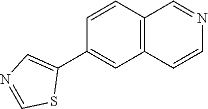 [SMILES: c1cc2cc(-c3cncs3)ccc2cn1] | 0.405 | 213.0 |
| 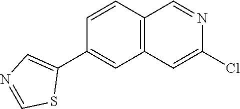 [SMILES: Clc1cc2cc(-c3cncs3)ccc2cn1] | 0.673 | 247.0 |
| 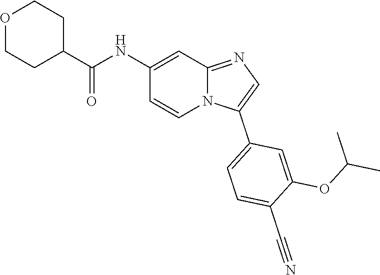 [SMILES: CC(C)Oc1cc(-c2cnc3cc(NC(=O)C4CCOCC4)ccn23)ccc1C#N] | 0.50 | 405.3 |
| 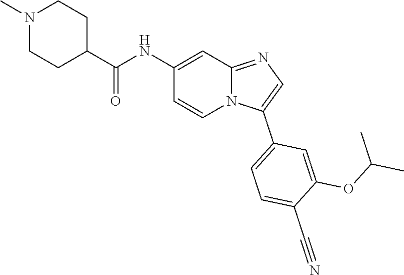 [SMILES: CC(C)Oc1cc(-c2cnc3cc(NC(=O)C4CCN(C)CC4)ccn23)ccc1C#N] | 0.44 | 418.3 |
| 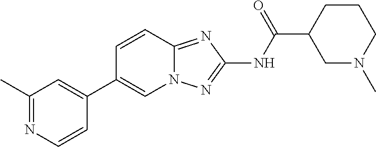 | 0.458 | 351.4 |
| 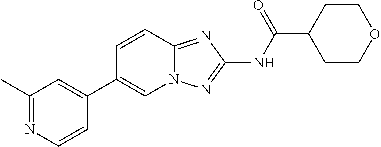 [SMILES: Cc1cc(-c2ccc3nc(NC(=O)C4CCOCC4)nn3c2)ccn1] | 0.435 | 338.2 |
| 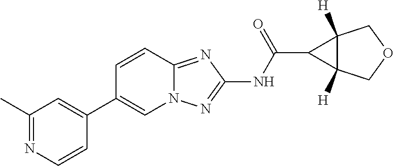 [SMILES: Cc1cc(-c2ccc3nc(NC(=O)C4[C@H]5COC[C@@H]45)nn3c2)ccn1] | 0.44 | 336.4 |
| 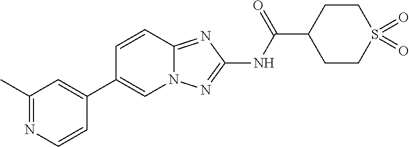 [SMILES: Cc1cc(-c2ccc3nc(NC(=O)C4CCS(=O)(=O)CC4)nn3c2)ccn1] | 0.432 | 386.4 |
| 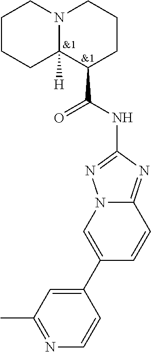 [SMILES: Cc1cc(-c2ccc3nc(NC(=O)[C@@H]4CCCN5CCCC[C@H]45)nn3c2)ccn1] | 0.494 | 391.5 |
| 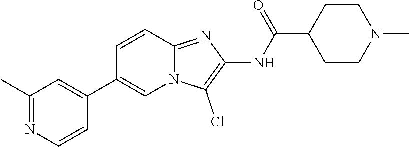 | 0.47 | 384.2 |
| 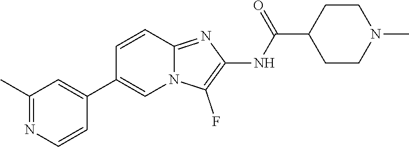 | 0.46 | 368.2 |
| 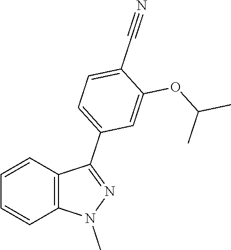 [SMILES: CC(C)Oc1cc(-c2nn(C)c3ccccc23)ccc1C#N] | 0.88 | 292.2 |
| 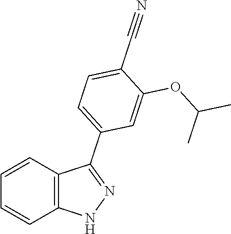 [SMILES: CC(C)Oc1cc(-c2n[nH]c3ccccc23)ccc1C#N] | 0.83 | 278.2 |
| 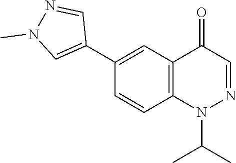 [SMILES: CC(C)n1ncc(=O)c2cc(-c3cnn(C)c3)ccc21] | 0.573 | 269.2 |
| 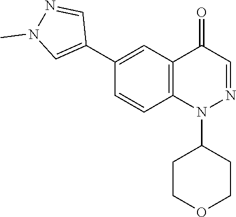 [SMILES: Cn1cc(-c2ccc3c(c2)c(=O)cnn3C2CCOCC2)cn1] | 0.513 | 311.2 |
| 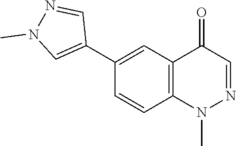 [SMILES: Cn1cc(-c2ccc3c(c2)c(=O)cnn3C)cn1] | 0.463 | 241.2 |
| 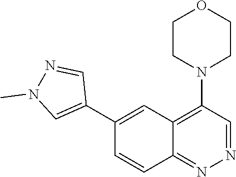 [SMILES: Cn1cc(-c2ccc3nncc(N4CCOCC4)c3c2)cn1] | 0.392 | 296.1 |
| 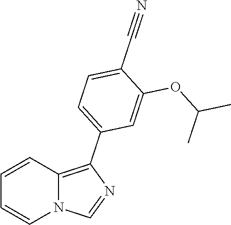 [SMILES: CC(C)Oc1cc(-c2ncn3ccccc23)ccc1C#N] | 0.68 | 278.2 |
| 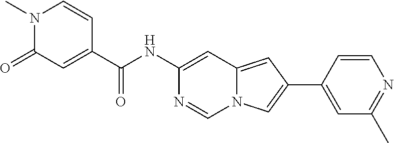 | 0.512 | 360.2 |
| 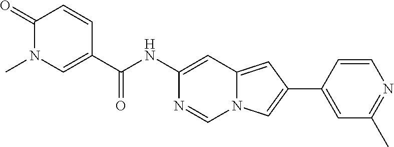 [SMILES: Cc1cc(-c2cc3cc(NC(=O)c4ccc(=O)n(C)c4)ncn3c2)ccn1] | 0.525 | 360.2 |
| 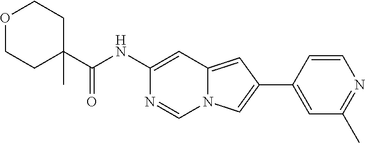 [SMILES: Cc1cc(-c2cc3cc(NC(=O)C4(C)CCOCC4)ncn3c2)ccn1] | 0.469 | 351.3 |
| 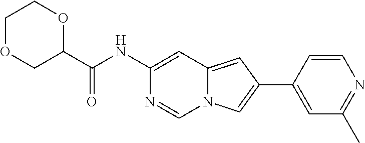 [SMILES: Cc1cc(-c2cc3cc(NC(=O)C4COCCO4)ncn3c2)ccn1] | 0.444 | 339.3 |
| 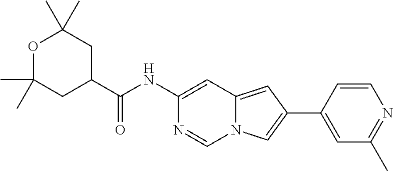 | 0.589 | 393.4 |
| 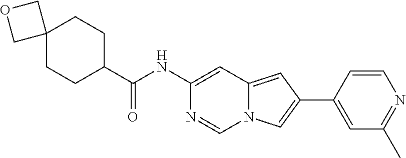 [SMILES: Cc1cc(-c2cc3cc(NC(=O)C4CCC5(CC4)COC5)ncn3c2)ccn1] | 0.497 | 377.3 |
| 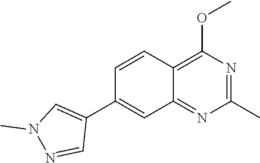 | 0.612 | 255.3 |
| 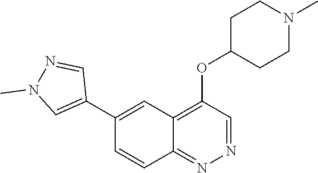 | 0.355 | 324.1 |
| 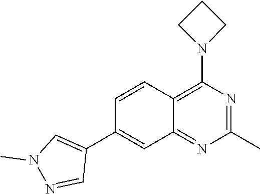 | 0.451 | 280.0 |
| 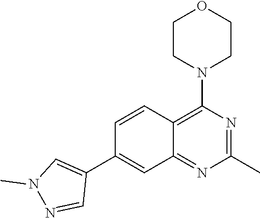 | 0.428 | 310.0 |
| 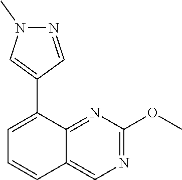 | 0.619 | 241.1 |
| 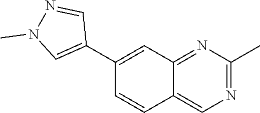 | 0.362 | 225.1 |
| 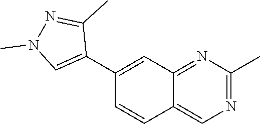 | 0.382 | 239.1 |
| 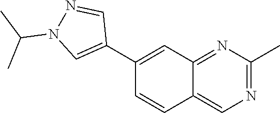 [SMILES: Cc1ncc2ccc(-c3cnn(C(C)C)c3)cc2n1] | 0.465 | 253.1 |
| 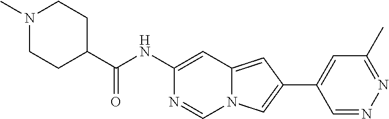 | 0.454 | 351.2 |
| 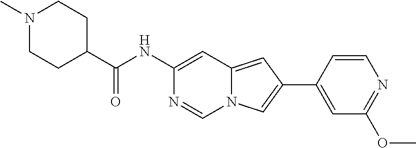 | 0.392 | 366.1 |
| 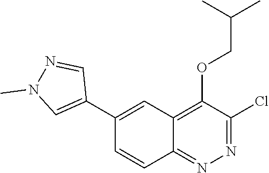 [SMILES: CC(C)COc1c(Cl)nnc2ccc(-c3cnn(C)c3)cc12] | 0.65 | 317.2 |
| 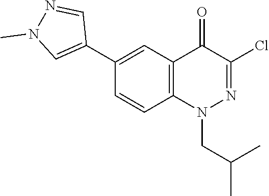 [SMILES: CC(C)Cn1nc(Cl)c(=O)c2cc(-c3cnn(C)c3)ccc21] | 0.78 | 317.1 |
| 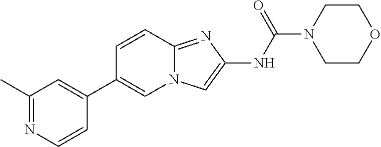 [SMILES: Cc1cc(-c2ccc3nc(NC(=O)N4CCOCC4)cn3c2)ccn1] | 0.495 | 338.1 |
| 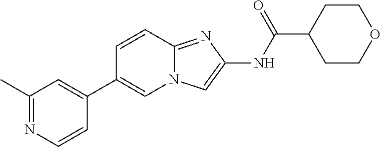 [SMILES: Cc1cc(-c2ccc3nc(NC(=O)C4CCOCC4)cn3c2)ccn1] | 0.503 | 337.3 |
| 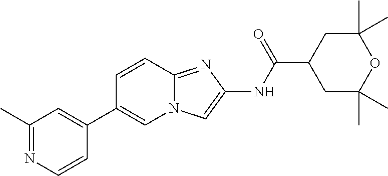 | 0.65 | 393.5 |
| 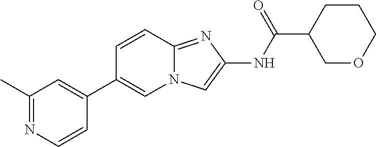 [SMILES: Cc1cc(-c2ccc3nc(NC(=O)C4CCCOC4)cn3c2)ccn1] | 0.53 | 337.3 |
| 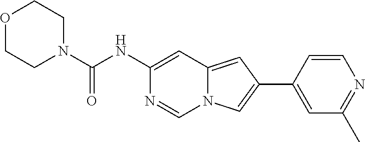 [SMILES: Cc1cc(-c2cc3cc(NC(=O)N4CCOCC4)ncn3c2)ccn1] | 0.420 | 338.2 |
| 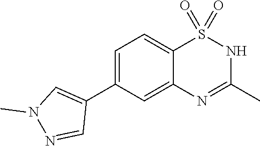 | 0.412 | 276.9 |
| 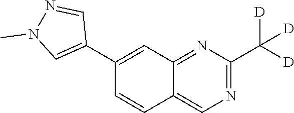 [SMILES: [2H]C([2H])([2H])c1ncc2ccc(-c3cnn(C)c3)cc2n1] | 0.475 | 228.3 |
| 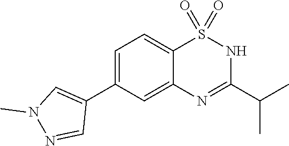 [SMILES: CC(C)C1=Nc2cc(-c3cnn(C)c3)ccc2S(=O)(=O)N1] | 0.517 | 305.1 |
| 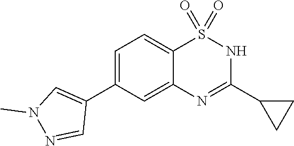 [SMILES: Cn1cc(-c2ccc3c(c2)N=C(C2CC2)NS3(=O)=O)cn1] | 0.488 | 303.0 |
| 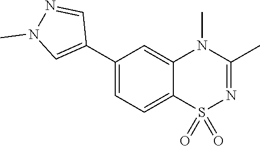 | 0.431 | 291.1 |
| 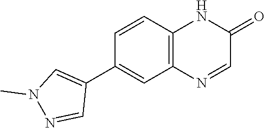 [SMILES: Cn1cc(-c2ccc3[nH]c(=O)cnc3c2)cn1] | 0.431 | 227.1 |
| 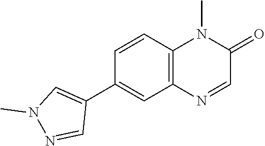 [SMILES: Cn1cc(-c2ccc3c(c2)ncc(=O)n3C)cn1] | 0.491 | 241.1 |
| 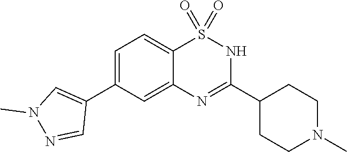 | 0.417 | 360.4 |
| 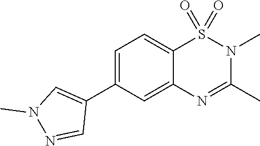 | 0.523 | 291.3 |
| 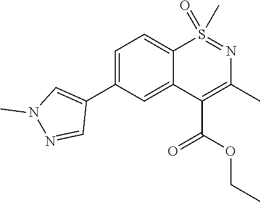 [SMILES: CCOC(=O)C1=C(C)N=S(C)(=O)c2ccc(-c3cnn(C)c3)cc21] | 0.626 | 346.0 |
| 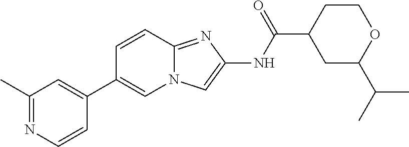 [SMILES: Cc1cc(-c2ccc3nc(NC(=O)C4CCOC(C(C)C)C4)cn3c2)ccn1] | 0.64 | 379.5 |

### DYRK1A Inhibition Assay

### Materials

DYRK1A—Invitrogen #PV4105
Ser/Thr 18 peptide—Invitrogen #PR8227U
Phos-Ser/Thr 18 peptide—Invitrogen #PR8229U
Assay plate—PerkinElmer #6007279
ATP—Sigma #A7699-5G
Development reagent A—Invitrogen #PR5194B
Development buffer—Invitrogen #PR4876B

### Compound Preparation

1. Test compounds were diluted to 1 mM in DMSO
2. Stock was further diluted 3-fold using Echo platform
3. 100 nL of DMSO into Columns 1 and 24 and 100 nL of compounds dilutions to Columns 2-23 in a plate.

### Assay Procedure

1. Add 5 μL enzyme & substrate mixture to each well in Column 2-23 and A24-H24 wells of the 384-well assay plate;
2. Add 5 μL 0% Phosphorylation control to A1-H1 and I24-P24 wells of the assay plate;
3. Add 5 μL 100% Phosphorylation control to I1-P1 wells of the assay plate;
4. Spin the assay plate (1000 rpm, 1 minute @23° C.);
5. Incubate enzyme with compounds for 15 minutes at 23° C.;
6. Add 5 μL ATP solution to each well of the assay plate;
7. Spin the plate (1000 rpm, 1 minute @23° C.);
8. Incubate the assay plate for 90 minutes at 23° C.;
9. Add 10 μL Development reagent A to each well of the assay plate;
10. Centrifuge the plate at 1000 rpm about 15 seconds and seal a film over assay plate. Incubate the assay plate for 30 minutes at 23° C.
11. Read assay plates on Envision (see Tables A and B).

### Final Compound Concentrations

Assay buffer: 50 mM Hepes pH7.5, 10 mM MgCl2, 1 mM EDTA, 0.01% Brij-35
DYRK1A: 1 nM
ATP: 20 μM
Ser/Thr 18 peptide: 2 μM
Reaction time: 90 minutes

TABLE A
DYRK1 Inhibition by Selected Compounds of Formula (I)

| Compound Number | DYRK1 IC₅₀ (nM) |
|---|---|
| 1 | B |
| 2 | A |
| 3 | A |
| 4 | A |
| 5 | B |
| 6 | B |
| 7 | B |
| 8 | B |
| 9 | C |
| 10 | D |
| 11 | D |
| 12 | D |
| 13 | D |
| 14 | A |
| 15 | C |
| 16 | D |
| 17 | B |
| 18 | B |
| 19 | B |
| 20 | B |
| 21 | B |
| 22 | C |
| 23 | C |
| 24 | C |
| 25 | D |
| 26 | D |
| 27 | C |
| 28 | C |
| 29 | C |
| 30 | B |
| 31 | C |
| 32 | B |
| 33 | C |
| 34 | A |
| 35 | D |
| 36 | C |
| 37 | C |
| 38 | B |
| 39 | C |
| 40 | C |
| 41 | D |
| 42 | A |
| 43 | B |
| 44 | A |
| 45 | B |
| 46 | B |
| 47 | A |
| 48 | C |
| 49 | B |
| 50 | D |
| 51 | A |
| 52 | C |
| 53 | A |
| 54 | A |
| 55 | B |
| 56 | C |
| 57 | C |
| 58 | B |
| 59 | B |
| 60 | B |
| 61 | C |
| 62 | B |
| 63 | B |
| 64 | B |
| 65 | B |
| 66 | B |
| 67 | A |
| 68 | A |
| 69 | A |
| 70 | C |
| 71 | A |
| 72 | B |
| 73 | B |
| 74 | B |
| 75 | B |
| 76 | B |
| 77 | ND |
| 78 | C |
| 79 | ND |
| 80 | C |
| 81 | D |
| 82 | D |
| 83 | C |
| 84 | B |
| 85 | B |
| 86 | ND |
| 87 | C |
| 88 | C |
| 89 | D |
| 90 | A |
| 91 | A |
| 92 | A |
| 93 | B |
| 94 | D |
| 95 | C |
| 96 | C |
| 97 | C |
| 98 | C |
| 99 | B |
| 100 | B |
| 101 | ND |
| 102 | B |
| 103 | ND |

(A ≤ 10 nM; 10 nM < B ≤ 100 nM; 100 nM < C ≤ 1 μM; D > 1 μM)
ND = not determined

TABLE B
DYRK1 Inhibition by Selected Compounds of Formula (II)

| Compound | |
|---|---|
| Number | DYRK1 IC₅₀ (nM) |
| 1 | ND |
| 2 | A |
| 3 | A |
| 4 | A |
| 5 | A |
| 6 | C |
| 7 | A |
| 8 | C |
| 9 | C |
| 10 | B |
| 11 | C |
| 12 | C |
| 13 | A |
| 14 | C |
| 15 | C |
| 16 | C |
| 17 | B |
| 18 | A |
| 19 | C |
| 20 | C |
| 21 | A |
| 22 | A |
| 23 | A |
| 24 | A |
| 25 | B |
| 26 | B |
| 27 | A |
| 28 | ND |
| 29 | ND |
| 30 | D |
| 31 | ND |
| 32 | D |
| 33 | D |
| 34 | D |
| 35 | D |
| 36 | D |
| 37 | D |
| 38 | D |
| 39 | D |
| 40 | D |
| 41 | D |
| 42 | D |
| 43 | D |
| 44 | D |
| 45 | D |
| 46 | D |
| 47 | D |
| 48 | D |
| 49 | D |
| 50 | D |
| 51 | D |
| 52 | D |
| 53 | D |
| 54 | D |
| 55 | D |
| 56 | D |
| 57 | D |
| 58 | D |
| 59 | D |
| 60 | D |
| 61 | D |
| 62 | D |
| 63 | D |
| 64 | D |
| 65 | D |
| 66 | D |
| 67 | D |
| 68 | D |
| 69 | D |
| 70 | D |
| 71 | D |
| 72 | D |
| 73 | ND |
| 74 | ND |
| 75 | ND |

(A ≤ 500 nM; 500 nM < B ≤ 1 μM; 1 μM < C ≤ 5 μM; D > 5 μM)
ND = not determined

What is claimed is:

1. A compound of Formula (I): 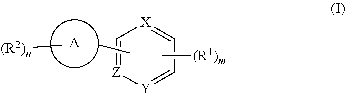
or a pharmaceutically acceptable salt thereof, wherein:
X is CH, N, N—O, or 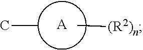
Y is CH, N, or 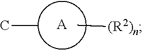
Z is CH, N, or CR¹;
wherein Formula (I) contains one Ring A and not more than two of X, Y, and Z are N or N—O;
each R¹ is independently hydroxyl, cyano, C1-C6 alkyl, 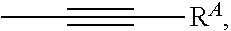
—NHC(═O)R^{A}, C1-C6 alkoxy, —(CH₂)ₚ—NR^{B}R^{C}, C3-C10 cycloalkyloxy optionally substituted with amino, -Q-(CH₂)_{q}—R^{D}, —CO₂R^{B}, —C(═O)NR^{B}R^{C}, —C(═O)-3-6 membered heterocyclyl, or phenoxy;
Ring A is a fused bicyclic 9 membered cycloalkyl, a fused bicyclic 9-10 membered heteroaryl, a 9 membered fused bicyclic heterocyclyl, or a fused tricyclic 13-14 membered heterocyclyl;
each R² is independently halogen, C1-C6 haloalkyl, C1-C6 alkyl optionally substituted with 1-2 substituents independently selected from hydroxyl, C1-C6 alkoxy, and 5-10 membered heteroaryl, —(CH₂)ₜ-Q¹-4-6 membered heterocyclyl optionally substituted with C1-C6 alkyl or —CO₂H, 4-9 membered heterocyclyl optionally substituted with R^{G}, 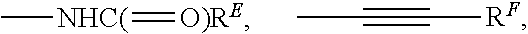
C1-C6 alkoxy, —C(═O)NHR^{H}, phenyl optionally substituted with 1-2 substituents independently selected from hydroxyl and cyano, 5-10 membered heteroaryl optionally substituted with C1-C6 alkyl, —NHSO₂(C1-C6 alkyl), C3-C9 cycloalkyl optionally substituted with hydroxyl or —NR^{B2}R^{C2}, —NR^{B1}R^{C1}, —C(═O)-3-6 membered heterocyclyl optionally substituted with C1-C6 alkyl, or benzyl optionally substituted with C1-C6 alkoxy;
each R^{A} and R^{F} is independently C3-C6 cycloalkyl or 3-6 membered heterocyclyl optionally substituted with 1-2 substituents independently selected from hydroxyl and C1-C6 alkyl;
each R^{B}, R^{B1}, R^{B2}, R^{C}, R^{C1}, and R^{C2} is independently hydrogen or C1-C6 alkyl optionally substituted with hydroxyl;
each R^{D} is independently a 4-6 membered heterocyclyl optionally substituted with C1-C6 alkyl or a phenyl optionally substituted with 1-2 independently selected halogen;
each R^{E} is independently 3-10 membered heterocyclyl optionally substituted with 1-3 independently selected C1-C6 alkyl or a —(CH₂)ᵥphenyl optionally substituted with cyano or halogen;
R^{G} is C1-C6 alkyl, —SO₂(R^{G1}), —C(═O)(C1-C6 alkyl), —C(═O)O—Benzyl, 4-6 membered heterocyclyl optionally substituted with 1-2 substituents independently selected from C1-C6 alkyl, and C3-C6 cycloalkyl optionally substituted with —CO₂H;
R^{G1} is C1-C6 alkyl or phenyl optionally substituted with cyano or halogen;
R^{H} is hydrogen, C1-C6 alkyl, or benzyl optionally substituted with halogen;
Q and Q¹ are independently 0, NH, or a bond;
m, n, p, and t are each independently 0, 1, 2, or 3;
v is 0 or 1; and
q is 1, 2, or 3.

2. The compound of claim 1, wherein not more than one of X, Y, and Z are N or N—O.

3. The compound of claim 1, wherein none of X, Y, and Z are N or N—O.

4. The compound of any one of claims 1-3, wherein X is CH.

5. The compound of any one of claims 1-3, wherein X is N.

6. The compound of any one of claims 1-3, wherein X is N—O.

7. The compound ofany one of claims 1-3, wherein X is

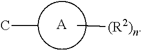

8. The compound of any one of claims 1-7, wherein Y is CH.

9. The compound of any one of claims 1-7, wherein Y is N.

10. The compound of any one of claims 1-6, wherein Y is

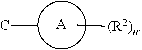

11. The compound of any one of claims 1-10, wherein Z is CH.

12. The compound of any one of claims 1-10, wherein Z is N.

13. The compound of any one of claims 1-10, wherein Z is CR¹.

14. The compound of any one of claims 1-13, wherein Ring A is pyrazolo[1,5-a]pyridinyl, 3H-imidazo[4,5-b]pyridine, pyrrolo[1,2-a]pyrazine, 7H-pyrrolo[2,3-c]pyridazine, 2H-indazolyl, imidazo[1,2-a]pyridine, benzo[d]thiazole, 1H-benzo[d]imidazole, 1H-pyrrolo[2,3-b]pyridine, imidazo[1,2-a]pyridine, 1H-pyrrolo[2,3-c]pyridinyl, pyrazolo[1,5-a]pyrazin-4(5H)-one, isoquinoline, quinoline, quinolin-2(1H)-one, 1,8-naphthyridin-2(1H)-one, 5H-pyrrolo[2,3-b]pyrazinyl, benzo[d]oxazolyl, thiazolo[5,4-b]pyridinyl, benzo[d]isothiazolyl, 2H-pyrazolo[3,4-c]pyridinyl, 1H-indazolyl, pyrazolo[1,5-a]pyrazin-4(5H)-one, furo[2,3-c]pyridinyl, [1,2,4]triazolo[4,3-a]pyridinyl, [1,2,4]triazolo[4,3-a]pyridin-3(2H)-one, benzo[d]isothiazolyl, imidazo[1,5-a]pyridinyl, 4H-pyrido[1,2-a][1,3,5]triazinyl, 1H-pyrrolo[2,3-c]pyridinyl, or 1H-pyrrolo[3,4-c]pyridine-1,3(2H)-dione.

15. The compound of any one of claims 1-13, wherein Ring A is 2,3-dihydrobenzofuranyl, 2-oxo-1,2-dihydro-1,8-naphthyridinyl, 2,3-dihydro-1H-indenyl, 4H-chromen-4-one, 1,3-dihydro-2H-benzo[d]imidazol-2-one, 2-oxo-1,2-dihydro-1,7-naphthyridinyl, 6-oxo-5,6,7,8-tetrahydroimidazo[1,2-a]pyrazinyl, 5,6,7,8-tetrahydroimidazo[1,2-a]pyrazinyl, imidazo[1,5-a]pyridinyl, 4H-pyrido[1,2-a][1,3,5]triazinyl, 4,5,6,7-tetrahydropyrazolo[1,5-a]pyrazinyl, 1,2-dihydro-3H-pyrrolo[3,4-c]pyridin-3-one, or is 1,2,3,4-tetrahydrobenzo[4,5]imidazo[1,2-a]pyrazine.

16. A compound of Formula (II): 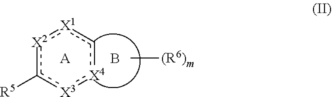
or a pharmaceutically acceptable salt thereof, wherein:
Ring A is aromatic;
Ring B is phenyl, 5-6 membered heteroaryl, or 5-7 membered monocyclic heterocyclyl such that together Ring A and Ring B form a 9-10 membered heteroaryl or a 9-10 membered heterocyclyl ring system;
X¹ is absent, CR¹, N, or NR^{A};
X² is CR², C═O, N, or NR, wherein when X² is C═O, X¹ is NR^{A}, and when X¹ is absent, X² is directly connected to the carbon atom shared by Ring A and Ring B;
X³ is C, CR³ or N;
X⁴ is C or N;
one of R¹, R², R³, and R⁵ is halogen, cyano, C1-C6 haloalkyl, C1-C6 alkyl optionally substituted with 1-2 substituents independently selected from (i) 5-6 membered heteroaryl optionally substituted with 1-2 independently selected C1-C6 alkyl or 3-6 membered heterocyclyl optionally substituted with C1-C6 alkyl, (ii) 4-6 membered heterocyclyl optionally substituted with benzyl, (iii) cyano, (iv) phenyl optionally substituted with halogen, or (v) —NR^{E}R^{F}; C1-C6 alkoxy, —(CH₂)ₙ-Q-(4-6 membered heterocyclyl optionally substituted with C1-C6 alkyl), —NHC(═O)(CH₂)ₙR^{C}, 4-10 membered heterocyclyl optionally substituted with phenoxy, C1-C6 alkyl, or 5-6 membered heteroaryl; —NR^{E}R^{F}, C3-C6 cycloalkyl, C3-C6 cycloalkyloxy, 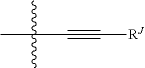
phenoxy optionally substituted with 1-2 substituents independently selected from halogen and C1-C6 haloalkyl; 5-6 membered heteroaryloxy optionally substituted with 1-2 independently selected C1-C6 alkyl, 5-10 membered heteroaryl optionally substituted with C1-C6 alkyl, —SO₂(C1-C6 alkyl), —(CH₂)ₙCO₂R^{D};
and the other of R¹, R², R³, and R⁵ are independently hydrogen, halogen, cyano, C1-C6 alkyl, C1-C6 alkoxy, C1-C6 haloalkyl, C3-C6 cycloalkyloxy, or 4-6 membered heterocyclyl optionally substituted with 5-6 membered heteroaryl; or
R³ and R⁵, together with the carbon atoms to which they are attached form a 6 membered heterocyclyl optionally substituted with C1-C6 alkyl or C3-C6 cycloalkyl; or
R³ and one R⁶ adjacent to Ring A, together with the carbon atoms to which they are attached form a 7 membered heterocyclyl; or
R¹ and one R⁶ adjacent to Ring A, together with the carbon atoms to which they are attached form a 7 membered heterocyclyl; or
R¹ and R², R² and R³, and R³ and R⁵, together with the carbon atoms to which they are attached form a C3-C5 cycloalkyl;
Q and Q¹ are each independently —O— or —C(═O)—;
R^{A} and R^{B} are independently hydrogen, C3-C6 cycloalkyl, or C1-C6 alkyl;
R^{C} is 4-6 membered heterocyclyl optionally substituted with C1-C6 alkyl, or 5-6 membered heteroaryl optionally substituted with C1-C6 alkoxy or C1-C6 alkyl,
R^{D} is hydrogen or C1-C6 alkyl;
R^{E} and R^{F} are independently hydrogen, C1-C6 alkyl, —C(═O)—C1-C6 alkyl, or 4-6 membered heterocyclyl;
R^{G} and R^{H} are independently hydrogen, C1-C6 alkyl, or C3-C6 cycloalkyl;
R^{I} is 5-6 membered heteroaryl optionally substituted with C1-C6 alkyl or halogen; C1-C6 alkyl substituted with 1-2 independently selected amino or phenyl optionally substituted with halogen; or phenyl optionally substituted with halogen;
R^{J} is a 4-6 membered heterocyclyl or a 5-6 membered heteroaryl;
R⁶ is C1-C6 alkyl optionally substituted with (i) 4-10 membered heterocyclyl optionally substituted C1-C6 alkyl or (ii) 9-10 membered heteroaryl; C1-C6 haloalkyl, C3-C6 cycloalkyl optionally substituted with —CO₂R^{D}, —NHC(═O)R^{C}, —NR^{G}R^{H}, 4-10 membered heterocyclyl optionally substituted C1-C6 alkyl, —(CH₂)ₚC(═O)NHR^{I}, 5-10 membered heteroaryl optionally substituted with C1-C6 alkyl, or —(CH₂)ₛ-Q¹-(4-6 membered heterocyclyl optionally substituted with C1-C6 alkyl); or
two geminal R⁶, together with the carbon atom to which they are attached form a C3-C6 spirocycloalkyl;
m is 0, 1, 2, or 3;
n is 0, 1, or 2;
p is 0, 1, 2, 3, or 4; and
s is 0, 1, or 2.

17. The compound of claim 16, wherein Ring B is phenyl.

18. The compound of claim 16, wherein Ring B is 5-6 membered heteroaryl.

19. The compound of claim 16, wherein Ring B is 5-7 membered monocyclic heterocyclyl.

20. The compound of claim 16, wherein Ring A and Ring B form a 9-10 membered heteroaryl ring system.

21. The compound of claim 16, wherein Ring A and Ring B form a 9-10 membered heterocyclyl ring system.

22. The compound of any one of claims 16-21, wherein X¹ is absent.

23. The compound of any one of claims 16-21, wherein X¹ is CR¹.

24. The compound of any one of claims 16-21, wherein X¹ is N.

25. The compound of any one of claims 16-21, wherein X¹ is NR^{A}.

26. The compound of any one of claims 16-21, wherein X² is CR².

27. The compound of any one of claims 16-21, wherein X² is C═O.

28. The compound of any one of claims 16-21, wherein X² is N.

29. The compound of any one of claims 16-21, wherein X² is NR.

30. The compound of any one of claims 16-21, wherein X³ is C.

31. The compound of any one of claims 16-21, wherein X³ is CR³.

32. The compound of any one of claims 16-21, wherein X³ is N.

33. The compound of any one of claims 16-21, wherein X⁴ is C.

34. The compound of any one of claims 16-21, wherein X⁴ is N.

35. The compound of claim 1, wherein Ring A and Ring B form a ring system selected from the group consisting of:

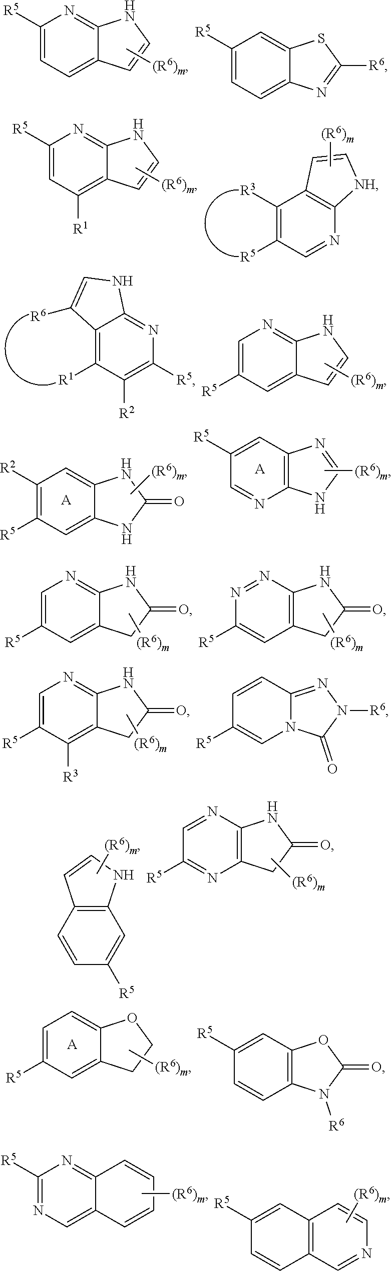

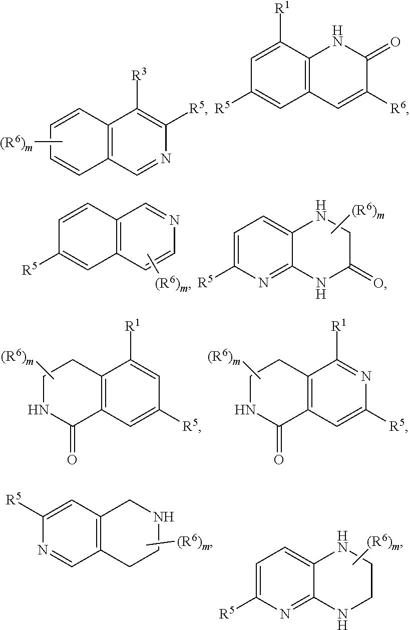

36. The compound of claim 1, wherein the compound is selected from a compound in Table 1 or Table 2, Table 3, or a pharmaceutically acceptable salt of any of the foregoing.

37. A pharmaceutical composition comprising a compound of any one of claims 1-36 or a pharmaceutically acceptable salt thereof, and pharmaceutically acceptable diluent or carrier.

38. A method for treating a neurological disorder in a subject in need thereof, the method comprising administering to the subject a therapeutically effective amount of a compound of any one of claims 1-36, or a pharmaceutically acceptable salt thereof, or a pharmaceutical composition according to claim 37.

39. The method of claim 38, wherein the neurological disorder is selected from the group consisting of Down Syndrome, Alzheimer's disease, and Alzheimer's disease associated with Down Syndrome.

40. The method of claim 38 or 39, wherein the neurological disorder is selected Alzheimer's disease associated with Down syndrome.

41. A method of treating a DYRK1A-associated neurological disorder in a subject, the method comprising administering to a subject identified or diagnosed as having a DYRK1A-associated neurological disorder a therapeutically effective amount of a compound of any one of claims 1-36, or a pharmaceutically acceptable salt thereof, or a pharmaceutical composition according to claim 37.

42. A method for modulating DYRK1A in a mammalian cell, the method comprising contacting the mammalian cell with a therapeutically effective amount of a compound of any one of claims 1-36, or a pharmaceutically acceptable salt thereof.
